(19) United States
(12) Patent Application Publication
(10) Pub. No.: US 2023/0204586 A1
(43) Pub. Date: Jun. 29, 2023

(54) METHOD FOR PREDICTING EFFICACY OF TREATMENT OF LUNG CANCER PATIENT USING IMMUNE CHECKPOINT INHIBITOR

(71) Applicants: JSR Corporation, Minato-ku (JP); KEIO UNIVERSITY, Minato-ku (JP)

(72) Inventors: Masaru UENO, Tokyo (JP); Seiki WAKUI, Tokyo (JP); Motoaki MIZUUCHI, Tokyo (JP); Araki WAKIUCHI, Tokyo (JP); Yutaka KAWAKAMI, Tokyo (JP); Shigeki OHTA, Tokyo (JP)

(73) Assignees: JSR Corporation, (omitted); KEIO UNIVERSITY, Minato-ku (JP)

(21) Appl. No.: 17/759,135

(22) PCT Filed: Mar. 25, 2021
(86) PCT No.: PCT/JP2021/012475

§ 371 Date: Jul. 20, 2022

(30) Foreign Application Priority Data
Mar. 26, 2020 (JP) 2020-056752

(51) Int. Cl. G01N 33/574; G01N 33/68

(52) U.S. Cl. CPC G01N 33/57423; G01N 33/6848; G01N 33/57484

(57) ABSTRACT

A method for predicting efficacy of treatment of a lung cancer patient using an immune checkpoint inhibitor includes isolating exosomes from a biological sample derived from the lung cancer patient, and determining an expression level of a protein present in the exosomes by a mass spectrometry method, in which the protein is one or more proteins selected from the group of proteins shown in Table 1-1 to Table 1-6.

### TECHNICAL FIELD

The present invention relates to a method for predicting the efficacy of treatment of a lung cancer patient using an immune checkpoint inhibitor.

Priority is claimed on Japanese Patent Application No. 2020-056752, filed Mar. 26, 2020, the content of which is incorporated herein by reference.

### BACKGROUND ART

As companion diagnoses using immune checkpoint inhibitors (ICI) such as anti-PD-1/PD-L1 antibodies, diagnosis using PD-L1 expression in tumor tissue, diagnosis using micro satellite instability (MSl) testing, and the like are used in practice. However, in diagnosis using PD-L1, there are cases where the determination results of expression differ depending on the antibody and where ICI administration is effective even in cases that are determined to be negative. In diagnosis using MSI testing, patients having microsatellite instability (MSI-High) are 10% or less for most cancer types and there are cases of MSI-High patients also exhibiting drug resistance. Thus, the sensitivity and specificity of these diagnostic methods are not sufficient and the search for biomarkers to predict treatment efficacy is a significant problem.

On the other hand, cells are known to release vesicles called exosomes, which are formed of a lipid bilayer membrane including various cellular contents such as proteins, nucleic acids, lipids and sugars in the inner portion thereof, to the outside of the cell. Exosomes are substances which are important clues to knowing the pathology of living organisms and there is active research on how exosomes are involved in various diseases.

Patent Document 1 discloses the use of perforin in blood samples as a biomarker for evaluating the efficacy of cancer treatment by ICI.

Non-Patent Document 1 discloses PD-L1 -positive exosomes as a biomarker for predicting the treatment efficacy of PD-1 inhibitors in metastatic gastric cancer.

However, although biomarkers for companion diagnoses using ICI in lung cancer exist, there is a demand for the development of a method for predicting the effects of ICI on lung cancer with even higher sensitivity and specificity.

### PRIOR ART LITERATURE

### Patent Document

Patent Document 1: Japanese Patent No. 6630026

Non Patent Document

Non Patent Document 1: Kim ST et al., “Comprehensive molecular characterization of clinical responses to PD-1 inhibition in metastatic gastric cancer.”, Nat med., Vol. 24, pp. 1449-1458, 2018

### DISCLOSURE OF INVENTION

### Problems to Be Solved by the Invention

The present invention was created in consideration of the circumstances described above and provides a novel method for predicting the efficacy of treatment of a lung cancer patient using an immune checkpoint inhibitor.

### Means for Solving the Problems

That is, the present invention includes the following aspects.

A method for predicting efficacy of treatment of a lung cancer patient using an immune checkpoint inhibitor, the method including isolating exosomes from a biological sample derived from the lung cancer patient, and determining an expression level of a protein present in the exosomes by a mass spectrometry method, in which the protein is one or more proteins selected from the group of proteins shown in Table 1-1 to Table 1-6.

TABLE 1-1

Protein Name
Band 4.1-like protein 2
Cullin-2
Protein Hikeshi
Hippocalcin-like protein 1
UTP–glucose-1-phosphate uridylyltransferase
Attractin
Annexin A3
Rap1 GTPase-activating protein 2
Radixin
Annexin A5
ADP-ribosylation factor 5
L-lactate dehydrogenase B chain
Rho GTPase-activating protein 1
Drebrin
Serinelthreonine-protein kinase PAK 2
Protein BRICK1
Integrin alpha-V
Protein kinase C and casein kinase substrate in neurons protein 2
Triokinase/FMN cyclase
Adhesion G-protein coupled receptor G1
Integrin beta-5
Isochorismatase domain-containing protein 1
Septin-2
Hydroxyacyl-coenzyme A dehydrogenase, mitochondrial
Serine/threonine-protein kinase 38
Transient receptor potential cation channel subfamily M member 4
Cytoskeleton-associated protein 5
FAS-associated factor 1
Insulin receptor
Tropomyosin alpha-4 chain
Programmed cell death protein 6
Leukocyte elastase inhibitor
ADP-ribosylation factor 6
Complement component C9
ATP-dependent (S)-NAD(P)H-hydrate dehydratase
Actin, alpha cardiac muscle 1
Heat shock 70 kDa protein 4
14-3-3 protein theta
Phosphatidylinositol transfer protein beta isoform
Phosphoribosyltransferase domain-containing protein 1
Costars family protein ABRACL
Ubiquitin-conjugating enzyme E2 L5
Sodium/potassium-transporting ATPase subunit alpha-1
14-3-3 protein epsilon

TABLE 1-2

Major prion protein
F-actin-capping protein subunit alpha-2
HLA class l histocompatibility antigen, Cw-4 alpha chain
GTP-binding protein SAR1a
Actin-related protein ⅔ complex subunit 5
Glutathione reductase, mitochondrial
Guanine nucleotide-binding protein subunit alpha-11
Protein disulfide-isomerase A5
MARCKS-related protein
Septin-7
Hsc70-interacting protein
COP9 signalosome complex subunit 8
Vacuolar protein sorting-associated protein 37C
Acidic fibroblast growth factor intracellular-binding protein
ADP-ribosyl cyclase/cyclic ADP-ribose hydrolase 1
Ribosome maturation protein SBDS
Fructose-bisphosphate aldolase A
BH3-interacting domain death agonist
Charged multivesicular body protein 4b
Guanine nucleotide-binding protein G(s) subunit alpha isoforms short
cAMP-dependent protein kinase catalytic subunit alpha
Cytosolic non-specific dipeptidase
Dynamin-1
Neuroplastin
IST1 homolog
Sorting nexin-5
Mitochondrial import inner membrane translocase subunit Tim 13
Exocyst complex component 4
Moesin
Serum albumin
Myristoylated alanine-rich C-kinase substrate
Sodium/potassium-transporting ATPase subunit beta-3
Vinculin
Actin-related protein ⅔ complex subunit 1B
Alcohol dehydrogenase class-3
Cysteine and glycine-rich protein 1
E3 ubiquitin-protein ligase TRIM23
Actin-related protein 3
Tetraspanin-15
Ras-related C3 botulinum toxin substrate 1
COP9 signalosome complex subunit 2
Myotrophin
Rho-related GTP-binding protein RhoF
COP9 signalosome complex subunit 3
Glutathione synthetase
DnaJ homolog subfamily C member 5

TABLE 1-3

Glutathione S-transferase omega-1
Glutathione S-transferase Mu 2
Ragulatorcomplex protein LAMTOR2
Cytochrome b5 type B
Progressive ankylosis protein homolog
Septin-11
Rho-associated protein kinase 1
Ras-related C3 botulinum toxin substrate 3
Isocitrate dehydrogenase [NAD] subunit alpha, mitochondrial
Inactive tyrosine-protein kinase transmembrane receptor ROR1
Abl interactor 2
Cell division control protein 42 homolog
Transgelin-2
Ras-related protein Rab-2A
Charged multivesicular body protein 6
ADP-ribosylation factor 3
Glyoxylate reductase/hydroxypyruvate reductase
Profilin-1
Ras-related protein Rab-11B
Rab GDP dissociation inhibitor alpha
CD97 antigen
Programmed cell death protein 10
Antithrombin-III
F-actin-capping protein subunit beta
Actin-related protein ⅔ complex subunit 2
Twinfilin-2
Ras-related protein Rab-21
Vacuolar-sorting protein SNF8
Caspase-6
Calcineurin subunit B type 1
Extended synaptotagmin-2
Leukocyte surface antigen CD47
COP9 signalosome complex subunit 1
26S proteasome non-ATPase regulatory subunit 7
Eukaryotic translation initiation factor 3 subunit C-like protein
Deoxycytidylate deaminase
CD276 antigen
Caspase-3
Leucine-rich repeat-containing protein 57
Aldose reductase
Diphosphoinositol polyphosphate phosphohydrolase 1
Coagulation factor XIII A chain
Hsp70-binding protein 1
Sorting nexin-4
Protein phosphatase 1L
Pleckstrin homology domain-containing family N member 1

TABLE 1-4

AP-2 complex subunit alpha-2
Trophoblast glycoprotein
Ras-related protein Rab-23
14-3-3 protein eta
Peroxiredoxin-6
14-3-3 protein beta/alpha
Copine-2
14-3-3 protein gamma
Superoxide dismutase [Cu-Zn]
Microtubule-associated protein RP/EB family member 1
Vasodilator-stimulated phosphoprotein
Phosphoglucomutase-1
Adenylyl cyclase-associated protein 1
Calpain small subunit 1
Dual specificity protein phosphatase 23
Dihydropteridine reductase
Ephrin-B1
Golgin subfamily A member 7
Xaa-Pro aminopeptidase 1
Actin-related protein ⅔ complex subunit 3
Sorting nexin-9
Pleckstrin homology domain-containing family O member 2
WD repeat-containing protein 44
N(G),N(G)-dimethylarginine dimethylaminohydrolase 2
COP9 signalosome complex subunit 6
Guanine nucleotide-binding protein G(I)/G(S)/G(O) subunit gamma-10
Rab-like protein 3
Phosphoacetylglucosamine mutase
Sorting nexin-2
DDB1- and CUL4-associated factor 11
Ras GTPase-activating protein 1
Ribose-5-phosphate isomerase
SHC-transforming protein 1
Glutaminyl-peptide cyclotransferase-likeprotein
Ras-related protein Rab-5C
Purine nucleoside phosphorylase
Delta-aminolevulinic acid dehydratase
Ras-related protein Rab-5B
Abl interactor 1
Beta-2-glycoprotein 1
Serine/threonine-protein phosphatase 2B catalytic subunit alpha isoform
Actin-related protein 2
Ras-related protein Rap-2a
Sorcin
Type l inositol 1,4,5-trisphosphate 5-phosphatase
Ras-related protein Rab-35

TABLE 1-5

Rho GTPase-activating protein 18
Ras-related protein Rab-1A
Deoxyribonuclease-1-like 1
Peptidyl-prolyl cis-trans isomerase A
Adenylate kinase isoenzyme 1
Equilibrative nucleoside transporter 1
GTPase NRas
Rho-related GTP-binding protein RhoG
Glycogen phosphorylase, brain form
UV excision repair protein RAD23 homolog B
Omega-amidase NIT2
Serine hydroxymethyltransferase, cytosolic
Sodium/potassium-transporting ATPase subunit beta-1
Endothelial cell-selective adhesion molecule
Isocitrate dehydrogenase [NADP] cytoplasmic
Peptidyl-prolyl cis-trans isomerase FKBP1A
Dystroglycan
Tumor susceptibility gene 101 protein
Peroxiredoxin-5, mitochondrial
Protein XRP2
Actin-related protein ⅔ complex subunit 4
Stathmin
NSFL1 cofactor p47
Folate receptor alpha
Aldo-keto reductase family 1 member A 1
Ran-specific GTPase-activating protein
Serine/threonine-protein phosphatase 2A catalytic subunit beta isoform
Vacuolar protein sorting-associated protein VTA1 homolog
Dual specificity protein phosphatase 3
Membrane cofactor protein
Calcium-binding protein 39
Argininosuccinate lyase
Mitochondrial import receptor subunit TOM34
Protein numb homolog
SUMO-conjugating enzyme UBC9
EH domain-containing protein 3
Ras-related protein Rab-1B
Adenine phosphoribosyltransferase
Coronin-1A
Annexin A4
F-actin-capping protein subunit alpha-1
Protein phosphatase 1 regulatory subunit 7
Peflin
Protein/nucleic acid deglycase DJ-1
Ras-related protein Rab-10
Ras-related protein Rap-2b

TABLE 1-6

RNA transcription, translation and transport factor protein
Crk-like protein
Serpin B6
6-phosphogluconolactonase
Guanine nucleotide-binding protein G(I)/G(S)/G(T) subunit beta-1
Lactadherin
Ras-related protein Ral-A
Protein transport protein Sec23A
Dihydropyrimidinase-related protein 2
NCK-interacting protein with SH3 domain
Vesicle-associated membrane protein 5
Protein phosphatase 1F
Nuclear receptor corepressor 2
Ubiquitin-conjugating enzyme E2 D3
WW domain-binding protein 2
POTE ankyrin domain family member I
Copine-1
Alpha-galactosidase A
Coronin-1B
ATP-dependent RNA helicase DDX1
Ubiquitin-conjugating enzyme E2 K
Syntaxin-binding protein 1
WD repeat-containing protein 37
MOB kinase activator 1B
Nodal modulator 3
Nucleoside diphosphate kinase B
Prostaglandin reductase 1
Phosphoglycerate mutase 2
TLD domain-containing protein 1
Calcium-binding protein 39-like
Glutathione S-transferase LANCL1
Prefoldin subunit 6
Cytosolic phospholipase A2
14-3-3 protein zeta/delta

(2) The method according to (1), in which the protein is one or more membrane proteins selected from the group of proteins shown in Table 2.

TABLE 2

Protein Name
Band 4.1-like protein 2
Attractin
Rho GTPase-activating protein 1
Integrin alpha-V
Adhesion G-protein coupled receptor G1
Integrin beta-5
Transient receptor potential cation channel subfamily M member 4
Insulin receptor
Sodium/potassium-transporting ATPase subunit alpha-1
Major prion protein
Fructose-bisphosphate aldolase A
Neuroplastin
Sodium/potassium-transporting ATPase subunit beta-3
Tetraspanin-15
DnaJ homolog subfamily C member 5
Cytochrome b5 type B
Progressive ankylosis protein homolog
Inactive tyrosine-protein kinase transmembrane receptor ROR1
CD97 antigen
Extended synaptotagmin-2
Leukocyte surface antigen CD47
CD276 antigen
Protein phosphatase 1L
Trophoblast glycoprotein
Ephrin-B1
Equilibrative nucleoside transporter 1
Sodium/potassium-transporting ATPase subunit beta-1
Endothelial cell-selective adhesion molecule
Peptidyl-prolyl cis-trans isomerase FKBP1A
Dystroglycan
Folate receptor alpha
Membrane cofactor protein
NCK-interacting protein with SH3 domain
Vesicle-associated membrane protein 5
Syntaxin-binding protein 1
Nodal modulator 3

(3) The method according to (1), in which the protein is one or more proteins selected from the group of proteins shown in Table 3.

TABLE 3

Protein Name
Integrin beta-5
Sorting nexin-9
Phosphoacetylglucosamine mutase
Stathmin
Ran-specific GTPase-activating protein
EH domain-containing protein 3
Adenine phosphoribosyltransferase
Protein phosphatase 1 regulatory subunit 7
Nuclear receptor corepressor 2
TLD domain-containing protein 1
Glutathione S-transferase LANCL1

(4) The method according to any one of (1) to (3), in which the immune checkpoint inhibitor is an anti-PD-1 antibody or an anti-PD-LI antibody.

The method according to any one of (1) to (4), in which the immune checkpoint inhibitor is one or more selected from the group consisting of nivolumab, pembrolizumab, and atezolizumab.

The method according to any one of (1) to (5), in which, as a method for isolating exosomes from the biological sample derived from the lung cancer patient, a mixture of magnetic particles bound to an anti-CD9 antibody, an anti-CD81 antibody, or an anti-CD63 antibody is used.

### EFFECTS OF THE INVENTION

According to the aspects described above, it is possible to provide a novel method for predicting the efficacy of treatment of a lung cancer patient using an immune checkpoint inhibitor.

### EMBODIMENTS FOR CARRYING OUT THE INVENTION

### Immune Checkpoint

In the present specification, “immune checkpoint (system)” refers to a mechanism that suppresses immune responses by controlling the activity of immune-related cells (including T cells). This system suppresses inappropriate immune responses and excessive inflammatory responses to the cells and tissues of the self. Immune checkpoint molecules are molecules that are expressed on immune-related cells and that transmit signals that suppress immune responses to such immune-related cells by binding to ligands. Examples of immune checkpoint molecules and ligands thereof include molecules such as PD-1, CTLA-4, TIM-3 (T cell immunoglobulin and mucin containing protein-3), LAG-3 (lymphocyte activation gene-3), PD-L1, PD-L2, BTNL2, B7-H3, B7-H4, B7-H5 (VISTA), CD48, CD80, 2B4, BTLA, CD160, CD60, CD86, TIGIT (T cell Immunoreceptor with Ig and ITIM domain), without being limited thereto.

In the present specification, ‘immune checkpoint inhibitor’ refers to an agent that inhibits signal transmission by immune checkpoint molecules by inhibiting the binding of immune checkpoint molecules to the ligands thereof. In many cancer cells, PD-L1, PD-L2, and CD80, which are ligands for immune checkpoint molecules, are expressed on the cell surface and this leads to evasion of attacks by immune-related cells such as T cells, thus, it is considered that, in a normal state, the body’s immune function alone is not able to eliminate cancer tissue. Immune checkpoint inhibitors enable efficient elimination of cancer by the body’s immune function by inhibiting ligand-receptor interactions and the like that control the transmission of such suppressive signals from cancer tissue to the immune function.

### Immune Checkpoint Inhibitors

Examples of immune checkpoint inhibitors include PD-1 inhibitors, PD-L1 inhibitors, CTLA-4 inhibitors, and the like.

Examples of PD-1 inhibitors include anti-PD-1 antibodies such as Nivolumab (marketed as Opdivo (registered trademark)), Pembrolizumab (marketed as Keytruda (registered trademark)), and the like, without being limited thereto.

Examples of PD-L1 inhibitors include anti-PD-L1 antibodies such as Avelumab, Durvalumab, Atezolizumab (marketed as Tecentriq (registered trademark)), and the like, without being limited thereto.

Examples of CTLA-4 inhibitors include Anti-CTLA-4 antibodies such as Ipilimumab and Tremelimumab, without being limited thereto.

Examples of other immune checkpoint inhibitors include agents that target immune checkpoint proteins such as TIM-3, LAG-3, B7-H3, B7-H4, B7-H5 (VISTA), and TIGIT.

Among these, as the immune checkpoint inhibitors applied to the method of the present embodiment, PD-1 inhibitors or PD-L1 inhibitors are preferable and anti-PD-1 antibodies or anti-PD-L1 antibodies are more preferable. With anti-PD-1 antibodies or anti-PD-L1 antibodies, applying one or more selected from the group consisting of Nivolumab, Pembrolizumab, and Atezolizumab described in the present Examples is also preferable in the same manner.

### Exosome Derived Proteins for Determining Sensitivity to Immune Checkpoint Inhibitors

The inventors analyzed exosomes in the blood of 10 responder patients with lung cancer with known sensitivity to immune checkpoint inhibitors and 10 non-responders, as shown in the Examples described below. As a result, 2406 proteins present in exosomes were identified and, among the above, exosome-derived proteins were found in which the value of the Area Under the Curve (AUC) of the ROC curve, which is an indicator of discriminatory ability in determining drug sensitivity, was 0.8 or higher. Furthermore, based on the AUC value of the ROC curve and the best cut-off value, an algorithm was constructed in Python and multi-markers having clinically usable sensitivity and specificity in which the proteins described above were combined were found.

### Method for Predicting Efficacy of Treatment of Lung Cancer Patient Using Immune Checkpoint Inhibitor

A method for predicting the efficacy of treatment of a lung cancer patient using an immune checkpoint inhibitor according to one embodiment of the present invention (may be simply referred to below as “the method of the present embodiment”) is a method including isolating exosomes from a biological sample derived from a lung cancer patient and determining the expression level of proteins present in the exosomes by a mass spectrometry method (may be referred to below as “the steps”). In the present specification and in the claims, “companion diagnostic agent” refers to a diagnostic agent used in a test performed before the actual initiation of medication to predict the effect of a drug for individual lung cancer patients, the risk of side effects, and the appropriate dosage of the medication.

The proteins are one or more proteins selected from the protein groups shown in Table 4-1 to Table 4-19 below. As shown in the Examples described below, these proteins have an AUC value of the ROC curve, which is an indicator of discriminatory ability in determining drug sensitivity, of 0.8 or higher and are biomarkers with excellent sensitivity and specificity for predicting the efficacy of treatment of a lung cancer patient using an immune checkpoint inhibitor. Therefore, according to the method of the present embodiment, it is possible to predict the efficacy of treatment of a lung cancer patient using an immune checkpoint inhibitor with high accuracy.

The protein groups shown in Table 4-1 to Table 4-19 below are identical to the protein groups shown in Table 1-1 to Table 1-6 above, and, for the protein groups shown in Table 1-1 to Table 1-6 described above, information is shown for the Genbank accession number of the mRNA base sequence encoding each protein in humans, the Genbank accession number of the amino acid sequence of each protein in humans, the name of the gene encoding each protein in humans, and the UniProt accession number of the amino acid sequence of each protein in humans.

[symbol]

TABLE 4-2

| | | | | |
|---|---|---|---|---|
| Rap1 GTPase-activating protein 2 | NM_001100398, NM_001330058, NM_015085, XM_005256542, XM_011523738, XM_011523739, XM_011523741, XM_011523742, XM_011523743, XM_011523744, XM_011523745, XM_017024371, XM_024450659 | NP_001093868, NP_001316987, NP_055900, XP_005256599, XP_011522040, XP_011522041, XP_011522043, XP_011522044, XP_011522045, XP_011522046, XP_011522047, XP_016879860, XP_024306427 | RAP1GAP2 | Q684P5 |
| Radixin | NM_001260492, NM_001260493, NM_001260494, NM_001260495, NM_001260496, NM_002906 | NP_001247421, NP_001247422, NP_001247423, NP_001247424, NP_001247425, NP_002897 | RDX | P35241 |
| Annexin A5 | NM_001154, XM_017008141 | NP_001145, XP_016863630 | ANXA5 | P08758 |
| ADP-ribosylation factor 5 | NM_001662 | NP_001653 | ARF5 | P84085 |
| L-lactate dehydrogenase B chain | NM_001174097, NM_001315537, NM_002300, XM_006719074 | NP_001167568, NP_001302466, NP_002291, XP_006719137 | LDHB | P07195 |
| Rho GTPase-activating protein 1 | NM_004308, XM_011520095, XM_024448520 | NP_004299, XP_011518397, XP_024304288 | ARHGAP1 | Q07960 |
| Drebrin | NM_001363541, NM_001364151, NM_001364152, NM_004395, NM_080881, XM_005265827, XM_006714826, XM_011534446, XM_011534447, XM_017009139, XM_017009140 | NP_001350470, NP_001351080, NP_001351081, NP_004386, NP_543157, XP_005265884, XP_006714889, XP_011532748, XP_011532749 XP_016864628, XP_016864629 | DBN1 | Q16643 |
| Serine/threonine-protein kinase PAK 2 | NM_002577, XM_011512870, XM_017006501 | NP_002568, XP_011511172, XP_016861990 | PAK2 | Q13177 |
| Protein BRICK1 | NM_018462 | NP_060932 | BRK1 | Q8WUW1 |
| Integrin alpha-V | NM_001144999, NM_001145000, NM_002210 | NP_001138471, NP_001138472, NP_{_}002201 | ITGAV | P06756 |
| Protein kinase C and casein kinase substrate in neurons protein 2 | NM_001184970, NM_001184971, NM_001349968, NM_001349969, NM_001349970, NM_001349971, NM_001349972, NM_001349973, NM_001349974, NM_007229, XM_005261319, XM_011529847, XM_011529848, XM_011529849, XM_011529850, XM_011529852, XM_017028562, XM_017028563, XM_017028564 | NP_001171899, NP_001171900, NP_001336897, NP_001336898, NP_001336899, NP_001336900, NP_001336901, NP_001336902, NP_001336903, NP_009160, XP_005261376, XP_011528149, XP_011528150, XP_011528151, XP_011528152, XP_011528154, XP_016884051, XP_016884052, XP_016884053 | PACSIN2 | Q9UNF0 |
| Triokinase/FMN cyclase | NM_001351976, NM_001351977, NM_001351978, NM_001351979, NM_001351980, NM_015533, XM_011544912, XM_017017517, XM_017017518, XM_017017519, XM_017017520 | NP_001338905, NP_001338906, NP_001338907, NP_001338908, NP_001338909, NP_056348, XP_011543214 XP_016873006, XP_016873007, XP_016873008, XP_016873009 | TKFC | Q3LXA3 |

TABLE 4-3

| | | | | |
|---|---|---|---|---|
| Adhesion G-protein coupled receptor G1 | NM_001145770, NM_001145771, NM_001145772, NM_001145773, NM_001145774, NM_001290142, NM_001290143, NM_001290144, NM_005682, NM_201524, NM_201525, XM_005256237, XM_005256238, XM_005256239, XM_005256240, XM_005256241, XM_005256242, XM_005256244, XM_005256245, XM_005256246, XM_005256247, XM_005256248, XM_005256249, XM_005256251, XM_005256252, XM_005256254, XM_005256255, XM_006721338, XM_006721339, XM_006721340, XM_006721341, XM_006721342, XM_006721343, XM_006721344, XM_006721345, XM_006721346, XM_006721347, XM_011523461, XM_011523462, XM_011523463, XM_011523464, XM_011523465, XM_011523466, XM_011523467, XM_011523468, XM_017023892 | NP_001139242, NP_001139243, NP_001139244, NP_001139245, NP_001139246, NP_001277071, NP_001277072, NP_001277073, NP_005673, NP_958932, NP_958933, XP_005256294, XP_005256295, XP_005256296, XP_005256297, XP_005256298, XP_005256299, XP_005256301, XP_005256302, XP_005256303, XP_005256304, XP_005256305, XP_005256306, XP_005256308, XP_005256309, XP_005256311, XP_005256312, XP_006721401, XP_006721402, XP_006721403, XP_006721404, XP_006721405, XP_006721406, XP_006721407, XP_006721408, XP_006721409, XP_006721410, XP_011521763, XP_011521764, XP_011521765, XP_011621766, XP_011521767, XP_011521768, XP_011521769, XP_011521770, XP_016879381 | ADGRG1 | Q9Y653 |
| Integrin beta-5 | NM_001354764, NM_001354765, NM_001354766, NM_002213, XM_005247436, XM_006713630, XM_017006353, XM_017006354, XM_017006355 | NP_001341693 NP_001341694, NP_001341695, NP_002204, XP_005247493, XP_006713693, XP_016861842, XP_016861843, XP_016861844 | ITGB5 | P18084 |
| Isochorismatase domain-containing protein 1 | NM_016048 | NP_057132 | ISOC1 | Q96CN7 |

TABLE 4-4

| | | | | |
|---|---|---|---|---|
| Septin-2 | NM_001008491, NM_001008492, NM_001282972, NM_001282973, NM_001321029, NM_001321030, NM_001321031, NM_001321032, NM_001321033, NM_001321034, NM_001321035, NM_001349287, NM_001349288, NM_001349289, NM_001349290, NM_001349291, NM_001349302, NM_001349304, NM_001349305, NM_001349306, NM_001349307, NM_001349308, NM_001349309, NM_001349310, NM_001349311, NM_001349312, NM_001349313, NM_001349314, NM_001349315, NM_004404, NM_006155, XM_017004205, XM_017004212, XM_017004213, XM_024452920, XM_024452921, XM_024452922, XM_024452923, XM_024452924, XM_024452925, XM_024452926 | NP_001008491, NP_001008492, NP_001269901, NP_001269902, NP_001307958, NP_001307959, NP_001307960, NP_001307961, NP_001307962, NP_001307963, NP_001307964, NP_001336216, NP_001336217, NP_001336218, NP_001336219, NP_001336220, NP_001336231, NP_001336233, NP_001336234,NP_001336235, NP_001336236, NP_001336237, NP_001336238, NP_001336239, NP_001336240, NP_001336241, NP_001336242, NP_001336243, NP_001336244, NP_004395, NP_006146, XP_016859694, XP_016859701, XP_016859702, XP_024308688, XP_024308689, XP_024308690, XP_024308691, XP_024308692, XP_024308693, XP_024308694 | SEPT2 | Q15019 |
| Hydroxyacyl-coenzyme A dehydrogenase, mitochondrial | NM_001184705, NM_001331027, NM_005327 | NP_001171634, NP_001317956, NP_005318 | HADH | Q16836 |
| Serine/threonine-protein kinase 38 | NM_001305102, NM_007271, XM_006714988, XM_006714989, XM_017010226 | NP_001292031, NP_009202, XP_006715051, XP_006715052, XP_016865715 | STK38 | Q15208 |
| Transient receptor potential cation channel subfamily M member 4 | NM_001195227, NM_001321281, NM_001321282, NM_001321283, NM_001321285, NM_017636, XM_005259017, XM_024451557 | NP_001182156, NP_001308210, NP_001308211, NP_001308212, NP_001308214, NP_060106, XP_005259074, XP_024307325 | TRPM4 | Q8TD43 |
| Cytoskeleton-associated protein 5 | NM_001008938, NM_014756 | NP_001008938, NP_055571 | CKAP5 | Q14008 |
| FAS-associated factor 1 | NM_007051, NM_131917, XM_017000136, XM_024452734, XM_024452736 | NP_008982, XP_016855625, XP_024308502, XP_024308504 | FAF1 | Q9UNN5 |
| Insulin receptor | NM_000208, NM_001079817, XM_011527988, XM_011527989 | NP_000199, NP_001073285, XP_011526290, XP_011526291 | INSR | P06213 |
| Tropomyosin alpha-4 chain | NM_001145160, NM_003290, XM_005260042, XM_006722865, XM_011528231, XM_011528232, XM_017027188, XM_017027189, XM_024451681 | NP_001138632, NP_003281, XP_005260099, XP_006722928, XP_011526533, XP_011526534, XP_016882677, XP_016882678, XP_024307449 | TPM4 | P67936 |
| Programmed cell death protein 6 | NM_001267556, NM_001267557, NM_001267558, NM_001267559, NM_013232 | NP_001254485, NP_001254486, NP_001254487, NP_001254488, NP_037364 | PDCD6 | O75340 |
| Leukocyte elastase inhibitor | NM_030666, XM_011514333, XM_011514334, XM_011514335 | NP_109591, XP_011512635, XP_011512636, XP_011512637 | SERPINB1 | P30740 |

TABLE 4-5

| | | | | |
|---|---|---|---|---|
| ADP-ribosylation factor 6 | NM_001663 | NP_001654 | ARF6 | P62330 |
| Complement component C9 | NM_001737 | NP_001728 | C9 | P02748 |
| ATP-dependent (S)-NAD(P)H-hydrate dehydratase | NM_001242881, NM_001242882, NM_001242883, NM_018210 | NP_001229810, NP_001229811, NP_001229812, NP_060680 | NAXD | Q8IW45 |
| Actin, alpha cardiac muscle 1 | NM_005159 | NP_005150 | ACTC1 | P68032 |
| Heat shock 70 kDa protein 4 | NM_002154, NM_198431 | NP_002145 | HSPA4 | P34932 |
| 14-3-3 protein theta | NM_006826 | NP_006817 | YWHAQ | P27348 |
| Phosphatidylinositol transfer protein beta isoform | NM_001284277, NM_001284278, NM_012399, XM_011530052, XM_017028707 | NP_001271206, NP_001271207, NP_036531, XP_011528354, XP_016884196 | PITPNB | P48739 |
| Phosphoribosyltransferase domain-containing protein 1 | NM_001282786, NM_020200 | NP_001269715, NP_064585 | PRTFDC1 | Q9NRG1 |
| Costars family protein ABRACL | NM_021243, XM_017011151 | NP_067066, XP_016866640 | ABRACL | Q9P1F3 |
| Ubiquitin-conjugating enzyme E2 L5 | NM_001355247 | NP_001342176 | UBE2L5 | A0A1 B0GUS4 |
| Sodium/potassium-transporting ATPase subunit alpha-1 | NM_000701, NM_01001586, NM_001160233, NP_001160234, XM_017001360, XM_017001361 | NP_000692, NP_001153705, NP_001153706, XP_016856849, XP_016856850 | ATP1A1 | P05023 |
| 14-3-3 protein epsilon | NM_006761, XM_005256784, XM_017025005 | NP_006752, XP_005256841, XP_016880494 | YWHAE | P62258 |
| Major prion protein | NM_000311, NM_001080121, NM_001080122, NM_001080123, NM_001271561, NM_183079 | NP_000302, NP_001073590, NP_001073591, NP_001073592, NP_001258490, NP_898902 | PRNP | P04156 |
| F-actin-capping protein subunit alpha-2 | NM_006136 | NP_006127 | CAPZA2 | P47755 |
| HLAclass I histocompatibility antigen Cw-4 alpha chain | NM_001243042, NM_002117 | NP_001229971, NP_002108 | HLA-C | P30504 |
| GTP-binding protein SAR1a | NM_001142648, NM_020150, | NP_001136120, NP_064535 | SAR1A | Q9NR31 |
| Actin-related protein ⅔ complex subunit 5 | NM_001270439, NM_005717 | NP_001257368, NP_005708 | ARPC5 | O15511 |
| Glutathione reductase, mitochondrial | NM_000637, NM_001195102, NM_001195103, NM_001195104 | NP_000628, NP_001182031, NP_001182032, NP_001182033 | GSR | P00390 |
| Guanine nucleotide-binding protein subunit alpha-11 | NM_002067 | NP_002058 | GNA11 | P29992 |
| Protein disulfide-isomerase A5 | NM_006810 | NP_006801 | PDIA5 | Q14554 |
| MARCKS-related protein | NM_023009 | NP_075385 | MARCKSL1 | P49006 |
| Septin-7 | NM_001011553, NM_001242956, NM_001363715, NM_001788, XM_006715806, XM_011515656, XM_011515661, XM_017012855, XM_017012856, XM_017012857, XM_017012858, XM_017012859, XM_017012860, XM_017012861, XM_017012862, XM_017012863, XM_24447010 | NP_001011553, NP_001229885, NP_001350644, NP_001779, XP_006715869, XP_011513958, XP_011513963, XP_016868344, XP_016868345, XP_016868346, XP_016868347, XP_016868348, XP_016868349, XP_016868350, XP_016868351, XP_016868352, XP_024302778 | SEPT7 | Q16181 |
| Hsc70-interacting protein | NM_001278589, NM_003932 | NP_001265518, NP_003923 | ST13 | P50502 |
| COP9 signalosome complex subunit 8 | NM_006710, NM_198189 | NP_006701, NP_937832 | COPS8 | Q99627 |
| Vacuolar protein sorting-associated protein 37C | NM_017966, XM_005274077, XM_017017955 | NP_060436, XP_005274134, XP_016873444 | VPS37C | A5D8V6 |
| Acidic fibroblast growth factor intracellular-binding protein | NM_004214, NM_198897, XM_024448763 | NP_004205, NP_942600, XP_024304531 | FIBP | 043427 |
| ADP-ribosyl cyclase/cyclic ADP-ribose hydrolase 1 | NM_001775 | NP_001766 | CD38 | P28907 |

TABLE 4-6

| | | | | |
|---|---|---|---|---|
| Ribosome maturation protein SBDS | NM_016038 | NP_057122 | SBDS | Q9Y3A5 |
| Fructose-bisphosphate aldolase A | NM_000034, NM_001127617, NM_001243175, NM_001243177, NM_001355562, NM_001355563, NM_001355564, NM_001355565, NM_184041, NM_184043, XM_024450192, XM_024450193 | NP_000025, NP_001121089, NP_001230106, NP_001342491, NP_001342492, NP_001342493, NP_001342494, NP_908930, NP_908932, XP_024305960, XP_024305961 | ALDOA | P04075 |
| BH3-interacting domain death agonist | NM_001196, NM_001244567, NM_001244569, NM_001244570, NM_001244572, NM_197966, NM_197967 | NP_001187, NP_001231496, NP_001231498, NP_001231499, NP_001231601, NP_932070, NP_932071 | BID | P55957 |
| Charged multivesicular body protein 4b | NM_176812 | NP_789782 | CHMP4B | Q9H444 |
| Guanine nucleotide-binding protein G(s) subunit alpha isoforms short. | NM_000516, NM_001077488, NM_001077489, NM_001077490, NM_001309840, NM_001309842, NM_00130861, NM_001309883, NM_016592, NM_080425, NM_080426, XM_017027812, XM_017027813, XM_017027814, XM_017027815, XM_017027816, XM_017027817, XM_017027818, XM_017027819, XM_017027820, XM_017027821, XM_017027822, XM_024451872, XM_024451873, XM_024461874, XM_024451875 | NP_000507, NP_001070956, NP_001070957, NP_001070958, NP_001296769, NP_001296771, NP_001296790, NP_001296812, NP_057676, NP_536350, NP_536351, XP_016883301, XP_016883302, XP_016883303, XP_016883304, XP_016883305, XP_016883306, XP_016883307, XP_016883308, XP_016883309, XP_016883310, XP_016883311, XP_024307640, XP_024307641, XP_024307642, XP_024307643 | GNAS | P63092 |
| cAMP-dependent protein kinase catalytic subunit alpha | NM_001304349, NM_002730, NM_207518, XM_017026948 | NP_001291278, NP_002721, NP_997401, XP_016882437 | PRKACA | P17612 |
| Cytosolic non-specific dipeptidase | NM_001168499, NM_018235, XM_005266728, XM_006722603, XM_011526072, XM_011526073 | NP_001161971, NP_060705, XP_005266785, XP_006722566, XP_011524374, XP_011524375 | CNDP2 | Q96KP4 |
| Dynamin-1 | NM_001005336, NM_001288737, NM_001288738, NM_001288739, NM_004408, XM_005251763, XM_005251768, XM_005251769, XM_006716992, XM_006716993, XM_011518335, XM_011518336, XM_017014369, XM_017014370, XM_017014371, XM_017014372, XM_017014373 | NP_001005336, NP_001275666, NP_001275667, NP_001275668, NP_004399, XP_005251820, XP_005251825, XP_005251826, XP_006717055, XP_006717056, XP_011516637, XP_011516638, XP_016869858, XP_016869859, XP_016869860, XP_016869861, XP_016869862 | DNM1 | Q05193 |
| Neuroplastin | NM_001161363, NM_001161364, NM_012428, NM_017455, XM_011521452 XM_024449899. XM_024449900 | NP_001154835, NP_001154836, NP_036560, NP_059429, XP_011519754, XP_024305667, XP_024305668 | NPTN | Q9Y639 |
| IST1 homolog | NM_001270975, NM_001270976, NM_001270977, NM_001270978, NM_001270979, NM_014761 | NP_001257904, NP_001257905, NP_001257906, NP_001257907, NP_001257908, NP_055576 | IST1 | P53990 |

TABLE 4-7

| Sorting nexin-5 | NM_001282454, NM_014426, NM_152227 | NP_001269383, NP_055241, NP_689413 | SNX5 | Q9Y5X3 |
|---|---|---|---|---|
| Mitochondrial import inner membrane translocase subunit Tim13 | NM_012458 | NP_036590 | TIMM13 | Q9Y5L4 |
| Exocyst complex component 4 | NM_001037126, NM_021807, XM_005250523, XM_017012494 | NP_001032203, NP_068579, XP_005250580, XP_016867983 | EXOC4 | Q96A65 |
| Moesin | NM_002444, XM_005262269, XM_011530959, XM_017029545, XM_017029546 | NP_002435 XP_005262326, XP_011529261, XP_016885034, XP_016885035 | MSN | P26038 |
| Serum albumin | NM_000477 | NP_000468 | ALB | P02768 |
| Myristoylated alanine-rich C-kinase substrate | NM_002356 | NP_002347 | MARCKS | P29966 |
| Sodium/potassium-transporting ATPase subunit beta-3 | NM_001679 | NP_001670 | ATP1B3 | P54709 |
| Vinculin | NM_003373, NM_014000 | NP_003364 NP_054706 | VCL | P18206 |
| Actin-related protein ⅔ complex subunit 1B | NM_005720, XM_006715825, XM_006715826, XM_024446628, XM_024446629 | NP_005711, XP_006715888, XP_006715889, XP_024302396, XP_024302397 | ARPC1B | 015143 |
| Alcohol dehydrogenase class-3 | NM_000671 | NP_000662 | ADH5 | P11766 |
| Cysteine and glycine-rich protein 1 | NM_001144773, NM_001193570, NM_001193571, NM_001193572, NM_004078 | NP_001180499, NP_001180500, NP_001180501, NP_004069 | CSRP1 | P21291 |
| E3 ubiquitin-protein ligase TRIM23 | NM_001656, NM_033227, NM_033228, XM_017009444 | NP_001647 NP_150230, NP_150231, XP_016864933 | TRIM23 | P36406 |
| Actin-related protein 3 | NM_001277140, NM_005721 | NP_001264069, NP_005712 | ACTR3 | P61158 |
| Tetraspanin-15 | NM_001351263, NM_012339, XM_005269666, XM_011539562, XM_011539563, XM_017016010 | NP_001338192, NP_036471, XP_005269723, XP_011537864, XP_011537865, XP_016871499 | TSPAN15 | 095858 |
| Ras-related C3 botulinum toxin substrate 1 | NM_006908, NM_018890, NM_198829 | NP_008839, NP_061485 | RAC1 | P63000 |
| COP9 signalosome complex subunit 2 | NM_001143887, NM_004236 | NP_001137359, NP_004227 | COPS2 | P61201 |
| Myotrophin | NM_145808 | NP_665807 | MTPN | P58546 |
| Rho-related GTP-binding protein RhoF | NM_019034 | NP_061907 | RHOF | Q9HBH0 |
| COP9 signalosome complex subunit 3 | NM_001199125, NM_001316354, NM_001316355, NM_001316356, NM_001316357, NM_001316358, NM_003653, XM_005256837, XM_005256840, XM_005256842, XM_017025246 | NP_001186054, NP_001303283, NP_001303284, NP_001303285, NP_001303286, NP_001303287, NP_003644, XP_005256894, XP_005256897, XP_005256899, XP_016880735 | COPS3 | Q9UNS2 |
| Glutathione synthetase | NM_000178, NM_001322494, NM_001322495 | NP_000169, NP_001309423, NP_001309424 | GSS | P48637 |
| DnaJ homolog subfamily C member 5 | NM_025219, XM_011529048, XM_011529049 | NP_079495 XP_011527350, XP_011527351 | DNAJC5 | Q9H3Z4 |
| Glutathione S-transferase omega-1 | NM_001191002, NM_001191003, NM_004832 | NP_001177931, NP_001177932, NP_004823 | GSTO1 | P78417 |
| Glutathione S-transferase Mu 2 | NM_000848, NM_001142368 | NP_000839 NP_001135840 | GSTM2 | P28161 |
| Ragulator complex protein LAMTOR2 | NM_001145264, NM_014017 | NP_001138736, NP_054736 | LAMTOR2 | Q9Y2Q5 |
| Cytochrome b5 type B | NM_030579 | NP_085056 | CYB5B | 043169 |

TABLE 4-8

| | | | | |
|---|---|---|---|---|
| Progressive ankylosis protein homolog | NM_054027, XM_011514067, XM_017009644 | MP_473368, XP_011512369, XP_016865133 | ANKH | Q9HCJ1 |
| Septin-11 | NM_001306147, NM_018243, XM_006714259, XM_017008400, XM_017008401, XM_017008402, XM_017008403, XM_017008404 | NP_001293076, NP_060713, XP_006714322, XP_016863889, XP_16863890, XP_016863891, XP_016863892, XP_016863893 | SEPT11 | Q9NVA2 |
| Rho-associated protein kinase 1 | NM_005406, XM_011526137 | NP_005397, XP_011524439 | ROCK1 | Q13464 |
| Ras-related C3 botulinum toxin substrate 3 | NM_001316307, NM_005052 | NP_001303236, NP_005043 | RAC3 | P60763 |
| Isocitrate dehydrogenase [NAD] subunit alpha, mitochondrial | NM_005530, XM_005254336, XM_024449911, XM_024449912 | NP_005521, XP_005254393, XP_024305679, XP_024305680 | IDH3A | P50213 |
| Inactive tyrosine-protein kinase transmembrane receptor ROR1 | NM_001083592, NM_005012, XM_011541526, XM_017001376, XM_017001377 | MP_001077061 NP_005003, XP_011539828, XP_016856865, XP_016856866 | ROR1 | Q01973 |
| Abl interactor 2 | NM_001282925, NM_001282926, NM_001282927, NM_001282932, NM_005759, XM_005246217, XM_005246218, XM_005246221, XM_005246225, XM_005246226, XM_006712174, XM_006712177, XM_006712179, XM_006712180, XM_006712181, XM_006712182, XM_006712185, XM_006712186, XM_011510472, XM_011510473, XM_017003118, XM_017003119, XM_017003120, XM_017003121, XM_017003122, XM_017003123, XM_017003124, XM_017003125, XM_017003126, XM_017003127, XM_017003128, XM_017003129, XM_017003130, XM_017003131, XM_017003132, XM_017003133, XM_017003134, XM_017003135, XM_017003136, XM_017003137, XM_017003138, XM_017003139, XM_017003140, XM_017003141, XM_017003142, XM_017003143, XM_017003144, XM-017003145, XM-017003146, XM-024452511 | NP_001269854, NP_001269855, NP_001269856, NP_001269861, NP_005750, XP_005246274, XP_005246275, XP_005246278, XP_005246282, XP_005246283, XP_006712237, XP_006712240, XP_006712242, XP_006712243, XP_006712244, XP_006712245, XP_006712248, XP_006712249 XP_011508774, XP_011508775, XP_016858607, XP_016858608, XP_016858609, XP_016858610, XP_016858611,XP_016858612, XP_016858613, XP_016858614, XP_016858615, XP_016858616, XP_016858617, XP_J16858618, XP_016858619, XP_016858620, XP_016858621, XP_016858622 XP_016858623, XP_016858624, XP_016858625, XP_016858626, XP_016858627, XP_016858628, XP_016858629, XP_016858630, XP_016858631, XP_016858632, XP_016858633, XP_016858634, XP_016858635, XP_024308279 | ABI2 | Q9NYB9 |
| Cell division control protein 42 homolog | NM_001039802, NM_001791, NM_044472 | NP_001034891, NP_001782, MP_426359 | CDC42 | P60953 |
| Transgelin-2 | NM_001271223, NM_001277224, NM_003564 | NP_001264152, NP_001264153, NP_003555 | TAGLN2 | P37802 |
| Ras-related protein Rab-2A | NM_001242644, NM_002865 | NP_001229573, MP_002856 | RAB2A | P61019 |
| Charged multivesicular body protein 6 | NM_024591, XM_005257668 | NP_078867, XP_005257725 | CHMP6 | Q96FZ7 |
| ADP-ribosylation factor 3 | NM_001659, XM_005268856, XM_006719391, XM_024448972 | NP_001660, XP_005268913, XP_006719454, XP_024304740 | ARF3 | P61204 |

TABLE 4-9

| | | | | |
|---|---|---|---|---|
| Glyoxylate reductase/hydroxypyruvate reductase | NM_012203, XM_005251631, XM_017015320, XM_017015321, XM_017015323, XM_024447716, XM_024447717 | NP_036335, XP_005251688, XP_016870809, XP_016870810, XP_016870812, XP_024303484, XP_024303485 | GRHPR | Q9UBQ7 |
| Profilin-1 | NM_005022, XM_017024761 | NP_005013, XP_016880250 | PFN1 | P07737 |
| Ras-related protein Rab-11B | NM_004218 | NP_004209 | RAB11B | Q15907 |
| Rab GDP dissociation inhibitor alpha | NM_001493 | NP_001484 | GDI1 | P31150 |
| CD97 antigen | NM_001025160, NM_001784, NM_078481, XM_011528451 | NP_001020331, NP_001775, NP_510966, XP_011526753 | CD97 | P4B960 |
| Programmed cell death protein 10 | NM_007217, NM_145859, NM_145860, XM_005247086, XM_005247087, XM_005247088, XM_006713485, XM_011512368, XM_011512369, XM_017005644, XM_017005645, XM_024453329, XM_024463330, XM_024453331 | NP_009148, NP_665858, NP_665859, XP_005247143, XP_005247144, XP_005247145, XP_006713548, XP_011510670, XP_011510671, XP_016861133, XP_016861134, XP_024309097 XP_024309098, XP_024309099 | PDCD10 | Q9BUL8 |
| Antithrombin-lll | NM_000488, NM_001365052, XM_005245198 | NP_000479, NP_001351981, XP_005245255 | SERPINC1 | P01008 |
| F-actin-capping protein subunit beta | NM_001206540, NM_001206541, NM_001282162, NM_001313932, NM_004930, XM_006710938, XM_011542228, XM_011542229, XM_011542230, XM_017002428, XM_017002429, XM_017002430, XM_024450096, XM_024450097 | NP_001193469 NP_001193470, NP_001269091, NP_001300861, NP_004921, XP_006711001, XP_011540530, XP_011540531, XP_011540532, XP_016857917, XP_016857918, XP_016857919, XP_024305864, XP_024305865 | CAPZB | P47756 |
| Actin-related protein ⅔ complex subunit 2 | NM_005731, NM_152862, XM_017003113, XM_017003114, XM_017003115, XM_024452509 | NP_005722, NP_690601, XP_016858602, XP_016858603, XP_016858604, XP_024308277 | ARPC2 | 015144 |
| Twinfilin-2 | NM_007284 | NP_009215 | TWF2 | Q6lBSO |
| Ras-related protein Rab-21 | NM_014999 | NP_055814 | RAB21 | Q9UL25 |
| Vacuolar-sorting protein SNF8 | NM_001317192, NM_001317193, NM_001317194, NM_007241, XM_017024075 | NP_001304121, NP_001304122, NP_001304123, NP_009172, XP_016879564 | SNF8 | Q96H20 |
| Caspase-6 | NM_001226, NM_032992 | NP_001 217, NP_116787 | CASP6 | P55212 |
| Calcineurin subunit B type 1 | NM_000945 | NP_000936 | PPP3R1 | P63098 |
| Extended synaptotagmin-2 | NM_020728, XM_024446845, XM_024446846, XM_024446847, XM_024446848, XM_024446849 | NP_065779, XP_024302613, XP_024302614, XP_024302615, XP_024302616, XP_024302617 | ESYT2 | A0FGR8 |
| Leukocyte surface antigen CD47 | NM_001025079, NM_001025080, NM_001777, NM_198793, XM_005247908, XM_005247909, XM_017007536 | NP_001768, NP_942088, XP_005247965, XP_005247966, XP_016863025 | CD47 | Q08722 |

TABLE 4-10

| | | | | |
|---|---|---|---|---|
| COP9 signalosome complex subunit 1 | NM_001321089, NM_001321090, NM_001321091, NM_001321092, NM_001321093, NM_001330539, NM_001330541, NM_004127, NM_212492, XM_005256350, XM_005256353, XM_005256359, XM_005256360, XM_017024524, XM_017024525, XM_017024526, XM_017024527, XM_017024531, XM_017024534, XM_017024535, XM_024450713, XM_024450714, XM_024450715, XM_024450716, XM_024450717, XM_024450718, XM_024450719, XM_024450720, XM_024450721, XM_024450722, XM_024450723, XM_024450724, XM_024450725, XM_024450726, XM_024450727, XM_024450728 | NP_001308018, NP_001308019, NP_001308020, NP_001308021, NP_001308022, NP_001317468, NP_001317470, NP_004118, NP_997657, XP_005256407, XP_005256410, XP_005256416, XP_005256417, XP_016880013, XP_016880014, XP_016680015, XP_016880016, XP_016880020, XP_016880023, XP_016880024, XP_024306481, XP_024306482, XP_024306483, XP_024306484, XP_024306485, XP_024306486, XP_024306487, XP_024306488, XP_024306489, XP_024306490, XP_024306491, XP_024306492, XP_024306493, XP_024306494, XP_024306495, XP_024306496 | GPS1 | Q13098 |
| 26S proteasome non-ATPase regulatory subunit 7 | NM_002811 | NP_002802 | PSMD7 | P51665 |
| Eukaryotic translation initiation factor 3 subunit C-like protein | NM_001099661, NM_001317856, NM_001317857, XM_017023620, XM_017023621 | NP_001093131, NP_001304785, NP_001304786, XP_016879109, XP_016879110 | EIF3CL | B5ME19 |
| Deoxycytidylate deaminase | NM_001012732, NM_001351743, NM_001351744, NM_001351745, NM_001351747, NM_001351748, NM_001351750, NM_001351753, NM_001921, XM_005262778, XM_005262781, XM_005262782, XM_006714115, XM_006714116, XM_011531674, XM_011531675, XM_011531676, XM_017007820, XM_017007822, XM_017007823, XM_017007824 | NP_001012750, NP_001338672, NP_001338673, NP_001338674, NP_001338676, NP_001338677, NP_001338679, NP_001338682, NP_001912, XP_005262835, XP_005262838, XP_005262839, XP_006714178, XP_006714179, XP_011529976, XP_011529977, XP_011529978 XP_016863309, XP_016863311 XP_016863312, XP_016863313 | DCTD | P32321 |
| CD276 antigen | NM_001024736, NM_001329628, NM_001329629,NM_025240, NM_001329629 NM_025240 XM_005254700, XM_011522096, XM_017022638 | NP_001019907, NP_001316557, NP_079516 NP_001316558 NP_079516 XP_005254757, XP_011520397, XP_016878127 | CD276 | Q5ZPR3 |
| Caspase-3 | | NP_001341706, NP_001341708, NP_001341709, NP_001341710, NP_001341711, NP_001341712, NP_001341713, NP_004337, NP_116786 | CASP3 - | P42574 |
| Leucine-rich repeat-containing protein 57 | NM_153260, XM_011521423, XM_011521424 | NP_694992, XP_011519725, XP_011519726 | LRRC57 | Q8N9N7 |
| Aldose reductase | NM_001346142, NM_001628, XM_005250234, XM_024446695 | NP_001333071, NP_001619, XP_005250291, XP_024302463 | AKR1B1 | P15121 |

TABLE 4-11

| | | | | |
|---|---|---|---|---|
| Diphosphoinositol polyphosphate phosphohydrolase 1 | NM_006703 | NP_006694 | NUDT3 | 095989 |
| Coagulation factor XIII A chain | NM_000129 | NP_000120 | F13A1 | P00488 |
| Hsp70-binding protein 1 | NM_001130106, NM_001297600, NM_012267, XM_005258700, XM_005258701, XM_011526691, XM_017026543, XM_017026544, XM_017026545, XM_024451436, XM_024451437 | NP_001123578, NP_001284529, NP_036399, XP_005258757, XP_005258758, XP_011524993, XP_016882032, XP_016882033, XP_016882034, XP_024307204, XP_024307205 | HSPBP1 | Q9NZL4 |
| Sorting nexin-4 | NM_003794, XM_017007414 | NP_003785, XP_016862903 | SNX4 | 095219 |
| Protein phosphatase 1L | NM_001317911, NM_001317912, NM_139245, XM_011512440 | NP_001304840, NP_001304841, NP_640338, XP_011510742 | PPM1L | Q5SGD2 |
| Pleckstrin homology domain-containing family N member 1 | NM_001160184, NM_032129, XM_006710944, XM_011542248, XM_017002474, XM_017002475, XM_017002476, XM_017002477, XM_017002478, XM_017002479 | NP_001153656, NP_115505, XP_006711007, XP_011540550, XP_016857963, XP_016857964, XP_016857965, XP_016857966, XP_016857967, XP_016857968 | PLEKHN1 | Q494U1 |
| AP-2 complex subunit alpha-2 | NM_001242837, NM_012305, XM_011519929, XM_011519930 | NP_001229766, NP_036437, XP_011518231 XP_011518232 | AP2A2 | 094973 |
| Trophoblast glycoprotein | NM_001166392, NM_006670 | NP_001159864, NP_006661 | TPBG | Q13641 |
| Ras-related protein Rab-23 | NM_001278666 NM_001278667, NM_001278668, NM-016277, NM_183227 | NP_001265595, NP_001265596 NP_001265597, MP_057361, NP_899050 | RAB23 | Q9ULC3 |
| 14-3-3 protein eta | NM_003405 | NP_003396 | YWHAH | Q04917 |
| Peroxiredoxin-6 | NM_004905 | NP_004896 | PRDX6 | P30041 |
| 14-3-3 protein beta/alpha | NM_003404, NM_139323, XM_017028039, XM_017028040 | NP_003395, NP_647539, XP_016883528, XP_016883529 | YWHAB | P31946 |
| Copine-2 | NM_152727 | NP_689940 | CPNE2 | Q96FN4 |
| 14-3-3 protein gamma | NM_012479 | NP_036611 | YWHAG | P61981 |
| Superoxide dismutase [Cu-Zn] | NM_000454 | NP_000445 | SOD1 | P00441 |
| Microtubule-associated protein RP/EB family member 1 | NM_012325, XM_011528696 | NP_036457, XP_011526998 | MAPRE1 | Q15691 |
| Vasodilator-stimulated phosphoprotein | NM_001008736, NM_003370, XM_005259199, XM_005259200, XM_017027200 | NP_003361, XP_005259256, XP_005259257, XP_016882689 | VASP | P50552 |
| Phosphoglucomutase-1 | NM_001172818, NM_001172819, NM_002633 | NP_001166289, NP_001166290, NP_002624 | PGM1 | P36871 |
| Adenylyl cyclase-associated protein 1 | NM_001105530, NM_001330502, NM_001350475, NM_001350476, NM_001350477 NM_001350478, NM_001350479, NM_001350480, NM_001350481, NM_001350482, NM_001350483, NM_001350484, NM_001350485, NM_006367, XM_011540510, XM_011540515, XM_017000069 | NP_001099000, NP_001317431, NP_001337404, NP_001337405, NP_01337406, NP_001337407, NP_001337408, NP_001337409, NP_001337410, NP_001337411, NP_001337412, NP_001337413, NP_001337414, NP_006358, XP_011538812, XP_011538817, XP_016855558 | CAP1 | Q01518 |

TABLE 4-12

| | | | | |
|---|---|---|---|---|
| Calpain small subunit 1 | NM_001003962, NM_001302632, NM_001302633, NM_001749, XM_005259295, XM_005259296, XM_011527359, XM_024451732 | MP_001003962 NP_001289561, NP_001289562, MP_001740, XP_005259352, XP_005259353 XP_011525661, XP_024307500 | CAPNS1 | P04632 |
| Dual specificity protein phosphatase 23 | NM_001319658, NM_001319659, NM_017823 | NP_001306587, NP_001306588, NP_060293 | DUSP23 | Q9BVJ7 |
| Dihydropteridine reductase | NM_000320, NM_001306140 | NP_000311, NP_001293069 | QDPR | P09417 |
| Ephrin-B1 | NM_004429 | NP_004420 | EFNB1 | P98172 |
| Golgin subfamily A member 7 | NM_001002296, NM_001174124, NM_001362979, NM_001362980, NM_016099, XM_024447174, XM_024447175 | NP_001002296, NP_001167595, NP 001349908 NP_001349909, NP_057183, XP_024302942, XP_024302943 | GOLGA7 | Q7Z5G4 |
| Xaa-Pro aminopeptidase 1 | NM_001167604, NM_001324128, NM_001324131, NM_001324132, NM_001324133, NM_001324134, NM_001324135, NM_001324136, NM_020383, XM_011540134, XM_017016613, XM_017016614, XM_017016615, XM_024448156 | NP_001161076, NP_001311057, NP_001311060, NP_001311061, NP_001311062, NP_001311063, NP_001311064, NP_001311065, NP_065116, XP_011538436, XP_016872102, XP_016872103 XP_016872104, XP_024303924 | XPNPEP1 | Q9NQW7 |
| Actin-related protein ⅔ complex subunit 3 | NM_001278556, NM_001287222 | NP_001265485 NP_001274151 | ARPC3 | 015145 |
| Sorting nexin-9 | NM_016224, XM_005267015, XM_011535886 | NP_057308, XP_005267072, XP_011534188 | SNX9 | Q9Y5X1 |
| Pleckstrin homology domain-containing family O member 2 | NM_001195059, NM_025201 | NP_001181988, NP_079477 | PLEKHO2 | Q8TD55 |
| WD repeat-containing protein 44 | NM_001184965, NM_001184966, NM_019045, XM_011531353, XM_017029600 | NP_001171894, NP_001171895, NP_061918 XP_011529655 XP_016885089 | WD R44 | Q5JSH3 |
| N(G),N(G)-dimethylarginine dimethylaminohydrolase 2 | NM_001303007, NM_001303008, NM_013974, XM_011514448 | NP_001289936, NP_001289937, NP_039268 XP_011512750 | DDAH2 | 095865 |
| COP9 signalosome complex subunit 6 | NM_006833 | NP_006824 | COPS6 | Q7L5N1 |
| Guanine nucleotide-binding protein G(I)/G(S)/G(O) subunit gamma-10 | NM_001017998, NM_001198664 | NP_001017998 NP_001185593 | GNG10 | P50151 |
| Rab-like protein 3 | NM_001363964, NM_001363965, NM_173825, XM_006713595, XM_017006227, XM_017006228 | NP_001350893 NP_001350894, NP_776186 XP_006713658, XP_016861716, XP_016861717 | RABL3 | Q5HYI8 |
| Phosphoacetylglucosamine mutase | NM_001199917, NM_001199918, NM_001199919, NM 015599, XM_017010935, XM_017010937, XM_024446459, XM_024446460 | NP_001186846, NP_001186847, NP_001186848, NP_056414, XP_016866424, XP_016866426, XP_024302227, XP_024302228 | PGM3 | 095394 |
| Sorting nexin-2 | NM_001278199, NM_003100 | NP_001265128 NP_003091 | SNX2 | 060749 |
| DDB1- and CUL4.associated factor 11 | NM_001163484, NM_025230, NM_181357 | NP_001156956, NP_079606, NP_852002 | DCAF11 | Q8TEB1 |
| Ras GTPase-activating protein 1 | NM_002890, NM_022650, XM_011543525, XM_011543527 | NP_002881 NP_072179, XP_011541827, XP_011541829 | RASA1 | P20936 |
| Ribose-5-phosphate isomerase | NM_144563 | NP_653164 | RPIA | P49247 |

TABLE 4-13

| | | | | |
|---|---|---|---|---|
| SHC-transforming protein 1 | NM_001130040, NM_001130041, NM_001202859, NM_003029, NM_183001, XM_005245449, XM_005245451, XM_011509892, XM_011509893, XM_011509894, XM_011509897, XM_011509898, XM_017002081, XM_017002082, XM_017002083, XM_024449083, XM_024449084 | NP_001123512, NP_001123513 NP_001189788, NP_003020, NP_892113, XP_005246606, XP_005245508, XP_011508194, XP_011508195, XP 011508196, XP_011508199, XP_011508200, XP_016857570 XP_016857571, XP_016857572 XP-024304651, XP_024304852 | SHC1 | P29353 |
| Glutaminyl-peptide cyclotransferase-like protein | NM_001163377, NM_017659, XM_011527048, XM_017026900 | NP_001166849, NP_060129, XP_011525350, XP_016882389 | QPCTL | Q9NXS2 |
| Ras-related protein Rab-5C | NM_001252039, NM_004583, NM_201434, XM_011525090 | NP_001238968, NP_004574, NP_958842, XP_011523392 | RAB5C | P51148 |
| Purine nucleoside phosphorylase | NM_000270 | NP_000261 | PNP | P00491 |
| Delta-aminolevulinic acid dehydratase | NM_000031, NM_001003945, NM_001317745, XM_011518364, XM_024447449 | NP_000022, NP_001003945, NP_001304674, XP_011516666 XP_024303217 | ALAD | P13716 |
| Ras-related protein Rab-5B | NM_001252036, NM_001252037, NM_002868, XM_005269051, XM_005269052 | NP_001238965, NP_001238966, NP_002859, XP_005269108, XP_005269109 | RAB5B | P61020 |
| Abl interactor 1 | NM_001012750, NM_001012751, NM_001012752, NM_001178116, NM_001178119, NM_001178120, NM_001178121, NM_001178122, NM_001178123, NM_001178124, NM_001178125, NM_001348029, NM_001348030, NM_001348031, NM_001348032, NM_001348033, NM_001348034, NM_005470, XM_005252332, XM_005252333, XM_011519289, XM_017015459 | NP_001012768, NP_001012769, NP_001012770, NP_001171587, NP_001171590, NP_001171591, NP_001171592, NP_001171593 NP_001171694, NP_001171595, NP_001171596, NP_001334958, NP_001334959, NP_001334960, NP 001334961, NP_001334962, NP_001334963, NP_005461, XP_005252389, XP_005252390, XP_011517591,XP_016870948 | ABI1 | Q8IZPO |
| Beta-2-glycoprotein 1 | NM_000042 | NP_000033 | APOH | P02749 |
| Serine/threonin-protein phosphatase 2B catalytic subunit alpha isoform | NM_000944, NM_001130691, NM_001130692, XM_017008365, XM_024454127 | NP_000935, NP_001124163, NP_001124164, XP_016863854, XP_024309895 | PPP3CA | Q08209 |
| Actin-related protein 2 | NM_001005386, NM_005722 | NP_001005386, NP_005713 | ACTR2 | P61160 |
| Ras-related protein Rap-2a | NM_021033 | NP_066361 | RAP2A | P10114 |
| Sorcin | NM_001256891, NM_001256892, NM_003130, NM_198901, XM_011516528 | NP_001243820, NP_001243821, NP_003121, NP_944490, XP_011514830 | SRI | P30626 |
| Type I inositol 1,4 5-trisphosphate 5-phosphatase | NM_001321042, NM_005539, XM_017016204, XM_017016205 | NP_001307971, NP_005530, XP_016871693 XP_016871694 | INPP5A | 014642 |
| Ras-related protein Rab-35 | NM_001167606, NM_006861, XM_011537779, XM_024448801 | NP_001161078, NP_006852, XP_011536081, XP_024304569 | RAB35 | Q15286 |
| Rho GTPase-activating protein 18 | NM_033515, XM_005267212, XM_005267213, XM_017011494 | NP_277050, XP_005267269, XP_005267270, XP_016866983 | ARHGAP18 | Q8N392 |

TABLE 4-14

| | | | | |
|---|---|---|---|---|
| Ras-related protein Rab-1A | NM_004161, NM_015543, XM_005264468 | NP_004152, NP_056358, XP_005264525 | RAB1A | P62820 |
| Deoxyribonuclease-1-like 1 | NM_001009932, NM_001009933, NM_001009934, NM_001303620, NM_006730, XM_005277829, XM_011531122, XM_017029332 | NP_001009932, NP_001009933, NP_0010D9934, NP_001290549, NP_006721, XP_005277886, XP_011529424, XP_016884821 | DNASE1 L1 | P49184 |
| Peptidyl-prolyl cis-trans isomerase A | NM_001300981, NM_021130, NM_203430, NM_203431, XM_024446808, XM_024446809 | NP_001287910, NP_066953, XP_024302576, XP_024302577 | PPIA | P62937 |
| Adenylate kinase isoenzyme 1 | NM_000476, NM_001318121, NM_001318122, XM_017014428, XM_024447439, XM_024447440 | NP_000467, NP_001305050, NP_001305051, XP_016869917, XP_024303207 XP_024303208 | AK1 | P00568 |
| Equilibrative nucleoside transporter 1 | NM_001078174, NM_001078175, NM_001078176, NM_001078177, NM_001304462, NM_001304463, NM_001304465, NM_001304466, NM_004955, XM_005248876, XM_005248878, XM_005248879, XM_005248880, XM_005248881, XM_005248882, XM_011514341, XM_024446348 | NP_001071643, NP_001071645, NP_001291391, NP_001291392, NP_001291394, NP_001291395, XP_005248933, XP_005248935, XP_005248936, XP_005248937, XP_005248938, XP_005248939, XP_011512643, XP_024302116 | SLC29A1 | Q99808 |
| GTPase NRas | NM_002524 | NP_002515 | NRAS | P01111 |
| Rho-related GTP-binding protein RhoG | NM_001665, XM_005252916, XM_017017719 | NP_001656, XP_005252973, XP_016873208 | RHOG | P84095 |
| Glycogen phosphorylase, brain form | NM_002862 | NP_002853 | PYGB | P11216 |
| UV excision repair protein RAD23 homolog B | NM_001244713, NM_001244724, NM_002874 | NP_001231642, NP_001231653, NP_002865 | RAD23B | P54727 |
| Omega-amidase NIT2 | NM_020202 | NP_064587 | NIT2 | Q9NQR4 |
| Serine hydroxymethyltransferase, cytosolic | NM_001281786, NM_004169, NM_148918, XM_005256767, XM_011523992, XM_017024957, XM_017024958, XM_024450887, XM_024450888, XM_024450889, XM_024450890 | NP_001268715, NP_004160, NP_68371B, XP_005256824, XP_011522294, XP_016880446, XP_016880447, XP_024306655, XP_024306656, XP_024306657, XP_024306658 | SHMT1 | P34896 |
| Sodium/potassium.transporting ATPase subunit beta-1 | NM_001001787, NM_001677 | NP_001668 | ATP1B1 | P05026 |
| Endothelial cell-selective adhesion molecule | NM_138961 | NP_620411 | ESAM | Q96AP7 |
| Isocitrate dehydrogenase [NADP] cytoplasmic | NM_001282386, NM_001282387, NM_005896 | NP_001269315, NP_001269316, NP_005887 | IDH1 | 075874 |
| Peptidyl-prolyl cis-trans isomerase FKBP1A | NM_000801, NM_001199786, NM_054014 | NP_000792, NP_001186715, NP_463460 | FKBP1A | P62942 |

TABLE 4-15

| | | | | |
|---|---|---|---|---|
| Dystroglycan | NM_001165928, NM_001177634, NM_001177635, NM_001177636, NM_001177631, NM_001177638, NM_001177639, NM_001177640, NM_001177641, NM_001177642, NM_001177643, NM_01177644, NM_004393 | NP_001159400, NP_001171105, NP_001171106, NP_001171107, NP_001171108, NP_001171109, NP_001171110, NP_001171111, NP_001171112, NP_001171113, NP_001171114, NP_001171115, NP_004384 | DAG1 | Q14118 |
| Tumor susceptibility gene 101 protein | NM_006292, XM_005253108 | NP_006283, XP_005253165 | TSG101 | Q99816 |
| Peroxiredoxin-5, mitochondrial | NM_001358511, NM_001368516, NM_012094, NM_181651, NM_181652 | NP_001345440, NP_001345445, NP_036226, NP_857634, NP_857635 | PRDX5 | P30044 |
| Protein XRP2 | NM_006915 | NP_008846 | RP2 | 075695 |
| Actin-related protein ⅔ complex subunit 4 | NM_001024959, NM_001024960, NM_001198780, NM_005718 | NP_001020130, NP_001020131, NP_001185709, NP_005709 | ARPC4 | P59998 |
| Stathmin | NM_001145454, NM_005563, NM_152497, NM_203399, NM_203401 | NP_001138926, NP_005554, NP_981944, NP_981946 | STMN1 | P16949 |
| NSFL1 cofactor p47 | NM_001206736, NM_016143, NM_018839, NM_182483, XM_006723592, XM_006723593, XM_006723594, XM-01 1529300, XM_011529301, XM_011529302, XM_011529304, XM_017027956, XM_017027957, XM_017027958 | NP_001193665, NP_057227, NP_061327, XP_006723655, XP_006723656, XP_006723657, XP_011527602, XP_011527603, XP_011527604, XP_011527606, XP_016883445, XP_016883446, XP_016883447 | NSFL1C | Q9UNZ2 |
| Folate receptor alpha | NM_000802, NM_016724, NM_016725, NM_016729, NM_016730 | NP_000793, NP_057936, NP_057937, NP_057941 | FOLR1 | P15328 |
| Aldo-keto reductase family 1 memberA1 | NM_001202413, NM_001202414, NM_006066, NM_153326, XM_011540491, XM_011540492 | NP_001189342, NP_001189343, NP_006057, NP_697021, XP_011538793, XP_011538794 | AKR1A1 | P14550 |
| Ran-specific GTPase-activating protein | NM_001278639, NM_001278640, NM_001278641, NM_002882, XM_011530289, XM_011530290, XM_011530291, XM_017028890, XM_017028891, XM_017028892 | NP_001265568, NP_001265569, NP_001265570, NP_002873, XP_011528591, XP_011528592, XP_011528593, XP_016884379, XP_016884380, XP_016884381 | RANBP1 | P43487 |
| Serine/threonine-protein phosphatase 2A catalytic subunit beta isoform | NM_001009552, NM_004156 | NP_001009552 | PPP2CB | P62714 |
| Vacuolar protein sorting-associated protein VTA1 homolog | NM_001286371, NM_001286372, NM_016485 | NP_001273300, NP_001273301, NP_057569 | VTA1 | Q9NP79 |
| Dual specificity protein phosphatase 3 | NM_004090 | NP_004081 | DUSP3 | P51452 |

TABLE 4-16

| | | | | |
|---|---|---|---|---|
| Membrane cofactor protein | NM_002389, NM_1 53826, NM_172350, NM_172351, NM_172352, NM_172353, NM_172354, NM_172355, NM_172356, NM_172357, NM_172358, NM_172359, NM_172360, NM_172361, XM_011509563, XM_011509564, XM_017001308, XM_017001309, XM_017001310 | NP_002380, NP_722548, 0 NP_758860, NP_758861, NP_758862, NP_758863, NP_758865, NP_758866, NP_758867, NP_758868, NP_758869, NP_758871, XP_011507865, XP_011507866, XP_016856797, XP_016856798, XP_016856799 | CD46 | P15529 |
| Calcium-binding protein 39 | NM_001130849, NM_001130850, NM_016289, XM_011511350 | NP_001124321, NP_001124322, NP_057373, XP_011509652 | CAB39 | Q9Y376 |
| Argininosuccinate lyase | NM_000048, NM_001024943, NM_001024944, NM_001024946 | NP_000039, NP_001020114, NP_001020115, NP_001020117 | ASL | P04424 |
| Mitochondrial import receptor subunit TOM34 | NM_006809, XM_005260254, XM_011528501, XM_017027600 | NP_006800, XP_005260311, XP_011526803, XP_016883089 | TOMM34 | Q15785 |
| Protein numb homolog | NM_001005743, NM_001005744, NM_001005745, NM_001320114, NM_003744 | NP_001005743, NP_001005744, NP_001005745, NP_001307043, NP_003735 | NUMB | P49757 |
| SUMO-conjugating enzyme UBC9 | NM_003345, NM_194259, NM_194260, NM_194261, XM_005255540, XM_005255541, XM_011522644, XM_011522645, XM_017023640, XM_024450432 | NP_003336, NP_919235, NP_919236, NP_919237, XP_005255597, XP_005255598, XP_011520946, XP_011520947, XP_016879129, XP_024306200 | UBE2l | P63279 |
| EH domain-containing protein 3 | NM-014600, XM_011532806 | NP_055415, XP_011531108 | EHD3 | Q9NZN3 |
| Ras-related protein Rab-1B | NM_030981, XM_017018378 | NP_112243, XP_016873867 | RAB1B | Q9H0U4 |
| Adenine phosphoribosyltransferase | NM_000485, NM_001030018 | NP_000476, NP_001025189 | APRT | P07741 |
| Coronin-1A | NM_001193333, NM_007074, XM_011545714, XM_017022885, XM_017022886 | NP_001180262, NP_009005, XP_011544016, XP_016878374, XP_016878375 | CORO1A | P31146 |
| Annexin A4 | NM_001153, NM_001320698, NM_001320700, NM_001320702, NM_001365496, XM_017003943, XM_017003944, XM_024452834, XM_024452835, XM_024452836 | NP _001144 NP_001307627, NP_001307629, NP_001307631, NP_001352425, XP_016859432, XP_016859433 XP_024308602, XP_024308603, XP_024308604 | ANXA4 | P09625 |
| F-actin-capping protein subunit alpha-1 | NM_006135, XM_011542225, XM_017002424 | NP_006126, XP_011540527, XP_016857913 | CAPZA1 | P52907 |
| Protein phosphatase 1 regulatory subunit 7 | NM_001282409, NM_001282410, NM_001282411, NM_001282412, NM_001282413, NM_001282414, NM_002712, XM_011511388 | NP_001269338, NP_001269339, NP_001269340, NP_001269341, NP_001269342, NP_001269343, NP_002703, XP_011509690 | PPP1R7 | Q15435 |
| Peflin | NM_001359651, NM_012392, XM_011541745, XM_011541746, XM_011541747, XM_017001680, XM_017001681 | NP_001 346580, NP_036524, XP_011540047, XP_011540048, XP_011540049, XP_016857169, XP_016857170 | PEF1 | Q9UBV8 |

TABLE 4-17

| | | | | |
|---|---|---|---|---|
| Proteinfnucleic acid deglycase DJ-1 | NM_001123377, NM_007262, XM_005263424 | NP_001 116849, NP_009193, XP_005263481 | PARK7 | Q99497 |
| Ras-related protein Rab-10 | NM_016131, XM_024452565 | NP_057215, XP_024308333 | RAB10 | P61026 |
| Ras-related protein Rap-2b | NM_002886 | NP_002877 | RAP2B | P61225 |
| RNA transcription, translation and transport factor protein | NM_016039 | NP_057123 | RTRAF | Q9Y224 |
| Crk-like protein | NM_005207 | NP_005198 | CRKL | P46109 |
| Serpin B6 | NM_001195291, NM_001271822, NM_001271823, NM_001271824, NM_001271825, NM_001297699, NM_001297700, NM_004568, XM_011514672, XM_011514674, XM_011514676, XM_017010940, XM_017010941, XM_024446462, XM_024446463, XM_024446464, XM_024446465 | NP_001182220, NP_001258751, NP_001258752, NP_001258753, NP_001258754, NP 001284628, NP_001284629, NP_004559, XP_011512974, XP_011512976, XP_011512978, XP_016866429, XP_016866430, XP_024302230, XP_024302231, XP_024302232, XP_024302233 | SERPINB6 | P35237 |
| 6-phosphogluconolactonase | NM_012088, XM_011527897 | NP_036220, XP_011526199 | PGLS | 095336 |
| Guanine nucleotide-binding protein G(I)/G(S)/G(T) subunit beta-1 | NM_001282538, NM_001282539, NM_002074, XM_017001059, XM_017001060, XM_017001061, XM_024446495 | NP_001269467, NP_001269468, NP_002065, XP_016856548, XP_016856549. XP_016856550, XP_024302263 | GNB1 | P62873 |
| Lactadherin | NM_001114614, NM_001310319, NM_001310320, NM_001310321, NM_005928, XM_017022206, XM_024449930 | NP_001108086, NP_001297248, NP_001297249, NP_001297250, NP_005919, XP_016877695, XP 024305698 | MFGE8 | 008431 |
| Ras-related protein Ral-A | NM_005402, XM_006715762, XM_011515466, XM_011515467 | NP_005393, XP_006715825, XP_011513768, XP_011513769 | RALA | P11233 |
| Protein transport protein Sec23A | NM_006364, XM_005267262, XM_011536355, XM_017020928 | NP_006355, XP_005267319, XP_011534657, XP_016876417 | SEC23A | Q15436 |
| Dihydropyrimidinase-related protein 2 | NM_001197293, NM_001244604, NM_001386 | NP_001184222, NP_001231533, NP_001377 | DPYSL2 | Q16555 |
| NCK-interacting protein with SH3 domain | NM_016453, NM_184231, XM_017006594, XM_017006595, XM_017006596, XM_017D06597; XM_017006598, XM_017006599, XM_017006600 | NP_C57537, NP_909119_{;} XP_016862083, XP_016862084, XP_016862085, XP_016862086, XP_016862087, XP_016862088, XP_016862089 | NCKIPSD | Q9NZQ3 |
| Vesicle-associated membrane protein 5 | NM_006634 | NP_006625 | VAMPS | 095183 |
| Protein phosphatase 1F | NM_014634, XM_011530556, XM_017029108 | NP_055449, XP_011528858, XP_016884597 | PPM1F | P49593 |
| Nuclear receptor corepressor 2 | NM_001077261, NM_001206654, NM_006312 | NP_001070729, NP_001193583, NP_006303 | NCOR2 | Q9Y618 |

TABLE 4-18

| | | | | |
|---|---|---|---|---|
| Ubiquitin-conjugating enzyme E2 D3 | NM_001300795, NM_003340, NM_181886, NM_181887, NM_181888, NM_181889, NM_181890, NM_181891, NM_181892, NM_181893, XM_017008584, XM_024454202 | NP_001287724, NP_003331, NP_871615, NP_871616, NP_871617, NP_871618, NP_871619, NP_871620, NP_871621, NP_871622, XP_016864073, XP_024309970 | UBE2D3 | P61077 |
| WW domain-binding protein 2 | NM_001330499, NM_001348170, NM_012478 | NP_001317428, NP_001335099, NP_036610 | WBP2 | Q969T9 |
| POTE ankyrin domain family member \| | NN_001277406, XM_011511670, XM_017004732, XM_017004733, XM_017004734, XM_024453046 | NP_001264335, XP_011509972, XP_016860221, XP_016860222, XP_016860223 XP_024308814 | POTEI | P0CG38 |
| Copine-1 | NM_001198863, NM_003915, NM_152925, NM_152926, NM_152927, NM_152928, NM_152930, NM_152931 | NP_001185792, NP_003906, NP_690902, NP_690903, NP_690904, NP_690905 | CPNE1 | Q99829 |
| Alpha-galactosidase A | NM_000169 | NP_000160 | GLA | P06280 |
| Coronin-1B | NM_001018070, NM_020441 | NP_001018080, NP_065174 | CORO1B | Q9BR76 |
| ATP-dependent RNA helicase DDX1 | NM_004939 | NP_004930 | DDX1 | Q92499 |
| Ubiquitin-conjugating enzyme E2 K | NM_001111112, NM_001111113, NM_001312646, NM_001312647, NM_001312648, NM_005339 | NP_001104582, NP_001104583, NP_001299575, NP_001299576, NP_001299577, NP_005330 | UBE2K | P61086 |
| Syntaxin-binding protein 1 | NM_001032221, NM_003165 | NP_001027392, NP_003156 | STXBP1 | P61764 |
| WD repeat-containing protein 37 | NM_014023 | NP_054742 | WDR37 | Q9Y21B |
| MOB kinase activator 1B | NM_001244766, NM_001244767, NM_173468, XM_005265709, XM_011532412, XM_017008837 | NP_001231695, NP_001231696, NP_775739, XP_005265766, XP_011530714, XP_016864326 | MOB1B | Q7L9L4 |
| Nodal modulator 3 | NM_001004067, XM_005255318 | NP_001004067, XP_005255375 | NOMO3 | P69849 |
| Nucleoside diphosphate kinase B | NM_001018137, NM_001018138, NM_001018139, NM_001198682, NM_002512, NM_001018136 | NP_001018147, NP_001018148, NP_001018149, NP_001186611, NP_002503, NP_001018146 | NME2 | P22392 |
| Prostaglandin reductase 1 | NM_001146108, NM_001146109, NM_012212, XM_011518394, XM_011518396, XM_017014485 | NP_001139580, NP_001139581, NP_036344, XP_011516696, XP_011516697, XP_016869974 | PTGR1 | Q14914 |
| Phosphoglycerate mutase 2 | NM_000290 | NP_000281 | PGAM2 | P15259 |
| TLD domain-containing protein 1 | NM_020947, XM_005256074, XM_005256075, XM_006721240, XM_011523248, XM_011523249, XM_017023510, XM_017023511, XM_017023512 | NP_065998, XP_005256131, XP_005256132, XP_006721303, XP_011521550, XP_011521551, XP_016878999, XP_016879000, XP_016879001 | TLDC1 | Q6P9B6 |
| Calcium-binding protein 39-like | NM_001079670, NM_001287337, NM_001287338, NM_001287339, NM_030925, XM_011535254, XM_011535255, XM_011535256, XM 011020785, XM_017020786, XM_017020787, XM_017020788 | NP_001073138, NP_001274266, NP_001274267, NP_001274268, NP_112187, XP_011533556, XP_011533557, XP_011533558, XP_016876274, XP_016876275, XP_016876276, XP_016876277 | CAB39L | Q9H9S4 |

TABLE 4-19

| | | | | |
|---|---|---|---|---|
| Glutathione S-transferase LANCL1 | NM_001136574, NM_001136575, NM_006055, XM_005246243 | NP_001130046, NP_001130047, NP_006046, XP_005246300 | LANCL1 | 043813 |
| Prefoldin subunit 6 | NM_001185181, NM_001265595, NM_001265596, NM_014260 | NP_001172110, NP_001252524, NP_001252525, NP_055075 | PFDN6 | 015212 |
| Cytosolic phospholipase A2 | NM_001311193, NM_024420, XM_005245267, XM_011509642 | NP_001298122, NP_077734, XP_005245324, XP_011507944 | PLA2G4A | P47712 |
| 14-3-3 protein zeta/delta | NM_001135699, NM_001135700, NM_001135701, NM_001135702, NM_003406, NM_145690, XM_005251061, XM_005251063, XM-011517290, XM_017013810, XM_017013811, XM_017013812, XM_024447266 | NP_001129171, NP_001129172, NP_001129173, NP_001129174, NP_003397, NP_663723, XP_005251118, XP_005251120, XP_011515592, XP_016869299, XP_016869300, XP_016869301, XP_024303034 | YWHAZ | P63104 |

Among the protein groups shown in Table 4-1 to Table 4-19 described above, for example, it is possible to preferably use one or more membrane proteins selected from the protein groups shown in Table 2 described above in the method of the present embodiment. As shown in the examples described below, the protein groups shown in Table 2 above are groups formed of 36 membrane proteins with an AUC value of the ROC curve, which is an indicator of discriminatory ability in determining drug sensitivity, of 0.8 or higher.

In addition, among the protein groups shown in Table 4-1 to Table 4-19 described above, for example, it is possible to preferably use one or more proteins selected from the protein groups shown in Table 3 described above in the method of the present embodiment. As shown in the examples described below, the protein group shown in Table 3 described above is a group consisting of 11 proteins with an AUC value of the ROC curve, which is an indicator of discriminatory ability in determining drug sensitivity, of 0.8 or higher and for which, for patients having drug sensitivity (responders), false negatives are not produced based on a threshold calculated by Youden index.

A detailed description will be given below of the method of the present embodiment.

Examples of the biological samples used in the method of the present embodiment include various fluids, such as body fluids, cell culture supernatants, and crushed tissue cell fluids. Among the above, body fluids or cell culture supernatants are preferable. In addition to whole blood, serum, plasma, various blood cells, blood clots, platelets, and the like, examples of body fluids include urine, semen, breast milk, sweat, interstitial fluid, interstitial lymph fluid, bone marrow fluid, tissue fluid, saliva, gastric fluid, joint fluid, pleural fluid, bile, ascites fluid, amniotic fluid, and the like. Among the above, plasma is preferable. Plasma may be treated with anticoagulants such as citric acid, heparin, and EDTA.

Biological samples may be pre-treated, such as by being added to a known buffer, and it is also possible to use samples ingested from a living organism as they are.

It is possible to appropriately adjust the amount of the sample, for example, the amount may be 1 µL or more and 1000 µL or less or, for example, the amount may be 1 µL or more and 500 µL or less.

Examples of methods for isolating exosomes from biological samples include a PEG precipitation method, an isolation method using an ultracentrifuge or the like, an immunoprecipitation method using molecules for exosome capture, and the like, without being limited thereto. Among the above, the immunoprecipitation method using molecules for exosome capture is convenient and enables the selective separation of exosomes without performing any pre-treatment and is thus preferable.

The molecules for exosome capture are formed of specific binding substances for antigens expressed on the exosome surface.

Examples of antigens expressed on the exosome surface include CD9, CD63, and CD81, without being limited thereto.

The molecules for exosome capture are formed of specific binding substances for these antigens.

Examples of specific binding substances include substances that bind specifically to the antigens. Examples of specific binding substances to antigens include antibodies and aptamers.

The antibodies may be any having a binding ability to antigenic sequences (epitopes) on the exosome surface and examples thereof include anti-CD9 antibodies, anti-CD63 antibodies, and anti-CD81 antibodies. These antibodies may be monoclonal antibodies or may be polyclonal antibodies, but monoclonal antibodies are preferable. In addition, it is also possible to use a mixture of a plurality of antibodies. Furthermore, these antibodies may be bispecific antibodies. Examples of the classes of antibodies include IgG and IgM and IgG is preferable. In addition, the antibodies may also be chimeric antibodies, such as humanized antibodies. Antibodies may be derived from animal species such as mammals such as humans, mice, rabbits, cattle, pigs, horses, sheep, goats, and the like, or birds such as chickens, without being particularly limited. Antibodies may, for example, be prepared from serum derived from the animal species described above by methods known in the related art, antibodies synthesized from known antibody gene sequence information may be used, or commercially available antibodies may be used.

It is also possible to use antibody fragments instead of antibodies. Examples of antibody fragments include Fv, Fab, Fab′, F(ab′)2, rIgG, and scFv.

Aptamers are substances having a specific binding ability with respect to a target substance. Examples of aptamers include nucleic acid aptamers, peptide aptamers, and the like. It is possible to sort nucleic acid aptamers having a specific binding ability to antigens, for example, by a systematic evolution of ligands by exponential enrichment (SELEX) method. In addition, it is possible to sort peptide aptamers having a specific binding ability to antigens, for example, by the Two-Hybrid method using yeast.

The molecules for exosome capture are preferably fixed on a solid-phase carrier.

Examples of the substances of solid-phase carriers include polymer compounds such as polystyrenes, polyethylenes, polypropylenes, polyesters, poly(meth)acrylonitrile, styrene-butadiene copolymers, poly(meth)acrylic esters, fluorine resins, cross-linked dextrans, and polysaccharides; glass; metals; magnetic materials; resin compositions including magnetic materials; and combinations thereof.

In addition, the shape of the solid-phase carrier is not particularly limited and examples thereof include tray shapes, sphere shapes, particle shapes, fiber shapes, rod shapes, plate shapes, container shapes, cells, microplates, microfluidic devices, and test tubes.

In this step, magnetic particles are preferable.

Examples of the magnetic particles described above include metals such as iron tetroxide (Fe₃O₄), iron sesquioxide (γ-Fe₂O₃), various ferrites, iron, manganese, nickel, cobalt, and chromium; magnetic material fine particles formed of alloys of cobalt, nickel, manganese, and the like; and magnetic particles including these magnetic materials in a resin. Examples of the resins include hydrophobic polymers, hydrophilic polymers, and the like.

Among the above, magnetic particles including magnetic materials in resin are preferable and magnetic particles in which a polymer layer is formed on the surface of a mother particle including superparamagnetic fine particles are more preferable. Examples thereof include magnetic particles in which, as described in Japanese Unexamined Patent Application, First Publication No. 2008-32411 (Patent Document 1), a hydrophobic first polymer layer is formed on the surface of a mother particle including superparamagnetic fine particles, a second polymer layer having at least a glycidyl group on the surface thereof is formed on the first polymer layer, and polar groups are introduced by chemical modification of the glycidyl group.

Here, as superparamagnetic fine particles, iron oxide-based fine particles with a particle diameter of 20 nm or less (preferably a particle diameter of 5 nm or more and 20 nm or less) are representative and examples thereof include ferrite expressed as XFe₂O₄ (X = M n, Co, Ni, Mg, Cu, Li_{0.5}Fe_{0.5}, and the like), magnetite expressed as Fe₃O₄, and y Fe2O3, and, in terms of strong saturation magnetization and low residual magnetization, including either γ-Fe2O₃ or Fe₃O₄, is preferable.

In addition, the monomers for forming the hydrophobic first polymer layer described above are broadly classified as monofunctional monomers and cross-linking monomers.

Examples of the monofunctional monomers described above include aromatic vinyl monomers such as styrene, α-methylstyrene, and halogenated styrene; and ethylenically unsaturated carboxylic acid alkyl ester monomers such as methyl acrylate, methyl methacrylate, ethyl acrylate, ethyl methacrylate, stearyl acrylate, stearyl methacrylate, cyclohexyl acrylate, cyclohexyl methacrylate, isobonyl acrylate, and isobonyl methacrylate.

In addition, examples of the cross-linking monomers described above include polyfunctional (meth)acrylates such as ethylene glycol diacrylate, ethylene glycol dimethacrylate, trimethylolpropane triacrylate, trimethylolpropane trimethacrylate, pentaerythritol triacrylate, pentaerythritol trimethacrylate, dipentaerythritol hexaacrylate, and dipentaerythritol hexamethacrylate; conjugated diolefins such as butadiene and iprene, as well as divinylbenzene, diarylphthalate, allyl acrylate, and allyl methacrylate.

In addition, the monomers for forming the second polymer layer described above are mainly intended for introducing functional groups to the particle surface and include glycidyl group-containing monomers. As for the content of the glycidyl group-containing monomer, 20% by mass or more in the monomer for forming the second polymer layer is preferable. Here, examples of copolymerizable monomers including glycidyl groups include glycidyl acrylate, glycidyl methacrylate, and allyl glycidyl ether.

The polar group introduced by chemical modification of the glycidyl group in the second polymer layer is preferably a functional group capable of reacting with specific binding substances for antigens expressed on the surface of cancer cells and is more preferably a polar group including at least one or more atoms selected from the group consisting of oxygen atoms, nitrogen atoms, and sulfur atoms. Among the above, amino groups, aldehyde groups, carboxy groups, or active ester groups are more preferable. In particular, in a case where the second polymer layer of the magnetic particles has the polar group and 2,3-dihydroxypropyl group, the binding property to specific binding substances for antigens expressed on the exosome surface (in particular, antibodies for antigens expressed on the exosome surface) is favorable.

As methods for binding specific binding substances for antigens expressed on the exosome surface to solid-phase carriers, it is possible to use physical adsorption methods, chemical binding methods such as covalent binding methods and ionic binding methods, and the like. Examples of physical adsorption methods include a method for directly fixing the specific binding substance for the antigen expressed on the exosome surface on the solid-phase carrier, a method for chemically binding to another protein such as albumin and then fixing by adsorption, and the like. Examples of chemical binding methods include a method for direct binding on a solid-phase carrier using a functional group introduced into the surface of the solid-phase carrier which is able to react with the specific binding substance for the antigen expressed on the exosome surface, a method for introducing and binding spacer molecules (such as a carbodiimide compound) between the solid-phase carrier and the specific binding substance for the antigen expressed on the exosome surface by chemical binding, a method for binding a specific binding substance for the antigen expressed on the exosome surface to another protein such as albumin and then carrying out chemical binding of the protein to the solid-phase carrier, and the like.

Immunoprecipitation methods using molecules for exosome capture may be performed using salts, proteins such as albumin, surfactants, and the like as necessary, in addition to each of the components described above.

The reaction temperature in the immunoprecipitation method using molecules for exosome capture is usually 2° C. or higher and 42° C. or lower and the reaction time is usually 5 minutes or more and 24 hours or less. In a case where the reaction is performed at 2° C. or higher and 8° C. or lower, the reaction time is preferably 8 hours or more and 30 hours or less and, in a case where the reaction is carried out at 8° C. or higher and 42° C. or lower, the reaction time is preferably 5 minutes or more and 24 hours or less.

In the immunoprecipitation method using molecules for exosome capture, the pH in the system is not particularly limited, but is preferably in a range of pH 5 or more and 10 or less and more preferably in a range of pH 6 or more and 8 or less. To maintain the desired pH, buffer solutions are usually used, for example, a phosphate buffer solution, a tris(hydroxymethyl) aminomethane buffer solution, a HEPES buffer solution, and an MES buffer solution may be used.

In addition, in a case where one or more types of solid-phase carrier with fixed molecules for exosome capture are used, the usage amount and the mixing ratio are not particularly limited, but 0.00001 to 10 w/v% with respect to the total amount of the liquid phase in the system is preferable, 0.00001 to 5 w/v% is more preferable, and 0.001 to 0.1 w/v% is even more preferable.

Next, a mass spectrometry method is used to determine the expression level of the proteins present in the isolated exosomes.

The proteins for which the expression levels are to be determined are one or more proteins selected from the group consisting of the groups of proteins shown in Table 4-1 to Table 4-19 described above.

In a case where a mass spectrometry method is used, a pre-treatment may be performed to separate the substances present in the exosomes before mass spectrometry. It is possible to use various chromatographic separation techniques as separation methods, more specifically, examples thereof include liquid chromatography (LC), high-performance liquid chromatography (HPLC), gas chromatography (GC), thin layer chromatography, and size exclusion chromatography, without being limited thereto. Among the above, liquid chromatography or high-performance liquid chromatography is preferable.

Examples of mass spectrometry methods include a tandem quadrupole mass spectrometry (MS/MS) method, a direct injection mass spectrometry method, a Fourier transform ion cyclotron resonance mass spectrometry (FT-ICR-MS) method, a capillary electrophoresis mass spectrometry (CE-MS) method, an inductively coupled plasma mass spectrometry (ICP-MS) method, a pyrolysis mass spectroscopy (Py-MS) method, an ion mobility mass spectrometry method, a time-of-flight mass spectrometry (TOF MS) method, and the like.

Among the above, as a mass spectrometry method, a mass spectrometry method coupled with the separation methods described above is preferable and examples thereof include a GC-MS method, a LC-MS method, a HPLC-MS method, a LC-MS/MS method, and a HPLC-MS/MS method, without being limited thereto.

It is possible to determine protein expression levels, for example, by analysis using a DIA (data-independent acquisition) method in a mass spectrometry method. The DIA method is a data-independent analysis method, which is an exhaustive analysis method that analyzes all ions included in a certain mass range by MS/MS analysis. Among the above, the SWATH method, which is one DIA method, for example, repeats an operation of acquiring MS/MS 10 times while shifting a 100 Da width window and records the MS/MS spectra of all ions within the total 1,000 Da width (the window width and number of acquisitions are variable).

The method of the present embodiment predicts the efficacy of treatment using an immune checkpoint inhibitor for lung cancer patients from whom the biological sample which is the evaluation target is derived, based on the determined protein expression levels.

Specifically, in a case where the protein expression level is a predetermined reference value or less, it is possible to evaluate or predict that the lung cancer patient from whom the biological sample which is the evaluation target is derived is potentially sensitive to the immune checkpoint inhibitor, that is, that the treatment with the immune checkpoint inhibitor is potentially effective. On the other hand, in a case where the protein expression level is over a predetermined reference value, it is possible to evaluate or predict that the lung cancer patient from whom the biological sample which is the evaluation target is derived is potentially not sensitive to the immune checkpoint inhibitors, that is, that the treatment with the immune checkpoint inhibitor is potentially not effective. The reference value is for distinguishing between a group of lung cancer patients who are sensitive to immune checkpoint inhibitors (responder group) and a group of lung cancer patients who are not sensitive to immune checkpoint inhibitors (non-responder group).

It is possible to experimentally determine the reference value, for example, as a threshold value able to distinguish between the two groups by measuring the expression levels of a specific protein present in the exosomes of the responder groups and non-responder groups. The method for determining the reference value for the expression level of a specific protein in the method of the present embodiment is not particularly limited and determination is possible, for example, using general statistical methods. Here, the specific protein means one or more proteins selected from the group of proteins shown in Tables 4-1 to 4-19 above.

Specifically, as a method for determining the reference value, for example, the expression levels of a specific protein present in exosomes in biological samples collected in advance before administration of an immune checkpoint inhibitor are measured for patients diagnosed as having lung cancer by a commonly performed method such as imaging diagnosis. After measurement for a plurality of patients, the expression levels of the specific proteins present in the exosomes in these biological samples are calculated from the mean values, the median values, or the like and it is possible to use values included therein as reference values. In addition, it is also preferable that the reference value is described in the instructions or the like of the kit relating to the evaluation or prediction method.

In addition, for a plurality of responder groups and a plurality of non-responder groups, the expression levels of a specific protein present in exosomes in biological samples collected in advance before administration of the immune checkpoint inhibitor are measured, respectively, and, from the mean values, the median values, or the like, the expression levels of the specific protein present in exosomes in biological samples of the responder group and non-responder group and variations thereof are each calculated, then, a threshold value able to distinguish between the two values, also taking into account variations, is determined and it is possible to use this as the reference value.

### Other Embodiments

In one embodiment, the present invention provides a biomarker for predicting or diagnosing the efficacy of treatment of a lung cancer patient using an immune checkpoint inhibitor, which is formed of one or more proteins selected from the protein groups shown in Tables 1-1 to 1-6 described above.

In one embodiment, the present invention provides a biomarker for predicting or diagnosing the efficacy of treatment of a lung cancer patient using an immune checkpoint inhibitor, which is formed of one or more membrane proteins selected from the group of proteins shown in Table 2 described above.

In one embodiment, the present invention provides a biomarker for predicting or diagnosing the efficacy of treatment of a lung cancer patient using an immune checkpoint inhibitor, which is formed of one or more proteins selected from the group of proteins shown in Table 3 described above.

In one embodiment, the present invention provides a method for diagnosing the efficacy of treatment of a lung cancer patient using an immune checkpoint inhibitor, that is, a method for companion diagnosis using an immune checkpoint inhibitor for a lung cancer patient, the method including isolating exosomes from a biological sample derived from the lung cancer patient, and determining an expression level of a protein present in the exosomes by a mass spectrometry method, in which the protein is one or more proteins selected from the groups of proteins shown in Table 1-1 to Table 1-6 described above.

In one embodiment, the present invention provides a method for diagnosing the efficacy of treatment of a lung cancer patient using an immune checkpoint inhibitor, that is, a method for companion diagnosis using an immune checkpoint inhibitor for a lung cancer patient, the method including isolating exosomes from a biological sample derived from the lung cancer patient, and determining an expression level of a protein present in the exosomes by a mass spectrometry method, in which the protein is one or more proteins selected from the group of proteins shown in Table 2 above.

In one embodiment, the present invention provides a method for diagnosing the efficacy of treatment of a lung cancer patient using an immune checkpoint inhibitor, that is, a method for companion diagnosis using an immune checkpoint inhibitor for a lung cancer patient, the method including isolating exosomes from a biological sample derived from the lung cancer patient, and determining an expression level of a protein present in the exosomes by a mass spectrometry method, in which the protein is one or more proteins selected from the group of proteins shown in Table 3 described above.

### EXAMPLES

A description will be given below of the present invention using Examples, but the present invention is not limited to the following Examples.

### Example 1

### 1. LC-MS Analysis of Exosomes

### Preparation of Biological Samples

EDTA plasma (before administration of nivolumab, Pembrolizumab, or atezolizumab) from 20 Japanese lung cancer patients was used in the following test. For the lung cancer patient group, according to RECIST guidelines, after administration of nivolumab (12 patients), pembrolizumab (6 patients), or atezolizumab (2 patients), 10 patients determined to be partial response (PR) (5 patients administered with nivolumab, 4 patients administered with pembrolizumab, and 1 patient administered with atezolizumab) were set as a responder group and 3 patients determined to be stable (SD) (3 patients administered with nivolumab) and 7 patients determined to be progressive (PD) (4 patients administered with nivolumab, 2 patients administered with pembrolizumab, and 1 patient administered with atezolizumab) were set as a non-responder group.

### Antibody-Bound Particles for Exosome Capture

Exosome-capturing antibodies (Anti-CD9 mAb (MEX001-3 manufactured by MBL Co., Ltd.), Anti-CD63 (LAMP-3) mAb (MEX002-3 manufactured by MBL Co., Ltd.), or Anti-CD81 (TAPA1) mAb (MEX003-3 manufactured by MBL Co., Ltd.)) were bound to magnetic particles (Magnosphere™ MS300/Carboxyl manufactured by JSR Life Sciences Corporation) to produce three types of antibody-bound particles. Next, the three types of antibody-bound particles were mixed in equal amounts and a solution with a final particle concentration of 0.06% by mass was used as the antibody-bound particle solution for the following tests.

### Isolation of Exosomes

Freeze-thawed EDTA plasma was filtered through a membrane filter with a pore diameter of 0.22 µm (SLGV033RS manufactured by Merck Millipore) and 0.5 mL thereof was added to a 15 mL low-adsorption tube (MS-52150 manufactured by Sumitomo Bakelite Co., Ltd.). Next, 4.5 mL of TRS was added and diluted 10-fold and 5 mL of the antibody-bound particle solution was added and then stirred overnight at room temperature.

The antibody-bound particles after the reaction were transferred to a 2 mL low-adsorption tube (MS-4220M manufactured by Sumitomo Bakelite Co., Ltd.) and washed four times with a washing solution (ExoCap™ Ultracentrifugation/Storage Booster (MEX-USB manufactured by MBL Co., Ltd.). The antibody-bound particles were transferred to a new 2 mL low-adsorption tube and washed twice with TBS. Thereafter, 50 µL of a 2% by mass sodium dodecyl sulfate (SDS) solution was added and shaken for 10 minutes at 25° C. The supernatant after the reaction was added to a 100 K ultrafiltration spin column (OD100 C34 manufactured by PALL), centrifuged at 10,000 x g for 5 minutes and then the filtrate was recovered in a 2 mL low-adsorption tube and used as an exosome extract.

### LC-MS/MS Analysis of Exosome Proteins

25 µL of the exosome extract was transferred to a 1.5 mL tube (0030123328 manufactured by Eppendorf) and an 8-fold volume of cold acetone was added thereto and allowed to stand at -20° C. for 2 hours. Next, after centrifugation at 15,000 x g for 15 minutes at 4° C., 20 µL of a 0.5% by mass sodium dodecanoate-containing 100 mM Tris (pH 8.5) solution was added to the precipitate and the protein was dissolved by a sealed ultrasonic crusher. To cleave the S-S bonds of the protein, dithiothreitol (DTT) was added to a final concentration of 10 mM and allowed to react at 50° C. for 30 min. To alkylate the cysteine residues, iodoacetamide was added to a final concentration of 30 mM and allowed to react for 30 minutes at room temperature under light-shielding conditions. To stop the reaction, cysteine was added to a final concentration of 60 mM and allowed to stand for 10 minutes at room temperature. Thereafter, 150 µL of 50 mM ammonium bicarbonate was added thereto, then 250 ng of Lys-C (125-05061 manufactured by Fujifilm Wako Pure Chemical Corporation) and 250 ng of trypsin (V5113 manufactured by Promega) were added and reacted overnight at 37° C. to break down the protein into peptides. After acidifying the solution by adding 5% by mass of trifluoroacetic acid (TFA) to remove sodium dodecanoate, the result was centrifuged at 15,000 x g for 10 minutes at room temperature and the supernatant was recovered. After desalting using a C18 column, the result was dried and solidified using a centrifugal evaporator, a solution containing 3% by mass acetonitrile-0.1% by mass formic acid was added thereto, the peptides were dissolved in a sample sealed ultrasonic crusher and LC-MS analysis was performed. LC-MS analysis was performed using the Q Exactive™ HF-X, manufactured by Thermo Fisher Scientific and Scaffold DIA software to identify proteins and calculate relative quantification values.

### 2. Calculation of Area Under Curve (AUC) of Receiver Operating Characteristic (ROC)

For the proteins analyzed by the Scaffold DIA software in “1. (4)”, proteins where the Protein FDR was 1% or less were extracted and normalization of the relative quantification values was performed after excluding the immunoglobulin groups and keratin groups, which were considered to be contaminants. 2406 proteins were identified from lung cancer patients. For these proteins, Scikit-Learn, which is an open-source library of the Python programming language, was used to calculate the AUC as an indicator of discriminatory ability and a method using the Youden index was used to calculate the threshold for discriminating between the two groups. The 825 proteins with AUC values of 0.7 or higher and the AUC values thereof are shown in Table 5-1 to Table 5-54. The amino acid sequence of ‘Putative nucleoside diphosphate kinase’ in Table 5-19 is shown in Sequence No. 1.

TABLE 5-1

| AUC Rank | AUC | Protein name | Gene name | Uniprot Ac. | RefSeq_NM | RefSeq_NP |
|---|---|---|---|---|---|---|
| 1 | 0.96 | Band 4.1-like protein 2 | EPB41L2 | 043491 | NM_001135554, NM_001135555, NM_001199388, NM_001199389, NM_001252660, NM_001350299, NM_001350301, NM_001350302, NM_001350303, NM_001350304, NM_001350305, NM_001350306, NM_001350307, NM_001350308, NM_001350309, NM_001350310, NM_001350311, NM_001350312, NM_001350313, NM_001350314, NM_001350315, NM_001350320, NM_001431, XM_011535524, XM_011535525, XM_011535527, XM_011535528, XM_017010350, XM_017010351, XM_017010352, XM_017010353, XM_017010356, XM_017010364, XM_024446349 | NP_001129026, NP_001129027, NP_001186317, NP_001239589, NP_001337228, NP_001337230, NP_001337231, NP_001337232, NP_001337233, NP 001317234, NP_001337235, NP_001337236, NP_001337237, NP_001337238, NP_001337239, NP_001337240, NP_001337241, NP_001337242, NP_001337243, NP_001337244, NP_001337249, NP_001422, XP_011533826, XP_011533827, XP_011533829, XP_011533830, XP_016865839, XP_016865840, XP_016865841, XP_016865842, XP_016865845, XP_016865853, XP_024302117 |
| 2 | 0.94 | Cullin-2 | CUL2 | Q13617 | NM_001198777, NM_001198778, NM_001198779, NM_001324375, NM_001324376, NM_003591, XM_011519743, XM_011519744, XM_011519745, XM_011519747 | NP_001185706, NP_001185707, NP_001185708, NP_001311304, NP_001311305, NP_003582, XP_011518045, XP_011518046, XP_011518047, XP_011518049 |
| 3 | 0.94 | Protein Hikeshi | HIKESHI | Q53FT3 | NM_001322404, NM_001322407, NM_001322409, NM_016401, XM_017017914, XM_017017915 | NP_001309333, NP_001309336, NP_001309338, NP_057485, XP_016873403, XP_016873404 |
| 4 | 0.93 | Hippocalcin-like protein 1 | HPCAL1 | P37235 | NM_001258357, NM_001258358, NM_001258359, NM_002149, NM_134421, XM_005246160, XM_005246161, XM_005246162, XM_005246163, XM_011510346, XM_011510347, XM_011510348, XM_011510349, XM_017003950, XM_017003951, XM_017003952, XM_017003953 | NP_001245286, NF_001245287, NP_001245288, NP_002140, NP_602293, XP_005246217, XP_005246218, XP_005246219, XP_005246220, XP_011508648, XP_011508649, XP_011508650, XP_011508651, XP_016859439, XP_016859440, XP_016859441, XP_016859442, |
| 5 | 0.92 | UTP-glucose-1-phosphate uridylyltransferase | UGP2 | Q16851 | NM_001001521, NM_006759, XM_005264537, XM_005264538, XM_006712088, XM_011533091, XM_017004855, XM_017004856, XM_017004857, XM_024453120, XM_024453121, XM_024453122, XM_024453123, XM_024453124 | NP_001001521, NP_006750, XP_005264594, XP_005264595, XP_006712151, XP_011531393, XP_016860344, XP_016860345, XP_016860346, XP_024308888, XP_024308889, XP_024308890, XP_024308891, XP_024308892 |
| 6 | 0.92 | Attractin | ATRN | 075882 | NM_001207047, NM_001323332, NM_012070, NM_139321, NM_139322 | NP_001193976, NP_001310261, NP_647537, NP_647538 |
| 7 | 0.91 | Annexin A3 | ANXA3 | P12429 | NM_005139 | NP_005130 |

TABLE 5-2

| | | | | | | |
|---|---|---|---|---|---|---|
| 8 | 0.90 | Rap1 GTPase-activating protein 2 | RAP1GAP2 | Q684P5 | NM_001100398, NM_001330058, NM_015085, XM_005256542, XM_011523738, XM_011523739, XM_011523741, XM_011523742, XM_011523743, XM_011523744, XM_011523745, XM_017024371, XM_024450659 | NP_001093868, NP_001316987, NP_055900, XP_005256599, XP_011522040, XP_011522041, XP_011522043, XP_011522044, XP_011522045, XP_011522046, XP_011522047, XP_016879860, XP_024306427 |
| 9 | 0.89 | Radixin | RDX | P35241 | NM_001260492, NM_C01260493, NM_001260494, NM_001260495, NM_001260496, NM_002906 | NP_001247421, NP_001247422, NP_001247423, NP_001247424, NP_001247425, NP_002897 |
| 10 | 0.89 | Annexin A5 | ANXA5 | P08758 | NM_001154, XM_017008141 | NP_001145, XP_016863630 |
| 11 | 0.89 | ADP-ribosylation factor 5 | ARF5 | P84085 | NM_001662 | NP_001653 |
| 12 | 0.89 | L-lactate dehydrogenase B chain | LDHB | P07195 | NM_001174097, NM_001315537, NM_002300, XM_006719074 | NP_001167568, NP_001302466, NP_002291, XP_006719137 |
| 13 | 0.89 | Rho GTPase-activating protein 1 | ARHGAP1 | Q07960 | NM_004308, XM-011520095, XM_024448520 | NP_004299, XP_011518397, XP_024304288 |
| 14 | 0.89 | Drebrin | DBN1 | Q16643 | NM_001363541, NM_001364151, NM_001364152, NM_004395, NM_080881, XM_005265827, XM_006114826, XM_011534446, XM_011534447, XM_017009139, XM_017009140 | NP_001350470,NP_001351080, NP_001351081, NP_004386, NP_543157, XP_005265884, XP_006714889, XP_011532748, XP_011532749, XP_016864628, XP_016864629 |
| 15 | 0.89 | Serne/threonine-protein kinase PAK 2 | PAK2 | Q13177 | NM_002577, XM_011512870, XM_017006501 | NP_002568, XP_011511172, XP_016861990 |
| 16 | 0.88 | Protein BRICK1 | BRK1 | Q8WUW1 | NM_018462 | NP_060932 |
| 17 | 0.88 | Integrin alpha-V | ITGAV | P06756 | NM_001144999, NM_001145000, NM_002210 | NP_001138471, NP_001138472, NP_002201 |
| 18 | 0.88 | Protein kinase C and casein kinase substrate in neurons protein 2 | PACSIN2 | Q9UNF0 | NM_001184970, NM_001184971, NM_001349968, NM_ 001349969, NM_001349970, NM_001349971, NM_001349972, NM_001349973, NM_001349974, NM_007229, XM_005261319, XM_011529847, XM_011529848, XM_011529849, XM_011529850, XM_011529852, XM_017028562, XM_017028563, XM_017028564 | NP_001171899, NP_001171900, NP_001336897, NP_001336898, NP_001336899, NP_001336900, NP_001336901, NP_001336902, NP_001336903, NP_009160, XP_005261376X XP_011528149, XP_011528150, XP_011528151, XP_011528152, XP_011528154, XP_016884051, XP_016884052, XP_016884053 |
| 19 | 0.88 | Triokinase/FMN cyclase | TKFC | Q3LXA3 | NM_001351976, NM_001351977, NM_001351978, NM_001351979, NM_001351980, NM_015533, XM_011544912, XM_017017517, XM_017017518, XM_017017519, XM_017017520 | NP_001338905, NP_001338906, NP_001338907, NP_001338908, NP_001338909, NP_056348, XP_011543214, XP_016873006, XP_016873007, XP_016873008, XP_016873009 |

TABLE 5-3

| | | | | | | |
|---|---|---|---|---|---|---|
| 20 | 0.88 | Adhesion G-protein coupled receptor G1 | AD6RG1 | Q9Y653 | NM_001145770, NM_001145771, NM_001145772, NM_001145773, NM_001145774, NM_001290142, NM_001290143, NM_001290144, NM_005682, NM_201524, NM_201525, XM_005256237, XM_005256238, XM_005256239, XM_005256240, XM_005256241, XM_005256242, XM_005256244, XM_005256245, XM_005256246, XM_005256247, XM_005256248, XM_005256249, XM_005256251, XM_005256252, XM_005256254 XM_005256255, XM_006721338 XM_006721339, XM_006721340, XM_006721341, XM_006721342, XM_006721343, XM_006721344, XM_006721345, XM_006721346, XM_006721347, XM_011523461, XM_011523462, XM_011523463, XM_011523464, XM_011523465, XM_011523466, XM_011523467 XM_011523468, XM_017023892 | NP_001139242, NP_001139243, NP_001139244, NP_001139245, NP_001139246, NP_001277071, NP_001277072, NP_001277073, NP_005673, NP_958932, NP_958933, XP_005256294, XM_005256295, XM_005256296, XP_005256297, XP_005256298, XP_005256299, XP_005256301, XP_005256302, XP_005256303, XP_005256304, XP_005256305, XP_005256306, XP_005256308, XP_005256309, XP_005256311, XP_005256312, XP_006721401, XP_006721402, XP_006721403, XP_006721404, XP_006721405, XP_006721406, XP_006721407, XP_006721408, XP_006721409, XP_006721410, XP_01152763, XP_011521764, XP_011521765, XP_01521766, XP_011521767, XP_011521768, XP_011521769, XP_011521770, XP_016879381 |
| 21 | 0.88 | Integrin beta-5 | ITGB5 | P18084 | NM_001354764, NM_001354765, NM_001354766, NM_002213, XM_005247436, XM_06713630, XM_017006353, XM_017006354, XM_017006355 | NP_001341693, NP_001341694, NP_001341695, NP_002204, XP_005247493, XP_006713693, XP_016861842, XP_016861843, XP_016861844 |
| 22 | 0.88 | Isochorismatasedomain-containing protein 1 | ISOC1 | Q96CN7 | NM_016048 | NP_057132 |

TABLE 5-4

| | | | | | | |
|---|---|---|---|---|---|---|
| 23 | 0.88 | Septin-2 | SEPT2 | Q15019 | NM_001008491, NM_001008492, NM_001282972, NM_001282973, NM_001321029, NM_001321030, NM_001321031, NM_001321032, NM_001321033, NM_001321034, NM_001321035, NM_001349287, NM_001349288, NM_001349289, NM_001349290, NM_001349291, NM_001349302, NM_001349304, NM_001349305, NM_001349306, NM_001349307, NM_001349308, NM_001349309, NM_001349310, NM_001349311, NM_001349312, NM_001349313, NM_001349314, NM_001349315, NM_004404, NM_006155, XM_017004205, XM_017004212, XM_017004213, XM_024452920, XM_024452921, XM_024452922, XM_024452923, XM_024452924, XM_024452925, XM_024452926 | NP_001008491, NP_001008492, NP_001269901, NP_001269902, NP_001307958, NP_001307959, NP_001307960, NP_001307961, NP_001307962, NP_001307963, NP_001307964, NP_001336216, NP_001336217, NP_001336218, NP001336219, NP_001336220, NP_001336231, NP_001336233, NP_001336234, NP_001336235, NP_001336236, NP_001336237, NP_001336238, NP_001336239, NP_001336240, NP_001336241, NP_001336242, NP_001336243, NP_001336244, NP_004395, NP_006146, XP_016859694, XP_016859701, XP_0168597022, XP_024308688, XP_024308689, XP_024308690, XP_0024308691, XP_024308692, XP_024308693, XP_024308694 |
| 24 | 0.88 | Hydroxyacyl-coenzymeA dehydrogenase, milochondrial | HADH | Q16836 | NM_001184705, NM_001331027, NM_005327 | NP_001171634, NP_001317956, NP_005318 |
| 25 | 0.88 | Serne/threonine-protein kinase 38 | STK38 | Q15208 | NM_001305102, NM_007271, XM_006714988, XM_006714989, XM_017010226 | NP_001292031, NP_009202, XP_006715051, XP_006715052, XP_01685715 |
| 26 | 0.88 | Transient receptorpotential cation channel subfamily M member 4 | TRPM4 | Q8TD43 | NM_001195227, NM_001321281, NM_001321282, NM_001321283, NM_001321285, NM_017636, XM_005259017, XM_024451557 | NP_001182156, NP_001308210, NP_001308211, NP_001308212, NP_001308214, NP_060106, XP_005259074, XP_024307325 |
| 27 | 0.88 | Cytoskeleton-associated protein 5 | CKAP5 | Q14008 | NM_001008938, NM_014756 | NP_001008938, NP_055571 |
| 28 | 0.88 | FAS-associated factor 1 | FAF1 | Q9UNN5 | NM_007051, NM_131917, XM_017000136, XM_024452734, XM_024452736 | NP_008982, XP_016855625, XP_024308502, XP_024308504 |
| 29 | 0.88 | Insulin receptor | INSR | P06213 | NM_00208, NM_001079817, XM_011527988, XM_011527989 | NP_000199, NP_001073285, XP_011526290, XP_011526291 |
| 30 | 0.87 | Tropomyosin alpha-4 chain | TPM4 | P67936 | NM_001145160, NM_003290, XM_005260042, XM_006722865, XM_011528231, XM_011528232, XM_017027188, XM_017027189, XM_024451681 | NP_001138632, NP_003281, XP_005260099, XP_006722928, XP_011526533, XP_011526534, XP_016882677, XP_016882678, XP_0224307449 |
| 31 | 0.87 | Programmedcell death protein 6 | PDCD6 | 075340 | NM_001267556, NM_001267557, NM_001267558, NM_001267559, NM_013232 | NP_001254485, NP_001254486, NP_001254487, NP_001254488, NP_037364 |
| 32 | 0.87 | Leukocyte elastase inhibitor | SERPINB1 | P30740 | NM_030666, XM_011514333, XM_011514334, XM_011514335 | NP_109591, XP_011512635, XP_011512636, XP_011512637 |
| 33 | 0.87 | ADP-ribosylation factor 6 | ARF6 | P62330 | NM_001663 | NP_001654 |
| 34 | 0.87 | Complement component C9 | C9 | P02748 | NM_001737 | NP_001728 |
| 35 | 0.87 | ATP-dependent (S)-NAD(P)H-hydrate dehydratase | NAXD | Q8W45 | NM_001242881, NM_001242882, NM_001242883, NM_018210 | NP_001229810, NP_001229811, NP_001229812, NP_060680 |
| 36 | 0.87 | Actin, alpha cardiac muscle 1 | ACTC1 | P68032 | NM_005159 | NP_005160 |
| 37 | 0.87 | Heat shock 70 kDa protein 4 | HSPA4 | P34932 | NM_002154, NM_198431 | NP_002145 |

TABLE 5-5

| | | | | | | |
|---|---|---|---|---|---|---|
| 38 | 0.87 | 14-3-3 protein theta | YWHAQ | P27348 | NM_006826 | NP_006817 |
| 39 | 0.87 | Phosphatidylinositol transfer protein beta isoform | PITPNB | P48739 | NM_001284277, NM_001284278, NM_012399, XM_011530052, XM_017028707 | NP_001271206, NP_001271207, NP_036531, XP_011528354, XP_016884196 |
| 40 | 0.87 | Phosphoribosyltransferase domain-containing protein 1 | PRTFDC1 | Q9NRG1 | NM_001282786, NM_020200 | NP_001269715, NP_064585 |
| 41 | 0.86 | Costars family protein ABRACL | ABRACL | Q9P1F3 | NM_021243, XM_017011151 | NP_067066, XP_016866640 |
| 42 | 0.86 | Ubiquitin-conjugating enzyme E2 L5 | UBE2L5 | ACA1B0GUS4 | NM_001355247 | NP_001342176 |
| 43 | 0.86 | Sodium/potassium-transporting ATPase subunit alpha-1 | ATP1A1 | P05023 | NM_000701, NM_001001586, NM_001160233, NM_001160234, XM_017001360, XM_017001361 | NP_000692, NP_001153705, NP_001153706, XP_016856849, XP_016856850 |
| 44 | 0.86 | 14-3-3 protein epsilon | YWHAE | P62258 | NM_006761, XM_005256784, XM_017025005 | NP_006762, XP_005256841, XP_016880494 |
| 45 | 0.86 | Major prion protein | PRNP | P04156 | NM_000311, NM_001080121, NM_001080122, NM_001080123, NM_001271561, NM_183079 | NP_000302, NP_001073590, NP_001073591, NP_001073592, NP_001258490, NP_898902 |
| 46 | 0.86 | F-actin-capping protein subunit alpha-2 | CAPZA2 | P47755 | NM_006136 | NP_006127 |
| 47 | 0.86 | HLAclass \| histocompatibility antigen, Cw-4 alpha chain | HLA-C | P30504 | NM_001243042, NM_002117 | NP_001229971, NP_002108 |
| 48 | 0.86 | GTP-binding protein SAR1a | SAR1A | Q9NR31 | NM_001142648, NM_020150 | NP_001136120, NP_064535 |
| 49 | 0.86 | Actin-related protein ⅔ complex subunit 5 | ARPC5 | C15511 | NM_001270439, NM_005717 | NP_001257368, NP_005708 |
| 50 | 0.86 | Glutathione reductase, mitochondrial | GSR | P00390 | NM_000637, NM_001195102, NM_001195103, NM_001195104 | NP_000628, NP_001182031, NP_001182032, NP_001182033 |
| 51 | 0.86 | Guanine nucleotide-binding protein subunit alpha-11 | GNA11 | P29992 | NM_002067 | NP_002058 |
| 52 | 0.86 | Protein disulfide-isomerase A5 | PD/A5 | Q14554 | NM_006810 | NP_006801 |
| 53 | 0.86 | MARCKS-related protein | MARCKSL1 | P49006 | NM_023009 | NP_075385 |
| 54 | 0.86 | Septin-7 | SEPT7 | Q16181 | NM_001011553, NM_001242956, NM_001363715, NM_001788, XM_006715806, XM_011515656, XM_011515661, XM_017012855, XM_017012856, XM_017012857, XM_017012858, XM_017012859, XM_017012860, XM_017012861, XM_017012862, XM_017012863, XM_024447010 | NP_001011553, NP_001229885, NP_001350644, NP_001779 XP_006715869, XP_011513958, XP_011513963, XP_016868344, XP_016868345, XP_016868346, XP_016868347, XP_016868348, XP_016868349, XP_016868350, XP_016868351, XP_016868352, XP_024302778 |
| 55 | 0.86 | Hsc70-interacting protein | ST13 | P50502 | NM_001278589, NM_003932 | NP_001265518, NP_003923 |
| 56 | 0.86 | COP9 signalosome complex subunit 8 | COPS8 | Q99627 | NM_006710, NM_198189 | NP_006701, NP_937832 |
| 57 | 0.86 | Vacuolar protein sorting-associated protein 37C | VPS37C | A5D8V6 | NM_017966, XM_005274077, XM_017017955 | NP_060436, XP_005274134, XP_016873444 |
| 58 | 0.86 | Acidic fibroblast growth factor intracellular-binding protein | FBP | O43427 | NM_004214, NM_198897, XM_024448763 | NP_004205, NP_942600, XP_024304531 |
| 59 | 0.86 | ADP-ribosyl cyclase/cyclic ADP-bose hydrolase 1 | CD38 | P28907 | NM_001775 | NP_001766 |
| 60 | 0.86 | Ribosome maturation protein SBDS | SBDS | Q9Y3A5 | NM_016038 | NP_057122 |
| 61 | 0.85 | Fructose-bisphosohate aldolase A | ALDOA | P04075 | NM_000034, NM_001127617, NM_001243175, NM_C01243177,NM_001355562, NM_C01355563, NM_001355564, NM_C01355565, NM_184041, NM_184043, XM_024450192, XM_024450193 | NP_000025, NP_C01121089, NP_001230106, NP_001342491, NP_001342492, NP_001342493, NP_001342494, NP_908930, NP_908932, XP_02435960, XP_024305961 |
| 62 | 0.85 | BH3_interacting domain death agonist | BID | P55957 | NM_001196, NM_001244567, NM_001244569, NM_001244570, NM_001244572, NM_197966, NM_197967 | NP_001187, NP_C01231496, NP_001231498, NP_001231499, NP_001231501, NP_932070, NP_932071 |
| 63 | 0.85 | Charged multives cular body protein 4b | CHMP4B | Q9H444 | NM_176812 | NP_789782 |

TABLE 5-6

| | | | | | | |
|---|---|---|---|---|---|---|
| 64 | 0.85 | Guanine nucleotide-binding protein G(s) subunit alpha isoforms short | GNAS | P63092 | NM_00C516, NM_0C1077488, NM_001077489, NM-001077490, NM_001309840, NM_001309842, NM_001309861, NM_001309883, NM_016592, NM_080425, NM_080426, XM_017027812, XM_017027813, XM_017027814, XM_017027815, XM_017027816, XM_017021817, XM_017027818, XM_017027819, XM_017027820, XM_017027821, XM_017027822, XM_024451872, XM_024451873, XM_024451874, XM_024451875 | NP_000507, NP_001070956, NP_001070957, NP_001070958, NP_001296769, NP_001296771, NP_001296790, NP_001296812, NP_057676, NP_536350, NP_536351, XP_016883301, XP_016883302, XP_016883303, XP_016883304, XP_016883305, XP_016883306, XP_016883307, XP_016883308, XP_016883309, XP_016883310, XP_016883311, XP_024307640, XP_024307641, XP_024307642, XP_024307643 |
| 65 | 0.85 | cAMP-dependent protein kinase catalytic subunit alpha | PRKACA | P17612 | NM_001304349, NM_C02730, NM_207518, XM_017026948 | NP_001291278, NP_002721, NP_997401, XP_016882437 |
| 66 | 0.85 | Cytosolic non-specific dipeotidase | CNDP2 | Q96KP4 | NM_001168499, NM_C18235 XM_005266728, XM_006722503, XM_011526072, XM_011526073 | NP_001161971, NP_060705, XP_005266785 XP_006722566, XP_011524374, XP_011524375 |
| 67 | 0.85 | Dynamin-1 | DNM1 | Q05193 | NM_001005336, NM_001288737, NM_001288738, NM_001288739, NM_004408, XM_005251763, XM_005251768, XM_005251769, XM_006716992, XM_006716993, XM_011518335, XM_011518336, XM_017014369, XM_017014370, XM_017014371, XM_017014372, XM_017014373 | NP_00105336, NP_001275666, NP_001275667, NP_001275668, NP_004399, XP_005251820, XP_005251825, XP_005251826, XP_006717055, XP_006717056, XP_011516637, XP_011516638, XP_016869858, XP_016869859, XP_016869860, XP_016869861, XP_016869862 |
| 68 | 0.85 | Neuroplastin | NPTN | Q9Y639 | NM_001161363, NM_001161364, NM_012428, NM_017455, XM_011521452, XM_02449899, XM_024449900 | NP_001154835, NP_001154836, NP_036560, NP_059429, XP_011519754, XP_024305667, XP_024305668 |
| 69 | 0.85 | IST1 homolog | IST1 | P53990 | NM_001270975, NM_001270976, NM_001270977, NM_C01270978, NM_001270979, NM_014761 | NP_001257904, NP_001257905, NP_001257906, NP_001257907, NP_001257908, NP_055576 |
| 70 | 0.85 | Sorting nexin-5 | SNX5 | Q9Y5X3 | NM_001282454, NM_014426, NM_152227 | NP_001269383, NP_055241, NP_689413 |
| 71 | 0.85 | Mitochondral import inner membrane translocase subunit Tim13 | TIMM13 | Q9Y5L4 | NM_012458 | NP_036590 |
| 72 | 0.85 | Exocyst complex component 4 | EXOC4 | Q96A65 | NM_001037126, NM_021807, XM_005250523, XM_17012494 | NP_001032203, NP_068579 XP_005250580, XP_016867983 |
| 73 | 0.84 | Moesin | MSN | P26038 | NM_002444, XM_005262269, XM_011530959, XM_017029545, XM_017029546 | NP_002435, XP_005262326, XP_011529261, XP_016885034, XP_016885035 |
| 74 | 0.84 | Serum albumin | ALB | P02768 | NM_000477 | NP_000468 |
| 75 | 0.84 | Myrstoylated alanne-rich C-kinase substrate | MARCKS | P29966 | NM_002356 | NP_002347 |
| 76 | 0.84 | Sodium/potassium-transporting ATPase subunit beta-3 | ATP1B3 | P54709 | NM_001679 | NP_001670 |
| 77 | 0.84 | Vinculin | VCL | P18206 | NM_003373, NM_014000 | NP_003364, NP_054706 |
| 78 | 0.84 I | Actin-related protein ⅔ complex subunit 1B | ARPC1B | 015143 | NM_005720, XM_006715825, XM_006715826, XM_024446628, XM_024446629 | NP_005711, XP_006715888, XP_006715889, XP_024302396, XP_02402397 |
| 79 | 0.84 | Alcohol dehydrogenase class-3 | ADH5 | P11766 | NM_000671 | NP_000662 |
| 80 | 0.84 | Cysteine and glycine-rich protein 1 | CSRP1 | P21291 | NM_001144773, NM_001193570, NM_001193571, NM_C01193572, NM_004078 | NP_001180499, NP_001180500, NP_001180501, NP_004069 |

TABLE 5-7

| | | | | | | |
|---|---|---|---|---|---|---|
| 81 | 0.84 | E3 ubiquitin-protein ligase TRIM23 | TRIM23 | P36406 | NM_001656, NM_033227, NM_033228, XM_017009444 | NP_001647, NP_150230, NP_150231, XP_016864933 |
| 82 | 0.84 | Actin-related protein 3 | ACTR3 | P61158 | NM_001277140, NM_005721 | NP_01204069, NP_005712 |
| 83 | 0.84 | Tetraspan n-15 | TSPAN15 | 096858 | NM_001351263, NM_012339, XM_005269666, XM_011539562, XM_011539563, XM_017016010 | NP_001338192, NP_036471, XP_005269723, XP_011537864, XP_011537865, XP_016871499 |
| 84 | 0.84 | Ras-related C3 botulinum toxin substrate 1 | RAC1 | P63000 | NM_006908, NM_018890, NM_198829 | NP_008839, NP 061485 |
| 85 | 0.84 | COP9 signalosome complex subunit 2 | COPS2 | P61201 | NM_001143887, NM_C04236 | NP_001137359, NP_004227 |
| 86 | 0.84 | Myotrophin | MTPN | P58546 | NM_145808 | NP_665807 |
| 87 | 0.84 | Rho-related GTP-ainding protein RhoF | RHOF | Q9HBH0 | NM_019034 | NP_061907 |
| 88 | 0.84 | COP9 signalosome compex subunit 3 | COPS3 | Q9UNS2 | NM_001199125, NM_001316354, NM_001316355, NM_001316356, NM_001316357, NM_001316358, NM_03653, XM_005256837, XM_005256840, XM_005256842, XM_017025246 | NP_001186054, NP_001303283, NP_001303284, NP_001303285, NP_001303286, NP_001303287, NP_003644, XP_005256894, XP_005256897, XP_005256899, XP_016880735 |
| 89 | 0.84 | Glutahione synthetase | GSS | P48637 | NM_000178, NM_001322494, NM_001322495 | NP_000189, NP_001309423, NP_001309424 |
| 90 | 0.84 | DnaJ homolog subfamily C member 5 | DRAJC5 | Q9H3Z4 | NM_025219, XM_011529048, XM_011529049 | NP_079495, XP_011527350, XP_011011527351 |
| 91 | 0.84 | Glutathione S-trans-erase omega-1 | GSTO1 | Pi8417 | NM_001191002, NM_001191003, NM_004832 | NP_001117931, NP_001177932, NP_004823 |
| 92 | 0.84 | Glutathione S-trans^{′}erase Mu 2 | GSTM2 | P28161 | NM_000848, NM_001142368 | NP_000839, NP_001135840 |
| 93 | 0.84 | Ragulator complex protein LAMTOR2 | LAMTOR2 | Q9Y2Q5 | NM_001145264, NM_014017 | NP_001138736, NP_054736 |
| 94 | 0.84 | Cytochome b5 type B | CYB5B | 043169 | NM_030579 | NP_085056 |
| 95 | 0.84 | Progressive ankylosis protein homolog | ANKH | Q9HCJ1 | NM_054027, XM_011514067, XM_017009644 | NP_473368, XP_011512369, XP_016865133 |
| 96 | 0.84 | Seotin-11 | SEPT11 | Q9NVA2 | NM_001306147, NM_018243 XM_00674259, XM_017008400, XM_017008401, XM_017008402, XM_017008403, XM_017008404 | NP_0012976, NP_060713, XP_006714322, XP_016863889, XP_016863890, XP_016863891, XP_016863892, XP_016863893 |
| 97 | 0.84 | Rho-assocaled protein kinase 1 | ROCK1 | Q13464 | NM_005406, XM_011526137 | NP_005397, XP_011524439 |
| 98 | 0.84 | Ras-related C3 botulinum toxin substrate 3 | RAC3 | P60763 | MM_001316307, NM_005052 | NP_001303236, NP_005043 |
| 99 | 0.84 | Isocitrate dahydrogenase [NAD] subunit alpha, mitochondrial | IDH3A | P50213 | NM_005530, XM_005254336, XM_02449911, XM_024449912 | NP_005521, XP_005254383, XP_024305679, XP_024305680 |
| 100 | 0.84 | Inactive tyrosine-protein kinase transmembrane receptor ROR1 | ROR1 | Q01973 | NM_001083592, NM_005012 XM_011541526, XM_017001376, XM_017001377 | NP_001077061, NP_005003, XP_011539828 XP_016856865, XP_016856866 |

TABLE 5-8

| | | | | | | |
|---|---|---|---|---|---|---|
| 101 | 0.84 | Abl interactor 2 | ABI2 | Q9NYB9 | NM_001282925, NM_001282926, NM_001282927, NM_01282932, NM_005759, XM_005246217, XM_005246218, XM_005246221, XM_005246225, XM_005246226, XM_006712174, XM_006712177, XM_006712179, XM_006712180, XM_006712181, XM_006712182, XM_006712185, XM_006712186, XM_011510472, XM_011510473, XM_017003118, XM_017003119, XM_017003120, XM_017003121, XM_017003122, XM_017003123, XM_017003124, XM_017003125, XM_017003126, XM_017003127, XM_017003128, XM_017003129, XM_017003130, XM J17003131, XM_017003132, XM_017003133, XM_017003134, XM_017003135, XM_017003136, XM_017003137, XM_017003138, XM_017003139, XM_017003140, XM_017003141, XM_017003142, XM_17003143, XM_017003144, XM_017003145, XM_017003146, XM_024452511 | NP_001269854, NP_001269855, NP_001269856, NP_001269861, NP_005750, XP_005246274, XP_005246275, XP_005246278, XP_005246282, XP_005246283, XP_006712237, XP_006712240, XP_006712242, XP_006712243, XP_006712244, XP_006712245, XP_006712248, XP_006712249, XP_011508774, XP_01158775, XP_016858607, XP_016858608, XP_016858609, XP_016858610, XP_016858611, XP_016858612, XP_016858613, XP_016858614, XP_016858615, XP_J16858616, XP_016858617, XP_016858618, XP_016858619, XP_016858620, XP_016858621, XP_016858622, XP_016858623, XP_016858624, XP_016858625, XP_016858626, XP_016858627, XP_016858628, XP_016858629, XP_016858630, XP_016858631, XP_01685832, XP_016858633, XP_016858634, XP_016858635, XP_024308279 |
| 102 | 0.83 | Cell division control protein 42 homolog | CDC42 | P80953 | NM_001039802, NM_001791, NM_044472 | NP_00134891, NP_001782, NP_426359 |
| 103 | 0.83 | Transgelin-2 | TAGLN2 | P37802 | NM_001277223, NM_001277224, NM_003564 | NP_001264152, NP_001264153, NP_003555 |
| 104 | 0.83 | Ras-related protein Rab-2A | RAB2A, | P61019 | NM_001242644, NM_002865 | NP_001229573, NP_002856 |
| 105 | 0.83 | Charged multivesic ɹlar ɔody protein 6 | CHMP6 | Q96FZ7 | NM_024591, XM_005257668 | NP_078867, XP_005257725 |
| 106 | 0.83 | ADP-ribosylation factor 3 | PRF3 | P61204 | NM_001659, XM_005268856, XM_006719391, XM_024448972 | NP_001650, XP_005268913, XP_006719454, XP_024304740 |
| 107 | 0.83 | Glyoxylate reductase/hydroxypyruvate reductase | GRHPR | Q9UBQ7 | NM_012203, XM_005251631, XM_017015320, XM_017015321, XM_017015323, XM_024447716, XM_024447717 | NP_036335, XP_005251688, XP_016870809, XP_016870810, XP_016870812, XP_024303484, XP_024303485 |
| 108 | 0.83 | Profilin-1 | PFN1 | P07737 | NM_005022, XM_017024761 | NP_005013, XP_016880250 |
| 109 | 0.83 | Ras-related protein Rab-11B | RAB11B | Q15907 | NM_004218 | NP_004209 |
| 110 | 0.83 | Rab GDP jissocialon inhibitor alpha | GD\|1 | P31150 | NM_001493 | NP_001484 |
| 111 | 0.83 | CD97 antigen | CD97 | P48960 | NM_001026160, NM_001784, NM_078481, XM_011528451 | NP_001020331, NP_001775, NP_510966, XP_011526753 |
| 112 | 0.83 | Programmed cell death protein 1C | PDCD10 | Q9BUL8 | NM_007217, NM_145859, NM_145860, XM_005247086, XM_005247087, XM_005247088, XM_006713485, XM_011512368, XM_011512369, XM_017005644, XM_017005645, XM_024453329, XM_024453330, XM_024453331 | NP_009148, NP_665858, NP_665859, XP_005247143, XP_005247144, XP_005247145, XP_006713548, XP_011510670, XP_011510671, XP_016861133, XP_016861134, XP_024309097, XP_24309098, XP_024309099 |

TABLE 5-9

| | | | | | | |
|---|---|---|---|---|---|---|
| 113 | 0.83 | Antithrombin-III | SERPINC1 | P01008 | NM_000488, NM_001365052, XM_005245198 | NP_00479, NP_001351981, XP_005245255 |
| 114 | 0.83 | F-actin-caoping protein subunit beta | CAPZB | P47756 | NM_001206540, NM_001206541, NM_001282162, NM_001313932, NM_004930, XM_006710938, XM_011542228, XM_011542229, XM_011542230, XM_017002428, XM_017002429, XM_017002430, XM_024450096, XM_024450097 | NP_001193469, NP_001193470, NP_001269091, NP_001300861, NP_004921, XP_006711001, XP_011540530, XP_011540531, XP_011540532, XP_016857917, XP_016857918, XP_016857919, XP_024305864, XP_024305865 |
| 115 | 0.83 | Actin-related protein ⅔ complex subunit2 | ARP02 | 015144 | NM_005731, NM_152862, XM_017003113, XM_017003114, XM_017003115, XM_024452509 | NP_005722, NP_690601, XP_016858602, XP_016858603, XP_016858604, XP_024308277 |
| 116 | 0.83 | Twinfilin-2 | TWF2 | Q6\|BS0 | NM_007284 | NP_009215 |
| 117 | 0.83 | Ras-related protein Rab-21 | RAB21 | Q9UL25 | NM_014999 | NP_055814 |
| 118 | 0.83 | Vacuolar-sorting protein SNF8 | SNF8 | Q96H20 | NM_001317192, NM_001317193, NM_001317194, NM_007241 XM_017024075 | NP_001304121, NP_001304122, NP_001304123, NP_009172, XP_016879564 |
| 119 | 0.83 | Caspase-6 | CASP6 | P55212 | NM_001226, NM_032992 | NP_001217, NP_116787 |
| 120 | 0.83 | Calcineunn subunit B type 1 | PPP3R 1 | P63098 | NM_000945 | NP_000936 |
| 121 | 0.83 | Extended synaptotagmin-2 | ESYT2 | A0FGR8 | NM_020728, XM_024446845, XM_024446846, XM_024446847, XM_024446848, XM_024446849 | NP_065779, XP_024302613, XP_024302614, XP_024302615, XP_024302616, XP_02430267 |
| 122 | 0.83 | Leukocyte surface antigen CD47 | CD47 | Q08722 | NM_001025079, NM_001025080, NM_001777 NM_198793, XM_005247908, XM_005247909, XM_017007536 | NP_001768, NP_942088, XP_005247965, XP_005247966 XP_016863025 |
| 123 | 0.83 | COP9 signalosome complex subunit 1 | GPS1 | Q13098 | NM_001321089, NM_001321090, NM_001321091, NM_001321092, NM_001321093, NM_001330539, NM_001330541, NM_004127, NM_212492, XM_005256350, XM_005258353, XM_005256359, XM_005258360, XM_017024524, XM_017024525, XM_017024526, XM_017021527, XM_017024531, XM_017024534, XM_017024535, XM_024450713, XM_024450714, XM_024450715, XM_024450716, XM_024450717, XM_024450718, XM_024450719, XM_024450720, XM_024450721, XM_024450722, XM_024450723, XM_024450724, XM_024450725, XM_ 024450726, XM_024450727, XM_024450728 | NP_001308018, NP_001308019, NP_001308020, NP_001308021, NP_001308022, NP_001317468, NP_001317470, NP_004118, NP_997657, XP_005256407, XP_005256410, XP_005256416, XP_005256417, XP_016880013, XP_016880014, XP_016880015, XP_016880016, XP_016880020, XP_016880023, XP_016880024, XP_024306481, XP_024306482, XP_024306483, XP_024306484, XP_024306485, XP_024306486, XP_024306487, XP_024306488, XP_024306469, XP_024306490, XP_024306491, XP_024306492, XP_024306493, XP_024306494, XP_024306495, XP_024306496 |
| 124 | 0.83 | 26S proteasome non-ATPase regulatory subunit 7 | PSMD7 | P51665 | NM_002811 | NP_002802 |
| 125 | 0.83 | Eukaryotic translaloninitiation factor 3 subunit C-like protein | EIF3CL | B5ME19 | NM_001099661, NM_001317856 NM_001317857, XM_017023620, XM_017023621 | NP_001093131, NP_001304785, NP_001304786, XP_016879109, XP_016879110 |

TABLE 5-10

| | | | | | | |
|---|---|---|---|---|---|---|
| 126 | 0.83 | Deoxycytidylate deaminase | DCTD | P32321 | NM_001012732, NM_001351743, NM_001351744, NM_001351745, NM_001351747, NM_001351748, NM_001351750, NM_001351753, NM_001921, XM_005262778, XM_005262781, XM_005262782, XM_006714115, XM_006714116, XM_011531674, XM_011531675, XM_011531676, XM_017007820, XM_017007822, XM_017007823, XM_017007824 | NP_001012750, NP_001338672, NP_01338673, NP_001338674, NP_001338676, NP_001338677, NP_001338679, NP_001338682, NP_001912, XP_005262835, XP_005262838, XP_005262839, XP_006714178, XP_006714179, XP_011529976, XP_011529977, XP_011529978, XP_016863309, XP_016863311, XP_016863312, XP_016863313 |
| 127 | 0.83 | CD276 antigen | CD276 | Q5ZPR3 | NM_001024736, NM_001329628, NM_001329629, NM_025240, XM_005254700. XM_011522095, XM_017022638 | NP_001019907, NP_001316557, NP_ 001316658, NP_ 079516, XP_005254757, XP_011520397, XP_016878127 |
| 128 | 0.83 | Caspase-3 | CASP3 | P42574 | NM_001354777, NM_001354779, NM_001354780, NM_001354781, NM_001354782, NM_001354783, NM_001354784, NM_004346, NM_032991 | NP_001341706, NP_001341708, NP_001341709, NP_001341710, NP_001341711, NP_001341712, NP_001341713, NP_004337, NP_116786 |
| 129 | 0.83 | Leucine-rich repeat-containing protein 57 | LRRC57 | Q8N9N7 | NM_153260, XM_011521423, XM_011521424 | NP_694992, XP_011519725, XP_011519726 |
| 130 | 0.83 | Mdose reductase | AKR1B1 | P15121 | NM_001346142, NM_001628, XM_005250234, XM_024446695 | NP_001333071, NP_001619, XP_005250291, XP_024302463 |
| 131 | 0.83 | Diphosphoinosital polyphosphate phosphohydrolase 1 | NUDT3 | 095989 | NM_006703 | NP_006694 |
| 132 | 0.83 | Coagulation factor XIII A chain | F13A1 | P00488 | NM_000129 | NP_00120 |
| 133 | 0.83 | Hso70-binding protein 1 | HSPBP1 | Q9NZ14 | NM_001130106, NM_001297600, NM_012267, XM_005258700, XM_005258701, XM_011526691, XM_017026543, XM_017026544, XM_017026545, XM_024451436, XM_024451437 | NP_001123578, NP_001284529, NP_036399, XP_005258757, XP_005258758, XP_011524993, XP_016882032, XP_016882033, XP_016882034, XP_024307204, XP_024307205 |
| 134 | 0.83 | Sortting nexin-4 | SNX4 | 095219 | NM_003794, XM_017007414 | NP_003785, XP_016862903 |
| 135 | 0.83 | Protein phosphatase 1L | PPM1L | Q5SGD2 | NM_001317911, NM_001317912, NM_139245, XM_011512440 | NP_001304840, NP_001304841, NP_640338, XP_011510742 |
| 136 | 0.833 | Pleckstrin homology domain-containing family N member 1 | PLEKHN1 | Q494U1 | NM_001160184, NM_032129, XM_006710944, XM_011542248, XM_017002474, XM_017002475, XM_017002476, XM_017002477, XM_017002478, XM_017002479 | NP_001153656, NP_115505, XP_006711007, XP_011540550, XP_016857963, XP_016857964, XP_016857965, XP_016857966, XP_016857967, XP_016857968 |
| 137 | 0.83. | AP-2 complex subunit alpha-2 | PP2A2 | 094973 | NM_001242837, NM_012305, XM_011519929, XM_011519930 | NP_001229766, NP_036437, XP_011518231, XP_011518232 |
| 138 | 0.83 | Trophoblast glycoprotein | TPBG | Q13641 | NM_001166392, NM_06670 | NP_001159864, NP_006661 |
| 139 | 0.825 | Ras-related protein Rab-23 | RAB23 | Q9ULC3 | NM_001278666, NM_001278667, NM_001278668, NM_016277, NM_183227 | NP_001285595, NP_001265596, NP_0012855697, NP_057361, NP_899050 |
| 140 | 0.82 | 14-3-3 protein eta | YWHAH | Q04917 | NM_003405 | NP_003396 |
| 141 | 0.82 | Peroxiredoxin-6 | PRDX6 | P30041 | NM_004905 | NP_004896 |
| 142 | 0.82 | 14-3-3 protein beta/alpha | YWHAB | P31946 | NM_003404, NM_139323, XM_017028039, XM_017028040 | NP_003395, NP_647539, XP_016883528, XP_016883529 |
| 143 | 0.82 | Copine-2 | CPNE2 | Q96FN4 | NM_152727 | NP_89940 |
| 144 | 0.82 | 14-3-3 protein gamma | YWHAG | P61981 | NM_12479 | NP_036611 |

TABLE 5-11

| | | | | | | |
|---|---|---|---|---|---|---|
| 145 | 0.82 | Superoxide dismutase [Cu-Zn] | SOD1 | P00441 | NM_000454 | NP_000445 |
| 146 | 0.82 | Microtubuls-associated protein RPIEB family member 1 | MAPRE1 | Q15691 | NM_012325, XM_011528696 | NP_036457, XP_011526998 |
| 147 7 | 0.82 | Vasodilator-stimulated phosphoprotein | VASP | P5055i | NM_001008736, NM_003370, XM_005259199, XM_005259200, XM_017027200 | NP_003361, XP_005259256, XP_005259251, XP_016882689 |
| 148 | 0.82 | Phosphoglucomutase-1 | PGM1 | P36871 | NM_001172818, NM_001172819, NM_002633 | NP_001166289, NP_001166290, NP_002624 |
| 149 | 0.82 | Adenylylcyclase-associated protein 1 | CAP1 | Q01518 | NM_001105530, NM_001330502, NM_001350475, NM_001350476, NM_001350177, NM_001350478, NM_001350479, NM_001350480, NM_001350481, NM_001350482, NM_001350483, NM_001350484, NM_001350485, MN_006367, XM_011540510, XM_011540515, XM_017000069 | NP_Q01099000, NP_001317431, NP_001337404, NP_001337405, NP_001337406, NP_001337407, NP_001337408, NP_001337409, NP_001337410, NP_001337411, NP_001337412, NP_001337413, NP_001331414, NP_006358, XP_011538812, XP_011538817, XP_016855558 |
| 150 | 0.82 | Caloain small subunit 1 | CAPNS1 | P04632 | NM_001003962, NM_001302532, NM_001302633, NM_001749, XM_005259295, XM_005259296, XM_011527359, XM_024451732 | NP_001003962, NP_001289561, NP_001289562, NP_001740, XP_005259352, XP_005259353, XP_ 011525661, XP_024307500 |
| 151 | 0.82 | Dual specficity protein pnosphatase 23 | DUSP23 | Q9BVJ7 | NM_001319658, NM_001319959, NM_017823 | NP_001306587, NP_001306588, NP_060293 |
| 152 | 0.82 2 | Dihydropteridine reductase | QDPR | P09417 | NM_000320, NM_001306140 | NP_000311, NP_001293069 |
| 153 | 0.82 | Ephmin-B1 | EFNB1 | P98172 | NM_004429 | NP_004420 |
| 154 | 0.82 | Golgin subfamily A member 7 | GOLGA7 | Q7Z5G4 | NM_001002296, NM_001174124, NM_001362979, NM_001362980, MM_016099, XM_02447174, XM_024447175 | NP_001002296, NP_001 167595, NP_001349908, NP_001349909, NP_057183, XP_024302942, XP_024302943 |
| 155 | 0.82 | Xaa-Proaminopeptidase 1 | XPNPEP1 | Q9NQW7 | NM_001167604, NM_001324128, NM_001324131, NM_001324132, NM_001324133, NM_001324134, NM_001324135, NM_001324136, NM_020383, XM_011540134, XM_017016613, XM_017016614, XM_017016615, XM_024448156 | NP 001161076, NP_001311057, NP_001311060, NP 001311961, NP_001311062, NP_001311063, NP_001311064, NP_01311065, NP_065116, XP_011538436, XP_016872102, XP_016872103, XP_016872104, XP_024303924 |
| 156 | 0.82 | Actin-related protein ⅔ complex subunit 3 | ARPC3 | 015145 | NM_001278556, NM_001287222 | NP_001265485, NP_001274151 |
| 157 | 0.82 | Sorting nexin-9 | SNX9 | Q9Y5X1 | NM_016224, XM_005267015, XM_011535886 | NP_057308, XP_005267072, XP_01153188 |
| 158 | 0.82 | Pleckstrin homology domain-containing family C member 2 | PLEKH02 | Q8TD55 | NM_001195059, NM_025201 | NP_001181988, NP_079477 |
| 159 | 0.82 | WD repeat-containing protein 44 | WDR44 | Q5JSH3 | NM_001184965, NM_001184966, NM_019045, XM_011531353, XM_017029600 | NP_01171894, NP_001111895, NP_061918, XP_011629655, XP_016886089 |
| 160 | 0.82 | N(G),N(G)-dimelhylarginine dimelhyam nohydrolase 2 | DDAH2 | 095865 | NM_001303007, NM_001303008, NM_013974, XM_011514448 | NP_001289936, NP_001289937, NP_039268, XP_011512750 |
| 161 | 0.82 | COP9 signalosome compex subunit6 | COPS6 | Q7L5N1 | NM_000833 | NP_006824 |
| 162 | 0.82 | Guanine nucleotide-binding protein G(I)G(S)/G(O)subunit gamma-10 | GNG10 | P50151 | NM_001017998, NM_001198664 | NP_00107998, NP_001185593 |
| 163 | 0.82 | Rab-like protein 3 | RABL3 | Q5HYI8 | NM_001363964, NM_001363965, NM_173825, XM_006713595, XM_017006227, XM_017006228 | NP_001350893 NP_001350894, NP_776186, XP_006713658, XP_016861716, XP_016861717 |
| 164 | 0.82 | Phosphoacetylglucosamine mubse | PGM3 | 096394 | NM_001199917, NM_001199918, NM_001199919, NM_015599, XM_017010935, XM_017010937, XM_024446459, XM_024446460 | NP_01186846, NP_001186847, NP_001186848, NP_056414, XP_016866424, XP_016866426, XP_024302227, XP_024302228 |

TABLE 5-12

| | | | | | | |
|---|---|---|---|---|---|---|
| 165 | 0.82 | Sorting nexin-2 | SNX2 | 060749 | NM_001278199, NM_003100 | NP_001265128, NP_003091 |
| 166 | 0.82 | DDB1- and CUL4-associated factor 11 | DCAF11 | Q8TEB1 | NM_001163484, NM_025230, NM_181357 | NP_00156956, NP_079506, NP_852002 |
| 167 | 0.82 | Ras GTPase-activating protein 1 | RASA1 | P20936 | NM_002890, NM_022650, XM_01154325, XM_011543527 | NP_002881, NP_072179, XP_011541827, XP_011541829 |
| 168 | 0.82 | Ribose-5-phospate isomerase | RPIA | P49247 | NM_144563 | NP_653164 |
| 168 | 0.82 | SHC-transforming protein 1 | SHC1 | P29353 | NM_001130040, NM_001130041, NM_001202859, NM_003029, NM_183001, XM_005245449, XM_00524541, XM_011509892, XM_011509893, XM_011509894, XM_011509897, XM_011509898, XM_017002081, XM_017002082, XM_017002083, XM_024449083, XM_024449084 | NP_001123512, NP_001123513, NP_001189788, NP_003020, NP_892113, XP_005245506, XP 00524550B, XP_011508194, XP_011608195, XP_011508196, XP_011508199, XP_011508200, XP_01685757C, XP_016857571, XP_016857572, XP_024304851, XP_024304852 |
| 170 | 0.82 | Glutaminyl-peptide cyclotransferase-like protein | QPCTL | Q9NXS2 | NM_001163377, NM_017659, XM_011527048, XM_017026900 | NP_001156849, NP_060129, XP_011525350, XP_016882389 |
| 171 | 0.81 | Ras-related protein Rab-5C | RAB5C | P51148 | NM_00125229, NM_004583, NM_201434 XM_011525090 | NP_001238968, NP_004574, NP_958842, XP_011523392 |
| 172 | 0.81 | Purine nucleoside phosphorylase | PNP | P00491 | NM_000270 | NP_000261 |
| 173 | 0.81 | Delta-aminolevulnic acid dehydratase | ALAD | P13716 | NM_000031, NM_001003945, NM_001317745, XM_011518364, XM_024447449 | NP_000022, NP_001003945, NP_001304874, XP_011516666, XP_024303217 |
| 174 | 0.81 | Ras-related protein Rab-5B | R.AB5B | P61020 | NM_001252038, NM_001252037, NM_002868, XM_005269051, XM_005269052 | NP_001238965, NP_001238966 NP_002859, XP005269108, XP_005269109 |
| 175 | 0.81 | Abl interactor 1 | ABl1 | Q8IZPC | NM_001012750, NM_001012751 NM_001012752, NM_001178116, NM_001178119, NM_001178120, NM_001178121, NM_001178122, NM_001178123, NM_001178124, NM_001178125, NM_001348029, NM_001348030, NM_001348031, NM_001348032, NM_001348033, NM_001348034, NM_005470, XM_005252332, XM_005252333, XM_011519289, XM_017015459 | NP_001012768, NP_001012769, NP_001012710, NP_001171587, NP_001171590, NP_001171591, NP_001171592, NP_001171593, NP_001171594, NP_001171595, NP_001171596, NP_00134958, NP_001334959, NP_001334960, NP_001334961, NP_001334962, NP_001334963, NP_005461, XP_005252389, XP_005252390, XP_011517591, XP_016870948 |
| 176 | 0.81 | Beta-2-glycoprotein 1 | APOH | P02749 | NM_000042 | NP_000033 |
| 177 | 0.81 | Serine/threonine-protein prosphatase 2B catalytic subunit alpha isoform | PPP3CA | Q08209 | NM_000944, NM_001130691, NM_001130692, XM_017008365, XM_024454127 | NP_000935, NP_001124163, NP_001124164, XP_016863854, XP_024309895 |
| 178 | 0.81 | Act n-related prole n 2 | ACTR2 | P61160 | NM_001005388, NM_005722 | NP_00105386, NP_005713 |
| 179 | 0.81 | Ras-related protein Rap-2a | RAP2A | P10114 | NM_021033 | NP_066361 |
| 180 | 0.81 | Sorcin | SRI | P30626 | NM_01256891, NM_01256892, NM_003130, NM_198901, XM_011516528 | NP_001243820, NP_001243821, NP_003121, NP_944490, XP_011514830 |
| 181 | 0.81 | Tyoel l inos tol 1,4,5-trisprosphate 5-phosphatase | NPP5A | Q14642 | NM_001321042, NM_005539, XM_017016204, XM_017016205 | NP_001307971, NP_005530, XP_016871693, XP_016871694 |
| 182 | 0.81 | Ras-related protein Rab-35 | RAB35 | Q15286 | NM_001167606, NM_006861, XM_011537779, XM_024448801 | NP_001161078, NP_006852, XP_011536081, XP-024304569 |
| 183 | 0.81 | Rho GTPase-activating protein 18 | ARHGAP18 | QBN392 | NM_033515, XM_005267212, XM_005267213, XM_017011494 | NP_277050, XP_005267269, XP_005267270, XP_016866983 |

TABLE 5-13

| | | | | | | |
|---|---|---|---|---|---|---|
| 184 | 0.81 | Ras-related protein Rab-1A | RAB1A | P62820 | NM_004161, NM_015543, XM_005264468 | NP_004152, NP_056358, XP_005264525 |
| 185 | 0.81 | Deoxyribonuclease-1-like 1 | DNASE1L1 | P49184 | NM_001009932, NM_001009933, NM_001009934, NM_001303620, NM_006730, XM_005277829, XM_011531122, XM_017029332 | NP_001009932, NP_001009933, NP_001009934, NP_001290549, NP_006721, XP_005277886, XP_011529424, XP_016884821 |
| 186 | 0.81 | Peptidyl-prdyl cis-trans isomerase A | PPIA | P62937 | NM_001300981, NM_021130, NM_203430, NM_203431, XM_024446808, XM_024446809 | NP_001287910, NP_066953, XP_02430576, XP_024302577 |
| 187 | 0.81 | Adenylate kinase soenzyrre 1 | AK1 | P00568 | NM_000476, NM_001318121, NM_001318122, XM_017014428, XM_024447439, XM_024447440 | NP_000467, NP_001305050, NP_001305051, XP_016869917, XP_024303207, XP_024303208 |
| 188 | 0.81 | Equil brative nucleoside transporter 1 | SLC29A1 | Q99808 | NM_001078174, NM_001078175, NM_001078175, NM_001078177, NM_001304462, NM_0013004463, NM_001304465, NM_00130466, NM_004955, XM_005248876, XM_005248878, XM_005248879, XM_005248880, XM_005248881, XM_005248882, XM_011514341, XM_024446348 | NP_001071643, NP_001071645, NP_001291391, NP_001291392, NP_001291394, NP_001291395, XP_005248933, XP_005248935, XP_005248936, XP_005248937, XP_005248938, XP_005248939, XP_011512643, XP_024302116 |
| 189 | 0.81 | GTPase NRas | NRAS | P01111 | NM_002524 | NP_002515 |
| 190 | 0.81 | Rho-related GTP-binding protein RhoG | RHOG | P84095 | NM_001665, XM_005252916, XM_017017719 | NP_001656, XP_005252973, XP_016873208 |
| 191 | 0.81 | Glycogen phosphorylase, brain form | PYGB | P11216 | NM_002862 | NP_002853 |
| 192 | 0.81 | UV excision repair protein RAD23 homolog B | RAD23B | P54727 | NM_001244713, NM_001244724, NM_002874 | NP_001231642, NP_001231653, NP_002865 |
| 193 | 0.81 | Omega-amidase NIT2 | NIT2 | Q9NQR4 | NM_020202 | NP_064587 |
| 194 | 0.81 | Serine hydroxymethyltrans⁻erase, cytosolic | SHMT1 | P34896 | NM_001281785, NM_004169, NM_148918, XM_005256767, XM_011523992, XM_017024957, XM_017024958, XM_024450887, XM_024450888, XM_024450889, XM_024450890 | NP_001268715, NP_004160, NP_683718, XP_005256824, XP_011522294, XP_016880446, XP_016880447, XP_024306655, XP_024306656, XP_024306657, XP_024306658 |
| 195 | 0.81 | Sodium/potassium-transporting ATPase subunit beta-1 | ATP1B1 | P05026 | NM_001001787, NM_001677 | NP_001668 |
| 196 | 0.81 | Endothelial cell-selective adhesion molecule | ESAM | Q96AP7 | NM_138961 | NP_620411 |
| 197 | 0.81 | Isocitrate dehydrogenase [NADP] cytoplasmic | IDH1 | O75874 | NM_001282386, NM_001282387, NM_005896 | NP_001268315, NP_001269316, NP_005887 |
| 198 | 0.81 | Peptidyl-prdyl cis-trans isomerase FKBP1A | FKBP1A | P62942 | NM_000801, NM_001199786, NM_054014 | NP_000792, NP_001186715, NP_463460 |
| 199 | 0.81 | Dystroglycan | DAG1 | Q14118 | NM_001165928, NM_001177634, NM_001177635, NM_001177636, NM_001177637, NM_001177638, NM_001177639, NM_001177640, NM_001177641, NM_001177642, NM_001177643, NM_001177644, NM_004393 | NP_001159400, NP_001171105, NP_001171106, NP_001171107, NP_001171108, NP_001171109, NP_001171110, NP_001171111, NP_001171112, NP_001171113, NP_001171114, NP_001171115, NP_004384 |
| 200 | 0.81 | Turror susceptibility gene 101 protein | TSG101 | Q99816 | NM_006292, XM_005253108 | NP_006283, XP_005253165 |
| 201 | 0.81 | Peroxiredoxin-5, mitochondrial | PRDX5 | P30044 | NM_001358511, NM_001358516, NM_012094, NM_181651, NM_181652 | NP_001345440, NP_001345445, NP_036226, NP_857634, NP_857635 |
| 202 | 0.81 | Protein XRP2 | RP2 | C75695 | NM_006915 | NP_008846 |
| 203 | 0.81 | Actin-related protein ⅔ complex subunit 4 | ARPC4 | P59998 | NM_001024959, NM_001024960, NM_001198780, NM_005718 | NP_001020130, NP_001020131, NP_001185709, NP_005709 |
| 204 | 0.81 | Stathmin | STMN1 | P16949 | NM_001145454, NM_005563, NM_152497, NM_203399, NM_203401 | NP_001138926, NP_005554, NP_981944, NP_981946 |

TABLE 5-14

| | | | | | | |
|---|---|---|---|---|---|---|
| 205 | 0.81 | NSFL1 cofactor p47 | NSFL1C | Q9UNZ2 | NM_001206736, NM_016143, NM_018839, NM_182483, XM_006723592, XM_006723593, XM_006723594, XM_011529300, XM_011529301, XM_011529302, XM_011529304, XM_017027956, XM_017027957, XM_017027958 | NP_001193665, NP_057227, NP_061327, XP_006723655, XP_006723656, XP_006723657, XP_011527602, XP_011527603, XP_011527604, XP_011527606, XP_016883445, XP_016883446, XP_016883447 |
| 206 | 0.81 | Folate receptor aloha | FOLR1 | P15328 | NM_000802, NM_016724, NM_016725, NM_016729, NM_016730 | NP_000793, NP_057936, NP_057937, NP_057941 |
| 207 | 0.81 | Aldo-keto reductase family 1 member A1 | AKR1A1 | P14550 | NM_001202413, NM_001202414, NM_006066, NM_153326, XM_011540491, XM_011540492 | NP_001189342, NP_001189343, NP_006057, NP_697021, XP_011538793, XP_011538794 |
| 208 | 0.81 | Ran-specific GTPase-activating protein | RANBP1 | P43487 | NM_001278639, NM_001278640, NM_001278641, NM_002882, XM_011530289, XM_011530290, XM_011530291, XM_017028890, XM_017028891, XM_017028892 | NP_001265568, NP_001265569, NP_001265570, NP_002873, XP_011528591, XP_011528592, XP_011528593, XP_016884379, XP_016884380, XP 016884381 |
| 209 | 0.81 | Serine/threonine-protein prosphatase 2A catalytic subunit beta isoform | PPP2CB | P62114 | NM_001009552, NM_004156 | NP_001009552 |
| 210 211 | 0.81 0.81 | Vacuolar protein sorting-associated protein VTA1 homolog Dual specificity protein phosphatase 3 | VTA1 DUSP3 | Q9NP79 P51452 | NM_001286371, NM_001286372, NM_016485 NM_004090 | NP_001273300, NP_001273301, NP_057569 NP_004081 |
| 212 | 0.81 | Membrane cofactor protein | CD46 | P15529 | NM_002389, NM_153826, NM_172350, NM_172351, NM_172352, NM_172353, NM_172354, NM_172355, NM_172356, NM_172357, NM_172358, NM_172359, NM_172360, NM_172361, XM_011509563, XM_011509564, XM_017001308, XM_017001309, XM_017001310 | NP_002380, NP_722548, NP_758860, NP_758861, NP_758862, NP_758863, NP_758865, NP_758866, NP_758867, NP_758868, NP_758869, NP_758871, XP_011507865, XP_011507866, XP_016856797, XP_016856798, XP_016856799 |
| 213 | 0.81 | Calcium-binding protein 39 | CAB39 | Q9Y376 | NM_001130849, NM_001130850, NM_016289, XM_011511350 | NP_001124321, NP_001124322, NP_057373, XP_011509652 |
| 214 | 0.81 | Argininosuccinate lyase | ASL | P04424 | NM_000048, NM_001024943, NM_001024944, NM_001024946 | NP_000039, NP_001020114, NP_001020115, NP_001020117 |
| 215 | 0.81 | Mtochondrial import receptor subunit TOM34 | TOMM34 | Q15785 | NM_006809, XM_005260254, XM_011528501, XM_017027600 | NP_006800, XP_005260311, XP_011526803, XP_016883089 |
| 216 | 0.81 | Protein numb homolog | NUMB | P49757 | NM_001005743, NM_001005744, NM_001005745, NM_001320114, NM_003744 | NP_001005743, NP_001005744, NP_001005745, NP_001307043, NP_003735 |
| 217 | 0.81 | SUMO-conjugating enzyme UBC9 | UBE21 | P63279 | NM_003345, NM_194259, NM_194260, NM_194261, XM_005255540, XM_005255541, XM_011522644, XM_011522645, XM_017023640, XM_024450432 | NP_003336, NP_919235, NP_919236, NP_919237, XP_005255597, XP_005255598, XP_011520946, XP_011520947, XP_016879129, XP_024306200 |
| 218 | 0.81 | EH domain-containing protein 3 | EHD3 | Q9NZN3 | NM_014600, XM_011532806 | NP_055415, XP_011531108 |
| 219 | 0.81 | Ras-related protein Rab-1B | RAB1B | Q9HOU4 | NM_030981, XM_017018378 | NP_112243, XP_016873867 |
| 220 | 0.81 | Adenine phosphoribosyltransferase | APRT | P07741 | NM_000485, NM_001030018 | NP_000476, NP_001025189 |
| 221 | 0.81 | Coronin-1A | CORO1A | P31146 | NM_001193333, NM_007074, XM_011545714, XM_017022885, XM_017022886 | NP_001180262, NP_009005, XP_011544016, XP_016878374, XP_016878375 |

TABLE 5-15

| | | | | | | |
|---|---|---|---|---|---|---|
| 222 | 0.8 | AnnexinA4 | ANXA4 | P09525 | NM_001153, NM_001320698, NM_001320700, NM_001320102, NM_001365496, XM_017003943, XM_017003944, XM_024452834, XM_024452835, XM_024452836 | NP_001144, NP_001307627, NP_001307629, NP_001307631, NP_001352425, XP_016859432, XP_016859433, XP_024308602, XP_024308603, XP_024308604 |
| 223 | 0.8 | F-actin-capping protein subunit alpha-1 | CAPZA1 | P5290i | NM_006135, XM_011542225, XM_017002424 | NP_006126, XP_011540527, XP_016857913 |
| 224 | 0.8 | Protein phosphalase 1 regulatory subunit 7 | PPPIRi | Q15435 | NM_001282409, NM_001282410, NM_001282411, NM_001282412, NM_001282413, NM_001282414, NM_002712, XM_011511388 | NP_001269338, NP_001269339, NP_001269340, NP_001269341, NP_001269342, NP_001269343, NP_002703, XP_011509690 |
| 225 | 0.8 | Peflin | PEF1 | Q9UBV8 | NM_001359651, NM_012392, XM_011541745, XM_011541746, XM_011541747, XM_017001680, XM_017001681 | NP_001348580, NP_036524, XP_011540047, XP_011540048, XP_011540049, XP_016857169, XP_016857170 |
| 226 | 0.8 | Protein/nucleic acid deglycase DJ-1 | PARK7 | Q99497 | NM_001123377, NM_007262, XM_005263424 | NP_001116849, NP_009193, XP_005263481 |
| 227 | 0.8 | Ras.relatedproteinRab-10 | RAB10 | P61026 | NM_016131, XM_024452565 | NP_057215, XP_024308333 |
| 228 | 0.8 | Ras-related proteinRap-2b | RAP2B | P61225 | NM_002886 | NP_002871 |
| 229 | 0.8 | RNA transcription, translation and transportfactor protein | RTRAF | Q9Y224 | NM_016039 | NP_057123 |
| 230 | 0.8 | Crx-like protein | CRKL | P46109 | NM_005207 | NP_005198 |
| 231 | 0.8 | Serpin B6 | SERPINB6 | P35237 | NM_001195291, NM_001271822, NM_001271823, NM_001271824, NM_001271825, NM_001297699, NM_001297700, NM_004568, XM_011514672, XM_011514674, XM_011514676, XM_017010940, XM_017010941, XM_024446462, XM_024446463, XM_024446464, XM_024446465 | NP_001182220, NP_001258751, NP_001258752, NP_001258753 NP_001258754, NP_001284628, NP_001284629, NP_004559, XP_011512914, XP_011512976, XP_011512978, XP_016866429, XP_016866430, XP_024302230, XP_024302231, XP_024302232, XP_024302233 |
| 232 | 0.8 | 6-phosphoglucondactonase | PGLS | 095336 | NM_012088, XM_011527897 | NP_036220, XP_011526199 |
| 233 | 0.8 | Guanine nucleotide-bindingprotein G(I)/G(S)/G(T) subunit beta-1 | GNB1 | P62873 | NM_001282538, NM_001282539, NM_002074, XM_017 001059, XM_017001060, XM_017001061, XM_024446495 | NP_001269467, NP_001269468, NP_002065, XP_016856548, XP_016856549, XP_016856550, XP_024302263 |
| 234 | 0.8 | Lactadherin | MFGE8 | Q08431 | NM_001114614, NM_0011310319, NM_001310320, NM_001310321, NM_005928, XM_011022206, XM_024449930 | NP_001108086, NP_001297248, NP_001297249, NP_001297250, NP_005919, XP_016877695, XP_024305698 |
| 235 | 0.8 | Ras-related proteinRal-A | RALA | P11233 | NM_005402, XM_006715762, XM_011515466, XM_011515467 | NP_005393, XP_006715825, XP_011513768, XP_011513769 |
| 236 | 0.8 | Protein transport protein Sec23A | SEC23A | 015436 | NM_006364, XM_005267262, XM_011536355, XM_017020928 | NP_006355, XP_005267319, XP_011534657, XP_016876417 |
| 237 | 0.8 | Dihydropyr midinase-relatedprotein2 | DPYSL2 | Q16555 | NM_0011917293, NM_001244604, NM_001386 | NP_001184222, NP_001231533, NP_001377 |
| 238 | 0.8 | NCK-interacting protein with SH3 domain | NCKIPSD | Q9NZQ3 | NM_016453, NM_184231, XM_017006594, XM_017006595, XM_017006596, XM_017006597, XM_017006598, XM_017006599, XM_017006600 | NP_057537, NP_909119, XP_016862083, XP_016862084, XP_016862085, XP_016862086, XP_016862087, XP_016862088, XP_016862089 |
| 239 | 0.8 | Vesicle-associated membrane protein 5 | VAMP5 | C95183 | NM_006634 | NP_006625 |
| 240 | 0.8 | Protein phosphatase 1F | PPM1F | P49593 | NM_014634, XM_011530556, XM_017029108 | NP_055449, XP_011528858, XP_016884597 |
| 241 | 0.8 | Nuclear receptorcorepressor2 | NC0R2 | Q9Y618 | NM_001077261, NM_001206654, NM_006312 | NP_001070729, NP_001193583, NP_006303 |

TABLE 5-16

| | | | | | | |
|---|---|---|---|---|---|---|
| 242 | 0.8 | Ubiquitin-conjugating enyme E2 D3 | UBE2D3 | P61077 | NM_001300795, NM_003340, NM_181886, NM_181887, NM_181888, NM_181889, NM_181890, NM_181891, NM_181892, NM_181893, XM_017008584, XM_024454202 | NP_001287724, NP_003331, NP_87615, NP_871616, NP_871617, NP_871618, NP_871619, NP_871620, NP_871621, NP_871622, XP_016864073, XP_024309970 |
| 243 | 0.8 | WW domain-bindingprotein 2 | WBP2 | Q969T9 | NM_001330499, NM_001348170, NM_012478 | NP_001317428, NP_001335099, NP_036610 |
| 244 | 0.8 | POTE ankyrn domain fam ily member \| | POTEI | P0CG38 | NM_001277406, XM_011511670, XM_017004132, XM_017004733, XM_017004734, XM_024453046 | NP_001264335, XP_011509972, XP_016860221, XP_016860222, XP_016860223, XP_024308814 |
| 245 | 0.8 | Copine-1 | CPNE1 | Q99829 | NM_001198863, NM_OD3915, NM_162926, NM_152926, NM_152927, NM_152928, NM_152930, NM_152931 | NP_001185792, NP_003906, NP_690902, NP_690903, NP_690904, NP_690905 |
| 246 | 0.8 | Alpha-galactosidase A | GLA | P06280 | NM_000169 | NP_000160 |
| 247 | 0.8 | Coronin-1B | CORO1B | Q9BR76 | NM_001018070, NM_020441 | NP_001018080, NP_065174 |
| 248 | 0.8 | ATP-dependent RNA helicase DDX1 | DDX1 | Q92499 | NM_004939 | NP_004930 |
| 249 | 0.8 | Ubiquitin-conjugatingenzyme E2 K | UBE2K | P61086 | NM_001111112, NM_001111113, NM_001312646, NM_001312647, NM_001312648, NM_005339 | NP_001104582, NP_001104583, NP_001299575, NP_001299576, NP_001299577, NP_005330 |
| 250 | 0.8 | Syntaxin-binding protein1 | STXBP1 | P61764 | NM_001032221, NM_003165 | NP_001027392, NP_003156 |
| 251 | 0.8 | WDrepeat-containingprotein 37 | WDR37 | Q9Y218 | NM_014023 | NP_054742 |
| 252 | 0.8 | MOB kinase activator 1B | MOB1B | Q7L9L4 | NM_001244766, NM_001244767, NM_173468, XM_005265709, XM_011532412, XM_ 011008837 | NP_001231695, NP_001231696, NP_775739, XP_005265766, XP_011530714, XP_016864326 |
| 253 | 0.8 | Nodal modulator 3 | NOM03 | P69849 | NM_001004067, XM_005255318 | NP _001004067, XP_005255375 |
| 254 | 0.8 | Nucleoside diphosphate kinase B | NME2 | P22392 | NM_001018137, NM_001018138, NM_001018139, NM_001198682, NM_002512, NM_001018136 | NP_001018147, NP_001018148, NP_001018149, NP_001185611, NP_002503, NP_001018146 |
| 255 | 0.8 | Prostaglandin reductase 1 | PTGR1 | Q14914 | NM_001146108, NM_001146109, NM_012212, XM_011518394, XM_011518395, XM_017014485 | NP_001139580, NP_001139581, NP_036344, XP_011516696, XP_011516697, XP-016869974 |
| 256 | 0.8 | Phosphoglyceratemutase 2 | PGAM2 | P15259 | NM_000290 | NP_000281 |
| 257 | 0.8 | TLD domain-containing protein 1 | TLDC1 | Q6P9B6 | NM_020947, XM_005256074, XM_005256075, XM-006721240, XM_011523248, XM_011523249, XM_017023510, XM_017023511, XM_017023512 | NP_065998, XP_005256131, XP_005256132, XP_006721303, XP_011521550, XP_011521551, XP_016878999, XP_016879000, XP_016879001 |
| 258 | 0.8 | Calcium-binding protein 39-like | CAB39L | Q9H9S4 | NM_001079670, NM_001287337, NM_001287338, NM_001287339, NM_030925, XM_011535254, XM_011535255, XM_011535256, XM_017020785, XM_011010786, XM_017020787, XM_017020788 | NP_001073138, NP_001274266, NP_001274267, NP_001274268, NP_112187, XP_011533556, XP_011533557, XP_011533558, XP_016876274, XP_016876275, XP_016876276, XP_016876277 |
| 259 | 0.8 | Glutathione S-transferase LANCL1 | LANCL1 | 043813 | NM_001136574, NM_001136575, NM_006055, XM_005246243 | NP_001130046, NP_001130047, NP_006046, XP_005246300 |
| 260 | 0.8 | Prefoldinsubunit 6 | PFDN6 | 015212 | NM_001185181, NM_001265595, NM_001265596, NM_014260 | NP_001172110, NP_001252524, NP_001252525, NP_055075 |
| 261 | 0.8 | Cytosolic phosphdipase A2 | PLA2G4A | P47712 | NM_001311193, NM_024420, XM_005245267, XM_011509642 | NP_001298122, NP_077734, XP_005245324, XP_011507944 |

TABLE 5-17

| | | | | | | |
|---|---|---|---|---|---|---|
| 262 | 0.8 | 14-1-3 protein zetidelta | YWHAZ | P63104 | NM_001135699, NM_001135700, NM_001135701, NM_001135702, NM_003405, NM_145690, XM_005251061, XM_005251063, XM_011517290, XM_017013810, XM_017013811, XM_017013812, XM_024441266 | NP_001129171, NP_001129172 NP_001129173, NP_001129173, NP_003397, NP_663723, XP_005251118, XP_005261120, XP_011515592, XP_01639299, XP_016869300, XP_016859301, XP_0243034 |
| 263 | 0.795 | Secemin-3 | SCRN3 | Q0VDG4 | NM_001193528, NM_024583, XM_005246853, XM_005246854, XM_005246855, XM_005246856, XM_005246857, XM_011511839, XM_017004910 | NP_001180457, NP_078859, XP_005246910, XP_005246911, XP_005246912, XP_005246913, XP_005246914, XP_011510141, XP_016850399 |
| 264 | 0.79 | Syntaxin-bindin protein 2 | STXBP2 | Q15833 | NM_001127396, NM_001272034, NM_006948, XM_011528210, XM_011528212 | NP_001120868, NP_001258963 NP_008880, XP_011526512, XP_011526514 |
| 265 | 0.79 | Receptor-type tyros ine-protein phosphatase eta | PTPRJ | Q12913 | NM_001098503, NM_002843, XM_017018083, XM_017018084, XM_017018085 | NP_001091973, NP_002834, XP_016873572, XP_016873573, XP_016873574 |
| 266 | 0.79 | CD109 ant gen | CD109 | Q6YHK3 | NM_001159587, NM_001159588, NM_133493 | NP_001153059, NP_001153060, NP_598000 |
| 267 | 0.79 | Integrin beta-1 | TGB1 | P05556 | NM_002211, NM_033666, NM_033667, NM_033668, NM_033669, NM_133376 | NP_002202, NP_391988, NP_596867 |
| 268 | 0.79 | LHFPL tetraspan subfamily merrber 2 protein | LHFPL2 | Q6ZUX7 | NM_005779, XM_006714515, XM_024454321, XM_024454322, XM_024454323, XM_024454324, XM_024454325 | NP_005770, XP_006714578, XP_024310089, XP_024310090, XP_024310091, XP_024310092, XP_024310093 |
| 269 | 0.79 | Ras-relaled protein Rab-7a | RAB7A, | P51149 | NM_004637, XM_024453745 | NP_004628, XP_024309513 |
| 270 | 0.79 | Glucose-6-phosphale isorrerase | GPI | P06744 | NM_000175, NM_001184722, NM_001289789, NM_001289790, NM_001329909, NM_001329910, NM_001329911, XM_011526754 | NP_000166, NP_001171651, NP_001276718, NP_001276719, NP_001315838, NP_001316839, NP_001316840, XP_011535056 |
| 271 | 0.79 | Choline transporter-like protein 2 | SLC44A2 | Q8IWA5 | NM_001145066, NM_001363611, NM_020428, XM_005259997, XM_005259999 | NP_01138528, NP_001350540, NP_065161, XP_005260054, XP_005260056 |
| 272 | 0.79 | Phosphoglycerate mutase 1 | PGAM1 | P18669 | NM_001317079, NM_002629 | NP_001304008, NP_002620 |
| 273 | 0.79 | Vacoolar protein sortlng-associated protein 28 homolog | VPS28 | Q9UK41 | NM_016208, NM_183057, XM_005272323, XM_005272324, XM_005272325, XM_011517099 | NP_057292, NP_898880, XP_005212380, XP_00572381, XP_005272382, XP_011515401 |
| 274 | 0.79 | Ras-related protein Ral-B | RALB | P11234 | NM_002881, XM_005263727, XM_005263728, XM_011511574 | NP_002872, XP_005263784, XP_05263785, XP_011509816 |
| 275 | 0.79 | Synaptosomal-associaied protein 23 | SNAP23 | O00161 | NM_003825, NM_130798, XM_006720725, XM_017022692, XM_017022693, XM_017022694 | NP_003816, NP_570710, XP_006720788, XP_016878181, XP_016878182, XP_016878183 |
| 276 | 0.79 | Beta-arrestin-1 | ARRB1 | P49407 | NM_004041, NM_020251, XM_011545034, XM_011545035, XM_017017750, XM_017017751, XM_017017752, XM_017017753, XM_0170117754 | NP_004032, NP_064647, XP_011543336, XP_011543337, XP_016873239, XP_016873240, XP_016873241, XP_016873242, XP_016873243 |
| 277 | 0.79 | Heat shock cognate 71 kDa protein | HSPA8 | P11142 | NM_006597, N_153201, XM_011542798 | NP_006688, NP_694881, XP_011541100 |
| 278 | 0.79 | Rho-related GTP-binding protein RhoC | RHOC | P08134 | NM_001042678, NM_01042679, NM_175744 | NP_00106143, NP_001036144, NP_786886 |
| 279 | 0.79 | Malate dehydrogenase cytoplasmic | MDH1 | P40925 | NM_001199111, NM_001199112, NM_001316374, NM_005917 | NP_001188040, NP_001188041, NP_001303303, NP_005908 |

TABLE 5-18

| | | | | | | |
|---|---|---|---|---|---|---|
| 280 | 0.79 | Amyloid-beta precursor protein | APP | P05067 | NM_000484, NM_001136016, NM_001136129, NM_001136130, NM_001136131, NM_001204301, NM_001204302, NM_001204303, NM_201413, NM_201414, XM_024452075 | NP_000475, NP_001129488, NF_001129601, NP_001129602, NP_001129603, NP_001191230, NP_001191231, NP_001191232, NP_958816, NP_958817, XP 024307843 |
| 281 | 0.79 | Vesicle-associated merrbrane protein 8 | VAMP8 | Q9BV40 | NM_003761, XM_017005170 | NP_003752, XP_016860659 |
| 282 | 0.79 | Lymphocyte function-associated antigen 3 | CD58 | P19256 | NM_001144822, NM_001779, XM_017002869 | NP_001138294, NP_001770, XP_016868358 |
| 283 | 0.79 | Ras-relatead proten Rab-13 | RAB13 | P51153 | NM_001272038, NM_002870, XM_017001959 | NP_001258967, NP_002861, XP_016857448 |
| 284 | 0.79 | Regulator G-protein signaling 19 | RGS19 | P49795 | NM_001039467, NM_005873, XM_005260182, XM_005260183, XM_011528484, XM_011528485, XM_011528486 | NP_001034656, NP_005864, XP_005260239, XP_005260240, XP_011526786, XP_011526787, XP_011526788 |
| 285 | 0.79 | Mtogen-activated protein k nase 14 | MAPK14 | Q16539 | NM_001315, NM_139012, NM_139013, NM_139014, XM_006714998, XM_011514310, XM_017010229, XM_017010300, XM_017010301, XM_017010302, XM_017010303, XM_017010304, XM_017010304 | NP_001306, NP_520581, NP_620582, NP_620583, XP_006715061, XP_011512612, XP_016865788, XP_016865789, XP_016865790, XP_016865791, XP_016865792, XP_016865793, |
| 286 | 0.79 | Cathepsin L2 | CTSV | 060911 | NM_001201575, NM_001333, | NP_001188504, NP_001324 |
| 287 | 0.79 ; | Guanine nudeotide-binding protein G(i) subunit alpha-1 | GNAI1 | P63096 | NM_001256414, NM_002069 | NP_001243343, NP_002060 |
| 288 | 0.79 | Heterogeneous nuclear ribonucleoprotein U | HNRNPU | Q00839 | NM_004501, NM_031844 | NP_004492, NP_114032 |
| 289 | 0.79 | V-type proton ATPase subunit F | ATP6V1F | Q16864 | NM_001198909, NM_004231 | NP_001185838, NP_004222 |
| 290 | 0.79 | CD151 antigen | CD151 | P48509 | NM_001039490, NM_004357, NM_139029, NM_139030, XM_024448780 | NP_001034579, NP_004348, NP_620598, NP_620599, XP_024304548 |
| 291 | 0.79 | Selenoprotein P | SELENOP | P49908 | NM_001085486, NM_00193726, NM_005410 | NP_001078955, NP_001087195, NP_005401 |
| 292 | 0.79 | Cleavage and polyadenylation specificity factor subunit 5 | NUDT21 | C43809 | NM_007006 | NP_008937 |
| 293 | 0.79 | Protein ABHD14B | ABHD14B | Q96IU4 | NM_001146314, NM_001254753, NM_032750 | NP_001139786, NP_001241682, NP_116139 |
| 294 | 0.79 | Phosphopantothenate-cysteine ligase | PPCS | Q9HAB8 | NM_001077447, NM_001287506, NM_001287507, NM_001287508, NM_001287509, NM_001287510, NM_001287511, NM_024664 | NP_001070915, NP_001274435, NP_001274436, NP_001274437, NP_001274438, NP_001274439, NP_001274440, NP_078940 |
| 295 | 0.79 | NAD-dependent malic enzyme, rritochondrial | ME2 | P23368 | NM_001168335, NM_002396 | NP_001161807, NP_002387 |
| 296 | 0.79 | PDZ and LlMdomain prole n1 | PDLIM1 | O00151 | NM_020992, XM_011540330 | NP_066272, XP_011536632 |
| 297 | 0.79 | Msshapen-like kinase 1 | MNK1 | Q8N4C8 | NM_001024937, NM_001321236, NM_015716, NM_153827, NM_170663, XM_006721531, XM_006721532, XM_006721536, XM_011523906, XM_017024704, XM_017024705, XM_017024706, XM_017024707, XM_017024708 | NP_001020108, NP_001308165, NP_056531, NP_722549, NP_733763, XP_006721594, XP_006721595, XP_006721599, XP_011522208, XP_016880193, XP_016880194, XP_016880195, XP_016880196, XP_016880197 |
| 298 | 0.79 | Probable aminopeptidase NPEPL1 | NPEPL1 | Q8NDH3 | NM_001204872, NM_001204873, NM_024663 | NP_001191801, NP_001191802, NP_078939 |
| 299 | 0.79 | Glyoxalase domain-containing protein 4 | GL0D4 | Q9HC38 | NM_001366247, NM_001366248, NM_001366249, NM_001366250, NM_016080, XM_006721540, XM_017024711, XM_017024712, XM_024450778 | NP_001353176, NP_001353177, NP_001353178, NP_001353179, NP_057164, XP_006721603, XP_01688020C, XP_016880201, XP_024306546 |

TABLE 5-19

| | | | | | | |
|---|---|---|---|---|---|---|
| 300 | 0.79 9 | Protein SEC13 homolog | SEC13 | P55735 | NM_001136026, NM_001136232, NM_001278946, NM_030673, NM_183352, XM_005265379, XM_017007019, XM_017007020, XM_017007021, XM_024453701, XM_024453702, XM_024453703, XM_024453704, XM_024453705 | NP_001129498, NP_001129704, NP_001265875, NP_109598, NP_899195, XP_005265436, XP_016862508, XP_016862509, XP_016862510, XP_024309469, XP_024309470, XP_024309471, XP_024309472, XP_024309473 |
| 301 | 0.79 | Serinethreonine-protein phosphatase 6 catalytic subunt | PPP6C | 000743 | NM_001123355, NM_001123369, NM_002721, XM_011518847, XM_024447607 | NP_001116827, NP_001116841, NP_002712, XP_011517149, XP_024303375 |
| 302 | 0.79 | Charged multivesicular body protein 3 | CHMP3 | Q9Y3E7 | NM_001005753, NM_001193517, NM_016079, NM_001198954 | NP_001005753, NP_001180446, NP_057163, NP_001185883 |
| 303 | 0.79 | Thiosulfate sulfurtransferase | TST | Q16762 | NM_001270483, NM_003312 | NP_001257412, NP_003303 |
| 304 | 0.79 | Syntaxin-4 | STX4 | Q12846 | NM_001272095, NM_001272096, NM_004604, XM_005255521 | NP_001259024, NP_001259025, NP_004595, XP_005255578 |
| 305 | 0.79 | Translin-associated protein X | TSNAX | Q99598 | NM_005999 | NP_005990 |
| 306 | 0.79 | E3 ubiquitin-protein ligase RNF128 | RNF128 | Q8TEB7 | NM_024539, NM_194463 | NP_078815, NP_919445 |
| 307 | 0.79 | ADP-ribosylation factor-ke protein 2 | ARL2 | P36404 | NM_001199745, NM_01667 | NP_001186674, NP_001658 |
| 308 | 0.79 | Stromal cell-derived factor 2-like protein 1 | SDF2L1 | Q9HCN8 | NM_022044 | NP_071327 |
| 309 | 0.79 | Geranylgeranyl transferase type-2 subunit alpha | RABGGTA | Q92696 | NM_004581, NM_182836 | NP_004572, NP_878256 |
| 310 | 0.79 | Large neutral amino acids transporter small subunit 3 | SLC43A1 | 075387 | NM_001198810, NM_003627, XM_005274358, XM_011545320, XM_011545321, XM_011545322, XM_017018451, XM_017018452, XM_017018453, XM_024448727, XM_024448728 | NP_001185739, NP_003618, XP_005274415, XP_011543622, XP_011543623, XP_011543624, XP_016873940, XP_016873941, XP_016873942, XP_024304495, XP_024304496 |
| 311 | 0.79 | Putative nucleoside diphosphate kinase | NME2P1 | 060361 | N/A | N/A |
| 312 | 0.79 | Interleukin-17 receptor A | L17RA | Q96F46 | NM_001289905, NM_014339 | NP_001276834, NP_055154 |
| 313 | 0.78 | Ras-related protein Rab-8B | RAB8B | Q92930 | NM_016530, XM_011521669, XM_017022312 | NP_057614, XP_011519971, XP_016877801 |
| 314 | 0.78 | Ceruloplasmin | CP | P00450 | NM_000096, XM_006713499, XM_006713500, XM_006713501, XM_011512435, XM_017005734, XM_017005735 | NP_000087, XP_006713562, XP_006713563, XP_006713564, XP_011510737, XP_016861223, XP_016861224 |
| 315 | 0.78 | Ras-related protein Rab-8A | RAB8A | P61006 | NM_005370, XM_011528020 | NP_005361, XP_011526322 |
| 316 | 0.78 | Growth factor receptor-bound protein 2 | GRB2 | P62993 | NM_002086, NM_203506 | NP_002077, NP_987102 |
| 317 | 0.78 | Triosephosphate somerase | TPI1 | P60174 | NM_000365, NM_001159287, NM_001258026 | NP_000356, NP_001152759, NP_001244955 |
| 318 | 0.78 | Guanine nucleotide-binding protein G(q) subunit alpha | GNAQ | P50148 | NM_002072, XM_017014628 | NP_002063, XP_016870117 |
| 319 | 0.78 | Lipopolysaccharide-binding protein | LBP | P18428 | NM_004139 | NP_004130 |
| 320 | 0.78 | GTP-binding protein Rheb | RHEB | Q15382 | NM_005614, XM_011516457, XM_024446854 | NP_005605, XP_015514759, XP_024302622 |
| 321 | 0.78 | S-formylglutathionehydrolase | ESD | P10768 | NM_001984, XM_005266278, XM_011534954, XM_017020414 | NP_001975, XP_005266335, XP_011533256, XP_016875903 |

TABLE 5-20

| | | | | | | |
|---|---|---|---|---|---|---|
| 322 | 0.78 | cAMP-dependent protein kinase catalytic subunit beta | PRKACB | P22694 | NM_001242857, NM_001242858, NM_001242859, NM_001242860, NM_001242861, NM_001242862, NM_001300915, NM_001300916, NM_001300917, NM_002731, NM_182948, NM_207578, NXM_005271015, XM_005271016, XM_005271017, XM_005271018, XM_005271019, XM_005271020, XM_006710758, XM_017001706, XM_017001707, XM_017001708, XM_017001709, XM_017001710, XM_017001711, XM_017001712, XM_017001713, XM_017001714, XM_017001715, XM_017001716, XM_017001717, XM_017001718, XM_017001719, XM_017001720 | NP_001229786, NP_001229787, NP_001229788, NP_001229789, NP_001229790, NP_001229791, NP_001287844, NP_001287846, NP_002722, NP_891993, NP_997461, XP_005271072, XP_005271073, XP_005271074, XP_005271075, XP_005271076, XP_005271077, XP_006710821, XP_016857195, XP_016857196, XP_016857197, XP_016857198, XP_016857199, XP_016857200, XP_016857201, XP_016857202, XP_016857203 XP_016857204, XP_016857205, XP_016857206, XP_016857207, XP_016857208, XP_016857209 |
| 323 | 0.78 | Multidrug resistance-associated protein 1 | ABCC1 | P33527 | NM_004996, NM_019862, NM_019898, NM_019899, NM_019900, NM_019901, NM_019902, XM_011522497, XM_011522498, XM_017023237, XM_017023238, XM_017023239, XM_017023240, XM_017023241, XM_017023242, XM_017023243 | NP_004987, XP_011520799, XP_011520800, XP_016878726, XP_016878727, XP_016878728, XP_016878729, XP_016878730, XP_0166878731, XP_016878732 |
| 324 | 0.78 | EH domain-containing protein 1 | EHD1 | Q9H4M9 | NM_001282444, NM_001282445, NM_006795, XM_011544739 | NP_001269373, NP_001269374, NP_006786, XP_011543041 |
| 325 | 0.78 | Ubiquitin-conjugating enzyme E2 N | UBE2N | P61088 | NM_003348 | NP_003339 |
| 326 | 0.78 | S-methyl-5′-thioadenosine phosohorylase | MTAP | Q13126 | NM_002451 | NP_002442 |
| 327 | 0.78 | Plasma membrane calcium-transporting ATPase 4 | ATP2B4 | P23634 | NM_001001396, NM_001365783, NM_001365784, NM_001684 | NP_001001396, NP_001352712, NP_001352713, NP_001675 |
| 328 | 0.78 | Hypoxanthine-guanine phosphoribosyltransferase | HPRT1 | P00492 | NM_000194 | NP_000185 |
| 329 | 0.78 ! | 2′,3′-cyclic-nucleotide 3′-phosphodiesterase | CNP | P09543 | NM_001330216, NM_033133, XM_011524340 | NP_001317145, NP_149124, XP_011522642 |
| 330 | 0.78 | Phospholipid scramblase 3 | PLSCR3 | Q9NRY6 | NM_001201576, NM_020360 | NP_001188505, NP_065093 |
| 331 | 0.78 | Dynamin-2 | DNM2 | P50570 | NM_001005360, NM_001005361, NM_001005362, NM_001190716, NM_004945 | NP_001005360, NP_001005361, NP_001005362, NP_001177645, NP_004936 |
| 332 | 0.78 | 4F2 cell-surface ant gen heavychain | SLC3A2 | P08195 | NM_001012661, NM_001012662, NM_001012663, NM_001012664, NM_001013251, NM_002394 | NP_001012680, NP_001012682, NP_001013269, NP_002385 |
| 333 | 0.78 | Overexpressed in coloncarcinoma 1 protein | OCC1 | Q8TAD7 | NM_001145199, NM_207376 | NP_001138671 |
| 334 | 0.78 | Ubiquitin-like modifier-activating enzyme 7 | UBA7 | P41226 | NM_003335, XM_005265430, XM_006713321, XM_011534070, XM_011534071, XM_011534072, XM_024453740 | NP_003326, XP_005265487, XP_006713384, XP_011532372, XP_011532373, XP_011532374, XP_024309508 |
| 335 | 0.78 | Ras-related protein Rap-1A | RAP1A | P62834 | NM_001010935, NM_001291896, NM_002884, XM_017001960, XM_017001961, XM_017001962, XM_017001963, XM_017001964, XM_024448786 | NP_001010935, NP_001278825, NP_002875, XP_016857449, XP_016857450, XP_016857451, XP_016857452, XP_016857453, XP_024304554 |
| 336 | 0.78 | Integrin alpha- | TGA2B | P08514 | NM_000419, XM_011524749, XM_011524750 | NP_000410, XP_011523051, XP_011523052 |

TABLE 5-21

| | | | | | | |
|---|---|---|---|---|---|---|
| 337 | 0.78 | Serine/threonine-protein phosphatase 2A activator | PTPA | Q15257 | NM_001193397, NM_001271832, NM_021131, NM_178000, NM_178001, NM_178002, NM_178003, XM_011518834, XM_011518835, XM_011518836, XM_011518837, XM_011518838, XM_017014888 | NP_001180326, NP_001258761, NP_066954, NP_821067, NP_821068, NP_821070, XP_011517136, XP_011517137, XP_011517138, XP_011517139, XP_011517140, XP_016870377 |
| 338 | 0.78 | Ragulator complex protein LAMTOR3 | LAMTOR3 | Q9UHA4 | NM_001243736, NM_O21970 | NP_001230665, NP_068805 |
| 339 | 0.78 | S-adenosylmethionine synthase isoform type-2 | MAT2A | P31153 | NM_005911 | NP_005902 |
| 340 | 0.78 | Protein CutA | CUTA | 060888 | NM_001014433, NM_001014837, NM_001014838, NM_001014840, NM_015921 | NP_001014433, NP_001014837, NP_001014838, NP_001014840, NP_057005 |
| 341 | 0.78 | Cytoplasm c protein NCK2 | NCK2 | C43639 | NM_001004720, NM_001004722, NM_003581, XM_006712797, XM_011511991, XM_011511992, XM_017005103, XM_017005104, XM_017005105 | NP_001004720, NP_001004722, NP_003572, XP_006712860, XP_011510293, XP_011510294, XP_016860592, XP_016860593, XP_016860594 |
| 342 | 0.78 | Tropomodulin-3 | TM0D3 | Q9NYL9 | NM_014547, XM_017022087, XM_017022088 | NP_055362, XP_016877576, XP_016877577 |
| 343 | 0.78 | COP9 signalosome complex subunit 5 | COPS5 | Q92905 | NM_006837 | NP_006828 |
| 344 | 0.78 | Torsin-1A | TOR1A | C14656 | NM_000113 | NP_000104 |
| 345 | 0.78 | Large proline-rich protien BAG6 | BAG6 | P46379 | NM_001098534, NM_001199697, NM_001199698, NM_004639, NM_080702, NM_080703, XM_011514892, XM_017011275, XM_017011276, XM_017011277, XM_017011278, XM_017011279, XM_017011280, XM_017011281, XM_017011282, XM_017011283, XM_017011284, XM_017011285, XM_017011286, XM_017011287, XM_017011289, XM_017011290, XM_017011291, XM_017011292, XM_017011293, XM_017011294, XM_017011295, XM_017011296, XM_017011297, XM_017011298, XM_024446545, XM_024446546, XM_024446547 | NP_001092004, NP_001186626, NP_00186627, NP_004630, NP_542433, NP_542434, XP_011513194, XP_016866764, XP_016866765, XP_016866766, XP_016866767, XP_016886768, XP_016866769, XP_016866770, XP_016866771, XP_016866772, XP_016866773, XP_016866774, XP_016866775, XP_016866776, XP_016866778, XP_016866779, XP_016866780, XP_016866781, XP_016866782, XP_016866783, XP_016866784, XP_016866785, XP_016866786, XP_016866787, XP_024302313, XP_024302314, XP_024302315 |
| 346 | 0.78 | 5′(3′)-deoxy bonucleotidase, cytosolic type | NT5C | Q8TCD5 | NM_001252377, NM_014595, XM_011524700 | NP_001239306, NP_055410, XP_011523002 |
| 347 | 0.78 | Heat shock 70 kDa protein 6 | HSPA6 | P17066 | NM_002155 | NP_002146 |
| 348 | 0.78 | Serine/threonine-protein Kinase24 | STK24 | Q9Y6E0 | NM_001032296, NM_001286649, NM_003576, XM_005254079, XM_017020794, XM_024449426 | NP_001027467, NP_001273578, NP_003567, XP_005254136, XP_016876283, XP_024305194 |
| 349 | 0.78 | Protein S100-A4 | S100A4 | P26447 | NM_002961, NM_019554 | NP_002952, NP_062427 |
| 350 | 0.78 | SCY1-like protein 2 | SCYL2 | Q6P3W7 | NM_001317784, NM_001330253, NM_001330254, NM_001330256, NM_017988 | NP_001304713, NP_001317182, NP_001317183, NP_001317185, NP_060458 |
| 351 | 0.78 | 5′-AMP-activated protein kinasesubunit gamma-1 | PRKAG1 | P54619 | NM_001206709, NM_001206710, NM_002733, NM_212461, XM_005269019, XM_005269020, XM_011538562, XM_017019622, XM_017019623 | NP_001193638, NP_001193639, NP_002724, XP_005269076, XP_005269077, XP_011536864, XP_016875111, XP_016875112 |
| 352 | 0.78 | Ubiquitin-conjugating enzyme E2 variant 2 | UBE2V2 | Q15819 | NM_003350, XM_011517583, XM_017013808 | NP_003341, XP_011515885, XP_016869297 |
| 353 | 0.78 | Tyrosine-protein phosphalase non-receptor type 1 | PTPN1 | P18031 | NM_001278618, NM_002827 | NP_001265547, NP_002818 |

TABLE 5-22

| | | | | | | |
|---|---|---|---|---|---|---|
| 354 | 0.78 | Disheveled-associated actvator of morphogenesis 1 | DAAM1 | Q9Y4D1 | NM_001270520, NM_014992, XM_005267430, XM_005267431 | NP_001257449, NP_055807, XP_005267487, XP_005267488 |
| 355 | 0.78 | Inositol monophosphatase 1 | IMPA1 | P29218 | NM_001144878, NM_001144879, NM_005536 | NP_001138350, NP_001138351, NP_005527 |
| 356 | 0.78 | Isoamyl acetate-hydrolyzing esterase 1 homolog | AH1 | Q2TAA2 | NM_001039613, NM_001320858, NM_001320859, NM_001320860, NM_001320863, XM_024452813 | NP_001034702, NP_001307787, NP_001307788, NP_001307789, NP_001307792, XP_024308581 |
| 357 | 0.78 | COP9 signalosome complex subunit 4 | COPS4 | Q9BT78 | NM_001258006, NM_001330727, NM_016129 | NP_001244935, NP_001317656, NP_057213 |
| 358 | 0.78 | Peptidyl-prdyl cis-trans isomerase NIMA-interacting 4 | PIN4 | Q9Y237 | NM_001170747, NM_006223 | NP_001164218, NP_006214 |
| 359 | 0.78 | Phosducin-like protein 3 | PDCL3 | Q9H2J4 | NM_024065, XM_017004890 | NP_076970, XP_016860379 |
| 360 | 0.77 | Alpha-soluble NSF attachment protein | NAPA | P54920 | NM_003827, XM_011527436, XM_011527437 | NP_003818, XP_011525738, XP_011525739 |
| 361 | 0.77 | Syntaxin-7 | STX7 | O15400 | NM_001326578, NM_001326579, NM_001326580, NM_003569 | NP_001313507, NP_001313508, NP_001313509, NP_003560 |
| 362 | 0.77 | Claudin-7 | CLDN7 | C95471 | NM_001185022, NM_001185023, NM_001307 | NP_001171951, NP_001171952, NP_001298 |
| 363 | 0.77 | Tandem C2 domans nuclear protein | TC2N | Q8N9U0 | NM_001128595, NM_001128596, NM_001289134, NM_152332 | NP_001122067, NP_001122068, NP_001276063, NP_689545 |
| 364 | 0.77 | Protein eva-1 homolog B | EVA1B | Q9NVM1 | NM_001304762, NM_018166, XM_017001627 | NP_001291691, NP_060636, XP_016857116 |
| 365 366 | 0.77 0.77 | Annexin A7 Ras-related oroten Rab-14 | ANXA7 RAB14 | P20073 P61106 | NM_001156, NM_001320879, NM_001320880, NM_004034, XM_017016162, XM_017016163 NM_016322 | NP_001147, NP_001307808, NP_001307809, NP_004025, XP_016871651, XP_016871652 NP_057406 |
| 367 | 0.77 | WD repeat-contianing protein 1 | WDR1 | 075083 | NM_005112, NM_017491, XM_017008880 | NP_005103, NP_059830, XP_016864369 |
| 368 | 0.77 | Cofilin-1 | CFL1 | P23528 | NM_005507 | NP_005498 |
| 369 | 0.77 | Calpain-1 catalytc subunit | CAPN1 | P07384 | NM_001198868, NM_001198869, NM_005186, XM_006718698, XM_011545292 | NP_001185797, NP_001185798, NP_005177, XP_006718761, XP_011543594 |
| 370 | 0.77 | Calcium and integrin-binding protein 1 | CIB1 | Q99828 | NM_001277764, NM_006384, XM_006720375 | NP_001264693, NP_006375, XP_006720438 |
| 371 | 0.77 | Ras-related oroten Rab-4B | RAB4B | P61018 | NM_016154 | NP_057238 |
| 372 | 0.77 | Ras-related orotein Rab-6A | RAB6A | P20340 | NM_001243718, NM_001243719, NM_002869, NM_198896 | NP_001230647, NP_001230648 NP_002860, NP_942599 |
| 373 | 0.77 | Glutathione S-transferase P | GSTP1 | P09211 | NM 000852 | NP_000843 |
| 374 | 0.77 | Actn, cytoplasmic 1 | ACTB | P60709 | NM_001101 | NP_001092 |
| 375 | 0.77 | Talin-2 | TLN2 | Q9Y4G6 | NM_015059, XM_005254708, XM_005254710, XM_005254711, XM_005254712, XM_005254713, XM_05254714, XM_005254715, XM_006720717, XM_017022665, XM_017022666, XM_0107022667, XM_017022668, XM_017022669, XM_024450087 | NP_055874, XP_005254765, XP_005254767, XP_005254768, XP_005254769, XP_005254770, XP_005254771, XP_005254772, XP_006720780, XP_016878154, XP_016878155, XP_016878156, XP_016878157, XP_016878158, XP_024305855 |
| 376 | 0.77 | CD59 glycoprotein | CD59 | P1398i | NM_000611, NM_001127223, NM_001127225, NM_001127226, NM_001127227, NM_203329, NM_203330, NM_203331 | NP_000602, NP_001120695, NP_001120697, NP_001120698, NP_001120699, NP_976074, NP_976075, NP_976076 |
| 377 | 0.77 | Plasma protease C1 inhbitor | SERPING1 | P05155 | NM_000062, NM_001032295 | NP_000053, NP_001027466 |
| 378 | 0.77 | Ubiquitin-like modifier-activating enzyme 1 | UBA1 | P22314 | NM_003334, NM_153280, XM_005272649, XM_011543954, XM_017029777, XM_0107029778, XM_017029779, XM_017029780, XM_017029781 | NP_003325, NP_695012, XP_005272706, XP_011542256, XP_016885266, XP_016885267, XP_016885268, XP_016885269, XP_016885270 |

TABLE 5-23

| | | | | | | |
|---|---|---|---|---|---|---|
| 379 | 0.77 | Grancalcin | GCA | P23676 | NM_001330266, NM_01330266, NM_001330267, NM_001330268, NM_001330270, NM_001330271, NM_012198, XM_005246446, XM_006712398, XM_006712400, XM_011510926, XM_011510928, XM_017003767, XM_017003769 | NP_001317194, NP_001317195, NP_001317196, NP_001317197, NP_001317199, NP_001317200, NP_036330, XP_005246503, XP_006712461, XP_006712463, XP_011509228, XP_011509230, XP_016859256, XP_016859258 |
| 380 | 0.77 | Canalicular multispecific organic anion transporter 2 | ABCC3 | 015438 | NM_001144070, NM_003786, NM_020037, NM_020038, XM_005257763, XM_011525422, XM_011525423, XM_011525424, XM_011525425, XM_017025265, XM_017025266 | NP_001137542, NP_003777, XP_005257820, XP_011523724, XP_011523725, XP_011523726, XP_011523727, XP_016880754, XP_016880755 |
| 381 | 0.77 | Vacuolar protein sorting-associated protein 45 | VPS45 | Q9NRW7 | NM_001279353, NM_001279354, NM_001279355, NM_007259, XM_024452791 | NP_001266282, NP_001266283, NP_009190, XP_024308559 |
| 382 | 0.77 | Heast shock protein HSP 9C-alpha | HSP90AA1 | P07900 | NM_001017963, NM_005348, XM_011536718 | NP_001017963, NP_005339, XP_011535020 |
| 383 | 0.77 | Tight junction protein ZO-2 | TJP2 | Q9UDY2 | NM_001170414, NM_001170415, NM_001170416, NM_001170630, NM_004817, NM_201629, XM_005252314, XM_011519204, XM_011519206, XM_011519207, XM_011519208, XM_011519209, XM_017015327, XM_017015328, | NP_001163885, NP_001163886, NP_001163887, NP_004808, NP_963923, XP_005252371, XP_011517506, XP_011517508, XP_011517509, XP_011517510, XP_011517511, XP_016870816, XP_016870817 |
| 384 | 0.77 | Ras-related protein R-Ras | RRAS | P10301 | NM_006270 | NP_006261 |
| 385 | 0.77 | Actin-related protein ⅔ complex subunit 5-like protein | ARPC5L | Q9BPX5 | NM_030978,XM_011519067 | NP_112240, XP_011517369 |
| 386 | 0.77 | Nucleosome assembly protein 1-like 1 | NAP1L1 | P55209 | NM_001307924, NM_001330231, NM_001330232, NM_004537, NM_139207, XM_011538393, XM_017019338, XM_017019339, XM_017019340, XM_024448983 | NP_001294853, NP_001317160, NP_001317161, NP_004528, NP_631946, XP_011536695, XP_016874827, XP_016874828, XP_016874829, XP_024304751 |
| 387 | 0.77 | Beta-1-syntrophin | SNTB1 | Q13884 | NM_021021, XM_011517239 | NP_066301, XP_011515541 |
| 388 | 0.77 | Monocarboxylate transporter 4 | SLC16A3 | O15427 | NM_001042422, NM_001042423, NM_001206950, NM_001206951, NM_001206952, NM-004207, XM_024451023 | NP_001035887, NP_001035888, NP_001193879, NP_001193880, NP_001193881, NP_004198, XP_024306791 |
| 389 | 0.77 | Dynein light chain roadblock-type 1 | DYNLRB1 | Q9NP97 | NM_001281727, NM_001281728, NM_001281729, NM_001319157, NM_014183, NM_177953 | NP_001268656, NP_001268657, NP_001268658, NP_001306086, NP_054902, NP_808852 |
| 390 | 0.77 | Prefoldin subunit 2 | PFDN2 | Q9UHV9 | NM_012394, XM_011509624 | NP_036526, XP_011507926 |
| 391 | 0.77 | Rab GDP dssociation inhbitor beta | GDl2 | P50395 | NM_001115156, NM_001494, XM_017016071 | NP_001108628, NP_001485, XP_016871560, |
| 392 | 0.77 | Podocalyxin | PODXL | O00592 | NM_001018111, NM_005397 | NP_001018121, NP_005388 |
| 393 | 0.77 | Vacuolar protein sorting-associated protein 4A | VPS4A | Q9UN37 | NM_013245 | NP_037377 |
| 394 | 0.77 | SH3 domain-binding glutamic ac d-rich-like protein 3 | SH3BGRL3 | Q9H299 | NM_031286 | NP_112576 |

TABLE 5-24

| | | | | | | |
|---|---|---|---|---|---|---|
| 395 | 0.77 | Galactosylceramide sulfotransferase | GAL3ST1 | Q99999 | NM_001318103, NM_001318104, NM_001318105, NM_001318106, NM_001318107, NM_001318108, NM_001318109, NM_001318110, NM_001318111, NM_001318112, NM_001318113, NM_001318114, NM_001318115, NM_001318116, NM_004861, XM_011530518, XM_011530522, XM_011530524, XM_011530528, XM_017029096, XM_017029097, XM_017029098, XM_024452304 | NP_001305032, NP_001305033 NP_001305034, NP_001305035, NP_001305036, NP_001305037, NP_001305038, NP_001305039, NP_001305040, NP_001305041, NP_001305042, NP_001305043, NP_001305044, NP_001305045, NP_004852, XP_011528820, XP_011528824, XP_011528826, XP_011528830, XP_016884585, XP_016884586, XP_016884587, XP_024308072 |
| 396 | 0.77 | Nuclear receptor-binding protein | NRBP1 | Q9UHY1 | NM_001321357, NM_001321358, NM_001321359, NM_001321361, NM_001321362, NM_001321363, NM_013392 | NP_001306286, NP_001308287, NP_0013082288, NP_001308290, NP_001308291, NP_001308292, NP_037524 |
| 397 | 0.77 | Nucleoside diphasphate kinase A | NME1 | P15531 | NM_000269, NM_198175 | NP_00026C, NP_937818 |
| 398 | 0.77 | 3-mercaplooyiruvate sulfurtransferase | MPST | P25325 | NM_001013436, NM _0C1013440, NM_001130517, NM_021126, XM_005261610, XM_011530195, XM_C11530196 | NP_001013454, NP_001123989, NP_066949, XP_005261667, XP_011528497, XP_011528498 |
| 399 | 0.77 | Lysoohospholipase-like protein 1 | LYPLAL1 | Q5VWZ2 | NM_001300169, NM_001300770, NM_001300771, NM 001300772, NM_001350628, NM_001350629, NM_138794, XM_011509183, XM_17000271, XU_01700027 | NP_001281698, NP_001287699, NP_001287700, NP_001287701, NP_001337557, NP_001337558 NP_620149, XP_011507485, XP_016855760, XP_016855761 |
| 400 | 0.77 | Ras suppressor protein 1 | RSU1 | Q15404 | NM_012425, NM_152724, XM_005252552 | NP_036557, NP_689937, XP_005252609 |
| 401 | 0.77 | Ras-related orote n Rab-4A | RAB4A | P20338 | NM_001271998, NM_004578 | NP_001258927, NP_004569 |
| 402 | 0.77 | Myosin-14 | MYH14 | Q77406 | NM_001077186, NM_001145809, NM_024729, XM-006723386, XM_011527320, XM_011527321, XM_011527323, XM_024451721 | NP_001070654, NP_001139281, NP_079005, XP_006723449, XP_01 1625622, XP_011525625, XP_011525625, XP_024307489 |
| 403 | 0.77 | Dynactin subunit 4 | DCTN4 | Q9UJW0 | NM_001135643, NM_001135644, NM_016221, XM_005268457, XM_011537644, XM_011537645, XM_011537646, XM_011537647 | NP_001129115, NP_001129116, NP_057305, XP_05268514, XP_011535946, XP_011535947, XP_011535948, XP_011535949 |
| 404 | 0.77 | Calcium release-activated calcium channel protein 1 | ORAI1 | Q96D31 | NM_032790 | NP_116179 |
| 405 | 0.77 | Procollagen c-endooept dase enhancer 2 | PCOLCE2 | Q9UKZ9 | NM_013363 | NP_037495 |
| 406 | 0.77 | Gamma-endase | ENO2 | P09104 | NM_001975 | NP_001966 |
| 401 | 0.77 | Annexin A8 | ANXA8 | P13928 | NM_001040084, NM_001271702, NM_001271703, XM_006717951, XM_011540096, XM_011540098, XM_011540099, XM_011540101, NM_001039801, NM_0C1098845, NM_001278923, NM_0C1278924, NM_00163C | NP_001035173, NP_001258631, NP_001258632, XP_006718014, XP_01153838, XP_011538400, XP_011538401, XP_011538403, NP_001091315, NP_001265852, NP_001265853 |
| 408 | 0.77 | CHLF-like MARVEL transmembrane domain-containing orote n 6 | CMTM6 | Q9NX76 | NM_017801 | NP_060271 |
| 409 | 0.77 | Carcinoembyon c antigen-related cell adhesion molecule 6 | CEACAM6 | P40199 | NM_002483, XM_011526990 | NP_002474, XP_011525292 |
| 410 | 0.77 | Heterogeneous nuclear ribonucleoprotein A0 | HNRNPA0 | Q13151 | NM_006805 | NP_006796 |

TABLE 5-25

| | | | | | | |
|---|---|---|---|---|---|---|
| 411 | 0.77 | Transmembrane channel ke 6 | TMC6 | | NM_001127198, NM_Ol 321185, NM_007267 | XP_011522558, XP_011522559, XP_011522560, XP_016879596, XP_016879597, XP_024306323, XP 024306324, XP 024306326 |
| 412 | 0.77 | Transmembane protein 222 | TMEM222 | Q9H0R3 | NM_032125 | NP-115501 |
| 413 | 0.77 | 7 Kdmine reductase mu-crystalli | CRYM | Q14894 | NM_001014444, NM_001888, XM_021450157 | NP_001871, XP_02430S925 |
| 414 | 0.77 | GMP reduclase 2 | GMPR2 | C9P2T1 | NM_001002000, NM_001002001, NM_001002002, NM_001283022, NM_0C1283023, NM_001351022, NM_001351023, NM_001351024, NM_001351025, NM_001351029, NM_016516, XM_005267742, XM_006720165, XM_011021356, XM_017021360 | NP_001002000, NP_001002001, NP_001002002, NP_001269950, NP_001269951, NP_001269952, NP_00133791, NP_001331952, NP_001337953, NP_001137954, NP_001331953, NP_057660, XP_00526779, XP_006720228, XP_016876845, XP_016876849 |
| 415 | 0.765 | Synembryn-A | RIC8A | Q9NPQ8 | NM_001286134, NM_021932, NM_005253052, XM_017018107 | NP_001273063, NM_068751, XP_005253109, XP_016873596 |
| 416 | 0.76 | Glyceraldehyde-3-phosphate dehydrogenase | GAPDH | P04406 | NM_001256199, NM_001289745, NM_001289146, NM_001357938, NM_001357943, NM_001357944, NM_002045 | NP_001243728, NP_001276674, NP_001276675, NP_001344872, NP_002037 |
| 417 | 0.76 | Vacuolar protein sorting-associated protein 37B | VPS37B | Q9H9H4 | NM_024667, XM_005253622, XM_006719602, XM_011538743, XM_311538745, XM_017019970, XM_017019971, XM_01701972 | NP_078943, XP_005253679, XP_006719665, XP_011537045, XP_011537041, XP_016875459, XP_016875460, XP_016875461 |
| 418 | 0.76 | Deoxyribose-phosohate aldolase | DERA | Q9Y315 | NM_001300779, NM_015954, XM_024449000, XM_024449001 | NP_001287708, NP_057038, XP_024304768, XP_024304769 |
| 419 | 0.76 | CD9 antigen | CD9 | P21926 | NM_001330312, NM_001769, XM_005253814 | NP_001317241, NP_001760, XP_005253871 |
| 420 | 0.76 | Chloride intracellular channel protein 1 | CLIC1 | C00299 | NM_001281593, NM_001287594, NM_001288 | NP_001214622, NP_0012744523, NP_001279 |
| 421 | 0.76 | Myeloid-associated differentiation marke | MYADM | Q96S97 | NM_001020818, NM_001020819, NM_001020820, NM_001020821, NM_001290189, NM_001290189, NM_001290190, NM_001290191, NM_001290192, NM_001290193, NM_001290194, MM_18373 | NP_001018654, NP_001018655, NP_001018656 NP_001018657, NP_001277117, NP_001277118, NP_001277119, NP_001277120, NP_001271121, NP_001271122 NP_001277123, NP_612182 |
| 422 | 0.76 | Phosphoglycerate kinase 1 | PGK1 | P00558 | NM_000291 | NP_000282 |
| 423 | 0.76 | Integrin beta-3 | TGB3 | P05106 | NM_000212 | NP_000201 |
| 424 | 0.76 | Alpha-enolase | EN01 | P06733 | NM_001201483, NM_001353346, NM_001428 | NP_001188412, NP_001340275, NP_001419 |
| 425 | 0.76 | Vestle-associated membrane protein 3 | VAMP3 | G15836 | NM_004781, XM_017002841 | NP_004772, XP_016858330 |
| 426 | 0.76 | Guanine nucleotide-binding protein G(I)/G(S)/G(O) subunit gamma- 5 | GNG5 | P63218 | NM_005274 | NP_006265 |
| 427 | 0.76 | Interleukin-5 receptor subunit beta | L6ST | P40189 | NM_001190981, NM_001364275, NM_001364276, NM_001364271, NM_0013642718, NM_001364279, NM_002184, NM_175767, XM_017000441, XM_017009442 | NP_001111910, NP_001351204, NP001351205, NP001351206, NP_01351207, NP_001351208, NP_002175, NP_786943, XP_016864930, XP_016864931 |

TABLE 5-26

| | | | | | | |
|---|---|---|---|---|---|---|
| 428 | 0.76 | Autophagy-related protein 16-1 | ATG16L1 | G676U5 | NM_001190269, NM_001190267, NM_001363742, NM_017974, NM_130803, NM_198890, XP_005246082, XM_006246064, XM_005246086, XM_306712608 | NP_001177195, NP_001177196, NP_001350671, NP_064444, NP_110430, NP_942593, XP_005246139, XP_005245141, XP_005246143, XP_006712671 |
| 429 | 0.76 | Osteoclast-slimulating factor 1 | OSTF1 | Q92882 | NM_012383, XM_006717053, XM_006717055, XM_011518524, XM_011518525, XM_017014622 | NP_036515, XP_006717116, NP_006717118, XP_011516826, XP_011516827, XP_16870111 |
| 430 | 0.76 | Phosphatidylinositd 5-phosohate 4-kinase type-2 alpha | PIP4K2A | P48426 | NM_001330062, NM_05028, XM_006717450, XM_017016330, XM_317016331, XM_011016332 | NP_001316991, NP_0051019, XP_006717513, XP_016871819, XP_016811820, XP_016871821 |
| 431 | 0.76 | Heat shock protein 75 kDa, mitochoidral | TRAP1 | Q12931 | NM_001272049, NM_016292, XM_011522345 | NP_001258918, NP_057375, XP_011520647 |
| 432 | 0.76 | Ras-relatea oroten Rap=1b | RAP1B | P61224 | NM_001010942, NM_001251917, NM_001251918, NM_001251921, NM_001251922, NM_015646 | NP_001010942, NP_001238846, NP_001238847, NP_001238850, NP_001238851, NP_056161 |
| 431 | 0.76 | Chlaride intracallular channel protein 6 | CLIC6 | Q96NY7 | NM_00131799, NM_053277, XM_017028406 | NP_001303938, NP_444507, XP_016883895 |
| 434 435 | 0.76 | Ras-related oroten Rab-27B Plasin.2 | RAB27B LCP1 | O00194 P13796 | NM_004163, XM_005266133, XM_006722618, XM_011526131, XM_017025913, XM_017025914, XM_024451232, NM_002298, XM_05266314 | NP_004154, XP_005266790, XP_006722581, XP_011524433, XP_016881402, XP_016881403, XP_024307000, NP_002289, XP_005266431 |
| 436 | 0.76 | LIMand SH3 domain protein 1 | LASP1 | Q14847 | NM_001271608, NM_006148 | NP_001258537, NP_006139 |
| 437 | 0.76 | Indian hedgehog protein | IHH | Q14623 | NM_002181 | NP_002172 |
| 438 | 0.76 | Sod um- and chloride-dependent taurine transporter | SLC6A6 | P31641 | NM_001134367, NM_001134368, NM_003043, XM_006713307, XM_011534030, XM_011534031 | NP_001121839, NP_001127840, NP_003034, XP_006713370, XP_011632332, XP_011532333 |
| 439 | 0.76 | Protein flightless-1 nomolog | FLII | Q13045 | NM_001256264, NM_001256265, NM_002018, XM_005256555, XM_00526556, XM_005256558 | NP_001243193, NP_001243194, NP_002009, XP_005256612, XP_006256613, XP_005256615 |
| 440 | 0.76 | D-aminoacyl-tRNA deacylase 1 | DTD1 | Q8TEA8 | NM_001318043, NM_080820, XM_005260872 | NP_001304912, NP_54301, XP_005260929 |
| 441 | 0.76 | Tyrosine-protien phosphatase non-receptor type 6 | PTPN6 | P29350 | NM_002831, NM_080548, NM_080549, XM_006718994, XM_011520988, XM_024449106 | NP_002822, NP_536858, NP_536859, XP_0067197, XP_011519290, XP_02430d874 |
| 442 | 0.76 | Syntaxin-12 | STX12 | Q86Y82 | NM_177424 | NP_803173 |
| 443 | 0.76 | Beta-actin-like protein 2 | ACTBL2 | Q562R1 | NM_001011992 | NP_001011992 |
| 444 | 0.76 | Ubiquitin carooxyl-terminal hydrolase isozyme L3 | UCHL3 | P15374 | NM_00127092, NM_006002, XM_011535212, XM_011535213, XM_011535214, XM_017020725, XM_317020726, XM_011020127 | NP_001251881, NP_005993, XP_011533514, XP_011533515, XP_011533516, XP_016876214, XP_016876215, XP_016876216 |
| 445 | 0.76 | Prefoldin subunit 5 | PFDN5 | Q99471 | NM_002624, XM_145896, NM_145897 | NP_002615, NP_665904 |
| 446 | 0.76 | Dehdrogenase/reductase SDR family member 7 | DHRS7 | Q9Y394 | NM_001322280, NM_001322281, NM_001322282, NM_016029, XM_017021368 | NP_001309209, NP_001309210, NP_001309211, NP_051113, XP_16876857 |
| 447 | 0.76 | Sortilin | SORT1 | Q99523 | NM_001205228, NM_002959, XM_005271100, XM_005271101, XM_105271102, XM_006710812 | NP_001192157, NP_002950, XP_005271157, XP_005271158, XP_005271159, XP_006710875 |
| 448 | 0.76 | Ragulator complex protein LAMTOR5 | LANTOR5 | O43504 | NM_006402 | NP_006391 |

TABLE 5-27

| | | | | | | |
|---|---|---|---|---|---|---|
| 449 | 0.76 | Elongation factor 1-delta | EEF1D | P29692 | NM_001130053, NM_001130054, NM_001130055, NM_001130056, NM_001130057, NM_001195203, NM_001289950, NM_001317743, NM_001330646, NM_001960, NM_C32378, XM_005250820, XM_005250823, XM_006716519, XM_006716520, XM_006716522, XM_006716523, XM_006716524, XM_006716525, XM_011516905, XM_011516906, XM_017013170, XM_017013171, XM_017013172, XM_024447088, XM_024447089 | NP_001123525, NP_001123527, NP_001123528, NP_001123529, NP_001181132, NP_001276879, NP_001304672, NP_001317575, NP_001951, NP_115754, XP_005250877, XP_005250880, XP_006716582, XP_006716583, XP_006716585, XP_006716586, XP_006716587, XP_006716588, XP_011515207, XP_011515208, XP_016868659, XP_016868660, XP_016868661, XP_024302856, XP_024302857 |
| 450 | 0.76 | Cyclin-Y | CCNY | Q8ND76 | NM_001282852, NM_001282853, NM_001282854, NM_145012, NM_181698 | NP_001268781, NP_001269782 NP_001269783, NP_659449, NP_859049 |
| 451 | 0.76 | Sul-otransferase 1A1 | SULT1A1 | P50225 | NM_001055, NM_177529, NM_177530, NM_177534, NM_177536, XM_017023604, XM_017023605, XM_017023607, XM_017023608, XM_017023609, XM_0171023610, XM_017023611, XM_017023612, XM_017023613, XM_024450408, XM_024450409, XM_024450410, XM_024450411 | NP_001046, NP_803565, NP_803566, NP_803878, NP_803880, XP_016879093, XP_016879094, XP_016879096, XP_016879097, XP_016879098, XP_016879099, XP_016879100, XP_016879101, XP_016879102, XP_024306176, XP_024306177, XP_024306178, XP_024306179 |
| 452 | 0.76 | L-xylulose reductase | DCXR | Q7Z4W1 | NM_001195218, NM_016286 | NP_001182147, NP_057370 |
| 453 | 0.76 | Phosphatidylinositol-glycan-specifc phospholipase D | GPLD1 | P80108 | NM_001503, NM_177483, XM_017010753, XM_024446403 | NP_001494, XP_016866242, XP_02430271 |
| 454 | 0.76 | Protein PRRC1 | PRRC1 | Q96M27 | NM_001286808, NM_130809, XM_006714529 | NP_001273737, NP_570721, XP_006714592 |
| 455 | 0.76 | GTPase KRas | KRAS | P01116 | NM_004985, NM_033360, XM_006719069, XM_ 011520653 | NP_004976, NP_203524, XP_006719132, XP_011518955 |
| 456 | 0.76 | Polypeptide N-acetylgalactosaminyltransferase 2 | GALNT2 | Q10471 | NM_001291868, NM_004481, XM_017000963, XM_017000964, XM_017000965, XM_ 017000966 | NP_001278795, NP_004472, XP_016856452, XP_016856453, XP_016856454, XP 016856455 |
| 457 0.76 | 0.76 | Src substrale cotactin | CTTN | Q14247 | NM_001184740, NM_005231, NM_138565, XM_-006718447, XM_006718448, XM_017017312 | NP_001171669, NP_005222, NP_612632, XP_006718510, XP_006718511, XP_016872801 |
| 458 | 0.76 | AP-1 comolexsubunit sigma-1A | AP1S1 | P61966 | NM_001283, NM_057089 | NP_001274 |
| 459 | 0.76 | Elongin-C | ELOC | Q15369 | NM_001204857, NM_001204858, NM_001204859, NM_001204860, NM_001204861, NM_001204862, NM_001204863, NM_001204864, NM_005648, XM_011517580, XM_011517581 | NP_001191786, NP_001191787, NP_001191788, NP_001191789, NP_001191790, NP_001191791, NP_001191792, NP_001191793 NP_005639, XP_011515882, XP_011515883 |
| 460 | 0.76 | cGMP-specific 3′,5′-cyclic phosphodiesterase | PDE5A | C76074 | NM_001083, NM_033430, NM_033437, XM_017008791 | NP_001074, NP_236914, NP_246273, XP_016864280 |
| 461 | 0.76 | DNA damage-binding protein 1 | DDB1 | Q16531 | NM_001923 | NP_001914 |

TABLE 5-28

| | | | | | | |
|---|---|---|---|---|---|---|
| 462 | 0.76 | Rab-like protein 2A | RABL2A | Q9UBK7 | NM_001306158, NM_001306159, NM_001306160, NM_001306161, NM_001354405, NM_001354406, NM_001354407, NM_001354408, NM_001354409, NM_001354410, NM_001354412, NM_001354413, NM_001354414, NM_001354416, NM_001354417, NM_001354418, NM_001354419, NM_001354421, NM_001354423, NM_001354424, NM_001354425, NM_001364426, NM_001354427, NM_001354428, NM_007082, NM_013412, XM_006712208, XM_006712209, XM_011510508, XM_017003203, XM_017003204, XM_017003205, XM_017003207, XM_017003210, XM_017003211, XM_017003212, XM_017003213, XM_017003214, XM_017003215, XM_017003216, XM_017003220, XM_017003222, XM_017003223, XM_017003226, XM_017003230, XM_017003234, XM_024452573, XM_024452575, XM_024452576 | NP_001293087, NP_001293088, NP_001293089, NP_001293090, NP_001341334, NP_001341335, NP_001341336, NP_001341337, NP_001341338, NP_001341339, NP_001341341, NP_001341342, NP_001341343, NP_00134145, NP_001341346, NP_001341347, NP_001341348, NP_001341350, NP_001341352, NP_001341353, NP_001341354, NP_001341355, NP_001341356, NP_001341357, NP_009013, NP_038198, XP_006712271, XP_006712272, XP_011508811, XP_016858692, XP_016858693, XP_016858694, XP_016858696, XP_016858699, XP_016858700, XP_016858701, XP_016858702, XP_016858703, XP_016858704, XP_016858705, XP_016858709, XP_016858711, XP_016858712, XP_016858715, XP_016858719, XP_016858723, XP_024308341, XP_024308343, XP_024308344 |
| 463 | 0.76 | U6 snRNA-associated Sm-like protein LSm6 | LSM6 | P62312 | NM_007080, XM_017007672, XM_017007673 | NP_009011, XP_016863161, XP_016863162 |
| 464 | 0.76 | PDZ and LIM doma n protein 7 | PDLIM7 | Q9NR12 | NM_005451, NM_203352, NM_203353, NM_213636, XM_011534698, XM_011534699 | NP_005442, NP_976227, NP_998801, XP_011533000, XP_011533001 |
| 465 | 0.76 | Secernin-1 | SCRN1 | Q12765 | NM_001145513, NM_001145514, NM_001145515, NM_014766, XM_024447007 | NP_001138985, NP_001138986 NP_001138987, NP_055581, XP_024302775 |
| 466 | 0.76 | Metastasis-associated protein MTA2 | MTA2 | C94776 | NM_001330292, NM_004739 | NP_001317221, NP_004730 |
| 467 | 0.76 | Developmentally-regulated GTP-binding protein 1 | DRG1 | Q9Y295 | NM_004147 | NP_004138 |
| 468 | 0.76 | Dehydrogenase/reductase SDR family member 4 | DHRS4 | Q9BTZ2 | NM_001282987, NM_001282988, NM_001282989, NM_001282990, NM_001282991, NM_021004, XM_006720005, XM_ 011536366 | NP_001261916, NP_001i69917, NP_001269918, NP_001269919, NP_001269920, NP_066284, XP_006720068, XP_011534668 |
| 469 | 0.76 | Calponin-2 | CNN2 | Q99439 | NM_001303499, NM_001303501, NM_004368, NM-201277 | NP_001290428, NP_001290430, NP_004359, NP_958434 |
| 470 | 0.76 | Ribosome-binding protein 1 | RRBP1 | Q9P2E9 | NM_001042576, NM_001365613, NM_004587 | NP_001036041, NP_001352542, NP_004578 |
| 471 | 0.76 | Tyrosine-protein phosphatase non-receptor type 11 | PTPN11 | Q06124 | NM_001330437, NM_002834, NM_080601, XM_011538613, XM_017019722 | NP_001317366, NP_002825, NP_542168, XP_011536915, XP_016875211 |
| 472 | 0.76 | NEDD8-conjugating enzyme Ubc12 | UBE2M | P61081 | NM_003969 | NP_003960 |
| 473 | 0.76 | Amidophosphoribosyltransferase | PPAT | Q06203 | NM_002703, XM_011534387 | NP_002694, XP_011532689 |
| 474 | 0.76 | Polovirus receptor | PVR | P15151 | NM_001135768, NM_001135769, NM_001135770, NM_006505 | NP_001129240, NP_001129241, NP_001129242, NP_006496 |
| 475 | 0.76 | FACT complex subunit SPT16 | SUPT16H | Q9Y5B9 | NM_007192, XM_011536381 | NP_009123, XP_011534683 |

TABLE 5-29

| | | | | | | |
|---|---|---|---|---|---|---|
| 476 | 0.755 | Sorting nexin-24 | SNX24 | Q9Y343 | NM_014035, XM_005271972, XM_006714592, XM_ 011543349, XM_011543350, XM_011543352, XM_017009394, XM_017009395, XM_017009396 | NP_054754, XP_005272029, XP_006714655, XP_011541651, XP_011541652, XP_011541654, XP_016864883, XP_016864884, XP_016864885 |
| 477 | 0.755 | NudC domain-containing protein 1 | NUDCD1 | Q96RS6 | NM_001128211, NM_032869, XM_017013910, XM_017013911 | NP_001121683, NP_116258, XP_016869399, XP_016869400 |
| 478 | 0.75 | Talin-1 | TLN1 | Q9Y490 | NM_006289 | NP_006280, |
| 479 | 0.75 | Integrin alpha-6 | TGA6 | P23229 | NM_000210, NM_001079818, NM_001316308, NM_001365529, NM_001365530, XM_006712510, XM_006712511, XM_017004005, XM_017004006, XM_017004007, XM_017004008 | NP_000201, NP_001073286, NP_001303235, NP_001352458, NP_001352459, XP_006712573, XP -006712574, XP_016859494, XP_016859495, XP_016859496, XP _016859497 |
| 480 | 0.75 | Tetraspanin-14 | TSPAN14 | Q8NG11 | NM_001128309, NM_001351266, NM_001351267, NM_0011351268, NM_001351269, NM_001351270, NM_001351271, NM_001351272, NM_030927, XM_011540219, XM_011540220, XM_011540221, XM_017016712, XM_017016713, XM_0244418211, XM_024448212 | NP_001121781, NP_001338195, NP_001338196, NP_001338197, NP001338198, NP_001338199, NP_001338200, NP_001338201, NP_112189, XP_011538521, XP_011538522, XP_011538523, XP_016872201, XP_016872202, XP_024303979, XP_024303980 |
| 481 | 0.75 | Annexin A11 | ANXA11 | P50995 | NM_001157, NM_001278407, NM_001278408, NM_001278409, NM_145868, NM_145869, XM_005269741, XM_005269742, XM_006717813, XM_011539736 | NP_001148, NP_001265336, NP_001265337, NP_001265338, NP _665875, NP_665876, XP_005269798, XP_005269799, XP_006717876, XP_011538038 |
| 482 | 0.75 | Monoglyceride lipase | MGLL | Q99685 | NM_001003794, NM_001256585, NM_007283, XM_011512377, XM_011512378, XM_011512379, XM_011512382, XM_ 011512383, XM_017005662, XM_017005663, XM_017005664, XM_017005665, XM_017005666, XM_024453333, XM_024453334 | NP_001003794, NP_001243514, NP_009214, XP_011510679, XP_011510680, XP_011510681, XP_011510684, XP_011510685, XP_016861151, XP_016861152, XP_016861153, XP_016861154, XP_016861155, XP_024309101, XP_024309102 |
| 483 | 0.75 | Tubulin alpia-iachain | TUBA1A | Q71U36 | NM_001270399, NM_001270400, NM_006009 | NP_001257328, NP_001257329, NP_006000 |
| 484 | 0.75 | Glycogen phosphorylase, liver form | PYGL | P0673i | NM_001163940, NM_002863 | NP_001157412, NP_002854 |
| 485 | 0.75 | Brain acid soluble protein 1 | BASP1 | P80723 | NM_001271606, NM_006317 | NP_001258535, NP_006308 |
| 486 | 0.75 | Protein disulide-somerase A6 | PDIA6 | Q15084 | NM_001282704, NM_001282705, NM_001282706, NM_001282707, NM_005742, XM_011510308 | NP_001269633, NP_001269634, NP_001269635, NP_001269636, NP _005733, XP_011508610 |
| 487 | 0.75 | RuvB-like 2 | RUVBL2 | Q9Y230 | NM_001321190, NM_001321191, NM_006666, XM_011526330 | NP_001308119, NP_001308120, NP_006657, XP_011524632 |
| 488 | 0.75 | Protein lifeguard 3 | TMBIM1 | Q969X1 | NM_001321427, NM_001321428, NM_001321429, NM_001321430, NM_001321432, NM_001321433, NM_001321435, NM_001321436, NM_001321438, NM_022152, XM_011511627 | NP_001308356, NP_001308357, NP_001308358, NP_001308359, NP_001308361, NP_001308362, NP_001308364, NP_001308365, NP_001308367, NP_071435, XP_011509929 |

TABLE 5-30

| | | | | | | |
|---|---|---|---|---|---|---|
| 489 | 0.75 | Platelet-activating factor acetylhydrolase B subunit beta | PAFAH1B2 | P68402 | NM_001184746, NM_001184747, NM_001184748, NM_001309431, NM_002572, XM_017017840 | NP_001171675, NP_001171676, NP_001171677, MP_001296360, NP_002563, XP_016873329 |
| 490 | 0.75 | Transaldolase | TALD01 | P37837 | NM_006755 | NP_006746 |
| 491 | 0.75 | Guanine nucleotide-binding protein subunit alpha-13 | GNA13 | Q14344 | NM_001282425, NM_006572, XM_011524202 | NP_001269354, NP_006563, XP_011522504 |
| 492 | 0.75 | Nuclear transport factor 2 | NUTF2 | P61970 | NM_001322038, NM_001322039, NM_001322040, NM_01322041, NM_005796 | NP_001308967, NP_001308968, NP_001308969, NP_001308970, NP_005787 |
| 493 | 0.75 | Neutral alpha-glucosidase AB | GANAB | Q14697 | NM_001278192, NM_001278193, NM_001278194, NM_001329222, NM_001329223, NM_001329224, NM_001329225, NM_014610, NM_198334, NM_198335, XM_017017412 | NP_001265121, NP_001265122, NP_001265123, NP_001316151, NP_001316152, NP_001316153, NP_001316154, NP_938148, NP_938149, XP_016872901 |
| 494 | 0.75 | Apolipoproten(a) | LPA | P08519 | NM_005577 | NP_005568 |
| 495 | 0.75 | ADP-ribosylation factor 4 | ARF4 | P18085 | NM_001660 | NP_001651 |
| 496 | 0.75 | Mtochondrial fission 1 protein | FIS1 | Q9Y3D6 | NM_016068 | NP_057152 |
| 497 | 0.75 | Ubiquitin thioesterase OTUB1 | OTUB1 | Q96FW1 | NM_017670 | NP_060140 |
| 498 | 0.75 | Mannose-1-phosphate guanyltransferase alpha | GMPPA | Q96IJ6 | NM_013335, NM_205847, XM_005246486, XM_024452823 | NP_037467, NP_995319, XP_005246543, XP_024308591 |
| 499 | 0.75 | Proteasome subunit beta type-8 | PSMB8 | P28062 | NM_004159, NM_148919 | NP_004150, NP_683720 |
| 500 | 0.75 | Eukaryotic translation initiation factor 3 subunit A | EIF3A | Q14152 | NM_003750 | NP_003741 |
| 501 | 0.75 | Serine/threonine-protein Kinase26 | STK26 | Q9P289 | NM_001042452, NM_001042453, NM_016542, XM_011531349, XM_011531350 | NP_001035917, NP_001035918, NP_057626, XP_011529651, XP_011529652 |
| 502 | 0.75 | Guanine nucleotide-binding protein G(k) subunit alpha | GNAI3 | P08754 | NM_006496 | NP_006487 |
| 503 | 0.75 | Translationally-controlled tumor protein | TPT1 | P13693 | NM_001286272, NM_001286273, NM_003295 | NP_001273201, NP_001273202, NP_003286 |
| 504 | 0.75 | Serine/threonine-protein kinase TA01 | TAOK1 | Q7L7X3 | NM_020791, NM_025142, XM_011525060 | NP_065842, NP_079418, XP_011523362 |
| 505 | 0.75 | Claudin-3 | CLDN3 | 015551 | NM_001306 | NP_001297 |
| 506 | 0.75 | Nuclear migration protein nudC | NUDC | Q9Y266 | NM_006600, XM_011540530, XM_017000094, XM_017000095, XM_17000096, XM_024452486 | NP_006591, XP_011538832, XP_016855583, XP_016855584, XP_016855585, XP_024308254 |
| 507 | 0.75 | Enolase-phosphatase E1 | ENOPH1 | Q9UHY7 | NM_001292017, NM_021204 | NP_001278946, NP_067027 |
| 508 | 0.75 | Proteasome activator complex subunit 2 | PSME2 | Q9UL46 | NM_002818, XM_006720213 | NP_002809, XP_006720276 |
| 509 | 0.75 | Transmembrane protease serine 4 | TMPRSS4 | Q9NRS4 | NM_001083947, NM_001173551, NM_001173552, NM_001290094, NM_001290096, NM_019894, NM_183247, XM_005271613, XM_005271614, XM_005271615, XM_011542901, XM_011542902, XM_011542903, XM_011542904 | NP_001077416, NP_001167022, NP_001167023, NP_001277023, NP_00177025, NP_063947, XP_005271670, XP_005271671, XP_005271672, XP_011541203, XP_011541204, XP_011541205, XP_011541206 |
| 510 | 0.75 | Syntaxin-2 | STX2 | P32856 | NM_001351049, NM_001351050, NM_001351051, NM_001351052, NM_001980, NM_194356, XM_017018978, XM_017018979, XM_017018980, XM_017018981, XM_017018982, XM_017018983, XM_017018984, XM_017018988, XM_017018989 | NP_001337978, NP_001337979, NP_001337980, NP_001337981, NP_001971 NP_919337, XP_016874467, XP 016814468, XP_016874469, XP_016874469, XP_016874470, XP_016874471, XP_0168744727, XP_016874473, XP_016874477, XP_016874478 |

TABLE 5-31

| | | | | | | |
|---|---|---|---|---|---|---|
| 511 | 0.75 | Ras-related protein Rab-15 | RAB15 | P59190 | NM_001308154, NM_001330182, NM_198889, XM_024449577 | NP_001295083, NP_001317111, NP_941959, XP_024305345 |
| 512 | 0.75 | Ras-related protein Rab-33B | RAB33B | Q9H082 | NM_031296, XM_011532299 | NP_112586, XP_011530601 |
| 513 | 0.75 | Ubiquilin-1 | UBQLN1 | Q9UMX0 | NM_013438, NM_053067, XM_005251948 | NP_038466, NP_444295, XP_005252005 |
| 514 | 0.75 | Cysteine-tRNAligase, cystoplasmic | CARS | P49589 | NM_001014437, NM_001014438, NM_001194997, NM_001751, NM_139273, XM_006718341, XM_017018389, XM_017018390, XM_017018391, XM_017018392, XM_024448701, XM_024448702, XM_24448703, XM_024448704, XM_024448705 | NP_001014437, NP_001181926, NP_001742, NP_644802, XP_006718404, XP_016873878, XP_016873879, XP_016873880, XP_0168873881, XP_024304469, XP_024304470, XP_024304471, XP_024304472, XP_024304473 |
| 515 | 0.75 | Palmitoyltransferase ZDHHC5 | ZDHHC5 | Q9C0B5 | NM_015457, XM_011544899, XM_011544900, XM_011544901, XM_017017498 | NP_056272, XP_011543201, XP_011543202, XP_011543203, XP_016872987 |
| 516 | 0.75 | Famesyl pyrophosphate synthase | FDPS | P14324 | NM_001135821, NM_001135822, NM_001242824, NM_001242825, NM_002004, XM_005244962, XM_005244963, XM_024454066, XM_024454070, XM_024454071, XM_024454072, XM_024454073, XM_024454074, XM_024454076 | NP_001129293, NP_001129294, NP_001229753, NP_001229754, NP-001995, XP_005245019, XP_005245020, XP_024309834, XP_024309838, XP_024309839, XP_024309840, XP_024309841, XP_024309842, XP_024309844 |
| 517 | 0.75 | DnaJ homolog subfamily B member6 | DNAJB6 | O75190 | NM_001363676, NM_005494, NM_058246, XM_005249515, XM_005249516, XM_006715823, XM_011515704, XM_017011633 | NP_001350605, NP_005485, NP_490647, XP_005249572, XP_005249573, XP_006715886, XP_011514006, XP_016867122 |
| 518 | 0.75 | Tetraspanin-31 | TSPAN31 | Q12999 | NM_001330168, NM_001330169, NM_005981, XM_024449123 | NP_001317097, NP_001317098, NP_005972, XP_024304391 |
| 519 | 0.75 | Charged multivesicular body protein 4a | CHMP4A | Q9BY43 | NM_014169 | NP_054888 |
| 520 | 0.75 | Charged multivesicular body protein 2a | CHMP2A | C43633 | NM_014453, NM_198426, XM_005258746, XM_005258747 | NP_055268, NP_940818, XP_005258803, XP_005258804 |
| 521 | 0.75 | 1-phosphatidylinositol 4,5-bisphophate phosphodiesterase beta-3 | PLCB3 | Q01910 | NM_000932, NM_001184883, NM_001316314, XM_011545101, XM_017017925 | NP_000923, NP_001171812, NP_001303243, XP_011543403, XP_016873414 |
| 522 | 0.75 | Calcyclin-binding protein | CACYBP | Q9HB71 | NM_001007214, NM_014412, XM_017001046 | NP_001007215, NP_055227, XP_016856535 |
| 523 | 0.75 | Apolipoprotein A-II | AP0A2 | P02652 | NM_001643 | NP_001634 |
| 524 | 0.75 | Epsin-1 | EPN1 | Q9Y613 | NM_001130071, NM_001130072, NM_001321263, NM_D13333, XM_005258829, XM_011526880, XM_011526881, XM_017026722, XM_017026723, XM_017026724, XM_017026725, XM_024451478 | NP_001123543, NP_001123544, NP_001308192, NP_037465, XP_005258886, XP_01152182, XP_011525183, XP_016882211, XP_ 0616882212, XP_016882213, XP_016882214, XP_024307246 |
| 525 | 0.75 | Vitamin D-binding protein | GC | P02774 | NM_000583, NM_00124306, NM_001204307, XM_006714177 | NP_000574, NP_001191235, NP_001191236, XP_006714240 |

TABLE 5-32

| | | | | | | |
|---|---|---|---|---|---|---|
| 526 | 0.75 | Glutamine amidotransferase-like class 1 domain-containing protein 1 | GATD1 | Q8NB37 | NM_001318818, NM_001318820, NM_001318821, NM_001318822, NM_001318823, NM_001318824, NM_182612, XM_005252898, XM_011520064, XM_017017662, XM_017017664, XM_017017667, XM_017017668, XM_024448490, XM_024448491, XM_024448492 | NP_001305747, NP_001305749, NP_001305750, NP_001305751, NP_001305752, NP_001305753, NP_872418, XP_005252955, XP_011518366, XP_016873151, XP_016873153, XP_016873156, XP_016873157, XP_024304258, XP_024304259, XP_024304260 |
| 527 | 0.75 | Synaptophysin-llke protein 1 | SYPL1 | Q16563 | NM_06754, NM_182715 | NP_00674, NP_874384 |
| 528 | 0.75 | Myeloid leukemia factor 2 | MLF2 | Q15773 | NM_005439, XM_024449201 | NP_005430, XP_024304969 |
| 529 | 0.75 | Phosphomevalonate Kinase | PMVK | Q15126 | NM_001323011, NM_001323012, NM_001348696, NM_006556 | NP_001309940, NP_001309941, NP_001335625, NP_006547 |
| 530 | 0.75 | Melantransferrin | MELTF | P08582 | NM_005929, NM_033316, XM_006713643, XM_011512850, XM_011512851 | NP_005920, NP_201573, XP_006713706, XP_011511152, XP_011511153 |
| 531 | 0.75 | Mtogen-actvated protein knase 3 | MAPK3 | P27361 | NM_001040056, NM_001109891, NM_002746 | NP_001035145, NP_001103361, NP_002737 |
| 532 | 0.75 | Galactose-1-phosphate uridylyltransferase | GALT | P07902 | NM_000155, NM_001258332 | NP_000146, NP_001245261 |
| 533 | 0.75 | Connective tissue growth factor | CTGF | P29279 | NM_001901 | NP_001892 |
| 534 | 0.75 | C2 domain-containing protein 5 | C2CD5 | Q86YS7 | NM_001286173, NM_001286174, NM_001286175, NM_001286176, NM_001286177, NM_014802, XM_005125N8, XM_005253539, XM_006719170, XM_017020264, XM_017020265, XM_017020266, XM_017020267, XM_017020268, XM_017020269, XM-017020270, XM_017020271, XM_017020272, XM_017020273, XM_017020274, XM_017020275, XM_017020276, XM_017020277, XM_017020278, XM_017020279, | NP_001273102, NP_001273103, NP_001273104, NP_001273105, NP_001273106, NP_055617, XP_005253595, XP_005253596, XP_006719233, XP_016875753, XP_016875754, XP_016875755, XP_16875756, XP_016875757, XP_016875758, XP_016875759, XP_016875760, XP_16875761, XP_16875762, XP_016875763, XP_016875764, XP_016875765, XP_016875766, XP_016875767 XP_016875768 |
| 535 | 0.75 | Interferon-induced,double-strandd RNA-activatedproteinkinase | EIF2AK2 | P19525 | NM_001135651, NM_001135652, NM_002759, XM_011532987, XM_017004503 | NP_001129123, NP_001129124, NP_002750, XP_011531289, XP_016859992 |
| 536 | 0.75 | Exocyst complex component 2 | EX0C2 | Q96KP1 | NM_018303, XM_017011018, XM_017011019, XM_017011020, XM_017011021, XM_017011022, XM_017011023, XM_017011024, XM_017011025, XM_017011026 | NP_060773, XP_0168665O7, XP_016866508, XP_016866509, XP_016866510, XP_016866511, XP_016866512, XP_016866513, XP_016866514, XP_016866515 |

TABLE 5-33

| | | | | | | |
|---|---|---|---|---|---|---|
| 537 | 0.745 | MAP kinase-activating death domain protein | MADD | Q8WXG6 | NM_001135943, NM_01135944, NM_003882, NM_130470, NM_130471, NM_130472, NM_130473, NM_130414, NM_130475, NM_130476, XM_005253189, XM_005253196, XM_005253199, XM_005253200, XM_005253201, XM_005253203, XM_005253204, XM_005253205, XM_017018478, XM_017018479, XM_017018480, XM_017018481, XM_017018482, XM_017018483, XM_017018484, XM_017018485, XM_017018486, XM_017018487, XM_017018488, XM_017018489, XM_017018490, XM_017018491, XM_017018492, XM_017018493, XM_017018498, XM_317018499, XM_017018500, XM_017018505, XM_017018506, XM_017018507, XM_024448745, XM_024448746, XM_024448747, XM_024448748, XM_024448749, XM_024448750, XM_024448751, XM_024448752, XM_024448753, XM_024448754, XM_024448755 | NP_001129415, NP_001129416, NP_003673, NP_569826, NP_569827, NP_569828, NP_569829, NP_569830, NP_569831, NP_569832, XP_005253246, XP_005253253, XP_005253256, XP_005253257, XP_005253258, XP_005253260, XP_005253261, XP_005253262, XP_016873967, XP_016873968, XP_016873969, XP_016873970, XP_016873971, XP_016873972, XP_016873973, XP_016813974, XP_016873975, XP_016873976, XP_016873977, XP_016873978, XP_016873979, XP_016873980, XP_016873981, XP_016873982, XP_16873987, XP_16873988, XP_016873989, XP_016873994, XP_016873995, XP_016873996, XP_024304513, XP_024304514, XP_024304515, XP_024304516, XP_024304517, XP_024304518, XP_024304519, XP_024304520, XP_024304521, XP_024304522, XP_024304523 |
| 538 | 0.74 | Melanoma-associated antgen D2 | MAGED2 | Q9UNF1 | NM_014599, NM_177433, NM_201222 | NP_055414, NP_803182, NP_957516 |
| 539 | 0.74 | Ras-elated protein Rab-6D | RAB6D | Q53S08 | NM_001077637 | NP_001071105 |
| 540 | 0.74 | Guanine nucleotide-binding protein G(i) subunit alpha-2 | GNAL2 | P04899 | NM_001166425, NM_001282617, NM_001282618, NM_001282619, NM_001282620, NM_002070 | NP_001159897, NP_001269546, NP_001269647, NP_001269548, NP_001269549, NP_002061 |
| 541 | 0.74 | Transketolase | TKT | P29401 | NM_001064, NM_001135055, NM_001135056, NM_001258028, XM_011534054, XM_011534055 | NP_001055, NP_001128527, NP_001244957, XP_011532356, XP_011532357 |
| 542 | 0.74 | Alpha-centractin | ACTR1A | P61163 | NM_005736 | NP_005727 |
| 543 | 0.74 | L-lactate dehydrogenase A chain | LDHA | P00338 | NM_001135239, NM_001165414, NM_001165415, NM_001165416, NM_005566 | NP_001128711, NP_001158886, NP_001158887, NP_00115888, NP_005557 |
| 544 | 0.74 | Heatshock protein beta-1 | HSPBI | P04792 | NM_001540 | NP_001531 |
| 545 | 0.74 | Histone H4 | HIST1H4A | P62805 | NM_003495, NM_003538, NM_003539, NM_003540, NM_003541, NM_021968, NM_003542, NM_003543, NM_003544, NM_003545, NM_003546, NM_003548, NM_175054, NM_001034077 | NP_003486, NP_003529, NP_003530, NP_003531, NP_003532, NP_068803, NP_003533, NP_003534, NP_003535, NP_003536, NP_003537, NP_003539, NP_778224, NP_001029249 |
| 546 | 0.74 | Filamin-A | FLNA | P21333 | NM_001110556, NM_001456 | NP_001104026, NP_001447 |
| 547 | 0.74 | Ras-related protein Rab-5A | RAB5A | P20339 | NM_001292048, NM_004162 | NP_001278977, NP_004153 |

TABLE 5-34

| | | | | | | |
|---|---|---|---|---|---|---|
| 548 | 0.74 | Cytoplasmic FMR1-interacting protein 1 | CYFIP1 | Q7L576 | NM_001033028, NM_001287810, NM_001324119, NM_001324120, NM_001324122, NM_001324123, NM_001324124, NM_001324125, NM_001324126, NM_014608, XM_011543873, XM_011543874, XM_011543876, XM_017022023, XM_017022024, XM_024449876, XM_024449877 | NP_001028200, NP_001274739, NP_001311048, NP_001311049, NP_001311051, NP_001311052, NP_001311053, NP_001311054, NP_001311055, NP_055423, XP_011542175, XP_011542176, XP_011542178, XP_016877512, XP_016877513, XP_024305644, XP_02430564, |
| 549 | 0.74 | | GFALS | P35858 | NM_001146006, NM_004970 | NP_001139478, NP_004961 |
| 550 | 0.74 | Aflatoxin B1 aldehyde reductase member 2 | AKR7A2 | 043488 | NM_001320979, NM_003689 | NP_001307908, NP_003680 |
| 551 | 0.74 | CD99 antigen | CD99 | P14209 | NM_001122898, NM_001277710, NM_001321367, NM_001321368, NM_001321369, NM_001321370, NM_002414 | NP_001118370, NP_001308296, NP_001308297, NP_001308298, NP_001308299, NP_002405 |
| 552 | 0.74 | Complement C4-A | C4A | P0C0L4 | NM_001252204, NM_007293, NM_001002029 | NP_001239133, NP_009224, NP_001002029 |
| 553 | 0.74 | Syntaxin-binding protein 3 | STXBP3 | 000186 | NM_007269 | NP_009200 |
| 554 | 0.74 | Cytoplasm c FMR1-interacting protein 2 | CYFIP2 | Q96F07 | NM_001037332, NM_001037333, NM_001291721, NM_1291722, NM_014376, XM_011534516, XM_017009341, XM_017009341 | NP_001032410, NP_001278650, NP_001278651, NP_055191, XP_011532818, XP_016864830, XP_016864831 |
| 555 | 0.74 | Junctional adhesionmolecule A | F11R | Q9Y624 | NM_001348091, NM_016946, NM_144501, NM_144502, NM_144503, NM_144504 | NP_001335020, NP_58642 |
| 556 | 0.74 | Low molecular weight phosphotyrosine protein phosphatase | ACP1 | P24666 | NM_001040649, NM_004300, NM_007099, NM_17754 | NP_001035739, NP_004291, NP_009030 |
| 557 | 0.74 | Nascent polypeptide-associated complex subunit alpha muscle-specificfor HACA | | E9PAV3 | NM_001113201, NM_001113202, NM_001113203, NM_001320193, NM_001320194, NM_001365896, NM_005594, XM_006719412, XM_006719413, XM_006719414, XM_006719418, XM_006719420, XM_011538389, XM_017019337 | NP_001106672, XP_001106673, NP_001106674, NP_001307122, NP_001307123, NP_001352825, NP_005585, XP_006719475, XP_006719476, XP_006719477, XP_006719481, XP_006719483, XP_011536691, XP_016874826 |
| 558 | 0.74 | Apoptosis regulator BAX | BAX | Q07812 | NM_001291428, NM_001291429, NM_001291430, NM_001291431, NM_004324, NM_138761, NM_138762, NM_138763, NM_138764, XM_017027077 | NP_001278357, NP_001278358, NP_001278359, NP_001278360, NP_004315, NP_620116, NP_620118, NP_20119, XP_016882566 |
| 559 | 0.74 | Midkine | MDK | P21741 | NM_001012333, NM_001012334, NM_H1270550, NM_001270551, NM_001270652, NM_002391, XM_011520116, XM_017017764 | NP_001012333, NP_001012334, NP_001257479, NP_001257480, NP_001257481, NP_002382, XP_011518418, XP_016873253 |
| 560 | 0.74 | Heat shock 70 kDa protein 1A | HSPA1A | P0DMV8 | NM_005345, NM_005346 | NP_005336, NP_005337 |
| 561 | 0.74 | Axn interactor, dorsalization-associated protein | AIDA | Q96BJ3 | NM_022831 | NP_073742 |
| 562 | 0.74 | Glucosidase 2 subunit beta | PRKCSH | P14314 | NM_001001329, NM_001289102, NM_001289103, NM_001289104, NM_002743, XM_011528130, XM_011528131, XM_011528132, XM_017026977, XM_024451602 | NP_001001329, NP_001278031, NP_001276032, NP_001276033, NP_002734, XP_011526432, XP_011526433, XP_011526434, XP_016882466, XP_024307370 |

TABLE 5-35

| | | | | | | |
|---|---|---|---|---|---|---|
| 563 | 0.74 | Proteasome activator complex subunit 1 | PSME1 | Q06323 | NM_001281528, NM_001281529, NM_006263, NM_176783 | NP_001268457, NP_001268458, NP_006254, NP_78895 |
| 564 | 0.74 | Electron transfer flavoprotein subunit alpha, mitochondrial | ETFA | P13804 | NM_000126, NM_001127716 | NP_000117, NP_001121188 |
| 565 | 0.74 | Filamin-C | FLNC | Q14315 | NM_001127487, NM_001458 | NP_001120959, NP_001149 |
| 566 | 0.74 | Phospholipid hydroperoxide glutathione peroxidase | GPX4 | P36969 | NM_001039847, NM_001039848, NM_002085 | NP_001034936, NP_001034937, NP_002076 |
| 567 | 0.74 | Heterogeneous nuclear ribonucleoprotein L | HNRNPL | P14866 | NM_001005335, NM_001533, XM_011526888, XM_024451479, XM_024451480, XM_024451481, XM_024451482, XM_024451483 | NP_001005335, NP_001524, XP_011525190, XP_024307247, XP_024307248, XP_024307249, XP_024307250, XP_024307251 |
| 568 | 0.74 | Formin-binding protein 1-like | FNBP1L | Q5TON5 | NM_001024948, NM_001164473, NM_017737, XM_011541625, XM_017001533 | NP_001020119, NP_001157945, NP_060207, XP_011539927, XP_016857022 |
| 569 | 0.74 | RAC-beta serine/threonine-orotein Kinase | AKT2 | P31751 | NM_001243027, NM_001243028, NM_001330511, NM_001626, XM_011526614, XM_011526615, XM_011526616, XM_011526618, XM_011526619, XM_011526620, XM_011526622, XM_017026470, XM_024451416, XM_024451417 | NP_001229956, NP_001229957, NP_001317440, NP_001617, XP_011524916, XP_011524917, XP_011524918, XP_011524920, XP_011524921, XP_011524922, XP_011524924, XP_016881959, XP_024307181, XP_024307185 |
| 570 | 0.74 | ADP-ribosylcyclaselcyclic ADP-r bose hydrolase 2 | BST1 | Q10588 | NM_004334, XM_005248185, XM_005248166, XM_011513878, XM_011513879, XM_011513881, XM_017008565, XM_017008566 | NP_004325, XP_005248242, XP_005248243, XP_011512180, XP_011512181, XP_011512183, XP_016864054, XP_016864055 |
| 571 | 0.74 | Ras-related oroten Rab-22A | RAB22A | Q9UL26 | NM_020673 | NP_065724 |
| 572 | 0.74 | Neutral amino acid transporter B(0) | SLC1A5 | Q15758 | NM_001145144, NM_001145145, NM_005628, XM_005259167 | NP_001138616, NP_001138617, NP_005619, XP_005259224 |
| 573 | 0.74 | Erbin | ERBIN | Q96RT1 | NM_001006600, NM_001253697, NM_001253698, NM_001253699, NM_001253701, NM_018695, XM_005248554, XM_005248555, XM_006714660, XM_017009635, XM_017009636, XM_017009637 | NP_001006600, NP_001240626, NP_001240627, NP_001240628, NP_001240630, NP_061165, XP_005248611, XP_005248612, XP_006714123, XP_016865124, XP_016865125, XP_016865126 |
| 574 | 0.74 | Eukaryotic translation initiation factor 3 subunit K | EIF3K | Q9UBQ5 | NM_001300992, NM_001308393, NM_013234 | NP_001287921, NP_001295322, NP_031366 |
| 575 | 0.74 | AP-2 complex subunit alpha-1 | AP2A1 | C95782 | NM_014203, NM_130787, XM_011526556, XM_011526557 | NP_055018, NP_570603, XP_011524858, XP_011524859 |
| 576 | 0.74 | Fructose-bisphosphate aldolase C | ALDOC | P09972 | NM_005165, XM_005257949, XM_011524556 | NP_005156, XP_005258006, XP_011522858 |
| 577 | 0.74 | Lactoylglutathionelyase | GL01 | Q04760 | NM_006708 | NP_006699 |
| 578 | 0.74 | Arrestin-C | ARR3 | P36575 | NM_004312, XM_017029518 | NP_004303, XP_016885007 |
| 579 | 0.74 | Fructose-1,6-bisphosphatase 1 | FBP1 | P09467 | NM_000507, NM_001127628, XM_006717005 | NP_000498, NP_001121100, XP_06717068 |
| 580 | 0.74 | Solute carrier family 12 member 2 | SLC12A2 | P55011 | NM_001046, NM_001256461, XM_011543588, XM_017009771 | NP_001037, NP_001243390, XP_011541890, XP_016865260 |
| 581 | 0.74 | Sulotransferase family cytosolic 2B member 1 | SULT2B1 | 000204 | NM_004605, NM_177973 | NP_004596, NP_814444 |

TABLE 5-36

| | | | | | | |
|---|---|---|---|---|---|---|
| 582 | 0.74 | UDP-glucuronic acid decarboxylase 1 | UXS1 | Q8NBZ7 | NM_001253875, NM_001253876, NM_025076, XM_006712775, XM_011511902, X_011511903, XM_011511904, XM_17005008, XM_017005009, XM_017005010, XM_017005011, XM_017005012, XM_017005013, XM_017005014, XM_024453157 | NP_001240804, NP_001240805, NP_079352, XP_006712838, XP_011510204, XP_011510205, XP_011510206, XP_016860497, XP_016860498, XP_016860499, XP_016860500, XP_016860501, XP_016860502, XP_016860503, XP_024308925 |
| 583 | 0.74 | NEDD8 | NEDD8 | Q15843 | NM_006156 | NP_006147 |
| 584 | 0.74 | Myotubularn | MTM1 | Q13496 | NM_000252, XM_005274687, XM_011531171, XM_011531172, XM_011531173, XM_017029547, XM_017029548, XM_017029549, XM_17029550, XM_017029551 | NP_000243, XP_005274744, XP_011529473, XP_011529474, XP_011529475, XP_016885036, XP_016885037, XP_016885038, XP_016885039, XP_016885040 |
| 585 | 0.74 | CTD smal phosphatase-llke protein | CTDSPL | 015194 | NM_001008392, NM_005808, XM_017005519, XM_017005520 | NP_001008393, NP_005799, XP_16861008, XP_016861009 |
| 586 | 0.74 | E3 UFM1-protein ligase 1 | UFL1 | 094814 | NM_015323 | NP_056138 |
| 587 | 0.74 | WD repeat-containing protein 48 | WDR48 | Q8TAF3 | NM_001303402, NM_001303403, NM_001346225, NM_001346226, NM_001346227, NM_001346228, NM_020839, XM_005265346, XM_011533971 | NP_001290331, NP_001290332, NP_001333154, NP_001333155, NP_001333156, NP_001333157, NP_065890, XP_005265403, XP_011532273 |
| 588 | 0.74 | 0.74 Protein EFR3 hamolog A | EFR3A | Q14156 | NM_001323553, NM_001323554, NM_001323555, NM_001323556, NM_001323557, NM_001323558, NM_015137 | NP_001310482, NP_001310483, NP_001310484, NP_001310485, NP_001310486, NP_001310487, NP_055952 |
| 589 | 0.74 | Porimin | TMEM123 | Q8N131 | NM_052932 | NP_443164 |
| 590 | 0.74 | STE20-likeserine/threonine-protein kinase | SLK | Q9H2G2 | NM_001304743, NM_014720, XM_0011540401 | NP_001291672, NP_055535, XP_011538103 |
| 591 | 0.74 | Ubiquitin-fold modifer 1 | UFM1 | P61960 | NM_001286703, NM_001286704, NM_001286705, NM_001286706, NM_016617 | NP_001273632, NP_001273633, NP_001273634, NP_001273635, NP_057701 |
| 592 | 0.74 | Ethanolam ne-phosphate Cytidylyltransferase | PCYT2 | Q99447 | NM_001184917, NM_001256433, NM_001256434, NM_001256435, NM_001282203, NM_001282204, NM_001330518, NM_002861, XM_005256386, XM_005256387, XM_006722287, XM_017024911, XM_017024912 | NP_001171846, NP_001243362, NP_001243363, NP_001243364, NP_001269132, NP_001269133, NP_001317447, NP_002852, XP_005256443, XP_005256444, XP_006722350, XP_016880400, XP_016880401 |
| 593 | 0.74 | Small nuclear ribonucleoprotein F | SNRPF | P62306 | NM_003095 | NP_003086 |
| 594 | 0.74 | Beta-hexosaminidase subunit beta | HEXB | P07686 | NM_000521, NM_001292004 | NP_00512, NP_001278933 |
| 595 | 0.74 | Secretory carrier-associated membrane oroten 4 | SCAMP4 | Q969E2 | NM_001329539, NM_001329540, NM_079834 | NP_001316468, NP_001316469, NP_524558 |
| 596 | 0.74 | Autophagy-relatedprotein 9A | ATG9A | Q7Z3C6 | NM_001077198, NM_024085 | NP_001070666, NP_076990 |
| 597 | 0.74 | Endoplasmic reticulum resident protein 29 | ERP29 | P30040 | NM_001034025, NM_006817, XM_017018720 | NP_001029197, NP_006808, XP_016874209 |
| 598 | 0.74 | Basic leucinezipper and W2 domain-containing protein 2 | BZW2 | Q9Y6E2 | NM_001159767, NM_1362717, NM_001362718, NM_001362719, NM_014038, XM_006715706, XM_006715707, XM_006715708, XM_024446735 | NP_001153239, NP_001349646, NP_001349647, NP_001349648, NP_054757, XP_006715769, XP_006715770, XP_006715771, XP_024302503 |
| 599 | 0.74 | Lipid droplet-associated hydrolase | LDAH | Q9H6V9 | NM_001282719, NM_001282720, NM_001282721, NM_001282722, NM_001282723, NM_001282724, NM_021925, XM_011533014 | NP_001269648, NP_001269649, NP_001269650, NP_001269651, NP_001269652, NP_001269653, NP_068744, XP_011531316 |

TABLE 5-37

| | | | | | | |
|---|---|---|---|---|---|---|
| 600 | 0.73 | Plexin-B2 | PLXNB2 | 015031 | NM_012401, XM_005261909, XM_005261910, XM_005261911, XM_006724413, XM_011530682, XM_017028703, XM_017028704 | NP_036533, XP_005261966, XP_005261967, XP_005261968, XP_006724476, XP_011528984, XP_016884192, XP 016884193 |
| 601 | 0.73 | Flotillin-2 | FLOT2 | Q14254 | NM_001330170, NM_004475, XM_005257953, XM_017024394, XM_017024395, XM_017024396, XM_024450666, XM_024450667 | NP_001317099, NP_004466, XP_005258010, XP_016879883, XP_016879884, XP_016879885, XP_024306434, XP_024306435 |
| 602 | 0.73 | Aldehyde dehydrogenase Family 16 member A1 | ALDH16A1 | Q8IZ83 | NM_001145396, NM_153329, XM_011526441, XM_011526442 | NP_001138868, NP_699160, XP_011524743, XP_011524744 |
| 603 | 0.73 | Retinal dehydrogenase 1 | ALDH1A1 | P00352 | NM_000689 | NP_000680 |
| 604 | 0.73 | Inter-alpha-trypsin inhibitor heavy chain H3 | ITIH3 | Q06033 | NM_002217, XM_005265105, XM_024453512 | NP_002208, XP_005265162, XP_024309280 |
| 605 | 0.73 | HLAclass Ihistocompatibility antigen, B-44 alpha chain | HLA-B | P30481 | NM_005514 | NP_005505 |
| 606 | 0.73 | BPI fold-containing family B member 1 | BPIFB1 | Q8TDL5 | NM_033197, XM_024452018 | NP_149974, XP_024307786 |
| 607 | 0.73 | Basigin | BSG | P35613 | NM_001322243, NM_001728, NM_198589, NM_198590, NM_198591, XM_017027173 | NP_001309172, NP_001719, NP_940991, NP_940992, NP_940993, XP_016882662 |
| 608 | 0.73 | Nectin-2 | NECTIN2 | Q92692 | NM_001042724, NM_002856 | NP_001036189, NP_002847 |
| 609 | 0.73 | Ragulatorcomplex protein LAMTOR1 | LAMTOR1 | Q6IAA8 | NM_017907 | NP_060377 |
| 610 | 0.73 | Tetraspanin-9 | TSPAN9 | O75954 | NM_001168320, NM_006675, XM_011520912, XM_011520913 | NP_001161792, NP_006666, XP_011519214, XP_011519215 |
| 611 | 0.73 | ATP-dependent 6-phosphofructokinase, platelet type | PFKP | Q01813 | NM_001242339, NM_001323067, NM_001323068, NM_001323069, NM_001323070, NM_001323071, NM_001323072, NM_001323073, NM_001323074, NM_001345944, NM_002627, XM_005252465, XM_005252466, XM_006717449, XM_024448038 | NP_001229268, NP_001309996, NP_001309997, NP_001309998, NP_001309999, NP_001310000, NP_001310001, NP_001310002, NP_001310003, NP_001332873, NP_002618, XP_005252522, XP_005252523, XP_006717512, XP_024303806 |
| 612 | 0.73 | Plasma membrane calcium-transporting ATPase 1 | ATP2B1 | P20020 | NM_001001323, NM_001366520, NM_001366521, NM_001366522, NM_001366523, NM_001366524, NM_001366525, NM_001366526, NM_001366527, NM_001366528, NM_001366529, NM_001366530, NM_001366531, NM_001366532, NM_001682, XM_011538407, XM_017019357, XM_024448989, XM_024448990, XM_024448991, XM_24448992, XM_024448993 | NP_001001323, NP_001353449, NP_001353450, NP_001353451, NP_001353452, NP_001353453, NP _001353454, NP_001353455, NP_001353456, NP_001353457, NP_001353458, NP_001353459, NP_001353460, NP_001353461, NP_001673, XP_011536709, XP_016874846, XP_024304757, XP_024304758, XP_024304759, XP_024304760, XP_024304761 |
| 613 | 0.73 | Nck-assocated protein 1 | NCKAP1 | Q9Y2A7 | NM_013436, NM_205842, XM_006712200, XM_006712201 | NP_038464, NP_995314, XP_006712263, XP_006712264 |
| 614 | 0.73 | Protein diaphanous homolog 1 | DIAPH1 | C60610 | NM_001079812, NM_001314007, NM_005219, XM_024454384, XM_024454385, XM_024454386, XM_024454387 | NP_001073280, NP_001300936, NP_005210, XP_024310152, XP_024310153, XP_024310154, XP_024310155 |
| 615 | 0.73 | Beta-enolase | EN03 | P13929 | NM_001193503, NM_001976, NM_053013, XM_005256521, XM_011523729, XM_017024346 | NP_001180432, NP_001967, NP_443739, XP_005256578, XP_011522031, XP_016879835 |

TABLE 5-38

| | | | | | | |
|---|---|---|---|---|---|---|
| 616 | 0.73 | Ras-related protein Rab-2B | RAB2B | Q8WUD1 | NM_001163380, NM_032846, XM_017021710, XM_017021712 | NP_001156852, NP_116235, XP_016877199, XP_016877201 |
| 617 | 0.73 | Alpha-1,6-mannosylglycoprotein 6-beta-N-acetylglucosaminyltransferase A | MGAT5 | Q09328 | NM_002410, XM_005263668, XM_005263669, XM_005263670, XM_006712534, XM_011511199, XM_011511200, XM_011511201, XM_011511202, XM_011511204, XM_017004147, XM_017004148, XM_017004149 | NP_002401, XP_005263725, XP_005263726, XP_005263727, XP_006712597, XP_011509501, XP_011509502, XP_011509503, XP_011509504, XP_011509506, XP_016859636, XP_016859637, XP_016859638 |
| 618 | 0.73 | Disintegrin and metalloproteinase domain-containing protein 9 | ADAM9 | Q13443 | NM_001005845, NM_003816, XM_011544682, XM_017013942 | NP_003807, XP_011542984, XP_016869431 |
| 619 | 0.73 | Small VCP/p97-lnteracting protein | SVIP | Q8NHG7 | NM_001320340, NM_001320341, NM_001320342, NM_148893, XM_024448423 | NP_001307269, NP_001307270, NP_001307271, NP_683691, XP_024304191 |
| 620 | 0.73 | Cell cycle control protein 50A | TMEM30A | Q9NV96 | NM_001143958, NM_018247 | NP_001137430, NP_060717 |
| 621 | 0.73 | Endoplasmin | HSP90B1 | P14625 | NM_003299 | NP_003290 |
| 622 | 0.73 | UMP-CMP kinase | CMPK1 | P30085 | NM_001136140, NM_001366135, NM_016308 | NP_001129612, NP_001353064, NP_057392 |
| 623 | 0.73 | Monocarboxylate transporter 1 | SLC16A1 | P53985 | NM_001166498, NM_003051 | NP_001159968, NP_003042 |
| 624 | 0.73 | Ras GTPase-activating protein 3 | RASA3 | Q14644 | NM_001320821, NM_001320822, NM_007368, XM_011534841, XM_011534842, XM_017020438, XM_017020439 | NP_001307750, NP_001307751, NP_031394, XP_011533143, XP_011533144, XP_016875927, XP_016875928 |
| 625 | 0.73 | Alpha-actinin-1 | ACTN1 | P12814 | NM_001102, NM_001130004, NM_001130005, XM_011537265, XM_011537266, XM_011537267, XM_011537268, XM_017021720, XM_017021721, XM_017021722, XM_017021723, XM_017021725, XM_017021726, XM_017021727, XM_017021728 | NP_001093, NP_001123476, NP_001123477, XP_011535567, XP_011535568, XP_011535569, XP_011535570, XP_016877209, XP_016877210, XP_016877211, XP_016877212, XP_016877214, XP_016877215, XP_016877216, XP_016877217 |
| 626 | 0.73 | Polyadenylate-binding protein 1 | PABPC1 | P11940 | NM_002568, XM_005250861 | NP_002559, XP_005250918 |
| 627 | 0.73 | Diphosphomevalonate decarboxylase | MVD | P53602 | NM_002461, XM_011523086, XM_011523087, XM_011523088, XM_011523089 | NP_002452, XP_011521388, XP_011521389, XP_011521390, XP_011521391 |
| 628 | 0.73 | ADP-ribosylation factor-like protein 13B | ARL13B | Q3SXY8 | NM-001174150, NM_001174151, NM_001321328, NM_144996, NM_182896, XM_006713532, XM_011512532, XM_011512533, XM_011512534, XM_011512535, XM_017005853 | NP_001167621, NP_001167622, NP_001308257, NP_659433, NP_878899, XP_006713595, XP_011510834, XP_011510835, XP_011510836, XP_011510837, XP_016861342 |
| 629 | 0.73 | Guanine nucleotide-binding protein G(I)/G(S)/G(T) subunit beta-2 | GNB2 | P62879 | NM_005273 | NP_005264 |
| 630 | 0.73 | Zyxin | ZYX | Q15942 | NM_001010972, NM_001362783, NM_003461, XM_011516569, XM_017012587 | NP_001010972, NP_001349712, NP_003452, XP_011514871, XP_016868076 |
| 631 | 0.73 | Ribonuclease 7 | RNASE7 | Q9H1E1 | NM_032572 | NP_115961 |
| 632 | 0.73 | Ras-related protein Rab-32 | RAB32 | Q13637 | NM_006834 | NP_006825 |
| 633 | 0.73 | Metalloproteinase inhibitor 1 | TIMP1 | P01033 | NM_003254, XM_017029766 | NP_003245, XP_016885255 |

TABLE 5-39

| | | | | | | |
|---|---|---|---|---|---|---|
| 634 | 0.73 | Tropomyosin alpha-1 chain | TPM1 | P09493 | NM_000366, NM_001018004, NM_001018005, NM_001018006, NM_001018007, NM_001018008, NM_001018020, NU_001301244, NM_001301289, NM_001330344, NM_001330346, NM_001330351, NM_001365776, NM_001365777, NM_001365778, NM_001365779, NM_001365780 NM_001365781, NM_001365782, XM_005254637, XM_005254638, XM_005254639, XM_005254645, XM_005254646, XM_005254647, XM_005254650, XM_005254651, XM_006720667, XM_017022534, XM_017022535, XM_017022536, XM_017022537, XM_017022538, XM_017022539, XM_017022540, XM_024450040 | NP_000357, NP_001018004, NP_001018005, NP_001018006, NP_001018007, NP_001018008, NP_001018020, NP_001288173, NP_001288218, NP_001317273, NP_001317275, NP_001317280, NP_001352705, NP_001352706, NP_001352706 NP_001352707, NP_001352708, NP_001352709 NP_001352710, NP_001352711, XP_005254694, XP_005254695, XP_005254696, XP_005254701, XP_005254703, XP_005254704, XP_005254707, XP_005254708, XP_006720730, XP_016878023, XP_016878024, XP_016878025, XP_016878026, XP_016878027, XP_016878028, XP_016878029, XP_024305808 |
| 635 | 0.73 | C-Maf-inducing protein | CMP | Q8lY22 | NM_030629, NM_198390, XM_005256179, XM_005256181, XM_005256182, XM_011523352, XM_011523353, XM_017023733 | NP_085132, NP_938204, XP_005256236, XP_005256238, XP_005256239, XP_01152164, XP_011521655, XP_016879222 |
| 636 | 0.73 | Inactive tyrosine-protein kinasa 7 | PTK7 | Q13308 | NM_001270398, NM_002821, NM_152880, NM_152881, NM_152882, NM_152883, XM_011514765, XM_011514766 | NP_001257327, NP_002812, NP_690619 NP_690620, NP_690621, XP_011513067, XP_011513068 |
| 637 | 0.73 | Protein phosphatase 1A | PPM1A | P35813 | NM_021003, NM_177951, NM_177952, XM_005267779, XM_005267781, XM_011536872, XM_011536876, XM_011536877, XM_011536878, XM_011536879, XM_011536880, XM_011536881, XM_011536882, XM_011536883, XM_017021381, XM_017021382, XM_017021383, XM_017021384, XM_017021385, XM_017021386, XM_017021387, XM_024449637 | NP_066283, NP_808820, NP_808821, XP_005267836, XP_005267838, XP_011535174, XP_011535178, XP_011535179, XP_011535180, XP_011535181, XP_011535182, XP_011535183, XP_011535184, XP_11535185, XP_016876870, XP_016876871, XP_016876872, XP_016876873, XP_016876874, XP_016876875, XP_016876876, XP_0240305405 |
| 638 | 0.73 | Tumor protein D54 | TPD52L2 | 043399 | NM_001243891, NM_001243892, NM_001243891, NM_001243895, NM_003288, NM_199359, NM_199360, NM_199361, NM_199362, NM_199363, XM_011529034, XM_011529035, XM_017028038 | NP_001230820, NP_001230821, NP_001230823, NP_001230824, NP_003279, NP_955391, NP_955392, NP_955393, NP_955394, NP_955395, XP_011527336, XP_011527337, XP_016883527 |
| 639 | 0.73 | Serpin B9 | SERPINB9 | P50453 | NM_004155, XM_005249184, XM_011514678, XM_017010942, XM_017010943 | NP_004146, XP_005249241, XP_011512980, XP_016866431, XP_016866432 |
| 64C | 0.73 | Serine incorporator3 | SERINC3 | Q13530 | NM_006811, NM_198941, XM_017027601 | NP_006802, NP_945179, XP_016883090 |

TABLE 5-40

| | | | | | | |
|---|---|---|---|---|---|---|
| 641 | 0.73 | Quinone oxidoreductase | CRYZ | Q08257 | NM_001130042, NM_001130043, NM_001134759, NM_001889, XM_005270491, XM_011540747, XM_017000367, XM_017000368 | NP_001123514, NP_001123515, NP_001128231, NP_001880, XP_005270548, XP_11539049, XP_016855856, XP_016855857 |
| 642 | 0.73 | Prostamide/prostaglandin F synthase | PRXL2B | Q8TBF2 | NM_001195736, NM_001195737, NM_001195738, NM_001195740, NM_001195741, NM_152371, XM_006710354, XM_011540665 | NP_001182665, NP_001182666, NP_001182667, NP_001182669, NP_001182670, NP_689584, XP_006710417, XP_011538967 |
| 643 | 0.73 | Secretory carrier-associated membrane protein 2 | SCAMP2 | 015127 | NM_001320778, NM_005697, XM_017021856 | NP_001307707, NP_005688, XP_016877345 |
| 644 | 0.73 | AP-2 comolex subunit mu | AP2M1 | Q96CW1 | NM_001025205, NM_001311198, NM_004068 | NP_001020376, NP_001298127, NP_004059 |
| 645 | 0.73 | Golgi-associated plant pathogenesis-related protein 1 | GLIPR2 | Q9H4G4 | NM_001287010, NM_001287011, NM_001287012, NM_001287013, NM_001287014, NM_022343, XM_024447416 | NP_001273939, NP_001273940, NP_001273941, NP_001273942, NP_001273943, NP_071738, XP_02430318 |
| 646 | 0.73 | HLAclass histocompatbility antigen, Cw4 alpha chain | HLA-C | P30504 | NM_001243042, NM_002117 | NP_001229971, NP_002108 |
| 647 | 0.73 | Phosphatidylinositol-binding clathrin assembly protein | PICALM | Q13492 | NM_001008660, NM_001206946, NM_001206947, NM_007166, XM_005274322, XM_005274323, XM_005274324, XM_005274325, XM_005274326, XM_005274327, XM_005274328, XM_005274329, XM_005274330, XM_005274331, XM_005274332, XM_005274333, XM_005274334, XM_005274335, XM_005274336, XM_005274337, XM_005274340, XM_006718699, XM_006718700, XM_006718701, XM_0011545293, XM_017018381, XM_017018382, XM_017018383, XM_017018384, XM_017018385, XM_017018386, XM_017018387, XM_017018388, XM_024448700 | NP_001008660, NP_001193875, NP_001193876, NP_009097, XP_005274379, XP_005274380, XP_005274381, XP_005274382, XP_005274383, XP_005274384, XP_005274385, XP_00527486, XP_005274387, XP_005274388, XP_005274389, XP_005274390, XP_005274391, XP_00527492, XP_005274393, XP_005274394, XP_005274397, XP-006718762, XP_006718763, XP_006718764, XP_011543595, XP_016873870, XP_016873871, XP_016873872, XP_016873873, XP_016873874, XP_016873875, XP_016873876, XP_016873877, XP_024304468 |
| 648 | 0.73 | CD166 antgen | ALCAM | Q13740 | NM_001243280, NM_001243281, NM_001243283, NM_001627 | NP_001230209, NP_001230210, NP_001230212, NP_001618 |
| 649 | 0.73 | Tyrosine-IRNAligase,cytoplasmic | YARS | P54577 | NM_003680, XM_011542347, XM_011542348, XM_017002651 | NP_003671, XP_011540649, XP_011540650, XP_016858140 |
| 650 | 0.73 | V-set and immunoglobulin domain-containing protein 10like | VSIG10L | Q86VR7 | NM_001163922 | NP_001157394 |
| 651 | 0.73 | Spectrin beta chain, non-erythrocytic 1 | SPTBN1 | Q01082 | NM_003128, NM_178313, XM_005264517, XM_005264518, XM_006712087, XM_017004779, XM_017004780, XM_017004781 | NP_003119, NP_842565, XP_005264574, XP_005264575, XP_006712150, XP_016860270 |
| 652 | 0.73 | CDK5 regulatory subunit-associated protein 3 | CDK5RAP3 | E96JB5 | NM_001278197, NM_1278198, NM_001278216, NM_001278217, NM_025197, NM_17095, NM_176096, XM_011525297, XM_017025164, XM_017025165, XM_017025166 | NP_001265126, NP_001265127, NP_001265145, NP_001265146, NP_788276, XP_011523599, XP_016880653, XP_016880654, XP_016880655 |
| 653 | 0.73 | Carbohydrate sulfotransferase 13 | CHST13 | Q8NET6 | NM_152889 | NP_690849 |

TABLE 5-41

| | | | | | | |
|---|---|---|---|---|---|---|
| 654 | 0.73 | 40S nibosomal protein S11 | RPS11 | P62280 | NM_001015 | NP_001006 |
| 655 | 0.73 | Developmetally-regulated GTP-binding protein 2 | DRG2 | P55039 | NM_001330144, NM_001388, XM_005256499, XM_005256500, XM_011523704, XM_011523706, XM_011523707, XM_017024296, XM_017024297 | NP_001317073, NP_001379, XP_005256556, XP_005256557, XP_011522006, XP_011522008, XP_011522009, XP_016879785, XP_016879786 |
| 656 | 0.73 | Small ubiquitin-related modifier3 | SUMO3 | P55854 | NM_001286418, NM_006936 | NP_001273345, NP_008867 |
| 657 | 0.73 | Protein transport protein Sec31A | SEC31A | 094919 | NM_001077206, NM_001077207, NM_001077208, NM_001191049, NM_001300744, NM_001300745, NM_001318119, NM_001318120, NM_014933, NM_016211 | NP_001070674, NP_001070675, NP_001070676, NP_001177978, NP_001287673, NP_001287674, NP_001305048, NP_001305049, NP_055748, NP_057295 |
| 658 | 0.73 | Comolement C1q tumor necrosis factor-related protein 3 | C1QTNF3 | Q9BXJ4 | NM_030945, NM_181435 | NP_112207, NP_852100 |
| 659 | 0.73 | Myosin light chain ⅓, skeletal muscle isoform | MYL1 | P05976 | NM_079420, NM_079422 | NP_524144, NP_524146 |
| 660 | 0.73 | POTE ankym domain family member F | POTEF | A5A3E0 | NM_001099771, XM_1017004833, XM_017004834, XM_17004835 | NP_001093241, XP_016860322, XP_016860323, XP_016860324 |
| 661 | 0.73 | Mtogen-activated protein Knase 1 | MAPK1 | P28482 | NM_002745, NM_138957 | NP_002736, NP_620407 |
| 662 | 0.73 | Programmed cell death protein 5 | PDCD5 | 014737 | NM_004708, XM_005259392 | NP_004699, XP_005259449 |
| 663 | 0.73 | Prominin-2 | PROM2 | Q8N271 | NM_001165977, NM_001165978, NM_001321070, NM_114707, XM_006712301, XM_011510672 | NP_001159449, NP_001159450, NP_001307999, NP_653308, XP_006712364, XP_011508974 |
| 664 | 0.73 | Ubiquitin-conjugating enzyme E2 variant 1 | UBE2V1 | Q13404 | NM_001032288, NM_001257393, NM_001257394, NM_001257395, NM_001261396, NM_001257397, NM_001257398, NM_001257399, NM_001282575, NM_001282576, NM_001282577, NM_001282578, NM_001282579, NM_001282580, NM_021988, NM_02242, NM_199144, NM_003349, NM_199203 | NP_001027459, NP_001244322, NP_001244323, NP_001244324, NP_001244325, NP_001244326, NP_001244327, NP_001244328, NP_001269504, NP_001269505, NP_001269506, NP_001269507, NP_001269508, NP_001269509, NP_068823, NP_071887, NP_954595, NP_954673 |
| 665 | 0.73 | Collagen alpna-2(Vl) chain | COL6A2 | P12110 | NM_001849, NM_058174, NM_058175, XM_011529451 | NP_001840, NP_478054, NP_478055, XP_011527753 |
| 666 | 0.73 | Choline transporter-like protein 4 | SLC44A4 | Q53GD3 | NM_001178044, NM_001178045, NM_025257 | NP_001171515, NP_001171516, NP_079533 |
| 667 | 0.73 | Utrophin | UTRN | P46939 | NM_007124, XM_005267127, XM_005267130, XM_005267133, XM_006715560, XM_011536101, XM_011536102, XM_011536106, XM_011536109, XM_017011243, XM_017011244, XM_017011245, XM_024446536 | NP_009055, XP_005267184, XP_005267187, XP_005267190, XP_006715623, XP_011534403, XP_011534404, XP_011534408, XP_011534411, XP_016866732, XP_016886733, XP_016866734, XP_024302304 |
| 668 | 0.73 | Acetyl-CoA carboxylase 1 | ACACA | Q13085 | NM_000664, NM_198834, NM_198835, NM_198836, NM_198837, NM_198838, NM_198839, XM_005257267, XM_006721853, XM_011524701, XM_011524703, XM_011524704, XM_017024553, XM_017024554, XM_017024555 | NP_942131, NP_942133, NP_942134, NP_942135, NP_942136, XP_005257324, XP_006721916, XP_011523003, XP_011523005, XP_011523006, XP_016880042, XP_016880043, XP_016880044 |

TABLE 5-42

| | | | | | | |
|---|---|---|---|---|---|---|
| 669 | 0.73 | Calpastatin | CAST | P20810 | NM_001042440, NM_001042441, NM_001042442, NM_001042443, NM_001042444, NM_001042445, NM_001042446, NM_001190442, NM_001284212, NM_001284213, NM_001330626, NM_001330627, NM_001330628, NM_001330629, NM_001330630, NM_001330631, NM_001330632, NM_001330633, NM_001330634, NM_001750, NM_173060, NM_173061, NM_173062, NM_173063 | NP_001035905, NP_001035906, NP_001035907, NP_001035908, NP_001035909, NP_001035910, NP_001035911, NP_001177371, NP_001271141, NP_001271142, NP_001317555, NP_001317556, NP_001317557, NP_001317558, NP_001317559, NP_001317560, NP_001317561, NP_001317562, NP_001317563, NP_001741, NP_775083 |
| 670 | 0.73 | Syndecan-4 | SDC4 | P31431 | NM_002999, XM_011528977 | NP_002990, XP_011527279 |
| 671 | 0.73 | Septin-9 | SEPT9 | Q9UHD8 | NM_001113491, NM_001113492, NM_001113493, NM_001113494, NM_001113495, NM_001113496, NM_001293695, NM_001293696, NM_001293697, NM_001293698, NM_006640 | NP_001106963, NP_001106964, NP_001106965, NP_001106966, NP_001106967, NP_001106968, NP_001280624, NP_001280625, NP_001280626, NP_001280627, NP_006631 |
| 672 | 0.73 | Interferon-inducible double-stranded RNA-dependent protein kinase activator | PRKRA | C75569 | NM_001139517, NM_001139518, NM_001316362, NM_003690, XM_011512063, XM_011512066, XM_017005159 | NP_001132989, NP_001132990, NP_001303291, NP_003681, XP_011510365, XP_011510368, XP_016860648 |
| 673 | 0.73 | TBC1 domain family member 9B | T6C1D9B | Q66K14 | NM_015043, NM_198868 | NP_055858, NP_942568 |
| 674 | 0.73 | Semaphorin-3B | SEMA3B | QI3214 | NM_001005914, NM_001290060, NM_001290061, NM_001290062, NM_001290063, NM_004636 | NP_001005914, NP_001276989, NP_001276990, NP_001276991, NP_001276992, NP_004627 |
| 675 | 0.725 | Adenosine kinase | ADK | P55263 | NM_001123, NM_001202449, NM_001202450, NM_006721, XM_017015698, XM_017015699, XM_017015700, XM_017015701, XM_017015702, XM_017015703, XM_017015704, XM_017015705, XM_017015706 | NP_001114, NP_001189378, NP_001189379, NP_006712, XP_016871187, XP_16871188, XP_016871189, XP_016871190, XP_016871191, XP_016871192, XP_016871193, XP_016871194, XP_016871195 |
| 676 | 0.725 | Corrinoid adenosyltransferase | MMAB | Q96EY8 | NM_052845, XM_011538267, XM_011538268, XM_011538269, XM_024448961 | NP_443077, XP_011536569, XP_011536570, XP_011536571, XP_024304729 |
| 677 | 0.72 | Beta-2-microglobulin | B2M | P61769 | NM_004048, XM_005254549 | NP_004039, XP_005254606 |
| 678 | 0.72 | Disintegrin and melalloproteinase domain-containing protein 10 | ADAM10 | C14672 | NM_001110, NM_001320570, XM_024449818 | NP_001101, NP_001307499, XP_024305586 |
| 679 | 0.72 | Integrin alpha-2 | TGA2 | P17301 | NM_002203 | NP_002194 |
| 680 | 0.72 | Synaptic vesicle membrane protein VAT-1 homolog | VAT1 | Q99536 | NM_006373 | NP_006364 |
| 681 | 0.72 | Dolichyl-dipnosphooligosaccharide-protein glycosyltransferase subunit 2 | RPN2 | P04844 | NM_001135771, NM_001324299, NM_001324301, NM_001324302, NM_001324303, NM_001324304, NM_001324305, NM_001324306, NM_002951, XM_006723851, XM_006723852 | NP_001129243, NP_001311228, NP_001311230, NP_001311231, NP_001311232, NP_001311233, NP_001311234, NP_001311235, NP_002942, XP_006723914, XP_006723915 |
| 682 | 0.72 | Coronin-1C | C0R01C | Q9ULV4 | NM_001105237, NM_001276471, NM_014325, XM_011538125, XM_017019121, XM_017019122, XM_017019123, XM_024448914, XM_024448915, XM_024448916, XM_024448917 | NP_001098707, NP_001263400, NP_055140, XP_011536427, XP_016874610, XP_016874611, XP_016874612, XP_024304682, XP_024304683, XP_024304684, XP_024304685 |

TABLE 5-43

| | | | | | | |
|---|---|---|---|---|---|---|
| 683 | 0.72 | Endothelin-converting enzyme 1 | ECE1 | P42892 | NM_001113347, NM_001113348, NM_001113349, XM_011540872, XM_011540873 | NP_001106818, NP_001106819, NP_001106820, NP_001388, XP_006710461, XP_011539174, XP_011539175 |
| 684 | 0.72 | Thymidine phosphorylase | TYMP | P19911 | NM_001113755, NM_001113756, NM_001257988, NM_001257989, NM_001953 | NP_001107227, NP_001107228, NP_001244917, NP_001244918, NP_001944 |
| 685 | 0.72 | Platelet factor 4 | PF4 | P02776 | NM_001363352, NM_002619, XM_005265696 | NP_001350281, NP_002610, XP_005265753 |
| 686 | 0.72 | Fermitin family homolog 3 | FERMT3 | Q86UX7 | NM_031471, NM_178443, XM_011545294, XM_011545295, XM_017018398, XM_017018399 | NP_113659, NP_848537, XP_011543596, XP_011543597, XP_016873887, XP_016873888 |
| 687 | 0.72 | Bone morphogenetic protein 1 | BMP1 | P13497 | NM_001199, NM_006128, NM_006129, NM_006130, NM_006131, NM_006132, XM_006716386, XM_011544617, XM_017013738 | NP_001190, NP_006120, XP_006716449, XP_011542919, XP_016869227 |
| 688 | 0.72 | Deleted in malignant brain tumors 1 protein | DMBT1 | Q9UGM3 | NM_001320644, NM_004406, NM_007329, NM_017579, XM_006717660, XM_006717655, XM_011539388, XM_011539389, XM_011539390, XM_011539391, XM_011539392, XM_011539393, XM_011539394, XM_011539395, XM_011539396, XM_011539398, XM_011539399, XM_011539400, XM_011539401, XM_011539402, XM_011539403, XM_011539405, XM_011539407, XM_011539408, XM_011539409, XM_011539410, XM_011539411, XM_011539413, XM_011539414, XM_011539415, XM_017015798, XM_024447854 | NP_001307573, NP_004397, NP_015568, NP_060049, XP_006717723, XP_006717728, XP_011537690, XP_011537691, XP_011537692, XP_011537693, XP_011537694, XP_011537695, XP_011537696, XP_011537697, XP_011537698, XP_011537700, XP_011537701, XP_011537702, XP_011537703, XP_011537704, XP_011537705, XP_011537707, XP_011537709, XP_011537710, XP_011537711, XP_011537712, XP_011537713, XP_011537715, XP_011537716, XP_011537717, XP_016871287, XP_024303622 |
| 689 | 0.72 | Pigment epithelium-derived factor | SERPINF1 | P36955 | NM_001329903, NM_001329904, NM_001329905, NM_002615 | NP_001316832, NP_001316833, NP_001316834, NP_002606 |
| 690 | 0.72 | Protein FAM234A | FAM234A | Q9HOX4 | NM_001284497, NM_032039, XM_005255622, XM_005255623, XM_006720957, XM_011522691, XM_011522692, XM_017023760, XM_017023761, XM_017023762, XM_017023763, XM_017023764, XM_017023765 | NP_001271426, NP_114428, XP_005255679, XP_005255680, XP_006721020, XP_011520993, XP_011520994, XP_016879249, XP_016879250, XP_016879251, XP_016879252, XP_016879253, XP_016879254 |
| 691 | 0.72 | Sorting nexin-1 | SNX1 | Q13596 | NM_001242933, NM_003099, NM_148955, NM_152826, XM_005254611 | NP_001229862, NP_003090, NP_683758, XP_005254668 |
| 692 | 0.72 | BRO1 domain-containing protein BROX | BROX | Q5VW32 | NM_001288579, NM_001288580, NM_001288581, NM_144695, XM_005273065, XM_005273069, XM_006711173, XM_011509212, XM_011509213, XM_011509214, XM_017000374, XM_017000375, XM_017000376 | NP_001275508, NP_001275509, NP_001275510, NP_653296, XP_005273122, XP_005273126. XP_006711236, XP_011507514, XP_011507515, XP_011507516, XP_016855863, XP_016855864, XP_016855865 |
| 693 | 0.72 | EKC/KEOPS complex subunit TP53RK | TP53RK | Q96S44 | NM_033550 | NP_291028 |
| 694 | 0.72 | Transitional endoplasmic reticulum ATPase | VCP | P55072 | NM_001354927, NM_001354928, NM_007126 | NP_001341856, NP_001341857, NP_009057 |

TABLE 5-44

| | | | | | | |
|---|---|---|---|---|---|---|
| 695 | 0.72 | Proteasome subunit beta type-9 | PSMB9 | P28065 | NM_002800, NM_148954 | NP_002791 |
| 696 | 0.72 | Tubulin beta-8 chain | TUBB8 | Q3ZCM7 | NM_001164154, NM_177987, XM_011519459, XM_011519460, XM_017016192, XM_017016193 | NP_817124, XP_011517761, XP_011517762, XP_016871681, XP_016871682 |
| 697 | 0.72 | Thioredoxin domain-containing protein 5 | TXNDC5 | Q8NBS9 | NM_001145549, NM_022085, NM_030810 | NP_001139021, NP_110437 |
| 698 | 0.72 | Wiskott-Aldrich syndrome protein family member 2 | WASF2 | C9Y6W5 | NM_001201404, NM_006990 | NP_001188333, NP_008921 |
| 699 | 0.72 | Histone H2B type 1-K | HIST1H2BK | 060814 | NM_001312653, NM_080593 | NP_001299582, NP_542160 |
| 700 | 0.72 | Chloride intracellular channel protein 4 | CLIC4 | Q9Y696 | NM_013943 | NP_039234 |
| 701 | 0.72 | 6-phosphogluconate dehydrogenase, decarboxylating | PGD | P52209 | NM_001304451, NM_001304452, NM_002631 | NP_001291380, NP_001291381, NP_002622 |
| 702 | 0.72 | Malectin | MLEC | Q14165 | NM_001303627, NM_001303628, NM_014730, XM_011539031, XM_011539032 | NP_001290556, NP_001290557, NP_055545, XP_011537333, XP_011537334 |
| 703 | 0.72 | Destrin | DSTN | P60981 | NM_001011546, NM_006870, XM_011529144 | NP_001011546, NP_006861, XP_011527446 |
| 704 | 0.72 | Heat shock protein HSP 90-beta | HSP90AB1 | P08238 | NM_001271969, NM_001271970, NM_001271971, NM_001271972, NM_007355, XM_024446421 | NP_001258898, NP_001258899, NP_001258900, NP_001258901, NP_031381, XP_024302189 |
| 705 | 0.72 | Kinesin-like protein KIF2A | KIF2A | O00139 | NM_001098511, NM_001243952, NM_001243953, NM_004520 | NP_001091981, NP_001230881, NP_001230882, NP_004511 |
| 706 | 0.72 | Transforming protein RhoA | RHOA | P61586 | NM_001313941, NM_001313943, NM_001313944, NM_001313945, NM_001313946, NM_001313947, NM_001664 | NP_001300870, NP_001300872, NP_001300873, NP_001300874, NP_001300875, NP_001300876, NP_001655 |
| 707 | 0.72 | Glutathione peroxdase 3 | GPX3 | P22352 | NM_001329790, NM_002084 | NP_001316719, NP_002075 |
| 708 | 0.72 | Alpha-1-antichymotrypsin | SERPINA3 | P01011 | NM_001085 | NP_001076 |
| 709 | 0.72 | Cartilage-associated protein | CRTAP | 075718 | NM_006371 | NP_006362 |
| 710 | 0.72 | Glutamine synthetase | GLUL | P15104 | NM_001033044, NM_001033056, NM_002065, XM_006711278 | NP_001028216, NP_001028228, NP_002056, XP_006711341 |
| 711 | 0.72 | Rhoguanine nucleotide exchange factor 1 | ARHGEF1 | Q92888 | NM_004706, NM_198977, NM_199002 | NP_004697, NP_945328, NP_945353 |
| 712 | 0.72 | EH domain-containing protein 2 | EHD2 | Q9NZN4 | NM_014601 | NP_055416 |
| 713 | 0.72 | Insulin-like growth factor | IGF2 | P01344 | NM_000612, NM_001007139, NM_001127598, NM_001291861, NM_001291862 | NP_000603, NP_001007140, NP_001121070, NP_001278790, NP_001278791 |
| 714 | 0.72 | Ubiquitin-like modifier-activating enzyme 5 | UBA5 | Q9GZZ9 | NM_001320210, NM_001321238, NM_001321239, NM_024818, NM_198329 | NP_001307139, NP_001308167, NP_001308168, NP_079094, NP_938143 |
| 715 | 0.72 | AP-2 complex subunit beta | AP2B1 | P63010 | NM_001030006, NM_001282, XM_005257937, XM_005257938, XM_005257941, XM_011524448, XM_011524449, XM_011524450, XM_011524451, XM_011524452, XM_011524453, XM_011524454, XM_011524455, XM_017024284, XM_017024285, XM_017024286, XM_017024287 | NP_001025177, NP_001273, XP_005257994, XP_005257995, XP_005257998, XP_011522750, XP_011522751, XP_011522752, XP_011522753, XP_011522754, XP_011522755, XP_011522756, XP_011522757, XP_016879773, XP_016879774, XP_016879775, XP_016879776 |
| 716 | 0.72 | Golgi phosphoprotein 3 | GOLPH3 | Q9H4A6 | NM_022130, XM_017009686 | NP_071413, XP_016865175 |
| 717 | 0.72 | Cullin-4B | CUL4B | Q13620 | NM_001079872, NM_001330624, NM_003588, XM_005262481, XM_006724784, XM_011531399, XM_011531400, XM_011531401 | NP_001073341, NP_001317553, NP_003579, XP_005262538, XP_006724847, XP_011529701, XP_011529702, XP_011529703 |
| 718 | 0.72 | Serine/threonine-protein phosphatase PP1-gamma catalytic subunit | PPP1CC | P36873 | NM_001244974, NM_002710, XM_011538504, XM_011538505 | NP_001231903, NP_002701, XP_011536806, XP_011536807 |

TABLE 5-45

| | | | | | | |
|---|---|---|---|---|---|---|
| 719 | 0.72 | Hepatocyte growth factor-regulated tyrosine kinase substrate | HGS | 014904 | NM_004712, XM_011525463, XM_017025297, XM_017025298, XM_024451024, XM_024451025 | NP_004703, XP_011523765, XP_016880786, XP_016880787, XP_024306792, XP_024306793 |
| 720 | 0.72 | ERO1-like protein alpha | ER01A | Q96HE7 | NM_014584, XM_011536684, XM_024449554 | NP_055399, XP_011534986, XP_024305322 |
| 721 | 0.72 | Arf-GAP with Rho-GAP domain, ANK repeat and PH domain-containing protein | ARAP1 | Q96P48 | NM_001040118, NM_001135190, NM_015242 | NP_001035207, NP_00112866, NP_056057 |
| 722 | 0.72 | Ephrin type-B receptor 2 | EPHB2 | P29323 | NM_001309192, NM_001309193, NM_004442, NM_017449, XM_006710441, XM_006710442, XM_024453895 | NP 001296121, NP_001296122, NP_004433, NP_059145, XP_006710504, XP_006710505, XP 024309663 |
| 723 | 0.72 | Interleukin-1 receptor accessory protein | L1RAP | Q9NPH3 | NM_001167928, NM_001167929, NM_001167930, NM_001167931, NM_001364879, NM_001364880, NM_001364881, NM_002182, NM_134470, XM_017006347, XM_017006348, XM_024453511 | NP_001161400, NP_001161401, NP_001161402, NP_001161403, NP_001351808, NP_001351809, NP_001351810, NP_002173, NP_608273, XP_016861836, XP_016881837, XP_024309279 |
| 724 | 0.72 | Transcriptional activator protein Pur-beta | PURB | C96QR8 | NM_033224 | NP_150093 |
| 725 | 0.72 | Collagen alpha-1(XVIII) chain | COL18A1 | P39060 | NM_030582, NM_130444, NM_130445 | NP_085059, NP_569711, NP_569712 |
| 726 | 0.72 | Eukaryotic translation elongation factor 1 epsilon-1 | EEF1E1 | O43324 | NM_001135650, NM_004280 | NP_001129122, NP_004271 |
| 727 | 0.72 | 26S proteasome non-ATPase regulatory subunit 4 | PSMD4 | P55036 | NM_001330692, NM_002810, NM_153822, XM_006711464, XM_017001856 | NP_001317621, NP_002801, XP_006711527, XP_016857345 |
| 728 | 0.72 | Eukaryotic peptide chain release factor GTP-binding subunit ERF3A | GSPT1 | P15170 | NM_001130008, NM_001130007, NM_002094 | NP_001123478, NP_001123479, NP_002085 |
| 729 | 0.72 | Septin-10 | SEPT10 | Q9P0V9 | NM_001321496, NM_001321498, NM_001321499, NM_001321500, NM_001321501, NM_001321502, NM_001321503, NM_001321504, NM_001321505, NM_001321506, NM_001321507, NM_001321508, NM_001321509, NM_001321510, NM_001321511, NM_001321512, NM_001321513, NM_001321514, NM_001321515, NM_144710, NM_178584, XM_006712317, XM_011510698, XM_011510699, XM_011510700, XM_011510701, XM_011510702, XM_011510703, XM_011510704 | NP_001308425, NP_001308427, NP_001308428, NP_001308429, NP_001308430, NP_001308431, NP_001308432, NP_001308433, NP_001308434, NP_001308435, NP_001308436, NP_001308437, NP_001308438, NP_001308439, NP_001308440, NP_001308441, NP_001308442, NP_001308443, NP_001308444, NP_653311, NP_848699, XP_006712380, XP_011509000, XP_011509001, XP_011509002, XP_011509003, XP_011509004, XP_011509005, XP_011509006 |
| 730 | 0.72 | Single lg IL-1-related receptor | SIGIRR | Q6IA17 | NM_001135053, NM_001135054, NM_021805, XM_005253044, XM_005253045, XM_005253046, XM_005253048, XM_017018099, XM_017018100 | NP_001128525, NP_001128526, NP_068577, XP_005253101, XP_005253102, XP_005253103, XP_005253105, XP_016873588, XP_016873589 |
| 731 | 0.72 | Muscleblind-like protein 2 | MBNL2 | Q5VZF2 | NM_001306070, NM_144778, NM_207304, XM_005254018, XM_005254019, XM_005254021, XM_005254022, XM_006719912, XM_024449300, XM_024449301 | NP_001292999, NP_659002, NP_997187, XP_005254075, XP_005254076, XP_005254078, XP_005254079, XP_006719975, XP_024305068, XP_024305069 |
| 732 | 0.72 | Pre-mRNA-processing factor 19 | PRPF19 | Q9UMS4 | NM_014502 | NP_055317 |

TABLE 5-46

| | | | | | | |
|---|---|---|---|---|---|---|
| 733 | 0.72 | Adolipoproten L2 | APOL2 | Q9BQE5 | NM_030882, NM_145637, XM_011530074, XM_011530075, XM_011530076, XM_011530077, XM_011530078, XM_017028724 | NP_112092, NP_663612, XP_011528376, XP_011528377, XP_011528378, XP_011528379, XP_011528380, XP_016884213 |
| 734 | 0.72 | Leucine-rich repeat protein SHCC-2 | SHOC2 | Q9UQ13 | NM_001269039, NM_001324336, NM_001324337, NM_007373 | NP_001255968, NP_001311265, NP_001311266, NP_031399 |
| 735 | 0.72 | Dynactin subunit 5 | DCTN5 | Q9BTE1 | NM_001199011, NM_001199743, NM_032486 | NP_001185940, NP_001185672, NP_115875 |
| 736 | 0.715 | Annexin A6 | ANXA6 | P08133 | NM_001155, NM_001193544, NM_001363114, NM_004033, XM_005268432 | NP_001146, NP_001180473, NP_001350043, XP_005268489 |
| 737 | 0.715 | Alpha-1,3-mannosyl-glycoprotein4-beta-N-acetylglucosaminyltransferase | BMGAT4B | Q9UQ53 | NM_014275, NM_054013, XM_024454347, XM_024454348, XM_024454349 | NP_055090, NP_463459, XP_024310115, XP_024310116, XP_024310117 |
| 738 | 0.715 | Elongin-B | ELOB | Q15370 | NM_007108, NM_207013 | NP_009039, NP_996896 |
| 739 | 0.71 | Carbonic anhydrase 2 | CA2 | P00918 | NM_000067, NM_001293675 | NP_000058, NP_001280604 |
| 740 | 0.71 | Integrin-linked protein kinase | ILK | Q13418 | NM_001014794, NM_001014795, NM_001278441, NM_001278442, NM_004517, XM_005252904, XM_005252905, XM_011520065, XM_017017672, XM_024448494, XM_24448495, XM_024448496, XM_024448497, XM_024448498, XM_024448499, XM_024448500 | NP_001014794, NP_001014795, NP_001265370, NP_001265371, NP_004508, XP_005252961, XP_005252962, XP_011518367, XP_016873161, XP_024304262, XP_024304263, XP_024304264, XP_024304265, XP_024304266, XP_024304267, XP_024304268 |

TABLE 5-47

| | | | | | | |
|---|---|---|---|---|---|---|
| 741 | 0.71 | Protein 4.1 | EPB41 | P11171 | NM_001166005, NM_001166006, NM_001166007, NM_004437, NM_203342, NM_203343, XM_005245753, XM_005245757, XM_005245760, XM_005245761, XM_005245763, XM_005245764, XM_005245765, XM_005245768, XM_005245769, XM_005245770, XM_005245772, XM_005245773, XM_005245774, XM_006710434, XM_006710439, XM_011540956, XM_011540957, XM_011540958, XM_011540959, XM_011540960, XM_011540961, XM_011540962, XM_011540963, XM_011540964, XM_011540965, XM_017000581, XM_017000582, XM_017000583, XM_017000584, XM_017000585, XM_017000586, XM_017000587, XM_017000588, XM_017000589, XM_017000590, XM_017000591, XM_017000592, XM_017000593, XM_017000594, XM_017000595, XM_017000596, XM_017000597, XM_017000598, XM_017000599, XM_017000600, XM_017000602, XM_017000603, XM_017000604, XM_024453880 | NP_001159477, NP_001159478, NP_001159479, NP_004428, NP_976217, NP_976218, XP_005245810, XP_005245814, XP_005245817, XP_005245818, XP_005245820, XP_005245821, XP_005245822, XP_005245825, XP_005245826, XP_005245827, XP_005245829, XP_005245830, XP_005245831, XP_006710497, XP_006710502, XP_011539258, XP_011539259, XP_011539260, XP_011539261, XP_011539262, XP_011539263, XP_011539264, XP_011539265, XP_011539266, XP_011539267, XP_016866070, XP_016856071, XP_016856072, XP_016856073, XP_016856074, XP_016856075, XP_016856076, XP_016856077, XP_016856078, XP_016856079, XP_016856080, XP_016856081, XP_016856082, XP_016856083, XP_016856084, XP_016856085, XP_016856086, XP_016856087, XP_016856088, XP_016856089, XP_016856091, XP_016856092, XP_016856093, XP 024309648 |
| 742 | 0.71 | Histidine, tRNAligase cytoplasm c | HARS | P12081 | NM_001258040, NM_001258041, NM_001258042, NM_001289092, NM_001289093, NM_001289094, NM_002109 | NP_001244969, NP_001244970, NP_001244971, NP_001276021, NP_001276022, NP_001276023, NP_002100 |
| 743 | 0.71 | Eukaryotic translation initiation factor 2 subunit 1 | EIF251 | P05196 | NM_004094 | NP_004085 |
| 744 | 0.71 | Complement C1r subcomponent | C1R | P00736 | NM_001354346, NM_001733 | NP_001341275, NP_001724 |
| 745 | 0.71 | CD99 antigen-like protein 2 | CD99L2 | Q8TCZ2 | NM_001184808, NM_001242614, NM_031462, NM_134445, NM_134446, XM_011531202, XM_017029890, XM_017029891 | NP_001171737, NP_001229543, NP_113650, NP_604394, NP_604395, XP_011529504, XP_016885379, XP_016885380 |
| 746 | 0.71 | Proto-oncogene tyrosine-protein kinase Src | SRC | P12931 | NM_005417, NM_198291, XM_011529013, XM_017028024, XM_017028025, XM_017028026, XM_017028027 | NP_005408, NP_938033, XP_011527315, XP_016883513, XP_016883514, XP_016883515, XP_016883516 |
| 747 | 0.71 | Cytosol aminopeptidase | LAP3 | P28838 | NM_015907 | NP_056991 |
| 748 | 0.71 | V-type proton ATPase 16 kDa proteolipid subunit | ATP6V0C | P27449 | NM_001198569, NM_001694 | NP_001185498, NP_001685 |
| 749 | 0.71 | Nicastrin | NCSTN | Q92542 | NM_001290184, NM_001290186, NM_001349729, NM_015331, XM_005245053 | NP_001277113, NP_001277115, NP_001336658, NP_056146, XP_005245110 |
| 750 | 0.71 | Flotillin-1 | FLOT1 | C75955 | NM_001318875, NM_005803, XM_005248780, XM_006714947, XM_017010157, XM_017010158 | NP_001305804, NP_005794, XP_005248837, XP_006715010, XP_016865646, XP_016865647 |

TABLE 5-48

| | | | | | | |
|---|---|---|---|---|---|---|
| 751 | 0.71 | TRAF2 and NCK-interactng protein Kinase | TNIK | G9UKE5 | NM_001161560, NM_001161561, NM_001161562, NM_001161563, NM_001161564, NM_001161565, NM_001161566, NM_015028 | NP_001155032, NP_001155033, NP_001155034, NP_001155035, NP_001155036, NP_001155037, NP_001155038, NP_055843 |
| 752 | 0.71 | Protein-L-isoaspartate(D-aspartate) O-methyltransferase | PCMT1 | P22061 | NM_001252049, NM_001252050, NM_001262051, NM_001252052, NM_001252053, NM_001360452, NM_001360458, NM_005389, XM_011535868, XM_024446451 | NP_001238978, NP_001238979, NP_001238980, NP_001238981, NP_001238982, NP_001347381, NP_001341385, NP_005380, XP_011534170, XP_024302219 |
| 753 | 0.71 | Dynactin subunit 2 | DCTN2 | Q13561 | NM_001261412, NM_001261413, NM_001348065, NM_001348066, NM_001348067, NM_001318068, NM_006400 | NP_001248341, NP_001248342, NP_001334994, NP_001334995, NP_001334996, NP_001334997, NP_006391 |
| 754 | 0.71 | Apolipoproten L1 | APOL1 | 014791 | NM_001136540, NM_001136541, NM_001362927, NM_003661, NM_145343, NM_145344, XM_005261796, XM_011530478 | NP_001130012, NP_001130013, NP_001349856, NP_003652, NP_663318, XP_005261853, XP_011528780 |
| 755 | 0.71 | Ras-related C3 botulinum toxin substrate 2 | RAC2 | P15153 | NM_002872, XM_006724286 | NP_002863, XP_006724349 |
| 756 | 0.71 | Metalloproteinase inhibitor 3 | TIMP3 | P35625 | NM_000362 | NP_000353 |
| 757 | 0.71 | Platelet-activating factor acetylhydrolase IB subunit alpha | PAFAH1B1 | P43034 | NM_000430, XM_011523901, XM_011523902, XM_011523903, XM_017024701, XM_017024702, XM_017024703 | NP_000421, XP_011522203, XP_011522204, XP_011522205, XP_016880190, XP_01016880191, XP_016880192 |
| 758 | 0.71 | Solute carrier family 2, facilitated glucose transporter member 1 | SLC2A1 | P11166 | NM_006516 | NP_006507 |
| 759 | 0.71 | Guanine nucleotide-binding protein G(I)/G(S)/G(O) subunit gamma-12 | GNG12 | Q9UBI6 | NM_018841, XM_17001809, XM_024448405 | NP_061329, XP_016857298, XP_024304173 |
| 760 | 0.71 | Inositol monophosphatase 3 | IMPAD1 | Q9NX62 | NM_017813 | NP_060283 |
| 761 | 0.71 | Xaa-Pro dipeptidase | PEPD | P12955 | NM_000285, NM_001166056, NM_001166057 | NP_000276, NP_001159528, NP_001159529 |
| 762 | 0.71 | Proteolipid protein 2 | PLP2 | Q04941 | NM_002668 | NP_002659 |
| 763 | 0.71 | Large neutral amino acids transporter small subunit 1 | SLC7A5 | Q01650 | NM_003486, XM_006721286, XM_017023735, XM_017023736, XM_017023737 | NP_003477, XP_006721349, XP_016879224, XP_016879225, XP_016879226 |
| 764 | 0.71 | CSC1-like protein 1 | TMEM63A | 094886 | NM_014698, XM_006711841, XM_011544328, XM_011544329, XM_011544330, XM_011544331, XM_011544332 | NP_055513, XP_006711904, XP_011542630, XP_011542631, XP_011542632, XP_011542633, XP_011542634 |
| 765 | 0.71 | Ubiquitin-like-conjugating enyme ATG3 | ATG3 | Q9NT62 | NM_001278712, NM_022488, XM_011513074 | NP_001265641, NP_071933, XP_011511376 |
| 766 | 0.71 | Dipeptidyl peptidase 3 | DPP3 | Q9NY33 | NM_001256670, NM_005700, NM_130443 | NP_001243599, NP_005691, NP_569710 |
| 767 | 0.71 | Lysosome-associated membrane glycoprotein 2 | LAMP2 | P13473 | NM_001122606, NM_002294, NM_013995 | NP_001116078, NP_002285, NP_054701 |
| 768 | 0.71 | V-type proton ATPase subunit D | ATP6V1D | Q9Y5K8 | NM_015994 | NP_057078 |
| 769 | 0.71 | Ras-related protein Rab-12 | RAB12 | Q6IQ22 | NM_001025300, XM_006722300 | NP_001020471, XP_006722363 |
| 770 | 0.71 | Oxysterol-bindig protein 2 | OSBP2 | Q969R2 | NM_001003812, NM_001282738, NM_001282739, NM_001282740, NM_001282741, NM_001282742, NM_030758 | NP_001269667, NP_001269668, NP_001269669, NP_001269670, NP_001269671, NP_11385 |
| 771 | 0.71 | Vacuolar protein sorting-associated protein 35 | VPS35 | Q96QK1 | NM_018206, XM_005256045, XM_011523227 | NP_060676, XP_005256102, XP_011521529 |
| 772 | 0.71 | Coatomer subunit zeta-1 | COPZ1 | P61923 | NM_001271734, NM_001271735, NM_001271738, NM_016057 | NP_001258663, NP_001258664, NP_001258665, NP_057141 |
| 773 | 0.71 | Glutathione S-transferase Mu 3 | GSTM3 | P21266 | NM_000849 | NP_000840 |

TABLE 5-49

| | | | | | | |
|---|---|---|---|---|---|---|
| 774 | 0.71 | Ribulase-phosphate 3-epimerase | RPE | Q96AT9 | NM_001278282, NM_001278283, NM_001278285, NM_001278286, NM_001278288, NM_001278289, NM_001318926, NM_001318927, NM_001318928, NM_001318929, NM_001318930, NM_001318931, NM_006916, NM_199229, XM_006712677, XM_024453035 | NP_001265211, NP_001265212, NP_001265214, NP_001265215, NP_001265217, NP_001265218, NP_001305855, NP_001305856, NP_001305857, NP_001305858, NP_001305859, NP_001305860, NP_008847, NP_954699, XP_006712740, XP_024308803 |
| 775 | 0.71 | 40S ribosomal protein S21 | RPS21 | P63220 | NM_001024, XM_024451959, XM_024451980 | NP_001015, XP_024307727, XP_024307728 |
| 776 | 0.71 | Heterogeneous nuclear ribonucleoprotein A3 | HNRNPA3 | P51991 | NM_001330247, NM_001330248, NM_001330249, NM_001330250, NM_001330251, NM_194247 | NP_001317176, NP_001317177, NP_001317178, NP_001317179, NP_001317180, NP_919223 |
| 777 | 0.71 | NPC intacellularcholesterol transporter 1 | NPC1 | 015118 | NM_000271, XM_005258277, XM_005258278, XM_005258279, XM_006722479, XM_017025784, XM_017025785, XM_017025786, XM_017025787 | NP_000262, XP_005258334, XP_005258335, XP_005258336, XP_006722542, XP_016881273, XP_016881274, XP_016881275, XP_016881276 |
| 778 | 0.71 | Eukaryotic translation initation factor 4 gamma 2 | EIF4G2 | P78344 | NM_001042559, NM_001172705, NM_001418 | NP_001036024, NP_001166176, NP_001409 |
| 779 | 0.71 | Furrarylacetoacetase | FAH | P16930 | NM_000131, XM_024449872 | NP_000128, XP_024305640 |
| 780 | 0.71 | Glycylpeptide N-tetradecanoyltransferase 1 | NMT1 | P30419 | NM_021079 | NP_066565 |
| 781 | 0.71 | Asparagine synthetase [glutamine-hydrolyzing] | ASNS | P08243 | NM_001178075, NM_001178076, NM_001178077, NM_001352496, NM_001673, NM_133436, NM_183356 | NP_001171546, NP_00171547, NP_001171548, NP_001339425, NP_001664, NP_597680, NP_899199 |
| 782 | 0.71 | Alpha-adducin | ADD1 | P35611 | NM_001119, NM_001286645, NM_001354754, NM_001354755, NM_001354756, NM_001354757, NM_001354758, NM_001354759, NM_001354761, NM_001354762, NM_014189, NM_014190, NM_176801, XM_005247934, XM-017007703, XM_017007704, XM_017007705, XM_017007706, XM_017007709, XM_024453887 | NP_001110, NP_001273574, NP_001341683, NP_001341684, NP_001341685, NP-001341686, NP_001341687, NP_001341688, NP_001341690, NP_001341691, NP_054908, NP_054909, NP_789771, XP_005247991, XP_016863192, XP_016863193, XP_016863194, XP_01016863195, XP_016863198, XP_024309655 |
| 783 | 0.71 | E3 ubiquitin-protein ligase HUWE1 | HUWE1 | Q7Z6Z7 | NM_031407, XM_005261965, XM_011530747, XM_011530751, XM_017029191, XM_017029192, XM_017029193, XM_017029194, XM_017029195, XM_017029196, XM_017029197, XM_017029198, XM_017029199, XM_017029200, XM_017029201, XM_017029202, XM_017029203, XM_017029204, XM_017029206, XM_024452322 | NP_113584, XP_005262022, XP_011529049, XP_011529053, XP_016884680, XP_016884681, XP_016884682, XP_016884683, XP_016884684, XP_016884685, XP_016884686, XP_016884687, XP_016884688, XP_01016884689, XP_01E88469D, XP_016884691, XP_016884692, XP_016884693, XP_01016884695, XP_024308090 |
| 784 | 0.71 | DnaJ homolog subfamily Amemoer 2 | DNAJA2 | 060884 | NM_005880 | NP_005871 |
| 785 | 0.71 | Tyrosine-protein kinase Fgr | FGR | P09769 | NM_001042729, NM_001042747, NM_005248, XM_006710452, XM_011541010, XM_011541011, XM_C11541012, XM_011541013, XM_011541014, XM_017000673, XM_017000674 | NP_001036194, NP_001036212, NP_005239, XP_006710515, XP_011539312, XP_011539313, XP_011539314, XP_011539315, XP_011539316, XP_016856162, XP-016856163 |

TABLE 5-50

| | | | | | | |
|---|---|---|---|---|---|---|
| 786 | 0.71 | Translin | TSN | Q15631 | NM_001261401, NM_004622 | NP_001248330, NP_004613 |
| 787 | 0.71 | Unconventional myosin-b | MYO1B | O43795 | NM_001130158, NM_001161819, NM_001330237, NM_001330238, NM_012223, XM_005246572, XM_071004158, XM_024452914, XM_024452915 | NP_001123630, NP_001155291, NP_001317166, NP_001317167, NP_036355, XP_005246629, XP_016859647, XP_024308682, XP_024308683 |
| 788 | 0.71 | Casein kinase 1 isoform gamma-3 | CSNK1G3 | Q9Y6M4 | NM_001031812, NM_001044722, NM_001044723, NM_001270572, NM_001270573, NM_001270574, NM_001364140, NM_001364141, NM_001364143, NM_001364144, NM_001364145, NM_001364146, NM_001364147, NM_001364148, NM_001364149, NM_001364150, NM_004384, XM_005271891, XM_005271892, XM_005271894, XM_005271895, XM_005271896, XM_017009057, XM_017009059, XM_017009060, XM_017009061, XM_017009062, XM_017009063, XM_017009064, XM_017009065, XM_017009066, XM_017009067, XM_017009068, XM_017009069, XM_017009070 | NP_001026982, NP_001038188, NP_001257501, NP_001257502, NP_001257503, NP_001351069, NP_001351070, NP_001351072, NP_001351073, NP_001351074, NP_001351075, NP_001351076, NP_001351077, NP_001351078, NP_001351079, NP_004375, XP_005271948, XP_005271949, XP_005271951, XP_005271952, XP_005271953, XP_016864546, XP_016864548, XP_016864549, XP_016864550, XP_016864551, XP_016864552, XP_016864553, XP_016864554, XP_016864555, XP_016864556, XP_016864557, XP_016864558, XP_016864559 |
| 789 | 0.71 | Adenylosuccinate synthetase isozyme 2 | ADSS | P30520 | NM_001126, NM_001365073, XM_011544108 | NP_001117, NP_001352002, XP_011542410 |
| 790 | 0.71 | ATP-binding cassette sub-family F member 3 | ABCF3 | Q9NUQ8 | NM_001351298, NM_001351299, NM_001351300, NM_018358, XM_024453624, XM_024453625 | NP_001338227, NP_001338228, NP_001338229, NP_060828, XP_024309392, XP_024309393 |
| 791 | 0.71 | Voltage-dependent calciurr channel subunit aloha-2/delta-1 | CACNA2D1 | P54289 | NM_000722, NM_001302890, XM_005250570, XM_005250572, XM_005250573, XM_005250574, XM_006716118, XM_006716119, XM_006716120, XM_006716121, XM_011516570, XM_011516571, XM_011516572, XM_017012588 | NP_000713, NP_001289819, XP_005250627, XP_005250629, XP_005250630, XP_005250631, XP_006716181, XP_006716182, XP_006716183, XP_006716184, XP_011514872, XP_011514873, XP_011514874, XP J16868077 |
| 792 | 0.71 | Galaclocerebros dase | GALC | P54803 | NM_000153, NM_001037525, NM_001201401, NM_001201402, XM_011536618 | NP_000144, NP_001188330, NP_001188331, XP 011534920 |
| 793 | 0.71 | AMP deaminase 2 | AMPD2 | Q01433 | NM_001257360, NM_001257361, NM_001308170, NM_004037, NM_139156, NM_203404, XM_024446431, XM_024446432 | NP_001244289, NP_001244290, NP_001295099, NP_004028, NP_631895, NP_981949, XP_024302199, XP_024302200 |
| 794 | 0.71 | Ras-related orote n Rab-20 | RAB20 | Q9NX57 | NM_017817 | NP_060287 |
| 795 | 0.71 | Placophilin-2 | PKP2 | Q99959 | NM_001005242, NM_004572 | NP_001005242, NP_004563 |
| 796 | 0.71 | Oxysterol-binding protein-related protein 2 | OSBPL2 | Q9HIP3 | NM_001001691, NM_001278649, NM_001363878, NM_014835, NM_144498, XM_017028163, XM_017028164, XM_017028165, XM_017028166, XM_017028167, XM_017028168, XM_017028169, XM_017028170 | NP_001265578, NP_001350807, NP_055650, NP_653081, XP_016883652, XP_016883653, XP_016883654, XP_016883655, XP_016883656, XP_016883657, XP_016883658, XP_016883659 |

TABLE 5-51

| | | | | | | |
|---|---|---|---|---|---|---|
| 797 | 0.71 | Zinc transporter ZIP10 | SLC39A10 | Q9ULF5 | NM_001127257, NM_020342, XM_005246689, XM_011511504, XM_011511505, XM_011511506, XM_011511507, XM_017004522, XM_017004523, XM_017005484 | NP_001120729, NP_065075, XP_005246746, XP_011509806, XP_011509807, XP_011509808, XP_011509809, XP_016860011, NP_016860012, XP_016860973 |
| 798 | 0.71 | ProteasomalATPase-associated factor 1 | PAAF1 | Q9BRP4 | NM_001267803, NM_001267804, NM_001267805, NM_001267806, NM_001363556, NM_025155, XM_005274308, XM_005274309, XM_017018357, XM_017018358, XM_017018359 | NP_001254732, NP_001254733, NP_001254734, NP_001254735, NP_001350485, NP_079431, XP_005274365, XP_005274366, XP_016873846, XP_016873847, XP_016873848 |
| 799 | 0.71 | Coronin-7 | COR07 | P57737 | NM_001201472, NM_001201473, NM_001351729, NM_024535 | NP_001188401, NP_001188402, NP_001338658, NP_078811 |
| 800 | 0.71 | Ras-related proteinRab-17 | RAB17 | Q9H0T7 | NM_022449, XM_006712689, XM_017004693, XM_017004694 | NP_071894, XP_006712752, XP_016860182, XP 016860183 |
| 801 | 0.705 | Bcl-2-like protein 1 | BCL2L1 | QC7817 | NM_001191, NM_001317919, NM_001317920, NM_001317921, NM_001322239, NM-001322240, NM_001322242, NM_138578, XM_011528964, XM_017027993 | NP_001182, NP_001304848, NP_001304849, NP_001304850, NP_001309168, NP_001309169, NP_001309171, NP_612815, XP_011527266, XP_016883482 |
| 802 | 0.7 | Protein tyrosine phosphatase type NA2 | PTP4A2 | Q12974 | NM_001195100, NM_001195101, NM_003479, NM_080391, NM_080392, XM_005271231, XM_005271232, XM_017002401 | NP_001182029, NP_001182030, NP_536316, XP_005271288, XP_005271289, XP_016857890 |
| 803 | 0.7 | Calreticulin | CALR | P27797 | NM_004343 | NP_004334 |
| 804 | 0.7 | Receptor-type tyrosine-protein phosphatase alpha | PTPRA | P18433 | NM_002836, NM_080840, NM_080841 | NP_002827, NP_543030, NP_543031 |
| 805 | 0.7 | Heterogeneous nuclear ribonucleoproteins A2/B1 | HNRNPA2B1 | P22626 | NM_002137, NM_031243, XM_005249729, XM_017012109, XM_017012110 | NP _002128, NP_112533, XP_005249786, XP_016867598, XP_016867599 |
| 806 | 0.7 | Heme-binding protein 2 | HEBP2 | Q9Y5Z4 | NM_001326380, NM_001328381, NM_014320 | NP_001313309, NP_001313310, NP_055135 |
| 807 | 0.7 | 26S oroteasome non-ATPase regulatory subunit 8 | PSMD8 | P48556 | NM_002812 | NP_002803 |
| 808 | 0.7 | Mannan-binding lectin serine protease 2 | MASP2 | O00187 | NM_006610, NM_139208, XM_017000097 | NP_006601, NP_631947, XP_016855586 |
| 809 | 0.7 | Protein FAM177A1 | FAM177A1 | Q8N128 | NM_001079519, NM_001289022, NP_173607 | NP_001072987, NP_001275951, NP_775878 |
| 810 | 0.7 | Drebrin-like protein | DBNL | Q9UJU6 | NM_001014436, NM_001122956, NM_001284313, NM_001284315, NM_001362723, NM_014063, XM_011515325, | NP_001014436, NP_001116428, NP_001271242, NP_001271244, NP_001349652, NP_054782, XP_011513627 |
| 811 | 0.7 | TBC1 domain family member 10B | TBC1D10B | Q4KMP7 | NM-015527, XM_011545789, XM_011545790 | NP_056342, XP_011544091, XP_011544092 |
| 812 | 0.7 | Tyrosine-protein kinase Lyn | LYN | P07948 | NM_001111097, NM_002350, XM_011517529, XM_017013415, XM_017013416 | NP_001104567, NP_002341, XP_011515831, XP_016868904, XP_016868905 |
| 813 | 0.7 | Secretory carrier-associated membrane protein 1 | SCAMP1 | 015126 | NM_001290229, NM_004866, NM_052822, XM_011543727 | NP_001277158, NP_004857, XP_011542029 |
| 814 | 0.7 | Calmodulin-1 | CALM1 | PODP23 | NM_001166106, NM_001363669, NM_001363670, NM_006888, XM_006720258, NM_001305624, NM_001305625, NM_001305626, NM_001743, NM_001329921, NM_001329922, NM_001329923, NM_001329924, NM_001329925, NM_001329926, NM_005184 | NP_001350598, NP_001350599, NP_008819, XP_006720321, NP_001292553, NP_001292554, NP_001292555, NP_001734, NP_001316850, NP_001316851, NP_001316852, NP_001316853, NP_001316854, NP_001316855, NP_005175 |
| 815 | 0.7 | Erythrocyte band 7 integral membrane protein | STOM | P27105 | NM_001270526, NM_001270527, NM_004099, NM_198194 | NP_001257455, NP_001257456, NP_004090, NP_937837 |

TABLE 5-52

| | | | | | | |
|---|---|---|---|---|---|---|
| 816 | 0.7 | Tripeptidyl-peptidase 2 | TPP2 | P29144 | NM_001330588, NM_0003291, XM_005254070, XM_005254071 | NP_001317517, NP_003282, XP_005254127, XP_005264128 |
| 817 | 0.7 | Tropomyosin alpha-3 chain | TPM3 | P06753 | NM_001043351, NM_001043352, NM_001043353, NM_001278188, NM_001278189, NM_001278190, NM_001278191, NM_001349679, NM_001364679, NM_001364680, NM_001364681, NM_001364682, NM_001364683, NM_152263, NM_153649 | NP_001036816, NP_001036817, NP_001036818, NP_001265117, NP_001265118, NP_001265119, NP_001265120, NP_001336608, NP_0011351608, NP_001351609, NP_001351610, NP_001351611, NP_001351612, NP_689476, NP_705935 |
| 818 I | 0.7 | Vacuolar protein sorting-associated protein 26B | VPS26B | Q4G0F5 | NM_052875, XM_011512565 | NP_443107, XP_011540867 |
| 819 I | 0.7 | Ribonuclease 4 | RNASE4 | P34096 | NM_001282192, NM_001282193, NM_002937, NM_194430, NM_194431 | NP_001269121, NP_001269122, NP_002928, NP_919412 |
| 820 | 0.7 | Major vault protein | MVP | Q14764 | NM_001293204, NM_00293205, NM_005115, NM_017458 | NP_001280133, NP_001280134, NP_005106, NP_059447 |
| 821 1 | 0.7 | Glucose-6-phosphate 1-dehydrogenase | G6PD | P11413 | NM_000402, NM_001042351, NM_001360016 | NP_000393, NP_001035810, NP_001346945 |
| 822 | 0.7 | Calc neurin B homologous protein 3 | TESC | Q86BS2 | NM_001168325, NM_017899, XM_011538503, XM_017019532, XM_017019533 | NP_001161797, NP_060369, XP_011536805, XP_01687502, XP_016875022 |
| 823 | 0.7 | E3 uoiquitin-protein ligase LRSAM1 | LRSAM1 | Q6UWE0 | NM_001005373 NM_001005374, NM_001190723 NM_138361, XM_006717316, XM_017015283, XM_017015284 | NP_001005373, NP_001005374, NP_001001177652, NP _612370, XP_006717379, XP_016870772, XP_016870773 |
| 824 | 0.7 | Heterogeneous nuclear ribonucleoproteins C1/C2 | HNRNPC | P07910 | NM_001077442 NM_001077443, NM_004500, NM_031314, XM_006720125, XM_011536708, XM_011536709, XM_011536710, XM_011536711, XM_011536712, XM_017021252, XM_017021253, XM02449556, XM_024449557, XM_024449558, XM_024449559 | NP_001070910, NP_001070911, NP_004491, NP_112604, XP_006720188, XP_011535010, XP_01153011, XP_011535012, XP_011535013, XP _011535014, XP_016876741, XP_016876742, XP_24305324, XP_024305325, XP_024305326, XP_024305327 |
| 825 | 0.7 | Lysosomal acid phosphatase | ACP2 | P11117 | NM_001131064 NM_001302489, NM_001302490, NM_001302491, NM_001302492 NM_001357016, NP_001610 | NP_001001289418, NP_01289419, NP_001289420, NP_001289421, NP_001343945, NP_001601 |
| 826 | 0.7 | NADP- NADP-dependent malic enzyme | ME1 | P48163 | NM_002395, XM_011535836 | NP_002386, XP_011534138 |
| 827 | 0.7 | F-box/LRR-repeat protein 20 | FBXL20 | Q96lG2 | NM_001184906, NM_032875, XM_005257746, XM_005257747 | NP_001171835, NP-16264, XP_005257803, XP_005157804 |
| 828 | 0.7 | Ras-related protein Rab-18 | RAB18 | Q9NP72 | NM_001256410 NM_001256411, NM_001256412 NM_001256415, NM_021252 | NP_001243339, NP_001243340, NP_001001243341, NP_001243344, NP_067075 |
| 829 | 0.7 | Kallikrein-7 | KLK7 | P49862 | NM_001207053, NM_001243126, NM_005046, NM_139277 | NP_001193982, NP_001230055, NP_005037, NP_644806 |
| 830 | 0.7 | Septn-8 | SEPT8 | Q92599 | NM_001098811, NM_001098812, NM_001098813, NM_001300798, NM_001300799, NM_0151146, XM_005271927, XM_005271928, XM_005271931, XM_011543288, XM_017009254, XM_017009255, XM_017009256, XM_017009257, XM_017009258, XM_017009259, XM_017009260, XM_017009261 | NP_001001092281, NP_001092282, NP_001092283, NP_001287727, NP_001287728, NP_055961, XP_005271984, XP_005271985, XP_005271988, XP_011541590, XP_016864743, XP_016864744, XP_016864745, XP_016864746, XP_016864747, XP_016864748, XP_016864749, XP_016864750 |

TABLE 5-53

| | | | | | | |
|---|---|---|---|---|---|---|
| 831 | 0.7 | Pituitary tumor-transforming gene 1 protein-interacting protein | PTTG1P | P53801 | NM_001286822 NM_004339 | NP_001001273751, NP-004330 |
| 832 | 0.7 | Golg apparatus protein 1 | GLG1 | Q92896 | NM_001145666, NM_001145667, NM_012201 | NP_001139138, NP_001139139, NP_036333 |
| 833 | 0.7 | Methionineadenosyltransferase 2 subunit beta | MAT2B | Q9NZL9 | NM_013283, NM_182796 | NP_037415, NP_877725 |
| 834 | 0.7 | Protein NDRG1 | NDRG1 | Q92597 | NM_001135242, NM_001258432, NM_001258433, NM_006096, XM_011516791, XM_011516792 | NP_001128714, NP_001245361, NP_001245362, NP_006087, XP_011515093, XP_011515094 |
| 835 | 0.7 | Ex000lyphosphatasePRUNE1 | PRUNE 1 | Q86TP1 | NM_001303229, NM_001303242, NM_001303243, NM_021222, XM_005245393, XM_011509832, XM_017001955, XM_017001956, XM_017001957 | NP_001290158, NP_001290171, NP_001290172, NP_067045, XP_005245450, XP_011508134, XP_016857444, XP_016857445, XP_016857446 |
| 836 | 0.7 | Beta-centractin | ACTR1B | P42025 | NM_005735, XM_005263854, XM_017003116 | NP_005726, XP_005263911, XP_016858605 |
| 837 | 0.7 | Katanin p60 ATPase-containing subunit A-like 2 | KATNAL2 | Q8lYT4 | NM_001353899, NM_001353900, NM_001353901, NM_001353902, NM 001353903, NM_001353904, NM_001353905, NM_001353906, NM_001353907, NM_001353908, NM_001353909, NM_031303, XM_005258361, XM_006722554, XM_011526219, XM_011526220, XM_011526221, XM_011526223, XM_017026028, XM_017026030, XM_017026031 | NP_001340828, NP_001340829, NP_001340830, NP_001340831, NP_001340832, NP_001340833, NP _001340834, NP_001340835, NP_001340836, NP_001340837, NP_001340838, NP_112593, XP_005258418, XP_006722617, XP_011524521, XP_011524522, XP_011524523, XP_011524525, XP_016881517, XP_016881519, XP_016881520 |
| 838 | 0.7 | Cystatin-C | CST3 | P01034 | NM_000099, NN_001288614 | NP_000090, NP_001275543 |
| 839 | 0.7 | Maleylacetoacetate isomerase | GSTZ1 | 043708 | NM_001312660, NM_001363703, NM_001513, NM_146870, NM_145871, XM_011536670, XM_011536671, XM_024449548, XM_024449549, XM_024449550, XM_024449551, XM_024449552 | NP_001299589, NP_001350632, NP_665877, NP_665878, XP_011534972, XP_011534973, XP_024305316, XP_024305317, XP_024305318, XP_024305319, XP_024305320 |
| 840 | 0.7 | Protein dpy-30 homolog | DPY30 | Q9C005 | NM_001321209, NM_001321210, NM_032574, XM_006712117, XM_006712118, XM_011533133, XM_017005111 | NP_001308138, NP_001308139, NP_115963, XP_006712180, XP_006712181, XP_011531435, XP_016860600 |
| 841 | 0.7 | MCB-like protein phocein | M0B4 | Q9Y3A3 | NM_001100819, NM_001204094, NM_015387, NM_199482 | NP_001094289, NP_001191023, NP_056202, NP_955776 |
| 842 | 0.7 | DNA-(apurinic or apyrimidinic site) lyase | APEX1 | P27695 | NM_0011244249 NM_001641, NM_080648, NM_080649 | NP_001231178, NP_001632, NP_542379, NP_542380 |
| 843 | 0.7 | Kallikrein-5 | KLK5 | Q9Y337 | NM_001077491 NM_001077492, NM_012427, XM_011526702, XM_011526703 | NP_001070959, NP_001070960, NP_036559, XP_011525004, XP_011525005 |
| 844 | 0.7 | Carbohydrate sulfotransferase 12 | CHST12 | Q9NRB3 | NM_001243794, NM_001243795, NM_018641, XM_0011515443, XM_011515444 | NP_001230723, NP_001230724, NP_061111, XP_011513745, XP_011513746 |

TABLE 5-54

| | | | | | | |
|---|---|---|---|---|---|---|
| 845 | 0.7 | OX-2 membrane glycoprotein | CD200 | P41217 | NM_001004196, NM_00194197, NM_001318826, NM_001318828, NM_001318830, NM_001365851, NM_001365852, NM_001365853, NM_001365854, NM_001365855, NM_005944, XM_005247481, XM_005247482, XM_05247485, XM_00524786, XM_005247488, XM_0017006452, XM_017006453, XM_017006454, XM_017006455, XM_017006456, XM_017006457, XM_017006458, XM_017006459 | NP_001004196, NP_001305755, NP_001305757, NP_001305759, NP_001352780, NP_001352781, NP_001352782, NP_001352783, NP_001352784, NP_005935, XP_005247538, XP_005247539, XP_005247542, XP_005247543, XP_005247545, XP_016861941, XP_016861942, XP_016861943, XP_016861944, XP_016861945, XP_016861946, XP_016861947, XP_016861948 |
| 846 | 0.7 | 3-hydroxybutyrate dehydrogenase type2 | BDH2 | Q9BUT1 | NM_020139, XM_05263140, XM_006714274, XM_011532127, XM_011532128, XM_017008461, XM_017008462 | NP_064524, XP_005263197, XP_006714337, XP_011530429, XP_011530430, XP_016863950, XP_016863951 |
| 847 | 0.7 | Phosphoribosyl pyrophosphate synthase-associated protein 2 | PRPSAP2 | O60256 | NM_001243936, NM_001243940, NM_001243941, NM_001243942, NM_001353096, NM_001353097, NM_001353098, NM_001353099, NM_001353100, NM_001353101, NM_001353102, NM_001353103, NM_001353104, NM_001353105, NM_001353106, NM_001353107, NM_002767, XM_017024865, XM_017024866, XM_017024871, XM_017024873, XM_017024874, XM_017024875 | NP_001230865, NP_001230869, NP_001230870, NP_001230871, NP_001340025, NP_001340026, NP_001340027, NP_001340028, NP_001340029, NP_001340030, NP_001340031, NP_001340032, NP_001340033, NP_001340034, NP_001340035, NP_001340036, NP_002758, XP_016880354, XP_016880355, XP_016680360 XP_016880362, XP_016680363, XP_016880364 |
| 848 | 0.7 | D-glucuronyl C5-epimerase | GLCE | O94923 | NM_001324091, NM_001324092, NM_001324093, NM_001324094, NM_015554, XM_005254298, XM_017022073, XM_017022074 | NP_001311020, NP_001311021, NP_001311022, NP_001311023, NP_056369, XP_005254355, XP_016877562, XP_016877563 |
| 849 | 0.7 | SLIT-ROBO Rho GTPase-activating protein 2 | SRGAP2 | O75044 | NM_001042158, NM_001170637, NM_001300952, NM_015326, XM_005277510, XM_005277511, XM_005277514, XM_005277515, XM_011509354, XM_011509355, XM_011509356, XM_011509359, XM-017000838, XM_017000840, XM_017000841, XM_024446014, XM_024446015, XM_024446016 | NP_001164108, NP_001287881, NP_056141, XP_005277567, XP_005277568, XP_005277571, XP_005277572, XP_011507656, XP_011507657, XP_011507658, XP_011507661, XP_016856327, XP_016856329, XP_016856330, XP_024301782, XP_024301783, XP_024301784 |
| 850 | 0.7 | Interferon regulatory factor 6 | IRF6 | O14898 | NM_001206696, NM_006147 | NP_001193625, NP_006138 |
| 851 | 0.7 | Lupus La protein | SSB | P05455 | NM_001294145, NM_003142 | NP_001281074, NP_003133 |

As shown in Table 5-1 to Table 5-54, a group of proteins able to be used to deter-mine drug sensitivity was found in proteins present in exosomes in the blood of lung cancer patients.

### 3. Extraction of Membrane Protein Groups

Based on the Predicted membrane proteins information in The Human Protein Atlas (https://www.proteinatlas.org/), membrane protein groups were extracted from the protein groups in Table 5-1 to Table 5-54 described above. The list of obtained membrane proteins is shown in Tables 6-1 to 6-15.

TABLE 6-1

| AUC Rank | AUC | Protein Name | Gene name | Uniprot Ac | ReSeq_NM | RefSeq_NP |
|---|---|---|---|---|---|---|
| 1 | 0.96 | Band 4.1-like protein 2 | EPB41L2 | 043491 | NM_001135554, NM_001135555, NM_001199388, NM_001199389, NM_001252660, NM_001350299, NM_001350301, NM_001350302, NM_001350303, NM_001350304, NM_001350305, NM_001350306, NM_001350307, NM_001350308, NM_001350309, NM_001350310, NM_001350311, NM_001350312, NM_001350313, NM_001350314, NM_001350315, NM_001350320, NM_001431, XM_011535524, XM_011535525, XM_011535527, XM_011535528, XM_017010350, XM_017010351, XM_017010352, XM_017010353, XM_017010356, XM_017010364, XM_024446349 | NP_001129026, NP_001129027, NP_001186317, NP_001239589, NP_001337228, NP_001337230, NP_001337231, NP_001337232, NP_001337233, NP_001337234, NP_001337235, NP_001337236, NP_001337237, NP_001337238, NP_001331239, NP_001337240, NP_001337241, NP_001337242, NP_001337243, NP_001337244, NP_001337249, NP_001422, XP_011533826, XP_011533827, XP_011333829, XP_011533830, XP_016865839, XP_016865840, XP_016865841, XP_016865842, XP_016865845, XP_016865853, XP_024302117 |
| 6 | 0.92 | Attractin | ATRN | O75882 | NM_001207047, NM_001323332, NM_012070, NM_139321, NM_139322 | NP_001193976, NP_001310261 NP_647537, NP_J47538 |
| 13 | 0.89 | Rho GTPase-activating protein 1 | ARHGAP1 | Q07960 | NM_004308, XM_011520095, XM_024448520 | NP_004299, XP_11518397, XP_024304288 |
| 17 | 0.81 | Integrin alpha-V | ITGAV | P06756 | NM_001144999, NM_001145000, NM_002210 | NP_001138471, NP_001138472, NP_002201 |

TABLE 6-2

| | | | | | | |
|---|---|---|---|---|---|---|
| 20 | 0.88 | Adhesion G-protein coupled receptor G1 | ADGRG1 | Q9Y653 | NM_001145770, NM_001145771, NM_001145772, NM_001145773, NM_001146774, NM_001290142, NM_001290143, NM_001290144, NM_005682, NM_201524, NM_201525, XM_005256237, XM_005256238, XM_005256239, XM_005256240, XM_005256241, XM_005256242, XM_005256244, XM_005256245, XM_005256246, XM_005256247, XM_005256248, XM_005256249, XM_005256251, XM_005256252, XM_005256254, XM_005256255, XM_006721338, XM_006721339, XM_006721340, XM_006721341, XM_006721342, XM_006721343, XM_006721344, XM_006721345, XM_006721346, XM_006721347, XM_011523461, XM_011523462, XM_011523463, XM_011523464, XM_011523465, XM_011523466, XM_011523467, XM_011523468, XM_017023892 | NP_001139242, NP_001139243, NP_001139244, NP_001139245, NP_001139246, NP_001277071, NP_001277072, NP_001277073, NP_005673, NP_958932, NP_958933, XP_005256294, XP_005256295, XP_005256296, XP_005256297, XP_005256298, XP_005256299, XP_005256301, XP_005256302, XP_005256303, XP_005256304, XP_005256305, XP_005256306, XP_005256308, XP_005256309, XP_005256311, XP_005256312, XP_006721401, XP_006721402, XP_006721403, XP_006721404, XP_006721405, XP_006721406, XP_006721407, XP_006721408, XP_006721409, XP_006721410, XP_011521763, XP_011521764, XP_011521765, XP_011521766, XP_011521767, XP_011521768, XP_011521769, XP_011521770, XP_016879381 |
| 21 | 0.88 | Integrin beta-5 | ITGB5 | P18084 | NM_001354764, NM_01354765, NM_001354766, NM_002213, XM_005247436, XM_006713630, XM_017006353, XM_017006354, XM_017006355 | NP_001341693, NP_001341694, NP_001341695, NP_002204, XP_005247493, XP_006713693, XP_016861842, XP_016861843, XP_016861844 |
| 26 | 0.88 | Transient receptor potential cation channel subfamily M member 4 | TRPM4 | Q81D43 | NM_001195227, NM_001321281, NM_001321282, NM_001321283, NM_001321285, NM_017636, XM_005259017, XM_024451557 | NP_001182156, NP_001308210, NP_001308211, NP_001308212, NP_001308214, NP_060106, XP_005259074, XP_024307325 |
| 29 | 0.88 | Insulin receptor | INSR | P06213 | NM_000208, NM_001079817, XM_011527988, XM_011527989, | NP_000199, NP_001073285, XP_011526290, XP_011526291 |
| 43 | 0.86 | Sodium/potassium-transporting ATPase subunit alpha-1 | ATP1A1 | P05023 | NM_000701, NM_001001586, NM_001160233, NM_001160234, XM_017001360, XM_017001361 | NP_000692, NP_001153705, NP_001153706, XP_016856849, XP_016856850 |
| 45 | 0.86 | Major prion protein | PRNP | P04156 | NM_000311, NM_001080121, NM_001080122, NM_001080123, NM_001271561, NM_183079 | NP_000302, NP_001073590, NP_001073591, NP_001073592, NP_001258490, NP_898902 |
| 61 | 0.85 | Fructose-bisphosphate aldolase A | ALDOA | P04075 | NM_000034, NM_001127617, NM_001243175, NM_001243177, NM_001355562, NM_001355563, NM_001355564, NM_001355565, NM_184041, NM_184043, XM_024450192, XM_024450193 | NP_000025, NP_001121089, NP_001230106, NP_001342491, NP_001342492, NP_001342493, NP_001342494, NP_908930, NP_908932, XP_024305960, XP_024305961 |

TABLE 6-3

| | | | | | | |
|---|---|---|---|---|---|---|
| 68 | 0.85 | Neuroplastin | NPTN | Q9Y639 | NM_001161363, NM_001161364, NM_012428, NM_017455, XM_011521452, XM_024449899, XM_024449900 | NP_001154835, NP_001154836, NP_036560, NP_059429, XP_011519754, XP_024305667, XP_024305668 |
| 76 | 0.84 | Sodium/potassium-transporting ATPase subunit beta-3 | ATP1B3 | P54709 | NM_001679 | NP_001670 |
| 83 | 0.84 | Tetraspanin-15 | TSPAN15 | 095858 | NM_001351263, NM_012339, XM_005269666, XM_011539562, XM_011539563, XM_017016010 | NP_001338192, NP_036471, XP_005269723, XP_011537864, XP_011537865, XP_016871499 |
| 90 | 0.84 | DnaJ homolog subfamily C member 5 | DNAJC5 | Q9H3Z4 | NM_025219, XM_011529048, XM_011529049 | NP_079495, XP_011527350, XP_011627351 |
| 94 | 0.84 | Cytochrome b5 type B | CYBSB | 043169 | NM_030579 | NP_085056 |
| 95 | 084 | Progressive ankylosis protein homolog | ANKH | Q9HCJ1 | NM_054027, XM_011514067, XM_017009644 | NP_473368, XP_011512369, XP_016865133 |
| 100 | 0.84 | Inactive tyrosine-protein kinase transmembrane receptor ROR1 | ROR1 | Q01973 | NM_001083592, NM_005012, XM_011541526, XM_017001376, XM_017001377 | NP_001077061, NP_005003, XP_011539828, XP_016856865, XP_016856866 |
| 111 | 0.83 | CD97 antigen | CD97 | P48960 | NM_001025160, NM_001784, NM_078481, XM_011528451 | NP_001020331, NP_001775, NP_510966, XP_011526753 |
| 121 | 0.83 | Extended synaptotagmin-2 | ESYT2 | A0FGR8 | NM_020728, XM_024446845, XM_024446846, XM_024446847, XM_024446848, XM_024446849 | NP_065779, XP_024302613, XP_024302614, XP_024302615, XP_024302616, XP_024302617 |
| 122 | 0.83 | Leukoeyte surface antigen CD47 | CD47 | Q08722 | NM_001025079, NM_001025080, NM_001777, NM_198793, XM_005247908, XM_005241909, XM_017007536 | NP_001768, NP_942088, XP_005247965, XP_005247966, XP_016863025 |
| 127 | 0.83 | CD276 antigen | CD276 | Q5ZPR3 | NM_001024736, NM_001329628, NM_001329629, NM_025240, XM_005254700, XM_011522095, XM_017022638 | NP_001019907, NP_001316557, NP_001316558, NP_079516, XP_005254757, XP _011520397, XP_016878127 |
| 135 | 0.83 | Protein phosphatase 1L | PPM1L | Q5SGD2 | NM_001317911, NM_001317912, NM_139245, XM_011512440 | NP_001304840, NP_001304841, NP_640338, XP_011510742 |
| 138 | 0.83 | Trophoblast glycoprotein | TPBG | Q13641 | NM_001166392, NM_006670 | NP_001159864, NP_006661 |
| 153 | 0.82 | Ephrin-B1 | EFNB1 | P98172 | NM_004429 | NP_004420 |
| 188 | 0.81 | Equilibrative nucleoside transporter 1 | SLC29A1 | Q99808 | NM_001078174, NM_001078175, NM_001078116, NM_001078177, NM_001304462, NM_001304463, NM_001304465, NM_001304466, NM_004955, XM_005248876, XM_005248878, XM_005248879, XM 005248880, XM_005248881, XM_005248882, XM_011514341, XM_024446348 | NP_001071643, NP_001071645, NP_001291391, NP_001291392, NP_001291394, NP_001291395, XP_006248933, XP_005248935, XP_005248936, XP_005248937, XP_005248938, XP_005248939, XP_011512643, XP_024302116 |
| 195 | 0.81 | Sodium/potassium-transportingATPase subunit beta-1 | ATP1B1 | P05026 | NM_001001787, NM_001677 | NP_001668 |
| 196 | 0.81 | Endothelial cell-selective adhesion molecule | ESAM | Q96AP7 | NM_138961 | NP_620411 |

TABLE 6-4

| | | | | | | |
|---|---|---|---|---|---|---|
| 198 | 0.81 | Peptidyl-prolyl cis-trans isomerase FKBP1A | FKBP1A | P62942 | NM_000801, NM_001199786, NM_054014 | NP_000792, NP_001186715, NP_463460 |
| 199 | 0.81 | Dystroglycan | DAG1 | Q14118 | NM_001165928, NM_001177634, NM_001177635, NM_001177636, NM_001177637, NM_001177638, NM_001177639, NM_001177640, NM_001177641, NM_001177642, NM_001177643, NM_001177644, NM_004393 | NP_001159400, NP_001171105, NP_001171106, NP_001171107, NP_001171108, NP_D01171109, NP_001171110, NP_001171111, NP_001171112, NP_001171113 NP_001171114, NP_001171115, NP_004384 |
| 206 | 0.81 | Folate receptor alpha | FOLR1 | P15328 | NM_000802, NM_016724, NM_016725, NM_016729, NM_016730 | NP_000793, NP_057936, NP_057937, NP_057941 |
| 212 | 0.81 | Membrane cofactor protein | CD46 | P15529 | NM_002389, NM_153826, NM_172350, NM_172351, NM_172352, NM_172353, NM_172354, NM_172355, NM_172356, NM_172357, NM_172358, NM_172359, NM_172360, NM_172361. XM_011509563, XM_011509564, XM_017001308, XM_017001309, XM_017001310 | NP_002380, NP_722548, NP_758860, NP_758861, NP_758862, NP_758863, NP_758865, NP_758866, NP_758867, NP_758868, NP_758869, NP_758871, XP_011507865, XP_011507866, XP_016856797, XP_16856798, XP_016856799 |
| 238 | 0.8 | NCK-interacting protein with SH3 domain | NCKIPSD | Q9NZQ3 | NM_016453, NM_184231, XM_017006594, XM_017006595, XM_017006596, XM_017006597, XM_017006598, XM_017006699, XM_017006600 | NP_057537, NP_909119, XP_016862083, XP_016862084, XP_016862085, XP_016862086, XP_016862087, XP_016862088, XP_016862089 |
| 239 | 0.8 | Vesicle-associated membrane protein 5 | VAMPS | 095183 | NM_006634 | NP_006625 |
| 250 | 0.8 | Syntaxin-binding protein 1 | STXBP1 | P61764 | NM_001032221, NM_003165 | NP_001027392, NP_003156 |
| 253 | 0.8 | Nodal modulator 3 | NOMO3 | P69849 | NM_001004067, XM_005255318 | NP_001004067,XP_005255375 |
| 265 | 0.79 | Receptor-type tyrosine-protein phosphatase eta | PTPRJ | Q12913 | NM_001098503, NM_002843, XM_017018083, XM _017018084, XM_017018085 | NP_001091973, NP_002834, XP_016873572, XP_016873673, XP_016873514 |
| 266 | 0.79 | CD109 antigen | CD109 | Q6YHK3 | NM_001159587, NM_001159588, NM_133493 | NP_001153059, NP_001153060, NP_598000 |
| 267 | 0.79 | Integrin beta-1 | ITGB1 | P05556 | NM_002211, NM_033666, NM_033667, NM_033668, NM_033669, NM_133376 | NP_002202, NP_391988, NP_596867 |
| 268 | 0.79 | LHFPL tetraspan subfamily member 2 protein | LHFPL2 | Q6ZUX7 | NM_005779, XM_006714615, XM_024454321, XM_024454322, XM_024454323, XM_024454324, XM_024454325 | NP_005170, XP_006714578, XP_024310089, XP_024310090, XP_024310091, XP_024310092, XP_024310093 |

TABLE 6-5

| | | | | | | |
|---|---|---|---|---|---|---|
| 270 | 0.79 | Glucose-6-phosphate isomerase | GPI | P06744 | NM_000175, NM_001184722, NM_001289789, NM_001289790, NM_001329909, NM_001329910, NM_001329911, XM_011526754 | NP_000166, NP_001171651, NP_001276718, NP_001276719, NP_001316838,NP_001316839, NP_001316840, XP_011525056 |
| 271 | 0.79 | Choline tansporter-like protein 2 | SLC44A2 | Q8IWA5 | NM_001145056, NM_001363611, NM_020428, XM_005259997, XM_005259999 | NP_001138528, NP_001350540, NP_065161, XP_005260054, XP_005260056 |
| 275 | 0.79 | Synaplosomal-associated protein 23 | SNAP23 | O00161 | NM_003825, NM_130798, XM_006720725, XM_017022692, XM_17022693, XM_017022694 | NP_003816, NP_570710, XP_006720788, XP_016818181, XP_016878182, XP_016878183 |
| 280 | 0.79 | Amyloid-beta precursor protein | APP | P05067 | NM_000484, NM_001136016, NM_001136129, NM_001136130, NM_01136131, NM_001204301 NM_001204302, NM_001204303, NM_201413, NM_201414, XM_024452015 | NP_000475, NP_001129488, NP_001129601, NP_001129602, NP_001129603, NP_001191230, NP_001191231, NP_001191232, NP_958816, NP_958817, XP_024307843 |
| 281 | 0.79 | Vesicle-associated membrane protein 8 | VAMPS | Q9BV40 | NM_003761, XM_017005170 | NP_003752, XP_016860659 |
| 282 | 0.79 | Lymphocyte function-associated antigen 3 | CD58 | P19256 | NM_001144822, NM_ 0017i9, XM_017002869 | NP_001138294, NP_001770, XP_016858358 |
| 290 | 0.79 | CD151 antigen | CD151 | P48509 | NM_001039490, NM_004357, NM_139029, NM_139030, XM_024448780 | NP_001034579, NP_004348, NP_620598, NP_620599, XP_0430448 |
| 291 | 0.79 | Selenoprotein P | SELENOF ^{:} | P49908 | NM_001085486, NM_ 001093726 NM_005410 | NP_001078955, NP_001087195, NP_005401 |
| 304 | 0.79 | Syntaxin- 4 | STX4 | Q12846 | NM_001272096, NM_001272096, NM_004604, XM_005255521 | NP_001259024, NP_001259025, NP_004595, XP_005255578 |
| 306 | 0.79 | E3 ubiquitin-protein ligase RNF128 | RNF128 | Q8TEB7 | NM_024539, NM_194463 | NP_078815, NP_919445 |
| 310 | 0.79 | Large neutral amino acids transporter small subunit 3 | SLC43A1 | 075387 | NM_001198810, NM_003627, XM_005274358, XM_011545320, XM_011545321, XM_011545322, XM_017018451, XM_017018452, XM_017018453, XM_024448727, XM_024448728 | NP_01185739, NP_003618, XP_005274415, XP_011543622, XP_011543623, XP_011543624, XP_016873940, XP_016873941, XP_016873942, XP_J24304495, XP_024304496 |
| 312 | 0.79 | Interleukin-17 receptor A | IL17RA | Q96F46 | NM_001289905, NM_014339 | NP_001276834, NP_055154 |
| 323 | 0.78 | Multidrug resistance-associated protein 1 | ABCC1 | P33527 | NM_004996, NM_019862, NM_019898, NM_019899, NM_019900, NM_019901, NM_019902, XM_011522497, XM_011522498, XM_017023237, XM_017023238, XM_017023239, XM_017023240, XM_017023241, XM_017023242, XM_017023243 | NP_004987, XP_011520799, XP_011520800, XP_016878726, XP_016878727, XP_016878728, XP_016878129, XP_016878730, XP_016878731, XP_016878732 |
| 327 | 0.78 | Plasma membrane calcium-transporting ATPase 4 | ATP2B4 | P23634 | NM_001001396, NM_001365783 NM_001365784, NM_001684 | NP_001001396 NP_001352712, NP_001352713 NP_001675 |

TABLE 6-6

| | | | | | | |
|---|---|---|---|---|---|---|
| 330 | 0.78 | Phospholipid scramblase 3 | PLSCR3 | Q9NRY6 | NM_001201576, NM_C2036C | NP_001188505 NP_065093 |
| 332 | 0.78 | 4F2 cell-surface antigen heavy chain | SLC3A2 | P08195 | NM_001012661, NM_CO1012662 NM 001012663, NM_001012664 NM_001013251, NM_002394 | NP_001012680 NP_001012682, NP_001013269. NP_002385 |
| 334 | 0.78 | Ubiquitin-like modifier-activating enzyme 7 | UBA7 | P41226 | NM_003335, XM_005265430, XM_006713321, XM_011534070, XM_011534071, XM_011534072, XM 024453740 | NP_003326, XP_005265487, XP_006713384,XP_011532372, XP_011532373, XP_011532374, XP_024309508 |
| 336 | 0.78 | Integrin alpha-Ib | ITGA2B | P08514 | NM_000419, XM_011524749, XM_011524750 | NP_000410, XP_11523051, XP_011523052 |
| 353 | 0.78 | Tyrosine-protein phosphatase non-receptor type 1 | PTPN1 | P18031 | NM_001278618, NM_002827 | NP_001265547, NP_002818 |
| 361 | 0.77 | Syntaxin-7 | STX7 | 015400 | NM_001326578, NM_001326579, NM_001326580, NM_003669 | NP_001313507, NP_001313508, NP_001313509, NP_003560 |
| 362 | 0.77 | Claudin-7 | CLDN7 | 095471 | NM_001185022, NM_01185023, NM_001307 | NP_001171951, NP_001171962, NP_001298 |
| 376 | 0.77 | CD59 glycoprotein | CD59 | P13987 | NM_000611, NM_001121223, NM_001127225, NM_ 001127226, NM_001127227, NM_203329, NM_203330, NM_203331 | NP_000602, NP_001120695, NP_01120697, NP_001120698, NP_001120699, NP_976074, NP_976075, NP_976076 |
| 378 | 0.77 | Ubiquifio-like mooifier-activating enzyme 1 | UBA1 | P22314 | NM_003334, NM_153280, XM_005272649, XM_011543954, XM_017029777, XM 017029778, XM_011029719,XM_017029180, XM_017029781 | NP_003326, NP_695012, XP_005272706, XP_011542256, XP_016885266, XP_016885267, XP_016885268,XP_016885269, XP_016885210 |
| 380 | 0.77 | Canalicular multispecific organic anion transporter 2 | ABCC3 | 015438 | NM_001144070, NM_003786, NM_020031, NM_020038, XM_05257763, XM_011525422, XM_D11525423,XM_011525424, XM_011525425, XM_017025265, XM_017025266 | NP_001137542, NP_003777, XP_005257820, XP_011623724, XP_011523725, XP_011623726, XP_011523727, XP_016880754, XP_016880755 |
| 388 | 0.77 | Monocarboxylate transporter 4 | SLC16A3 | 015427 | NM_001042422,NM_001042423, NM 001206960, NM_001206951, NM_001206952, NM_004207, XM_024451023 | NP_001035887, NP_001035888, NP_001192879, NP_001193880, NP_001193881, NP_004198, XP_024306791 |
| 392 | 0.77 | Podocalyxin | PODXL | 000592 | NM_001018111, NM_005397 | NP_001018121, NP_005388 |
| 404 | 0.77 | Calcium release-activated calcium channel protein 1 | ORAI1 | Q96D31 | NM_032790 | NP_116179 |
| 408 | 0.77 | CKLF-like MARVEL transmembrane doma n-containing protein 6 | CMTM6 | Q9NX76 | NM_017801 | NP_060211 |
| 409 | 0.77 | Carcinoembryonic antigen-related cell adhesion molecule 6 | CEACAM6 | P40199 | NM_002483, XM_011526990 | NP_002474, XP_011525292 |

TABLE 6-7

| | | | | | | |
|---|---|---|---|---|---|---|
| 411 | 0.77 | Transmembrane channel-like protein 6 | TMC6 | Q7Z403 | NM_001127198, NM_001321185, NM_007267, XM_005256995, XM_011624265, XM_011624266, XM_011524257, XM_011524258, XM_17024107, XM_011024108, XM_024460655, XM_024450566, XM_024460657 | NP_001120670, NP_001308114, NP_009198, XP_05257052, XP_011622561 XP_011622568, XP_011522559, XP_011522560, XP_016B79596, XP_016879597, XP_024306323, XP_024306324, XP_024306326 |
| 412 | 0.77 | Transmembrane protein 222 | TMEM222 | Q9H0R3 | NM_032125 | NP_115501 |
| 419 | 0.76 | CD9 antigen | CD9 | P21926 | NM_001330312, NM_001769, XM_005253814 | NP_001317241, NP_001760, XP_005263871 |
| 421 | 0.76 | Myeloid-associated differentiation marker | MYADM | Q96S97 | NM_001020818, NM_001020819, NM_001020820, NM_001020821, NM_001290188, NM_001290189, NM_001290190, NM_001290191 NM_001290192, NM_001290193 NM_001290194, NM_138373 | NP_001018654, NP_001018655, NP_D01018656, NP_001018657, NP_001277117, NP_001277118,NP_D01277119, NP_001277120, NP_001277121, NP_001277122, NP_001277123, NP_612382 |
| 423 | 0.76 | Integrin beta-3 | ITGB3 | P05106 | NM_000212 | NP_000203 |
| 425 | 0.76 | Vesicle-associated membrane protein 3 | VAMP3 | Q15836 | NM_004781, XM_017002841 | NP_004772, XP_016858330 |
| 427 | 0.76 | Interleukin-6 recepor subunit beta | IL6ST | P40189 | NM_001190981, NM_001364275, NM_001364276, NM_001364277 NM_001364278, NM_001364219, NM_002184, NM_175767, XM_017009441, XM_017009442 | NP_001177910, NP_001351204, NP_001351205, NP_001351206, NP_001351207, NP_001351208, NP_002175, NP_786943, XP_016864930, XP_016864931 |
| 438 | 0.76 | Sodium- and chloride-dependent taurine transporter | SLC6A6 | P31641 | NM_001134367, NM_01134368, NM_003043, XM_006713307, XM_011634030, XM_011634031 | NP_001127839, NP_001127B40, NP_003034, XP_006713370, XP_011532332 XP_011532333 |
| 442 | 0.76 | Syntaxin-12 | STX12 | Q86YB2 | NM_171424 | NP_803173 |
| 447 | 0.76 | Sortilin | SORT1 | Q99523 | NM_001205228, NM_002959, XM_005271100, XM_006271101, XM_005271102, XM_006710612 | NP_001192151,NP_002950, XP_005271151, XP_005271158, XP_005271159, XP_006710875 |
| 454 | 0.76 | Protein PRRC1 | PRRC1 | Q96M21 | NM_001286808, NM_130809, XM_006714529 | NP_001273737, NP_570721, XP_006714592 |
| 470 | 0.76 | Ribosome-binding protein 1 | RRBP1 | Q9P2E9 | NM_001042576, NM_001365613, NM_004587 | NP_001036041, NP_001352542, NP_004578 |
| 474 | 0.76 | Poliovirus receptor | PVR | P15151 | NM_001135768, NM_001135769, NM_001135770, NM_006505 | NP_001129240, NP_001129241, NP_001129242, NP_006496 |
| 479 | 0.75 | Integrin alpha-6 | ITGA6 | P23229 | NM_000210, NM_001079818, NM_ 001316306, NM_001365529, NM_001365630, XM_006712510, XM_006712511, XM_017004005, XM_017004006, XM_017004007, XM_017004008 | NP_000201, NP_001073286, NP_001303235, NP_001352458, NP_001352459,XP_006712513, XP_006712514,XP 016859494, XP_016859495, XP_016859496, XP_016B69497 |

TABLE 6-8

| | | | | | | |
|---|---|---|---|---|---|---|
| 480 | 0.75 | Tetraspanin-14 | TSPAN14 | Q8NG11 | NM_001128309, NM_001351266, NM_001351267, NM_001351268, NM_001351269, NM_001351270, NM_001351271, NM_001351272, NM_030927, XM_011540219, NM_011540220, XM_011640221, XM_017016712, XM_017016713, XM_024448211, XM_024448212 | NP_001121781, NP_001338195, NP_001338196, NP_001338197, NP_001338198, NP_001338199, NP_001338200, NP_001338201, NP_112189, XP_011538521, XP_011638522 XP_11638523, XP_016812201,XP_016812202, XP_024303979, XP_024303990 |
| 486 | 0.75 | Protein disulfide-isomerase A6 | PDIA6 | Q15084 | NM_001282704, NM_001282705, NM_001282106, NM_001282707, NM_005742, XM_011510308 | NP_001269633, NP_001269534, NP_001269635, NP_001269636, NP_005133, XP_011508610 |
| 488 | 0.75 | Protein lifeguard 3 | TMBIM1 | Q969X1 | NM_001321421, NM_001321428, NM_001321429, NM_001321430, NM_001321432, NM_001321433, NM_001321435, NM_001321436, NM_001321438, NM_022152, XM_011511627 | NP_001308356, NP_001308357, NP_001308358, NP_001308359, NP_001308361, NP_001308362, NP_001308364, NP_001308365, NP_001308367, NP_071435, XP_011509929 |
| 496 | 0.75 | Mitochondrial fission 1 protein | FIS1 | Q9Y3D6 | NM_016068 | NP_057152 |
| 505 | 0.75 | Claudin-3 | CLDN3 | O15551 | NM_ 001306 | NP_001297 |
| 506 | 0.75 | Nuclear migration protein nudC | NUDC | Q9Y266 | NM_006600, XM_011540530, XM_017000094, XM_017000095, XM_017000096, XM_024452486 | NP_006591, XP_011538832, XP_016855583, XP_016855584, XP_016855585, XP_024308254 |
| 509 | 0.75 | Transmembrane protease serine 4 | TMPRSS4 | Q9NRS4 | NM_001083947, NM_001173551, NM_001173552, NM_001290094, NM_001290096, NM_019894, NM_183247, XM_005271613, XM_005271614, XM_005271616, XM_011542901, XM_011642902, XM_011542903, XM_011642904 | NP_001077416, NP_001167022, NP_001167023, NP_001277023, NP_001277025, NP_063947, XP_005271610 XP_005271671, XP_005271672 XP_011541203, XP_011541204,XP_011541205, XP_011541206 |
| 510 | 0.75 | Syntaxin-2 | STX2 | P32856 | NM_001351049, NM_001351050, NM_001351051, NM_001351052, NM_001980, NM_194356, XM_017018978, XM_017018979, XM_017018980, XM_017018981, XM_011018982, XM_017018983, XM_17018984, XM_017018988, XM_011018989 | NP_001337978, NP_001337979, NP_001337980, NP_001337981, NP_001971 NP_919337, XP_016874467, XP_016874468, XP_016874469 XP_016814470, XP_016874471 XP_016874472, XP_016874473 XP_16874477, XP_016874478 |
| 515 | 0.75 | Palmitoyltransferase ZDHHC5 | ZDHHC5 | Q9C086 | NM_015457, XM_011544899, XM_01154900, XM_011544901, XM_017017498 | NP_056272, XP_011643201, XP_011543202 XP_011543203, XP_016872987 |
| 518 | 0.75 | Tetraspanin-31 | TSPAN31 | Q12999 | NM_001330168, NM_001330169, NM_006981, XM_024449123 | NP_001317097, NP_001317098, NP_005972, XP_024304891 |
| 527 | 0.75 | Synaptophysin-like protein 1 | SYPL1 | Q16563 | NM_006154, NM_182715 | NP_006745, NP_874384 |

TABLE 6-9

| | | | | | | |
|---|---|---|---|---|---|---|
| 537 | 0.745 | MAP kinase-activating death domain protein | MADD | Q8WXG6 | NM_001135943,NM_001135944, NM_003682, NM_130470 NM_130471, NM_130472 NM_130473, NM_130474 NM_130475, NM_130476, XM_005253189, XM_005253196, XM_005253199, XM_005253200, XM_005253201, XM_005253203, XM_005253204, XM_005253205, XM_017018478, XM_017018479, XM_017018480, XM_017018481, XM_017018482, XM_017018483, XM_017018484,XM_017018485, XM_017018486, XM_017018487, XM_017018488, XM_017018489, XM_017018490, XM_017018491, XM_017018492, XM_017018493, XM_017018498, XM_017018499, XM_017018500, XM_017018505, XM_017018506, XM_017018507, XM_024448745, XM_024448746, XM_024448747, XM_024448748, XM_024448749, XM_024448750, XM_024448751, XM_024448752, XM_024448753, XM_024448754, XM_024448755 | NP_001129415, NP_001129416, NP_003673, NP_569826, NP_569827, NP_569828, NP_569829, NP_569830, NP_569831, NP_569832, XP_005253246, XP_005253253, XP_005253256, XP_005253257, XP_005253258, XP_005253260, XP_005253261, XP_005253262, XP_016873967, XP_016873968, XP_016813969, XP_016873970, XP_016873971,XP_016873972, XP_016873973, XP_016873974, XP_016873975, XP_016873976, XP_016873977, XP_016873978, XP_016873979, XP_016873980, XP_016873981, XP_016873982, XP_016873987, XP_016873988, XP_016873989, XP_016873994, XP_016873995,XP_016873996, XP_024304513, XP_024304514, XP_024304515, XP_024304516, XP_024304517,XP_024304618, XP_024304519, XP_024304520, XP_024304521, XP_024304522, XP_024304523 |
| 549 | 0.74 | Insulin-like growth factor-binding protein com plex acid labile subunit | IGFALS | P35858 | NM_001146006, NM_004970 | NP_001139478, NP_004961 |
| 551 | 0.74 | CD99 antigen | CD99 | P14209 | NM_001122898, NM_001277710, NM_001321367, NM_001321368, NM_001321369, NM_001321370, NM_002414 | NP_001116370, NP_001308296, NP_001308297, NP_001308298, NP_001308299, NP_002405 |
| 555 | 0.74 | Junctional adhesion molecule A | F11R | Q9Y624 | NM_001348091, NM_016946, NM_144501, NM_144502 NM_144503, NM_144504 | NP_001335020, NP_058642 |
| 572 | 0.74 | Neutral amino acid transporter B(0) | SLC1A5 | Q15758 | NM_001145144, NM_001145145, NM_005628, XM_005259167 | NP_001138616,NP_001138617, NP_005619, XP_005259224 |
| 580 | 0.74 | Solute carrier family 12 member 2 | SLC12A2 | P55011 | NM_001046, NM_001256461, XM_011543588, XM_017009771 | NP_001037, NP_001243390, XP_011541890, XP_016865260 |
| 589 | 0.74 | Porimin | TMEM123 | Q8N131 | NM_052932 | NP_443164 |
| 593 | 0.74 | Small nuclear ribonucleoprotein F | SNRPF | P62300 | NM_003095 | NP_003086 |
| 595 | 0.74 | Secretory carrier-associated membrane protein 4 | SCAMP4 | Q969E2 | NM_001329639, NM_001329540, NM_079834 | NP_001316468, NP_001316469, NP_524558 |
| 596 | 0.74 | Autophagy-related protein 9A | ATG9A | Q7Z3C6 | NM_001077198, NM_024085 | NP_001070666, NP_076990 |

TABLE 6-10

| | | | | | | |
|---|---|---|---|---|---|---|
| 599 | 0.74 | Lipid droplet-associated hydrolase | LDAH | Q9H6V9 | NM_0012B2719, NM_001282720, NM_001282721, NM_001282722, NM_001282723, NM_001282724, NM_021925, XM_011533014 | NP_001269648, NP_001269649, NP_001269650, NP_001269651, NP_001269652, NP_001269653, NP_068744, XP_011531316 |
| 600 | 0.73 | Plexin-B2 | PLXNB2 | 015031 | NM_012401, XM_005261909 XM_005261910, XM_005261911, XM_006724413, XM_011530682, XM_017028703, XM_017028704 | NP_036533, XP_005261966, XP_005261967, XP_005261968, XP_006724476, XP_011528984, XP_016884192, XP_016884193 |
| 602 | 0.73 | Aldehyde dehydrogenase family 16 member A1 | ALDH16A | Q8LZ83 | NM_001145396, NM_153329, XM_011526441, XM_011526442 | NP_001138868, NP_699160, XP_011624743, XP_011524744 |
| 607 | 0.73 | Basigin | BSG | P35613 | NM_001322243, NM_001728, NM_198589, NM_198590, NM_198591, XM_017027173 | NP_001309172, NP_001719, NP_940991, NP_940992, NP_940993, XP_016882662 |
| 608 | 0.73 | Nectin-2 | NECTIN2 | Q92692 | NM_001042724 NM_002856 | NP_001036189, NP_002841 |
| 610 | 0.73 | Tetraspanin-9 | TSPAN9 | 075954 | NM_001168320 NM_006675, XM_011520912, XM_011520913 | NP_001161792, NP_006666, XP_011519214, XP_011519215 |
| 612 | 0.73 | Plasma membrane calcium-transporting ATPase 1 | ATP281 | P20020 | NM_001001323, NM_001366520, NM_001366321, NM_001366522, NM_001366523, NM_001366524, NM_001366525, NM_001366526, NM_001366527, NM_001366528, NM_001366629, NM_001366530, NM_01366531, NM_001366532, NM_001682, XM_011538407 XM_017019357, XM_024448989, XM_024448990, XM_024448991, XM_024448992, XM_024448993 | NP_001001323, NP_001353449, NP_001353450, NP_001353451, NP_001353452, NP_001353453, NP_001353454, NP_001353455, NP_001353456, NP_001353457, NP_001353458, NP_001353459, NP_001353460, NP_001363461, NP_001673, XP_011536709, XP_016814846, XP_024304757, XP_024304758, XP_024304759, XP_024304760, XP_024304761 |
| 613 | 0.73 | Nck-associated protein 1 | NCKAP1 | Q9Y2A7 | NM_013436, NM_205842, XM_006712200, XM_006712201 | NP_038464, NP_995314, XP_006712263, XP_006712264 |
| 617 | 0.73 | Alpha-1,6-mannosylglycoprotein 6-beta-N-acetylglucosaminyltransferase | MGAT5 | Q09328 | NM_002410, XM_005263668, XM_005263669, XM_005263670, XM_006712534, XM_011511199, XM_01151120, XM_011511201, XM_011511202, XM_011511204, XM_017004147, XM_017004148, XM_017004149 | NP_002401, XP_005263725, XP_005263726, XP_005263727, XP_006712597, XP_011509501, XP_011509502, XP_011509503, XP_011509504, XP_011509506, XP_016859636, XP_016859637, XP_016859638 |
| 618 | 0.73 | Disintegnin and metalloproteinase domain-containing protein 9 | ADAM9 | Q13443 | NM_001005845, NM_003816, XM_011544682, XM_017013942 | NP_003807, XP_011542984, XP_016869431 |
| 620 | 0.73 | Cell cycle control protein 50A | TMEM30A | Q9NV96 | NM_001143958, NM_018247 | NP_001137430, NP_060717 |
| 623 | 0.73 | Monocarboxylate transporter 1 | SLC16A1 | P53985 | NM_001166496, NM_003051 | NP_001159968, NP_003042 |
| 627 | 0.73 | Diphosphomevalonate decarboxylase | MVD | P53602 | NM_002461, XM_11523086, XM_011523087, XM_011523088, XM_011523089 | NP_002452, XP_011521388, XP_011521389, XP_011521390, XP_011521391 |

TABLE 6-11

| | | | | | | |
|---|---|---|---|---|---|---|
| 634 | 0.73 | Tropomyosin alpha-1 chain | TPM1 | P09493 | NM_000366, NM_001018004, NM_001018005, NM_001018006, NM_001018007, NM_001018008, NM_001018020, NM_001301244, NM_001301289, NM_001330344, NM_001330346, NM_001330351, NM_001365776, NM_001365777, NM_001365778, NM_001365779, NM_001365780, NM_001365781, NM_001365782, XM_005254637, XM_005254638, XM_005254639, XM_005254645, XM_005254646, XM_005254647, XM_005254650, XM_005254651, XM_006720667, XM_017022534, XM_017022535, XM_017022536, XM_017022537, XM_017022538, XM_017022539, XM_017022540, XM_024450040 | NP_000357, NP_001018004, NP_001018005, NP_001018006, NP_001018007, NP_001018008, NP_001018020, NP_001288173, NP_001288218, NP_001317273, NP_001317275, NP_001317280, NP_001352705, NP_001352706, NP_001352707, NP_001352708, NP_001352709, NP_001352710, NP_001352711, XP_005254694, XP_005254695, XP_005254696, XP_005254702, XP_005254703, XP_005254704, XP_005254707, XP_005254708, XP_006720730, XP_016878023, XP_016878024, XP_016878025, XP_16878026, XP_016878027, XP_016878028, XP_016878029, XP_024305808 |
| 636 | 0.73 | Inactive tyrosine-protein kinase 7 | PTK7 | Q13308 | NM_001270398, NM_002821, NM_152880, NM_152881, NM_152882, NM_152883, XM_011514765, XM_011514766 | NP_001257327, NP_002812, NP_690619, NP_690620, NP_690621, XP_011513067, XP_011513068 |
| 640 | 0.73 | Serine incorporator 3 | SERINC3 | Q13530 | NM_006811, NM_198941, XM_017027601 | NP_006802 NP_945179, XP_016883090 |
| 643 | 0.73 | Secretory carrier-associated membrane protein 2 | SCAMP2 | 015127 | NM_001320778, NM_005697, XM_017021856 | NP_001307707, NP_005688, XP_016877345 |
| 648 | 0.73 | CD166 antigen | ALCAM | Q13740 | NM_001243280, NM_001243281, NM_001243283, NM_001627 | NP_001230209, NP_001230210, NP_001230212, NP_001618 |
| 650 | 0.73 | V-set and immunoglobulin domain-containing protein 10-like | VSIG10L | Q86VR7 | NM_001163922 | NP_001157394 |
| 663 | 0.73 | Prominin-2 | PROM2 | Q8N271 | NM_001165977, NM_001166978, NM_001321070, NM_144707, XM_006712301, XM_011510672 | NP_001159449, NP_001159450, NP_001307999, NP_653308, XP_006712364, XP_011508974 |
| 666 | 0.73 | Choline transporter-like protein 4 | SLC44A4 | Q53GD3 | NM_001178044, NM_001178045, NM_025257 | NP_001171515, NP _001171516, NP_079533 |
| 670 | 0.73 | Syndecan-4 | SDC4 | P31431 | NM_002999, XM_011528977 | NP_002990, XP_011527279 |
| 678 | 0.72 | Disintegrin and metalloproteinase domain-containing protein 10 | ADAM10 | 014672 | NM_001110, NM_001320570 XM_024449818 | NP_001101, NP_001307499, XP_024305586 |
| 679 | 0.72 | Integrin alpha-2 | ITGA2 | P17301 | NM_002203 | NP_002194 |
| 681 | 0.72 | Dolichyl-diphosphooligosaccharide-protein glycosyltransferase subunit 2 | RPN2 | P04844 | NM 001136771, NM_001324299, NM_001324301, NM_001324302, NM_001324303, NM_001324304, NM_001324305, NM_01324306, NM_002951, XM_006723851, XM_006723852 | NP_001129243, NP_001311228, NP_001311230, NP_001311231, NP_001311232, NP_001311233, NP_001311234, NP_001311235, NP_002942, XP_006723914, XP_006723915 |

TABLE 6-12

| | | | | | | |
|---|---|---|---|---|---|---|
| 683 | 0.72 | Endothelin-converting enzyme 1 | ECE1 | P42892 | NM_001113347, NM_001113348, NM_001113349, NM_001397, XM_006710398, XM_011540872, XM_011540873 | NP_001106818, NP_001106819, NP_001106820, NP_001388, XP_006710461, XP_011539174, XP_011539175 |
| 690 | 0.72 | Protein FAM34A | FAM234A | Q9H0X4 | NM_001284497, NM_032039, XM_005255622, XM_005255623, XM_006720957, XM_011522691, XM_011522692, XM_017023760, XM_017023761, XM_017023762, XM_017023763, XM_017023764, XM_017023765 | NP_001271426, NP_114428, XP_005255679, XP_005255680, XP_006721020, XP_011520993, XP_011520994, XP_016879249, XP_016879250, XP_016879251, XP_016879252, XP_016879253, XP_016879254 |
| 702 | 0.72 | Malectin | MLEC | Q14165 | NM_001303627, NM_001303628, NM_014730, XM_011539031, XM_011539032 | NP_001290556, NP_001290557, NP_055545, XP_011537333, XP_011537334 |
| 713 | 0.72 | Insulin-like growth factor II | IGF2 | P01344 | NM_000612, NM_001007139, NM_001127598 NM_001291861, NM_001291862 | NP_000603, NP_001007140, NP_001121070, NP_001278790, NP_001278791 |
| 714 | 0.72 | Ubiquitin-like modifier-activating enzyme 5 | UBA5 | Q9GZZ9 | NM_001320210 NM_001321238, NM_001321239, NM_024818, NM_198329 | NP_001307139, NP_001308167, NP_001308168, NP_079094, NP_938143 |
| 722 | 072 | Ephrin type-B receptor 2 | EPHB2 | P29323 | NM_001309192, NM_001309193, NM_004442, NM_017449, XM_006710441, XM_006710442, XM_024453895 | NP_001296121, NP_001296122, NP_004433, NP_059145, XP_006710504, XP_006710505, XP_024309663 |
| 723 | 0.72 | Interleukin-1 receptor accessory protein | IL1RAP | Q9NPH3 | NM_001167928, NM_001167929, NM_001167930, NM_001167931, NM_001364879, NM_001364880, NM_001364881, NM_002182, NM_134470, XM_017006347, XM_017006348, XM_024453511 | NP_001161400, NP_001161401, NP_001161402, NP_001161403, NP_001351808, NP_001351809, NP_001351810, NP_002173, NP_608273, XP_016861836, XP_016861837, XP_024309279 |
| 726 | 0.72 | Eukaryotic translation elongation factor 1 epsilon-1 | EEF1E1 | 043324 | NM_001135650, NM_004280 | NP_001129122, NP_004271 |
| 730 | 0.72 | Single Ig IL-1-related receptor | SIGIRR | Q6/A17 | NM_001135053, NM_001135054, NM_021805, XM_005253044, XM_005253045, XM_005253046, XM_005253048, XM_017018099, XM_017018100 | NP_001128525, NP_001128526, NP_068577, XP_005253101, XP_005253102, XP_005253103, XP_005253105, XP_016873588, XP_016873589 |
| 733 | 0.72 | Apolipoprotein L2 | APOL2 | Q9BQE5 | NM_030882, NM_145637, XM_011530074, XM_011530075, XM_011530076, XM_011530077, XM_011530078, XM_017028724 | NP_112092, NP_663612, XP_011528376, XP_011528377, XP_011628378, XP-011528379, XP_011528380, XP_016884213 |

TABLE 6-13

| | | | | | | |
|---|---|---|---|---|---|---|
| 741 | 0.71 | Protein 4.1 | EPB41 | P11171 | NM_001166005, NM_001166006, NM_001166007, NM_004437, NM_203342, NM_203343, XM_005245753, XM_005245757, XM_005245760, XM_005245761, XM_005245763, XM_005245764, XM_005245765, XM_005245768, XM_005245769, XM_005245770, XM_005245772, XM_005245773, XM_005245774, XM_006710434, XM_006710439, XM_011540956, XM_011540967, XM_011540958, XM_011540959, XM_011540960, XM_011540961, XM_011540962, XM_011540963, XM_011540964, XM_011540965, XM_017000581, XM_017000582, XM_017000583, XM_017000584, XM_017000585, XM_017000586, XM_017000587, XM_017000588, XM_017000589, XM_017000590, XM_017000591, XM_017000592, XM_017000593, XM_017000594, XM_017000595, XM_017000596, XM_017000597, XM_017000598, XM_017000599, XM_017000600, XM_017000602, XM_017000603, XM_017000604, XM_024453880 | NP_001159477, NP_001159478, NP_001159479, NP_004428, NP_096217, NP_976218, XP_005245810, XP_005245814, XP_005245817, XP_005245818, XP_005245820, XP_005245821, XP_005245822, XP_005245825, XP_005245826, XP_005245827, XP_005245829, XP_005245830, XP_005245831, XP_006710497, XP_006710502, XP_011539258, XP_011539259, XP_011539260, XP_011539261, XP_011539262, XP_011539263, XP_011539264, XP_011539265, XP_011539266, XP_011539267, XP_016856070, XP_016856071, XP_016856072, XP_016856073, XP_016856074, XP_016856075, XP_016856076, XP_016856077, XP_016856078, XP_016856079, XP_016856080, XP_016856081, XP_016856082, XP_016856083, XP_016856084, XP_016856085, XP_016856086, XP_016856087, XP_016856088, XP_016856089, XP_016856091, XP_016856092, XP_016856093, XP_024309648 |
| 745 | 0.71 | CD99 antigen-like protein 2 | CD99L2 | Q8TCZ2 | NM_001184808, NM_001242614, NM_031462, NM_134445 NM_134446, XM_011531202, XM_017029890, XM_017029891 | NP_001171737, NP_001229543, NP_113650, NP_604394, NP_604395, XP_011529504, XP_016885379, XP_016885380 |
| 748 | 0.71 | V-type proton ATPase 16 kDa proteolipid subunit | ATP6VOC | P27449 | NM_001198569, NM_001694 | NP_001185498, NP_001685 |
| 749 | 0.71 | Nicastrin | NCSTN | Q92542 | NM_001290184, NM_001290186, NM_001349729, NM_015331, XM_005245053 | NP_001277113, NP_001277115, NP_001336658, NP_056146, XP_005245110 |
| 752 | 0.71 | Protein-L-isoaspartate(D-aspartate) O-methyltransferase | PCMT1 | P22061 | NM_001252049, NM_001252050, NM_001252051, NM_001252052, NM_001252053, NM_001360452, NM_001360456, NM_005389, XM_011535868, XM_024446451 | NP_001238978, NP_001238979, NP_001238930, NP_001238981, NP_001238982, NP_001347381, NP_001347385, NP_005380, XP_011534170, XP_024302219 |
| 754 | 0.71 | Apo ipoprotein L1 | APOL1 | 014791 | NM_001136540, NM_001136541, NM_001362927, NM_003661, NM_145343, NM_145344 XM_005261796, XM_011530478 | NP_001130012, NP_001130013, NP_001349856, NP_003652, NP_663318, XP_005261853, XP_011528780 |

TABLE 6-14

| | | | | | | |
|---|---|---|---|---|---|---|
| 758 | 0.71 | Solute carrier family 2, facilitated glucose transporter member 1 | SLC2A1 | P11166 | NM_006516 | NP_006507 |
| 760 | 0.71 | Inositol monophosphatase 3 | IMPAD1 | Q9NX62 | NM_017813 | NP_060283 |
| 762 | 0.71 | Proteolipid protein 2 | PLP2 | Q04941 | NM_002668 | NP_002659 |
| 763 | 0.71 | Large neutral amino acids transporter small subunit 1 | SLC7A5 | Q01650 | NM_003486, XM_006721286, XM_017023735 XM_017023736, XM_017023737 | NP_003477, XP_006721349, XP_016879224, XP_016879225, XP_016879226 |
| 764 | 0.71 | CSC1-like protein 1 | TMEM63A | 094886 | NM_014698, XM_006711841, XM_011544328, XM_011544329, XM_011544330, XM_011544331, XM_011544332 | NP_055513, XP_006711904, XP_011542630, XP_011542631, XP_011542632, XP_011542633, XP_011542634 |
| 767 | 0.71 | Lysosome-associated membrane glycoprotein 2 | LAMP 2 | P13473 | NM_001122606, NM_002294, NM_013995 | NP_001116078, NP_002285, NP_054701 |
| 777 | 0.71 | NPC Intracellular cholesterol transporter 1 | NPC1 | 015118 | NM_000271, XM_005258277, XM_005258278, XM_005258279, XM_006722479, XM_017025784, XM_017025785, XM_017025786, XM_017025787 | NP_000262, XP_005258334, XP-005258335, XP_005258336, XP_006722542, XP_016881273, XP_016881274, XP_016881275, XP_016881276 |
| 783 | 0.71 | E3 ubiquitin-protein ligase HUWE1 | HUWE1 | Q7Z6Z7 | NM_031407, XM_005261965 XM_011530747, XM_011530751, XM_017029191, XM_017029192, XM_017029193, XM_017029194, XM_017029195, XM_017029196, XM_017029197, XM_017029198, XM_017029199, XM_017029200, XM_017029201, XM_017029202, XM_017029203, XM_017029204, XM_017029206 XM_024452322 | NP_113584, XP_005262022, XP_011529049, XP_011529053, XP_016884680, XP_016884681, XP_016884682, XP_016884683, XP_016884684, XP_016884685, XP_016884686, XP_016884687, XP_016884688, XP_016884689, XP_016884690, XP_016884691, XP_016884692, XP_016884693, XP_016884695, XP_024308090 |
| 786 | 0.71 | Translin | TSN | Q15631 | NM_001261401, NM_004622 | NP_001248330, NP_004613 |
| 791 | 0.71 | Voltage-dependent calcium channel subunit alpha-2/delta-1 | CACNA2D | P54289 | NM_000722, NM_001302890, XM_005250570, XM_005250572, XM_005250573, XM_005250574, XM_006716118, XM_006716119, XM_006716120, XM_006716121, XM_011516570, XM_011516571, XM_011616572, XM_017012588 | NP_000713, NP_001289819, XP_005250627, XP_005250629, XP_005250630, XP_005250631, XP_006716181, XP_006716182, XP_006716183, XP_006716184, XP_011514872, XP_011514873, XP_011514874, XP_016868077 |
| 792 | 0.71 | Galaclocerebrosidase | GALC | P54803 | NM_000153, NM_001037525, NM_001201401, NM_001201402, XM_011536618 | NP _000144, NP_001188330, NP_001188331, XP_011534920 |
| 797 | 0.71 | Zinc transporter ZIP10 | SLC39A10 | Q9ULF5 | NM_001127257, NM_020342, XM_005246689 XM_011511504, XM_011511505, XM_011511506, XM_011511507, XM_017004522, XM_017004523, XM_017005484 | NP_001120729, NP_065075, XP_005246746, XP_011509806, XP_011509807, XP_011509808, XP_011509809, XP_016860011, XP_016860012, XP_016860973 |

TABLE 6-15

| | | | | | | |
|---|---|---|---|---|---|---|
| 798 | 0.71 | Proteasomal ATPase-associated factor 1 | PAAF1 | Q9BRP4 | NM_001267803, MM_001267804, NM_001267805, NM_001267806, NM_001363556, NM_025155, XM_005274308, XM_005274309, XM_017018357, XM_017018358, XM_017018359 | NP_001254732, NP_001254733, NP_001254734, NP_001254735, NP_001350485, NP_079431, XP_005274365, XP_005274366, XP_016873846, XP_016873847, XP_016873848 |
| 801 | 0.705 | Bcl-2-like protein 1 | BCL2L1 | Q07817 | NM_001191, NM_001317919, NM_001317920, NM_001317921, NM_001322239, NM_001322240, NM_001322242, NM_138578, XM_011528964, XM_017027993 | NP_001182, NP_001304848, NP_001304849, NP_001304850, NP 001309168, NP_001309169, NP_001309171, NP_612815, XP_011527266 XP_016883482 |
| 804 | 0.7 | Receptor-type tyrosine-protein phosphatase alpha | PTPRA | P18433 | NM_002836, NM_080840, NM_080841 | NP_002827, NP_543030, NP_543031 |
| 813 | 0.7 | Secretory carrier-associated membrane protein 1 | SCAMP1 | O15126 | NM_001290229, NM_004866, NM_052822, XM_011543727 | NP_001277158, NP_004857, XP_011542029 |
| 815 | 0.7 | Erythrocyte band 7 integral membrane protein | STOM | P27105 | NM_001270526, NM_001270527 NM_004099, NM_198194 | NP_001257455, NP_001257456, NP_004090, NP_937837 |
| 825 | 0.7 | Lysosamal acid phosphatase | ACP2 | P11117 | NM_001131064, NM_001302489, NM_001302490, NM_001302491, NM_001302492, NM_001357016, NM_001610 | NP_001289418, NP_001289419, NP_001289420, NP_001289421, NP_001343945, NP_001601 |
| 828 | 0.7 | Ras-related protein Rab-18 | RAB18 | Q9NP72 | NM_001256410, NM_001256411, NM_001256412, NM_001256415, NM_021252 | NP_001243339, NP_001243340, NP_001243341, NP_001243344, NP_067075 |
| 831 | 0.7 | Pituitary tumor-transforming gene 1 protein-interacting protein | PTTG1IP | P53801 | NM_001286822, NM_004339 | NP_001273151, NP_004330 |
| 832 | 0.7 | Golgi apparatus protein 1 | GLG1 | Q92896 | NM_001145666, NM_001145667 NM_012201 | NP_001139138 NP_001139139, NP_036333 |
| 844 | 0.7 | Carbohydrate sulfotransferase 12 | CHST12 | Q9NRB3 | NM_001243794, NM_001243795, NM_018641, XM_011515443, XM_011515444 | NP_001230723, NP_001230724, NP_061111, XP_011513745, XP_011513746 |
| 845 | 0.7 | OX-2 membrane glycoprotein | CD200 | P41217 | NM_001004196, NM_001004197, NM_001318826, NM_001318828, NM_001318830, NM_001365851 NM_001365862, NM_001365853, NM_001365854, NM_001365855, NM_005944, XM_005247481, XM_005247482, XM_005247485, XM_005247486, XM_005247488, XM_017006452, XM_017006453, XM_017006454, XM_017006455, XM_017006456, XM_017006457, XM_017006458, XM_017006459 | NP_001004196, NP_001305755, NP_001305757, NP_001305759, NP_001352780, NP_001352781, NP_001352782, NP_001352783, NP_001352784, NP_005935, XP_005247538 XP_005247539, XP_005247542, XP_005247543, XP_005247545, XP_016861941, XP_016861942,XP_016861943, XP_016861944, XP_016861945, XP_016861946, XP_016861947, XP_016861948 |

As shown in Table 6-1 to Table 6-15, 36 membrane proteins were detected for AUC 0.8 or higher and 168 membrane proteins were detected for 0.7 or higher.

### 4. Extraction of Protein Groups That Did Not Generate False Negatives for Patients (Responders) having Drug-Sensitivity

For 10 responders, protein groups for which false negatives were not generated based on the threshold calculated by the Youden index were extracted and the results sorted in descending order of AUC are shown in Table 7-1 to Table 7-4.

TABLE 7-1

| AUC Rank | AUC | Gene name | Uniprot AC | Protein Name | RefSeq_NM | RefSeq_NP |
|---|---|---|---|---|---|---|
| 16 | 0.88 | ITGB5 | P18084 | Integrin beta-5 | NM_001354764, NM_001354765, NM_001354766, NM_002213, XM_005247436, XM_006713630 XM_017006353 XM_017006354, XM_017006355 | NP_001341693, NP_001341694, NP_001341695, NP_002204, XP_005247493, XP_006713693, XP_016861842, XP_016861843, XP_016861844 |
| 140 | 0.82 | SNX9 | Q9Y5X1 | Sorting nexin-9 | NM_016224, XM_005267015, XM_011535886 | NP_057308, XP_005267072, XP_011534188 |
| 142 | 0.82 | PGM3 | O95394 | Phosphoacetylglucosamine mutase | NM_001199917, NM_001199918, NM_001199919, NM_015599, XM_017010935, XM_017010937 XM_024446459 XM_024446460 | NP_001186846, NP_001186847, NP_001186848, NP_056414, XP_016866424, XP_016866426 XP_024302227, XP_024302228 |
| 175 | 0.81 | STMN1 | P16949 | Stathmin | VM_001145454, NM_005563, NM_152497, VM_203399, NM_203401 | NP_001138926, NP_005554, NP_981944, NP_981946 |
| 176 | 0.81 | RANBP1 | P43487 | Ran-specific GTPase-activating protein | NM_001278639, NM_001278640, NM_001278641, NM_002882, XM_011530289, XM_011530290, XM_011530291, XM_017028890, XM_017028891, XM_017028892 | NP_001265568, NP_001265569, NP_001265570, NP_002873, XP_011528591, XP_011528592, XP_011528593, XP_016884379, XP_016884380, XP_016884381 |
| 179 | 0.81 | EHD3 | Q9NZN3 | EH domain-containing protein 3 | NM_014600, XM_011532806 | NP_055415, XP_011531108 |
| 180 | 0.81 | APRT | P07741 | Adenine phosphoribosyltransferase | NM_000485, NM_001030018 | NP_000476, NP_001025189 |
| 224 | 0.8 | PPP1R7 | Q15435 | Protein phosphatase 1 regulatory subunit 7 | NM_001282409, NM_001282410, NM_001282411, NM_001282412, NM_001282413, NM_001282414, NM_002712 XM_011511388 | NP_001269338, NP_001269339, NP_001269340, NP_001269341, NP_001269342, NP_001269343, NP_002703, XP_011509690 |
| 226 | 0.8 | NCOR2 | Q9Y618 | Nuclear receptor corepressor 2 | NM_001077261, NM_001206654, NM_006312 | NP_001070729, NP_001193583, NP_006303 |
| 227 | 0.8 | TLDC1 | Q6P9B6 | TLD domain-containing protein 1 | VM_020947, XM_005256074, XM_005256075, XM_006721240, XM_011523248 XM_011523249, XM_017023510 XM_0017023511, XM_017023512 | NP_065998, XP_005256131, XP_005256132, XP_006721303, XP_011521550, XP_011521551, XP_016878999, XP_016879000, XP_016879001 |
| 228 | 0.8 | LANCL1 | O43813 | Glutathione S-transferase LANCL1 | NM_001136574 NM_001136575, NM_006055, XM_005246243 | NP_001130046, NP_001130047, NP_006046, XP_005246300 |
| 276 | 0.79 | SELENOP | P49908 | Selenoprotein P | NM_001085486, NM_001093726, NM_005410 | NP_001078955, NP_001087195, NP_005401 |
| 277 | 0.79 | ME2 | P23368 | NAD-dependent malic enzyme, mitochondrial | NM_001168335, NM_002396 | NP_001161807, NP_002387 |
| 279 | 0.79 | RABGGTA | Q92696 | Geranylgeranyltransferase type-2subunit alpha | NM_004581, NM_182836 | NP_004572, NP_878256 |
| 280 | 0.79 | IL17RA | Q96F46 | Intereukin-17 receptor A | NM_001289905, NM_014339 | NP_001276834, NP_055154 |
| 322 | 0.78 | TMOD3 | Q9NYL9 | Tropomodulin-3 | NM_014547, XM_017022087, XM_017022088 | NP_055362, XP_016877576, XP_016877577 |
| 323 | 0.78 | SCYL2 | Q6P3W7 | SCY1-like protein 2 | NM_001317784 , NM_001330253, NM_001330254, NM_001330256, NM_017988 | NP_001304713, NP_001317182, NP_001317183, NP_001317185, NP_060458 |
| 375 | 0.77 | CLDN7 | O95471 | Claudin-7 | NM_001185022, NM_001185023, NM_001307 | NP_001171951, NP_001171952, NP_001298 |
| 377 | 0.77 | NRBP1 | Q9UHY1 | Nuclear receptor-binding protein | NM_001321357, NM_001321358 NM_001321359, NM_001321361, NM_001321362, NM_001321363, NM_013392 | NP_001308286, NP_001308287, NP_001308288, NP_001308290, NP_001308291 NP_001308292, NP_037524 |
| 435 | 0.76 | RRBP1 | Q9P2E9 | Ribosome-binding protein 1 | NM_001042576 NM_001365613, NM_004587 | NP_001036041, NP_001352542, NP_004578 |

TABLE 7-2

| | | | | | | |
|---|---|---|---|---|---|---|
| 476 | 0.755 | NUDCD1 | Q96RS6 | NudC domain-containing protein 1 | NM_001128211, NM_032869, XM_017013910, XM_017013911 | NP_001121683, NP_116258, XP_016869399, XP_016869400 |
| 500 | 0.75 | PAFAH1B2 | P68402 | Platele:-activating factor acetylhydrolase lB subunit beta | NM_001184746, NM_001184747 NM_001184148, NM_001309431 NM_002572, XM_017017840 | NP_001171675, NP_01171676, NP_001171677, NP_001296360, NP_002563, XP_016873329 |
| 501 | 0.75 | NUDC | Q9Y266 | Nuclear migration protein nudC | NM_006600, XM_011540530, XM_017000094, XM_017000095, XM_017000096, XM_024452486 | NP_006591, XP_011538832, XP_016855583, XP_016855584, XP_016855585, XP_024308254 |
| 502 | 0.75 | STX2 | P32856 | Symaxin-2 | NM_001351049, NM_001351050 NM_001351051, NM_001351052 NM_001980, NM_194356, XM_017018978, XM_017018979, XM_017018980, XM_017018981, XM_17018982, XM_017018983, XM_017018984, XM_017018988, XM_017018989 | NP_001337978, NP_001337979 NP_001337980, NP_001337981 NP_001971, NP_919337, XP_016874467, XP_016874468, XP_016874469, XP_016874470, XP_016874471 XP_016874472, XP_016874473, XP_016874477, XP_016874478 |
| 503 | 0.75 | CHMP2A | 043633 | Charged multivesicular body protein 2a | NM_014453, NM_198426 XM_005258746, XM_005258747 | NP_055268, NP_940818, XP_005258803, XP_005258804 |
| 559 | 0.74 | ACP1 | P24666 | Low molecular weight phosphotyrosine protein phosphatase | NM_001040649 NM_004300, NM_007099, NM_177554 | NP_001035739, NP_004291, NP_009030 |
| 561 | 0.74 | MTM1 | Q13496 | Myotubularin | NM_000252, XM_005274687, XM_011531171, XM_011531172, XM_011531173, XM_017029547, XM_017029548, XM_017029549 XM_017029550, XM_017029551 | NP_000243, XP_005274744, XP_011529473, XP_011529474, XP_011529475, XP_016865036, XP_016885037, XP_016885038, XP_016885039, XP_016885040 |
| 563 | 0.74 | ATG9A, | Q7Z3C6 | Autophagy-related protein 9A | NM_001077198, NM_024085 | NP_001070666, NP_076990 |
| 565 | 0.74 | BZW2 | Q9Y6E2 | Basic leucine zipper and W2 domain-containing protein 2 | NM_001159767, NM_001362717 NM_001362718, NM_001362719 NM_014038 XM_006715706, XM_006715707, XM_006715708, XM_024446735 | NP_001153239, NP_001349646, NP_001349647, NP_001349648 NP_054757, XP_006715769, XP_006715770, XP_006715771, XP_024302503 |
| 628 | #N/A | HLA-B | P30464 | HLA class I histocompatibility antigen, 3-15 alpha chain | | #NA |
| 629 | 0.73 | YARS | P54577 | Tyrosine-tRNAligase, cytoplasmic | NM_003680, XM_011542347, XM_011542348, XM_017002651 | NP_003671, XP_011540649, XP_011540650, XP_016858140 |
| 630 | 0.73 | VSIG10L | Q86VR7 | V-sec and immunoglobulin domain-containing protein 10-like | NM_001163922 | NP_001157394 |
| 631 | 0.73 | SEC31A | 094979 | Protein transport protein Sec31A | NM_001077206, NM_001077207 NM_001077208, NM_001191049 NM_001300744, NM_001300745 NM_001318119, NM_001318120 NM_014933, NM_016211 | NP_001070674, NP_001070675, NP_001070676, NP_001177978, NP_001287673, NP_001287674, NP_001305048, NP_001305049, NP_055148, NP_057295 |

TABLE 7-3

| | | | | | | |
|---|---|---|---|---|---|---|
| 633 | 0.73 | UBE2V1 | Q13404 | Ubiquizin-conjugating enzyme E2 variant 1 | NM_001032288, NM_001257393, NM_001257394, NM_001257395, NM_001257396, NM_001257397, NM_001257398 NM_001257399, NM_001282575 NM_001282576, NM J01282577, NM_001282578, NM_001282579 NM_001282580, NM_02199, NM_022442 NM_199144, NM_003349, NM_199203 | NP_001027459, NP_001244322, NP_001244323, NP_001244324, NP_001244325, NP_001244326, NP_001244327, NP_001244328, NP_001269504, NP001269505, NP_001269506, NP_001269507, NP_001269508. NP_001269509, NP_068823, NP_071887, NP_J54595, NP_954673 |
| 675 | 0.725 | MMAB | Q96EY8 | Corrinoid adenosyltransferase | NM_052845, XM_011538267, XM_011538268, XM_011538269 XM_024448961 | NP_443077, XP_011536569, XP_011536570, XP_011536571, XP_024304729 |
| 705 | 0.72 | CUL4B | Q13620 | Cullin-4B | NM_001079872, NM_001330624, NM_003588, XM_005262481, XM_006724784, XM_011531399, XM_011531400, XM_011531401 | NP_001073341, NP_001317553, NP_03579, XP_005262538, XP_006724847, XP_011529701, XP_011529702, XP_011529703 |
| 706 | 0.72 | DCTN5 | Q9BTE1 | Dynactin subunit 5 | NM_001199011, NM_001199743, NM_032486 | NP_001185940, NP_01186672, NP_115875 |
| 761 | 0.71 | PCMT1 | P22061 | Protein-L-isoaspartate(D-aspartate) O-mezhyltransferase | NM_001252049, NM_001252050, NM_001252051, NM_001252052, NM_001252053, NM_001360452, NM_001360456, NM_005389, XM_011535868, XM_024446451 | NP_001238978, NP_001238979, NP_001238980, NP_001238981, NP_001238982, NP_001347381, NP_001347385, NP_005380, XP_011534170, XP_024302219 |
| 762 | 0.71 | GNG12 | Q9JBI6 | Guanine nucleotide-binding protein G()/G(S)/G(0) subunit gamma-12 | NM_018841, XM_017001809, XM_024448405 | NP_061329, XP_016857298, XP_024304173 |
| 763 | 0.71 | S_C7A5 | Q01650 | Large neural amino acids transporter small subunit 1 | NM_003486, XM_006721286, XM_017023735 XM_017023736XM_017023737 | NP_003477, XP_006721349, XP_016879224, XP_016879225, XP_616879226 |
| 764 | 0.71 | GSTM3 | P21266 | Glutathione S-transferase Mu 3 | NM_000849 | NP_000840 |
| 765 | 0.71 | ADSS | P30520 | Adenylosuccinate syntherase isozyme 2 | NM_001126, NM_001365073, XM_011544108 | NP_001117, NP_001352002, XP_011542410 |
| 766 | 0.71 | RAB20 | Q9NX57 | Ras-related protein Rab-20 | NM_017817 | NP_060287 |
| 767 | 0.71 | PKP2 | Q99959 | Plakophilin-2 | NM_001005242, NM_094572 | NP_001005242, NP_004563 |
| 820 | 0.7 | FBXL2C | Q96IG2 | F-box/LRR-repeat protein 20 | NM_01184906, NM_032875, XM_005257746, XM_005257747 | NP_001171835, NP_116264, XP_005257803, XP_005257804 |
| 822 | 0.7 | GSTZ1 | 043708 | Maleylacetoacetate isomerase | NM_001312660, NM_001363703, NM_001513, NM_145870, NM_145871, XM_011536670, XM_011536671, XM_024449548, XM_024449549, XM_024449550, XM_024449551, XM_024449552 | NP_001299589, NP_001350632, NP_665877, NP_665878, XP_011534972, XP_011334973, XP_024305316, XP_024305317, XP_024305318, XP_024305319, XP_024305320 |

TABLE 7-4

| | | | | | | |
|---|---|---|---|---|---|---|
| 823 | 0.7 | CD200 | P41217 | OX-2 membrane glycoprotein | NM_001004196, NM_001004197, NM_001318826, NM_001318828, NM_001318830, NM_001365851, NM_001365852, NM_001365853, NM_001365854, NM_001365855, NM_005944, XM_005247481, XM_005247482, XM_005247485, XM_005247486, XM_005247488, XM_017006452 XM_017006453, XM_017006454, XM_017006455, XM_017006456, XM_017006457, XM_017006458, XM_017006459 | NP_001004196, NP_001305755, NP_001305757, NP_001305759, NP_001352780, NP_001352781 NP_001352782, NP_001352783, NP_001352784, NP_005935, XP_005247538, XP 005247539 XP_005247542, XP_005247543 XP_005241545, XP_016861941 XP_016861942, XP_016861943, XP_016861944, XP_016861945, XP_016861946, XP_016861947, XP_016861948 |

As shown in Table 7-1 to Table 7-4, 11 proteins were present for AUC 0.8 or higher and 47 for AUC 0.7 or higher. It was considered that it was possible to construct an error-resistant multi-marker by combining these markers.

### INDUSTRIAL APPLICABILITY

According to the method of the present embodiment, it is possible to predict the efficacy of treatment of a lung cancer patient using an immune checkpoint inhibitor with high accuracy.

1. A method for predicting efficacy of treatment of a lung cancer patient using with an immune checkpoint inhibitor, the method comprising:
administering the immune checkpoint inhibitor to the lung cancer patient;
taking a biological sample from the lung cancer patient which has been administered with the immune checkpoint inhibitor;
isolating exosomes from the biological sample derived from the lung cancer patient; and
determining an expression level of a protein present in the exosomes by a mass spectrometry method,
wherein the protein is one or more proteins selected from the group of proteins shown in Table 1-1 to Table 1-6:
TABLE 1-1
Protein Name
Band 4.1 -like protein 2
Cullin-2
Protein Hikeshi
Hippocalcin-like protein 1
UTP–glucose-1 -phosphate uridylyltransferase
Attractin
Annexin A3
Rap1 GTPase-activating protein 2
Radixin
Annexin A5
ADP-ribosylation factor 5
L-lactate dehydrogenase B chain
Rho GTPase-activating protein 1
Drebrin
Serine/threonine-protein kinase PAK 2
Protein BRICK1
Integrin alpha-V
Protein kinase C and casein kinase substrate in neurons protein 2
Triokinase/FMN cyclase
Adhesion G-protein coupled receptor G1
Integrin beta-5
Isochorismatase domain-containing protein 1
Septin-2
Hydroxyacyl-coenzyme A dehydrogenase, mitochondrial
Serine/threonine-protein kinase 38
Transient receptor potential cation channel subfamily M member 4
Cytoskeleton-associated protein 5
FAS-associated factor 1
Insulin receptor
Tropomyosin alpha-4 chain
Programmed cell death protein 6
Leukocyte elastase inhibitor
ADP-ribosylation factor 6
Complement component C9
ATP-dependent (S)-NAD(P)H-hydrate dehydratase
Actin, alpha cardiac muscle 1
Heat shock 70 kDa protein 4
14-3-3 protein theta
Phosphatidylinositol transfer protein beta isoform
Phosphoribosyltransferase domain-containing protein 1
Costars family protein ABRACL
Ubiquitin-conjugating enzyme E2 L5
Sodium/potassium-transporting ATPase subunit alpha-1
14-3-3 protein epsilon
TABLE 1-2
Major prion protein
F-actin-capping protein subunit alpha-2
HLA class I histocompatibility antigen, Cw-4 alpha chain
GTP-binding protein SAR1a
Actin-related protein ⅔ complex subunit 5
Glutathione reductase, mitochondrial
Guanine nucleotide-binding protein subunit alpha-11
Protein disulfide-isomerase A5
MARCKS-related protein
Septin-7
Hsc70-interacting protein
COP9 signalosome complex subunit 8
Vacuolar protein sorting-associated protein 37C
Acidic fibroblast growth factor intracellular-binding protein
ADP-ribosyl cyclase/cyclic ADP-ribose hydrolase 1
Ribosome maturation protein SBDS
Fructose-bisphosphate aldolase A
BH3-interacting domain death agonist
Charged multivesicular body protein 4b
Guanine nucleotide-binding protein G(s) subunit alpha isoforms short
cAMP-dependent protein kinase catalytic subunit alpha
Cytosolic non-specific dipeptidase
Dynamin-1
Neuroplastin
IST1 homolog
Sorting nexin-5
Mitochondrial import inner membrane translocase subunit Tim13
Exocyst complex component 4
Moesin
Serum albumin
Myristoylated alanine-rich C-kinase substrate
Sodium/potassium-transporting ATPase subunit beta-3
Vinculin
Actin-related protein ⅔ complex subunit 1B
Alcohol dehydrogenase class-3
Cysteine and glycine-rich protein 1
E3 ubiquitin-protein ligase TRIM23
Actin-related protein 3
Tetraspanin-15
Ras-related C3 botulinum toxin substrate 1
COP9 signalosome complex subunit 2
Myotrophin
Rho-related GTP-binding protein RhoF
COP9 signalosome complex subunit 3
Glutathione synthetase
DnaJ homolog subfamily C member 5
TABLE 1-3
Glutathione S-transferase omega-1
Glutathione S-transferase Mu 2
Ragulator complex protein LAMTOR2
Cytochrome b5 type B
Progressive ankylosis protein homolog
Septin-11
Rho-associated protein kinase 1
Ras-related C3 botulinum toxin substrate 3
Isocitrate dehydrogenase [NAD] subunit alpha, mitochondrial
Inactive tyrosine-protein kinase transmembrane receptor ROR1
Abl interactor 2
Cell division control protein 42 homolog
Transgelin-2
Ras-related protein Rab-2A
Charged multivesicular body protein 6
ADP-ribosylation factor 3
Glyoxylate reductase/hydroxypyruvate reductase
Profilin-1
Ras-related protein Rab-11 B
Rab GDP dissociation inhibitor alpha
CD97 antigen
Programmed cell death protein 10
Antithrombin-III
F-actin-capping protein subunit beta
Actin-related protein ⅔ complex subunit 2
Twinfilin-2
Ras-related protein Rab-21
Vacuolar-sorting protein SNF8
Caspase-6
Calcineurin subunit B type 1
Extended synaptotagmin-2
Leukocyte surface antigen CD47
COP9signalosomecomplex subunit 1
26S proteasome non-ATPase regulatory subunit 7
Eukaryotic translation initiation factor 3 subunit C-like protein
Deoxycytidylate deaminase
CD276 antigen
Caspase-3
Leucine-rich repeat-containing protein 57
Aldose reductase
Diphosphoinositol polyphosphate phosphohydrolase 1
Coagulation factor XIII A chain
Hsp70-binding protein 1
Sorting nexin-4
Protein phosphatase 1L
Pleckstrin homology domain-containing family N member 1
TABLE 1-4
AP-2 complex subunit alpha-2
Trophoblast glycoprotein
Ras-related protein Rab-23
14-3-3 protein eta
Peroxiredoxin-6
14-3-3 protein beta/alpha
Copine-2
14-3-3 protein gamma
Superoxide dismutase [Cu-Zn]
Microtubule-associated protein RP/EB family member 1
Vasodilator-stimulated phosphoprotein
Phosphoglucomutase-1
Adenylyl cyclase-associated protein 1
Calpain small subunit 1
Dual specificity protein phosphatase 23
Dihydropteridine reductase
Ephrin-B1
Golgin subfamily A member 7
Xaa-Pro aminopeptidase 1
Actin-related protein ⅔ complex subunit 3
Sorting nexin-9
Pleckstrin homology domain-containing family O member 2
WD repeat-containing protein 44
N(G),N(G)-dimethylarginine dimethylaminohydrolase 2
COP9 signalosome complex subunit 6
Guanine nucleotide-binding protein G(I)/G(S)/G(O) subunit gamma-10
Rab-like protein 3
Phosphoacetylglucosamine mutase
Sorting nexin-2
DDB1- and CUL4-associated factor 11
Ras GTPase-activating protein 1
Ribose-5-phosphate isomerase
SHC-transforming protein 1
Glutaminyl-peptide cyclotransferase-like protein
Ras-related protein Rab-5C
Purine nucleoside phosphorylase
Delta-aminolevulinic acid dehydratase
Ras-related protein Rab-5B
Abl interactor 1
Beta-2-glycoprotein 1
Serine/threonine-protein phosphatase 2B catalytic subunit alpha isoform
Actin-related protein 2
Ras-related protein Rap-2a
Sorcin
Type I inositol 1,4.5-trisphosphate 5-phosphatase
Ras-related protein Rab-35
TABLE 1-5
Rho GTPase-activating protein 18
Ras-related protein Rab-1A
Deoxyribonuclease-1-like 1
Peptidyl-prolyl cis-trans isomerase A
Adenylate kinase isoenzyme 1
Equilibrative nucleoside transporter 1
GTPase NRas
Rho-related GTP-binding protein RhoG
Glycogen phosphorylase, brain form
UV excision repair protein RAD23 homolog B
Omega-amidase NIT2
Serine hydroxymethyltransferase, cytosolic
Sodium/potassium-transporting ATPase subunit beta-1
Endothelial cell-selective adhesion molecule
Isocitrate dehydrogenase [NADP] cytoplasmic
Peptidyl-prolyl cis-trans isomerase FKBP1A
Dystroglycan
Tumor susceptibility gene 101 protein
Peroxiredoxin-5, mitochondrial
Protein XRP2
Actin-related protein ⅔ complex subunit 4
Stathmin
NSFL1 cofactor p47
Folate receptor alpha
Aldo-keto reductase family 1 member A1
Ran-specific GTPase-activating protein
Serine/threonine-protein phosphatase 2A catalytic subunit beta isoform
Vacuolar protein sorting-associated protein VTA1 homolog
Dual specificity protein phosphatase 3
Membrane cofactor protein
Calcium-binding protein 39
Argininosuccinate lyase
Mitochondrial import receptor subunit TOM34
Protein numb homolog
SUMO-conjugating enzyme UBC9
EH domain-containing protein 3
Ras-related protein Rab-1B
Adenine phosphoribosyltransferase
Coronin-1A
Annexin A4
F-actin-capping protein subunit alpha-1
Protein phosphatase 1 regulatory subunit 7
Peflin
Protein/nucleic acid deglycase DJ-1
Ras-related protein Rab-10 Ras-related protein Rap-2b
TABLE 1-6
RNA transcription, translation and transport factor protein
Crk-like protein
Serpin B6
6-phosphogluconolactonase
Guanine nucleotide-binding protein G(I)/G(S)/G(T) subunit beta-1
Lactadherin
Ras-related protein Ral-A
Protein transport protein Sec23A
Dihydropyrimidinase-related protein 2
NCK-interacting protein with SH3 domain
Vesicle-associated membrane protein 5
Protein phosphatase 1F
Nuclear receptor corepressor 2
Ubiquitin-conjugating enzyme E2 D3
WW domain-binding protein 2
POTE ankyrin domain family member I
Copine-1
Alpha-galactosidase A
Coronin-1B
ATP-dependent RNA helicase DDX1
Ubiquitin-conjugating enzyme E2 K
Syntaxin-binding protein 1
WD repeat-containing protein 37
MOB kinase activator 1B
Nodal modulator 3
Nucleoside diphosphate kinase B
Prostaglandin reductase 1
Phosphoglycerate mutase 2
TLD domain-containing protein 1
Calcium-binding protein 39-like
Glutathione S-transferase LANCL1
Prefoldin subunit 6
Cytosolic phospholipase A2
14-3-3 protein zeta/delta
.

2. The method according to claim 1,
wherein the protein is one or more membrane proteins selected from the group of proteins shown in Table 2:
TABLE 2
Protein Name
Band 4.1-like protein 2
Attractin
Rho GTPase-activating protein 1
Integrin alpha-V
Adhesion G-protein coupled receptor G1
Integrin beta-5
Transient receptor potential cation channel subfamily M member 4
Insulin receptor
Sodium/potassium-transporting ATPase subunit alpha-1
Major prion protein
Fructose-bisphosphate aldolase A
Neuroplastin
Sodium/potassium-transporting ATPase subunit beta-3
Tetraspanin-15
DnaJ homolog subfamily C member 5
Cytochrome b5 type B
Progressive ankylosis protein homolog
Inactive tyrosine-protein kinase transmembrane receptor ROR1
CD97 antigen
Extended synaptotagmin-2
Leukocyte surface antigen CD47
CD276 antigen
Protein phosphatase 1L
Trophoblast glycoprotein
Ephrin-B1
Equilibrative nucleoside transporter 1
Sodium/potassium-transporting ATPase subunit beta-1
Endothelial cell-selective adhesion molecule
Peptidyl-prolyl cis-trans isomerase FKBP1A
Dystroglycan
Folate receptor alpha
Membrane cofactor protein
NCK-interacting protein with SH3 domain
Vesicle-associated membrane protein 5
Syntaxin-binding protein 1
Nodal modulator 3
.

3. The method according to claim 1,
wherein the protein is one or more proteins selected from the group of proteins shown in Table 3:
TABLE 3
Protein Name
Integrin beta-5
Sorting nexin-9
Phosphoacetylglucosamine mutase
Stathm in
Ran-specific GTPase-activating protein
EH domain-containing protein 3
Adenine phosphoribosyltransferase
Protein phosphatase 1 regulatory subunit 7
Nuclear receptor corepressor 2
TLD domain-containing protein 1
Glutathione S-transferase LANCL 1
.

4. The method according to claim 1,
wherein the immune checkpoint inhibitor is an anti-PD-1 antibody or an anti-PD-L1 antibody.

5. The method according to claim 1,
wherein the immune checkpoint inhibitor is one or more selected from the group consisting of nivolumab, pembrolizumab, and atezolizumab.

6. The method according to claim 1, comprising:
isolating the exosomes from the biological sample derived from the lung cancer patient with a mixture of magnetic particles bound to an anti-CD9 antibody, an anti-CD81 antibody, or an anti-CD63 antibody is used.


SEQUENCE LISTING (1 sequence)

SEQ ID NO: 1 - protein, 137 aa, Homo sapiens
MQCGLVGKIIKRFEQKGFRLVAMKFLPASEEHLKQHYIDLKDRPFFPGLVKYMNSGPVVAMVWEGLNVVKTGRVMLGETNPADSKPGTIRGDFCIQVGRNIIHGSDSVKSAEKEISLRFKPEELVDYKSCAHDWVYE